(19) United States
(12) Patent Application Publication
(10) Pub. No.: US 2023/0121059 A1
(43) Pub. Date: Apr. 20, 2023

(54) HIGH DOSE FLU VACCINE IN PEDIATRIC SUBJECTS

(71) Applicant: Sanofi Pasteur Inc., Cambridge, MA (US)

(72) Inventors: Lee-Jah Chang, Easton, PA (US); Victoria Landolfi, Nazareth, PA (US); Therese Quinn, Hellertown, PA (US)

(73) Assignee: Sanofi Pasteur Inc., Cambridge, MA (US)

(21) Appl. No.: 17/802,758

(22) PCT Filed: Feb. 26, 2021
(86) PCT No.: PCT/US2021/019781

§ 371 Date: Aug. 26, 2022

Related U.S. Application Data
(60) Provisional application No. 62/982,993, filed on Feb. 28, 2020.
(60) Provisional application No. 63/080,931, filed on Sep. 21, 2020.

(51) Int. Cl. A61K 39/145; A61P 31/14

(52) U.S. Cl. CPC A61K 39/145; A61P 31/14; A61K 2039/70

(57) ABSTRACT

Disclosed herein are immunogenic compositions and vaccination regimes for immunizing humans against influenza disease.

This application claims the benefit of priority to U.S. Provisional Application No. 62/982,993, filed Feb. 28, 2020, and U.S. Provisional Application No. 63/080,931, filed Sep. 21, 2020, the entire contents of each of which are incorporated by reference herein for any purpose.

This disclosure relates to the field of vaccines and details a study to evaluate the safety and immunogenicity of different dosages of quadrivalent influenza vaccine (QIV) in subjects 6 months to less than 18 years of age.

Influenza is a contagious, acute viral respiratory disease caused by influenza type A and type B viruses. Influenza is typically characterized by the rapid onset of fever, myalgia, sore throat, and non-productive cough, and can also cause severe malaise lasting for several days. While influenza affects all age groups, infants and young children are at increased risks for influenza and its complications due to their maturing immune system and lack of prior exposure and thus lack of immunity. Influenza complications in the pediatric population include, for example, secondary bacterial pneumonia, acute otitis media, bronchitis, febrile seizures, Reye's syndrome, myositis, neurologic conditions, and exacerbations of underlying conditions (Munoz, F M (2002) Semin. Pediatr. Infect. Dis. 13(2): 72-8; Peltola V. et al. (2003) Clin. Infect. Dis. 36(3):299-305; Antonova E N et al. (2012) BMC Public Health 12:968).

Currently, vaccination represents the most effective medical intervention against influenza and its severe complications. However, the development of an effective influenza vaccine for members of high-risk groups such as the very young (neonates and pediatric individuals) and the very old (greater than 65 years) still faces challenges as these populations show reduced responsiveness to influenza vaccination when compared to adults. In the elderly, poor vaccine responsiveness is likely due to an age-related decline in immune functions (Weinberger et al. (2008) Clin. Infect. Dis. 46(7):1078-84). For example, in the elderly population, reduced B cell responses appear to be the result of both intrinsic and extrinsic changes such as far fewer naive B cells, fewer germinal center reactions, and diminished CD4 helper T cell function (Crooke S N et al. (2019) Immun Ageing 16, 25; Ciabattini A. et al. (2018) Semin Immunol. 40:83-94). In contrast, insufficient vaccine protection in infants and young children may be attributed to an immature immune system associated with lower BCR activation, different cytokine milieu during priming (i.e., less Th1-like, more anti-inflammatory), and reduced persistence of long-lived plasma cells in the bone marrow (Basha S. et al., (2014) Expert Rev Clin Immunol. 10(9):1171-1184; Saso A. et al. (2017) Semin Immunopathol. 39(6):627-642). A further complicating factor in the development of influenza vaccines for infants and young children is the concern over safety (e.g., increased reactogenicity, febrile seizure risk), especially when influenza vaccines are given concomitantly with standard of care, routine pediatric vaccines. Due to such safety concerns, for more than 30 years, influenza vaccines for children less than 3 years of age have contained a half dose of antigen (i.e., a 0.25 mL dose containing 7.5 μg of HA/strain/dose) instead of the standard dose (i.e., a 0.5 mL dose containing 15 μg of HA/strain/dose) recommended for older children and adults. It was until 2019 that the US FDA approved the use of Sanofi Pasteur's 0.5 mL Fluzone® Quadrivalent standard dose (QIV-SD) vaccine in children 6-35 months of age.

Accordingly, an influenza vaccine with increased safety and efficacy remains urgently needed in infants and young children. Moreover, children with immunocompromising conditions are at even higher risk of influenza and its complications and could also benefit from an improved influenza vaccine.

### SUMMARY

Provided herein, inter alia, are the following embodiments:
Embodiment 1. A method for immunizing a pediatric subject against influenza virus comprising administering to the pediatric subject a QIV-HD vaccine, wherein the pediatric subject is aged 6 months to less than 18 years.
Embodiment 2. A method for immunizing a pediatric subject against influenza virus comprising administering to the pediatric subject a QIV-HD vaccine comprising:
   a. about 30 μg, about 45 μg, or about 60 μg of hemagglutinin from an H1N1 influenza A virus strain per dose;
   b. about 30 μg, about 45 μg, or about 60 μg of hemagglutinin from an H3N2 influenza A virus strain per dose;
   c. about 30 μg, about 45 μg, or about 60 μg of hemagglutinin from a Yamagata lineage of influenza B virus strain per dose; and
   d. about 30 μg, about 45 μg, or about 60 μg of hemagglutinin from a Victoria lineage of influenza B virus strain per dose; and
   e. wherein the pediatric subject is aged 6 months to less than 18 years.
Embodiment 3. The method of embodiment 1 or 2, wherein the method prevents influenza virus infection in the pediatric subject.
Embodiment 4. The method of embodiment 1 or 2, wherein the method raises a protective immune response in the pediatric subject.
Embodiment 5. The method of embodiment 4, wherein the immune response is an antibody response.
Embodiment 6. The method of embodiment 1 or 2, wherein the vaccine comprises about 30 μg of hemagglutinin from an H1N1 influenza A virus strain per dose, about 30 μg of hemagglutinin from an H3N2 influenza A virus strain per dose; about 30 μg of hemagglutinin from a Yamagata lineage of influenza B virus strain per dose; and about 30 μg of hemagglutinin from a Victoria lineage of influenza B virus strain per dose.
Embodiment 7. The method of embodiment 1 or 2, wherein the vaccine comprises about 45 μg of hemagglutinin from an H1N1 influenza A virus strain per dose, about 45 μg of hemagglutinin from an H3N2 influenza A virus strain per dose; about 45 μg of hemagglutinin from a Yamagata lineage of influenza B virus strain per dose; and about 45 μg of hemagglutinin from a Victoria lineage of influenza B virus strain per dose.
Embodiment 8. The method of embodiment 1 or 2, wherein the vaccine comprises about 60 μg of hemagglutinin from an H1N1 influenza A virus strain per dose, about 60 μg of hemagglutinin from an H3N2 influenza A virus strain per dose; about 60 μg of hemagglutinin from a Yamagata lineage influenza B virus strain per dose; and about 60 μg of hemagglutinin from a Victoria lineage of influenza B virus strain per dose.
Embodiment 9. A method for immunizing a pediatric subject against influenza virus comprising administering to the pediatric subject aged 6 months to less than 18 years a QIV-HD vaccine comprising:
   a. about 60 μg of hemagglutinin from an H1N1 influenza A virus strain per dose;
   b. about 60 μg of hemagglutinin from an H3N2 influenza B virus strain per dose;
   c. about 60 μg of hemagglutinin from a Yamagata lineage of influenza B virus strain per dose; and
   d. about 60 μg of hemagglutinin from a Victoria lineage of influenza B virus strain per dose.
Embodiment 10. The method of any one of embodiments 1-9, wherein the vaccine is administered intramuscularly.
Embodiment 11. The method of any one of embodiments 1-10, wherein the pediatric subject is
   a. 6 months to less than 36 months of age;
   b. 3 years to less than 5 years of age;
   c. 5 years to less than 9 years of age; and/or
   d. 9 years to less than 18 years of age.
Embodiment 12. The method of any one of embodiments 1-11, wherein the pediatric subject is 6 months to less than 24 months of age.
Embodiment 13. The method of any one of embodiments 1-12, wherein the vaccine is administered once or twice to the same subject.
Embodiment 14. The method of embodiment 131 wherein the dose is administered in a volume of 0.70 mL.
Embodiment 15. The method of any one of embodiments 1-14, wherein the vaccine is administered in a prefilled syringe.
Embodiment 16. The method of any one of embodiments 1-15, wherein the pediatric subject has not been previously vaccinated against influenza.
Embodiment 17. The method of embodiment 16, wherein the previously unvaccinated subject is 6 months to less than 9 years and is provided two doses of vaccine.
Embodiment 18. The method of embodiment 17, wherein the two doses of vaccine are provided about 28 days apart.
Embodiment 19. The method of any one of embodiments 1-15, wherein the subject has been previously vaccinated against influenza.
Embodiment 20. The method of embodiment 19, wherein the subject is administered a single dose of vaccine.
Embodiment 21. The method of any one of embodiments 1-20, wherein administration reduces the incidence of laboratory confirmed influenza-like illness as compared to vaccination of a similar-aged subject with QIV-SD, wherein confirmed influenza-like illness is the occurrence of fever greater than or equal to 38° C. for at least 24 hours and at least one of cough, sputum production, wheezing, difficulty breathing, nasal congestion, rhinorrhea, pharyngitis, otitis, vomiting, diarrhea, sore throat, chills (shivering), tiredness (fatigue), headache, and myalgia (muscle aches).
Embodiment 22. The method of any one of embodiments 1-21, wherein administration reduces the occurrence of laboratory-confirmed influenza-like illness caused by viral types/subtypes antigenically similar to those contained in the vaccine composition.
Embodiment 23. The method of any one of embodiments 1-22, wherein administration reduces the occurrence of acute otitis media (AOM), acute lower respiratory tract infection (ALRI, e.g., pneumonia), hospitalization, and/or medication use.
Embodiment 24. The method of any one of embodiments 1-23, wherein two doses of the vaccine are administered to subjects who are unvaccinated for influenza, and wherein administration of the two doses of the vaccine results in higher geometric mean titers (GMTs) against each of the strains used to vaccinate as compared to vaccination with QIV-SD.
Embodiment 25. The method of any one of embodiments 1-24, wherein administration of the vaccine results in higher seroneutralization geometric mean titers (GMTs) against each of the strains used to vaccinate as compared to vaccination with QIV-SD.
Embodiment 26. The method of any one of embodiments 1-25, wherein the administration results in a higher geometric mean HI antibody titer (GMT) ratio (QIV-HD/QIV-SD) than the GMT ratios of TIV-HD/TIV-SD in adults aged 65 or older.
Embodiment 27. The method of any one of embodiments 24-26, wherein the subject is aged 6 months to less than 3 years.
Embodiment 28. The method of any one of embodiments 1-27, wherein the vaccine is produced in avian eggs.
Embodiment 29. The method of any one of embodiments 1-27, wherein the vaccine is not produced in avian eggs.
Embodiment 30. The method of any one of embodiments 1-27, wherein the vaccine is made by recombinant DNA techniques.
Embodiment 31. The method of any one of embodiments 1-27, wherein the vaccine is inactivated or live attenuated.
Embodiment 32. The method of embodiment 31, wherein the vaccine is inactivated.
Embodiment 33. The method of embodiment 31, wherein the vaccine is live attenuated.
Embodiment 34. The method of any one of embodiments 1-33, wherein the vaccine is a split virus vaccine.
Embodiment 35. The method of any one of embodiments 1-34, wherein the vaccine contains adjuvant.
Embodiment 36. The method of any one of embodiments 1-34, wherein the vaccine does not contain adjuvant.
Embodiment 37. The method of any one of embodiments 1-36, wherein the pediatric subject is immune compromised.
Embodiment 38. The method of any one of embodiments 1-37, wherein the pediatric subject is high risk.
Embodiment 39. The method of any one of embodiments 1-38, wherein the pediatric subject has or had asthma, diabetes, heart disease, HIV, AIDS, or cancer.
Embodiment 40. The method of any one of embodiments 1-39, wherein the vaccine is safe and well tolerated in the pediatric subject.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### FIGURE LEGENDS

FIGS. 1A-1D show a schematic of the first three stages of the clinical trial for subjects less than 5 years old.

FIGS. 2A-2C show a schematic of the first three stages of the clinical trial for subjects 5-8 years old.

FIG. 3 shows a schematic of stage 1 of the trial for subjects 9-17 years.

FIG. 4 shows the participant disposition for the US cohort ages 6 months through less than 18 years.

FIG. 5 shows two doses of QIV-HD (60 μg) administered one month apart generated higher GMTs than one dose of QIV-HD (60 μg) in subjects 6 to less than 36 months of age (previously unvaccinated). QIV-SD is Fluarix®. Y axis indicates the geometric mean titer (GMT).

FIGS. 6A-6D show solicited reactions for the US cohort ages 6 months through less than 3 years (FIG. 6A) and 3 years through less than 5 years (FIG. 6B); 5 years through less than 9 years (FIG. 6C); 9 years through less than 18 years (FIG. 6D). Solicited reactions were recorded for up to 7 days after vaccination. Proportions of participants with solicited reactions after any vaccine dose are presented. Results are for the safety analysis set. In this figure and throughout, “IIV4” and “QIV” are used interchangeably. QIV-SD was provided at 15 μg hemagglutinin/strain.

FIGS. 7A-7E show the geometric mean titer (GMT) at 28 days after the last vaccine dose for the US cohort. 6 months through less than 18 years (FIG. 7A); 6 months through less than 3 years (FIG. 7B); 3 years through less than 5 years (FIG. 7C); 5 years through less than 9 years (FIG. 7D); 9 years through less than 18 years (FIG. 7E). Results are for the immunogenicity analysis set. CI, confidence interval. QIV-SD was provided at 15 μg hemagglutinin/strain.

FIGS. 8A-8E show the ratio of geometric mean titers for QIV-HD versus QIV-SD at 28 days after the last vaccine dose for the US cohort. 6 months through less than 18 years (FIG. 8A); 6 months through less than 3 years (FIG. 8B); 3 years through less than 5 years (FIG. 8C); 5 years through less than 9 years (FIG. 8D); 9 years through less than 18 years (FIG. 8E). Results are for the immunogenicity analysis set. QIV-SD was provided at 15 μg hemagglutinin/strain.

FIGS. 9A-9E show the seroconversion rate at 28 days after the last vaccine dose for the US cohort. 6 months through less than 18 years (FIG. 9A); 6 months through less than 3 years (FIG. 9B); 3 years through less than 5 years (FIG. 9C); 5 years through less than 9 years (FIG. 9D); 9 years through less than 18 years (FIG. 9E). Results are for the immunogenicity analysis set. QIV-SD was provided at 15 μg hemagglutinin/strain.

FIGS. 10A-10E show the ratios of seroneutralization antibody geometric mean titers 28 days after the last vaccine dose for the US cohort. Ratios are for 28 days after the last vaccine dose versus day 0. Six months through less than 18 years (FIG. 10A); 6 months through less than 3 years (FIG. 10B); 3 years through less than 5 years (FIG. 10C); 5 years through less than 9 years (FIG. 10D); 9 years through less than 18 years (FIG. 10E). Results are for the immunogenicity analysis set. QIV-SD was provided at 15 μg hemagglutinin/strain.

FIGS. 11A-11B show solicited reactions by vaccine dose and grade in U S participants aged 6 months through <3 years at dose 1 (FIG. 11A) and dose 2 (FIG. 11B). Solicited reactions were recorded for up to 7 days after vaccination and graded 1 for mild, 2 for moderate, and 3 for severe (see Table 20A and Table 20D). Results are for the safety analysis set. QIV-SD was provided at 15 μg hemagglutinin/strain.

FIGS. 12A-12E show solicited reactions by vaccine dose and grade in U S participants aged 3 through <18 years. Solicited reactions were recorded for up to 7 days after vaccination and graded 1 for mild, 2 for moderate, and 3 for severe (see Table 20A and Table 20D). Results are for the safety analysis set. Three years through less than 5 years, dose 1 (FIG. 12A); Three years through less than 5 years, dose 2 (FIG. 12B); 5 years through less than 9 years, dose 1 (FIG. 12C); 5 years through less than 9 years, dose 2 (FIG. 12D); 9 years through less than 18 years (FIG. 12E).

FIGS. 13A-13B show geometric mean titer for participants aged 6 months through <3 years at 28 days after vaccination in the US cohort by previous influenza vaccination status—previously vaccinated (FIG. 13A) and unvaccinated (FIG. 13B). Results are for the immunogenicity analysis set. QIV-SD was provided at 15 μg hemagglutinin/strain. CI=confidence interval.

FIGS. 14A-14E shows ratios of geometric mean post-/pre-vaccination titers for the US cohort at 28 days after last vaccination. Results are for the immunogenicity analysis set. Six months through less than 18 years (FIG. 14A); 6 months through less than 3 years (FIG. 14B); 3 years through less than 5 years (FIG. 14C); 5 years through less than 9 years (FIG. 14D); 9 years through less than 18 years (FIG. 14E).

FIGS. 15A-15B show solicited reactions by vaccine dose and grade in the Canadian cohort. Solicited reactions were recorded on the day of each vaccine dose and for 7 days after vaccination and were graded. Results are for the safety analysis set. Six months through less than 2 years, dose 1 (FIG. 15A); six months through less than 2 years, dose 2 (FIG. 15B).

FIGS. 16A-16B show a safety overview for local reactions in the US cohort ages 6 months to less than 3 years after first vaccination (FIG. 16A) and after second vaccination (FIG. 16B). No increase in solicited injection site reactions or Grade 3 reactions was seen with increasing QIV-HD dose.

FIGS. 17A-17B show a safety overview for systemic reactions in the US cohort ages 6 months to less than 3 years after first vaccination (FIG. 17A) and after second vaccination (FIG. 17B). No increase in systemic reactions or Grade 3 reactions was seen with increasing QIV-HD dose.

FIG. 18 shows seroneutralization GMTs for the US cohort at QIV-HD 60 μg in ages 6 months through 3 years.

### DETAILED DESCRIPTION

### Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. In some embodiments, an influenza hemagglutinin (HA) polypeptide or immunogenic fragment thereof is an antigen.

As used herein, “hemagglutinin” or “HA” protein refers to an integral membrane glycoprotein on the surface of the influenza viral membrane. Specifically, the HA protein is usually expressed as a homotrimeric complex on the surface of influenza virions. Individual HA monomeric polypeptides can be further segregated into the membrane distal globular head region and the membrane proximal stem region. The HA protein is responsible for mediating virus attachment and subsequent membrane fusion with target cells. Currently, there are at least 18 known HA subtypes (i.e., H1, H2, H3, H4, H5, H6, H7, H8, H9, H10, H11, H12, H13, H14, H15, H16, H17, and H18) which are defined by their interaction with antibodies. Humans are generally infected by viruses of the H1, H2, and H3 subtypes. In some embodiments, the HA protein may be monomeric and comprises a single HA polypeptide. In other embodiments, the HA protein is trimeric and comprises three HA polypeptides. As used herein, “hemagglutinin polypeptide” or “HA polypeptide” refers to a polypeptide whose amino acid sequence includes at least one characteristic sequence of HA. HA polypeptides can include full length influenza HA polypeptide sequences and fragments thereof. Those of ordinary skill in the art can readily identify sequences that are characteristic of HA polypeptides generally, and/or of particular HA polypeptides (e.g., H1, H2, or H3 polypeptides), or of HAs that mediate infection of particular hosts (e.g., avian, camel, canine, cat, civet, equine, human, leopard, mink, mouse, seal, stone martin, swine, tiger, whale, etc.). The National Center for Biotechnology Information (NCBI) maintains a database of HA polypeptide sequences.

“Influenza virus,” as used herein, refers to a segmented negative-strand RNA virus that belongs to the Orthomyxoviridae family.

“Influenza vaccine,” as used herein, refers to an immunogenic composition capable of stimulating an immune response, administered for the prevention, amelioration, or treatment of influenza virus infection. An influenza vaccine may include, for example, attenuated or killed (e.g., split) influenza virus, virus-like particles (VLPs) and/or antigenic polypeptides or proteins (e.g., the HA polypeptides or trimeric HA proteins described herein) or DNA derived from them, or any recombinant versions of such immunogenic materials. Influenza vaccines also include DNA and viral-vector based vaccines. Vaccines contemplated herein may optionally include one or more adjuvants.

“Immune response,” as used herein, refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response. As used herein, a “protective immune response” refers to an immune response that protects a subject from infection (e.g., prevents infection or prevents the development of disease associated with infection). Methods of measuring immune responses are well known in the art and include, for example, by measuring proliferation and/or activity of lymphocytes (such as B or T cells), secretion of cytokines or chemokines, inflammation, antibody production and the like. An “antibody response” is an immune response in which antibodies are produced.

“Prevention,” as used herein, refers to prophylaxis, avoidance of disease manifestation, a delay of onset, and/or reduction in frequency and/or severity of one or more symptoms of a particular disease, disorder or condition (e.g., infection for example with influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

As used herein, the term “vaccination” or “vaccinate” refers to the administration of a composition intended to generate an immune response, for example to a disease-causing agent. Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccinating composition.

“Adjuvant,” as used herein refers to a substance or vehicle that non-specifically enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists and costimulatory molecules. Exemplary biological adjuvants include, but are not limited to, IL-2, RANTES, GM-CSF, TNF-a, IFN-γ, G-CSF, LFA-3, CD72, B7-1, B7-2, OX-40L, 4-1BBL, or combinations thereof.

The phrase “pharmaceutically-acceptable” or the like refers to molecular entities and compositions that do not produce an allergic or similar untoward reaction when administered to a mammal, and in particular, when administered to a human. Typically, a pharmaceutically acceptable composition retains the desired biological activity of the parent compound and does not impart any undesired toxicological effects.

The serum virus neutralization (SVN) assay (also referred to herein as seroneutralization assay/test) is a serological test to detect the presence and magnitude of functional systemic antibodies that prevent infectivity of a virus. The SVN assay is a highly sensitive and specific test to measure the titer of neutralizing antibodies post-infection or after vaccination. Conventional SVN methods are performed in vitro and are based on inhibition of virus infectivity in cell culture in the presence of neutralizing antibodies. Titer determination may be based on the presence or the absence of cytopathic effect or the evidence of viral infection using an immunoreactive technique.

A pediatric subject may be considered “high risk” if they have one or more of the following conditions: asthma, neurologic and/or neurodevelopment conditions (including disorders of the brain, spinal cord, peripheral nerve and muscle, such as cerebral palsy, epilepsy (seizure disorders), stroke, intellectual disability (mental retardation), moderate to severe developmental delay, muscular dystrophy, or spinal cord injury), chronic lung disease (such as cystic fibrosis), heart disease (such as congenital heart disease, congestive heart failure and coronary artery disease), blood disorders (such as sickle cell disease), endocrine disorders (such as diabetes mellitus), kidney disorders, liver disorders, metabolic disorders (such as inherited metabolic disorders and mitochondrial disorders), weakened immune system due to disease or medication (such as children or adolescents with HIV or AIDS, cancer, or those on chronic steroids), children who are taking aspirin or salicylate-containing medicines, extreme obesity, which has been associated with severe influenza illness in some studies of adults, may also be a risk factor for children, childhood obesity is defined as a body mass index (BMI) at or above the 95 percentile, for age and sex.

Standard-dose trivalent influenza vaccines (“TIV-SDs”) contain 15 micrograms (μg) hemagglutinin (HA) or less of each of three virus strains recommended by the WHO for use in that hemisphere's upcoming influenza season, for a total of 45 μg or less of HA antigen per dose. TIV-SDs comprise one HA from an influenza A/H1N1 strain, another HA from an influenza A/H3N2 strain, and one HA from an influenza B Victoria or B Yamagata lineage strain.

Fluad® is an adjuvanted (MF59 in oil-in-water emulsion of squalene oil) TIV-SD produced by Seqirus UK Limited. It is provided as a 0.25 mL liquid solution comprising 7.5 μg HA for each of the 3 virus strains recommended by the WHO for use in that hemisphere's upcoming influenza season, for a total of 22.5 μg of HA antigen per dose.

High-dose trivalent influenza vaccines (“TIV-HDs”) are similar to TIV-SDs in that they contain three HA's (one HA from an influenza A/H1N1 strain, another HA from an influenza A/H3N2 strain, and one HA from an influenza B Victoria or Yamagata lineage strain), but different in that they contain greater than 15 μg HA per strain. An exemplary TIV-HD may comprise, e.g., 60 μg HA of each of the three virus strains (4 times more antigen than TIV-SD, for a total of 180 μg of HA antigen per dose).

An exemplary TIV-HD, Fluzone® High-Dose, was developed by Sanofi Pasteur and subsequently licensed in the US, Canada, Australia, Brazil, and the United Kingdom to improve vaccine efficacy in adults 65 years of age and older (Centers for Disease Control and Prevention. Prevention and control of seasonal influenza with vaccines: recommendations of the Advisory Committee on Immunization Practices (ACIP), 2009. MMWR. 2009; 58(RR-8):1-52). Fluzone® High-Dose is the only influenza vaccine licensed in adults 65 years of age and older to have demonstrated superior efficacy against laboratory-confirmed influenza illness versus a TIV-SD influenza vaccine (DiazGranados et al. (2014) NEJM 371:635-45).

TIV-SD and TIV-HD (i.e., trivalent influenza vaccines) contain a single influenza B strain. However, two distinct genetic lineages of influenza B virus (the Victoria and the Yamagata lineages) have been co-circulating worldwide; both are responsible for influenza illnesses. However, the B strain included in seasonal influenza vaccines has not been the dominant circulating B lineage (mismatched strains) in approximately 25% of the seasons between 2000 and 2013 (Caini S. et al. (2015) Influenza Other Respir. Viruses. 9(Suppl. 1):3-12. To overcome the problem of B-strain selection and improve protection of the population against seasonal influenza virus strains, marketed influenza vaccines have been transitioning to quadrivalent formulations comprising an additional B-strain HA. Thus, the issue of having to choose a strain from only one B lineage for the seasonal influenza vaccine and the resulting risk posed by the potential widespread circulation of a strain from the alternate B lineage is eliminated with quadrivalent formulations (Gorse G J et al. (2015) Vaccine 33(9):1151-9).

As used herein, “QIV-SD vaccine” refers to a standard dose quadrivalent influenza vaccine comprising 15 μg HA or less of each of the four virus strains recommended by the WHO for use in that hemisphere's upcoming influenza season, wherein the four strains are: one Victoria lineage of influenza B strain, one Yamagata lineage of influenza B strain, one HIN1 influenza A strain, and one H3N2 influenza A strain. In some embodiments, a QIV-SD comprises 15 μg HA per strain for a total of 60 μg of HA antigen per dose.

As described herein, Fluarix® Quadrivalent is a unadjuvanted QIV-SD that is manufactured by GlaxoSmithKline (GSK), and is referred to herein as “Unadjuvanted QIV-SD.”

As used herein, “QIV-HD vaccine” refers to a high dose quadrivalent influenza vaccine comprising more than 15 μg HA of each of the four virus strains recommended by the WHO for use in that hemisphere's upcoming influenza season, wherein the four strains are: one Victoria lineage of influenza B strain, one Yamagata lineage of influenza B strain, one HIN1 influenza A strain, and one H3N2 influenza A strain. In some embodiments, a QIV-HD comprises 30 μg HA, 45 μg HA, or 60 μg HA per strain for a total of 120 μg, 180 μg, or 240 μg HA antigen, respectively per dose.

In some embodiments, a QIV-HD vaccine may be formulated in a sterile aqueous suspension of inactivated influenza virus for intramuscular (IM) injection prepared from influenza viruses propagated in embryonated chicken eggs. In such an embodiment, the virus-containing fluid may be harvested and inactivated with formaldehyde. Influenza virus may be concentrated and purified in a linear sucrose density gradient solution using a continuous flow centrifuge. The virus may then be chemically disrupted using a non-ionic surfactant, octylphenol ethoxylate (Triton® X-100 also named octoxinol-9) producing a “split-virus.” Formaldehyde may then be added for the second time to complete the inactivation of the influenza virus and ensure Avian Leucosis Virus clearance. Lastly, the split virus may then be further purified by diafiltration against phosphate buffered saline (PBS) solution and sterile filtered. Antibiotics and preservatives may or may not be used in the manufacturing process of QIV-HD.

Below is a Table showing the qualitative and quantitative composition of one exemplary formulation of a QIV-HD vaccine in a 0.7 mL dose:

TABLE 1
Qualitative and quantitative composition
of an exemplary QIV-HD per 0.7 mL dose

| | Quantity | | |
|---|---|---|---|
| Component | (per 0.7 mL dose) | | Function |
| Split influenza virus, | 240 μg HA total | | Active substance |
| inactivated strains: | | | |
| A (H1N1) | 60 | μg HA | Active substance |
| A (H3N2) | 60 | μg HA | Active substance |
| B (Victoria lineage) | 60 | μg HA | Active substance |
| B (Yamagata lineage) | 60 | μg HA | Active substance |

Other:

| Sodium phosphate-buffered | QS to | | Diluent |
|---|---|---|---|
| isotonic sodium chloride | appropriate volume | | |
| solution: | | | |
| Sodium chloride | 6.6 | g/L | |
| Monobasic sodium phosphate | 0.410 | g/L | |
| Dibasic sodium phosphate | 3.83 | g/L | |
| Water for injections | QS to 1 L | | |
| Formaldehyde | ≤140 | μg | Residual |
| Ovalbumin | ≤1 | μg | Residual |
| Triton ® X-100 (octylphenol | ≤350 | μg | Excipient |
| ethoxylate) | | | |

### Exemplary Vaccine Compositions

Disclosed herein are methods for immunizing a pediatric subject against influenza virus comprising administering a QIV-HD vaccine. In some embodiments, the pediatric subject is aged 6 months to less than 18 years. In some embodiments, the method prevents influenza, and in some embodiments, the method raises a protective immune response in the pediatric subject. In some embodiments, a vaccine composition comprising QIV-HD is provided for use in any one of the methods described herein, e.g., for immunizing a pediatric subject against influenza virus. Exemplary QIV-HD vaccine compositions are provided herein for use in any one of the methods disclosed herein.

Many influenza vaccines are currently available and are generally based either on live virus or on inactivated (killed) virus. In some embodiments, the QIV-HD vaccine is live attenuated. In some embodiments, the QIV-HD vaccine comprises an inactivated virus. Inactivated vaccines may be based on whole virions, split virions, or on purified surface antigens. Influenza antigens can also be presented in the form of virosomes. In some embodiments, the QIV-HD vaccine comprises a live virus. In some embodiments, the QIV-HD vaccine comprises an inactivated virus. In some embodiments, the QIV-HD vaccine comprises a whole virion. In some embodiments, the QIV-HD vaccine comprises a split virion. In some embodiments, the QIV-HD vaccine comprises purified surface antigens. In some embodiments, the QIV-HD vaccine comprises a virosome. In some embodiments, the QIV-HD vaccine comprises a split virion and is inactivated.

Chemical means for inactivating a virus include treatment with an effective amount of one or more of the following agents: detergents, formaldehyde, β-propiolactone, methylene blue, psoralen, carboxyfullerene (C60), binary ethylamine, acetyl ethyleneimine, or combinations thereof. Non-chemical methods of viral inactivation are known in the art, such as, e.g., UV light or gamma irradiation.

Virions may be harvested from virus-containing fluids by various methods. For example, a purification process may involve zonal centrifugation using a linear sucrose gradient solution that includes detergent to disrupt the virions. Antigens may then be purified, after optional dilution, by diafiltration, for example.

Split virions may be obtained by treating purified virions with detergents (e.g., ethyl ether, polysorbate 80, deoxycholate, tri-N-butyl phosphate, Triton X-100, Triton N101, cetyltrimethylammonium bromide, Tergitol NP9, etc.) to produce subvirion preparations, and are well known in the art. In some embodiments, the QIV-HD vaccine comprises a split virion.

In some embodiments, a QIV-HD vaccine comprises purified surface antigen. In some embodiments, QIV-HD vaccines comprise the influenza surface antigens haemagglutinin and optionally neuraminidase. Processes for preparing these proteins in purified form are well known in the art. In some embodiments, the QIV-HD vaccine comprises purified surface antigens.

Another form of inactivated influenza antigen is the virosome (nucleic acid free viral-like liposomal particles). Virosomes may be prepared by solubilization of influenza virus with a detergent followed by removal of the nucleocapsid and reconstitution of the membrane containing the viral glycoproteins. An alternative method for preparing virosomes involves adding viral membrane glycoproteins to excess amounts of phospholipids, to give liposomes with viral proteins in their membrane. In some embodiments, the QIV-HD vaccine is a virosome.

### Strain Selection

Exemplary vaccines of the disclosure include hemagglutinin from at least two different influenza A virus strains and at least two influenza B virus strains. Strains are based on the WHO or the Vaccines and Related Biological Products Advisory Committee (VRBPAC) (in the US) recommendations for the particular influenza season in question. Strains will change based on WHO/VRBPAC recommendations per season. The different strains will typically be grown separately and then mixed after the viruses have been harvested and antigens have been prepared. Strains used in the vaccines of the disclosure may have a natural HA as found in a wild-type virus, or a modified HA.

In some embodiments, a vaccine composition of the disclosure comprises an influenza virus of a reassortant strain. In some embodiments, the reassortant strain is obtained by reverse genetics techniques. In some embodiments, the reassortant strain is not obtained by reverse genetics techniques. Typically, the vaccines disclosed herein comprises influenza strains that are capable of human-to-human transmission. In some embodiments, the strain's genome comprises at least one RNA segment that originated in a mammalian (e.g., in a human) influenza virus. In some embodiments, the strain's genome comprises at least one NS segment that originated in an avian influenza virus.

In some embodiments, the vaccine compositions disclosed herein comprise strains that may be resistant to antiviral therapy (e.g., resistant to oseltamivir and/or zanamivir), including resistant pandemic strains.

In some embodiments, the vaccine compositions disclosed herein comprise strains that have not been passaged through eggs at any stage between isolation from a patient and replication in a cell culture system, inclusive. In some embodiments, the vaccine compositions disclosed herein comprise strains that have been passaged through eggs. In some embodiments, the eggs are avian. In some embodiments, the avian eggs are from chicken (e.g., hen).

In some embodiments, the vaccine compositions disclosed herein comprise hemagglutinin with a binding preference for oligosaccharides with a Sia(α2,6)Gal terminal disaccharide. In some embodiments, the vaccine compositions disclosed herein comprise hemagglutinin with a binding preference for oligosaccharides with a Sia(α2,3)Gal terminal disaccharide. Human influenza viruses bind to receptor oligosaccharides having a Sia(a 2,6)Gal terminal disaccharide (sialic acid linked α-2,6 to galactose), but eggs and Vero cells have receptor oligosaccharides with a Sia(α 2,3)Gal terminal disaccharide. Growth of human influenza viruses in cells such as MDCK provides selection pressure on hemagglutinin to maintain the native Sia(α 2,6)Gal binding, unlike egg passaging.

In some embodiments, the vaccine compositions disclosed herein comprise glycoproteins (including hemagglutinin) with a different glycosylation pattern from egg-derived viruses, and in such instances, will comprises glycoproteins that include glycoforms that are not seen in chicken eggs.

Influenza A virus currently displays sixteen HA subtypes: H1, H2, H3, H4, H5, H6, H7, H8, H9, H10, H11, H12, H13, H14, H15 and H16. In some embodiments, the vaccine compositions disclosed herein protect against one or more of these subtypes. In some embodiments, the vaccine compositions disclosed herein protect against one or more of influenza A virus NA subtypes N1, N2, N3, N4, N5, N6, N7, N8 or N9. In some embodiments, the vaccine compositions disclosed herein comprise an H1 strain and a H3 strain. In some embodiments, the vaccine compositions disclosed herein comprise three influenza A virus strains, e.g., a H1 strain, a H3 strain and a pandemic-associated strain.

Characteristics of a pandemic-associated influenza strain include: (a) it contains a new hemagglutinin compared to the hemagglutinins in currently-circulating human strains, i.e. one that has not been evident in the human population for over a decade (e.g., H2), or has not previously been seen at all in the human population (e.g., H5, H6 or H9, that have generally been found only in bird populations), such that the vaccine recipient and the general human population are immunologically naive to the strain's hemagglutinin; (b) it is capable of being transmitted horizontally in the human population; and (c) it is pathogenic to humans. In some embodiments, the vaccine compositions disclosed herein comprise a pandemic-associated influenza virus strain comprising a H2, H5, H7 or H9 subtype (e.g., H5N1, H5N3, H9N2, H2N2, H7N1 or H7N7). Pandemic strains can have a H1 subtype (e.g., H1N1). In some embodiments, the vaccine compositions disclosed herein comprise an HA from an H1N1 influenza A virus strain, and an HA from an H3N2 influenza A virus strain.

Influenza B virus strains fall into two distinct lineages, which emerged in the late 1980s and have HAs which can be antigenically and/or genetically distinguished from each other. Current influenza B virus strains are either B/Victoria/2/87-like or B/Yamagata/16/88-like (referred to herein as “Victoria lineage” or “Yamagata lineage”, respectively). These strains are usually distinguished antigenically, but differences in amino acid sequences have also been described for distinguishing the two lineages.

In some embodiments, the vaccine compositions disclosed herein comprise an antigen from at least two influenza B virus strains. In some embodiments, the vaccine compositions disclosed herein comprise an HA from a Yamagata lineage of influenza B virus strain and/or an HA from a Victoria lineage of influenza B virus strain.

In some embodiments, the vaccine compositions disclosed herein comprise an HA from a Yamagata lineage of influenza B virus strain, an HA from a Victoria lineage of influenza B virus strain, an HA from an H1N1 influenza A virus strain, and an HA from an H3N2 influenza A virus strain. In some embodiments, at least two of the influenza B virus strains may have distinct hemagglutinins but related neuraminidases. For instance, they may both have a B/Victoria/2/87-like neuraminidase or may both have a B/Yamagata/16/88-like neuraminidase.

In some embodiments, the vaccine compositions disclosed herein comprise 2, 3, 4, 5, 6, 7, 8, 9, 10, or more HAs. In some embodiments, the vaccine compositions disclosed herein comprise 2, 3, 4, 5, 6, 7, 8, 9, 10, or more HAs and one or more neuraminidase (NA).

### Dosing

Hemagglutinin (HA) is the main immunogen in current inactivated influenza vaccines, and vaccine doses are typically standardized by reference to HA levels. The QIV-HD vaccine compositions for use in the methods disclosed herein comprise a “high dose” of HA per strain (as compared to a “standard dose”).

In some embodiments, the QIV-HD vaccine compositions comprise about 30 μg to about 60 μg HA per strain. In some embodiments, the QIV-HD vaccine compositions comprise about 30 μg, about 35 μg, about 40 μg, about 45 μg, about 50 μg, about 55 μg, or about 60 μg HA per strain. In some embodiments, the QIV-HD vaccine compositions comprise about 30 μg HA per strain. In some embodiments, the QIV-HD vaccine compositions comprise about 45 μg HA per strain. In some embodiments, the QIV-HD vaccine compositions comprise about 60 μg HA per strain. In some embodiments, the QIV-HD vaccine compositions comprise more than 15 μg HA per strain. In some embodiments, the QIV-HD vaccine compositions comprise more than 15 μg HA per strain, wherein the μg HA per strain differs among the four HA's (e.g., 30 μg HA for one strain and 45 μg HA for another strain, etc.).

In some embodiments, the QIV-HD vaccine compositions comprise an amount of HA per dose of about 30 μg HA per strain in about a 0.1, 0.15, 0.2, 0.25, 0.3, 0.35, 0.4, 0.45, 0.5 mL volume. In some embodiments, the QIV-HD vaccine compositions comprise an amount of HA per dose of about 30 μg HA per strain in about a 0.35 mL volume. In some embodiments, the QIV-HD vaccine compositions comprise an amount of HA per dose of about 30 μg HA per strain in about a 0.35 mL volume, wherein the vaccine composition comprises an HA from a Yamagata lineage of influenza B virus strain, an HA from a Victoria lineage of influenza B virus strain, an HA from an HIN1 influenza A virus strain, and an HA from an H3N2 influenza A virus strain. In some embodiments, the QIV-HD vaccine compositions comprise an amount of HA per dose of about 30 μg HA per strain in about a 0.35 mL volume in a pre-filled syringe. In some embodiments, the QIV-HD vaccine compositions comprise an amount of HA per dose of about 30 μg per strain in about a 0.35 mL volume in a pre-filled syringe, wherein the vaccine comprises an HA from a Yamagata lineage of influenza B virus strain, an HA from a Victoria lineage of influenza B virus strain, an HA from an HIN1 influenza A virus strain, and an HA from an H3N2 influenza A virus strain.

In some embodiments, the QIV-HD vaccine compositions comprise an amount of HA per dose of about 45 μg per strain in about a 0.2, 0.25, 0.3, 0.35, 0.4, 0.45, 0.5, 0.52, 0.55, 0.6, 0.65, or 0.7 mL volume. In some embodiments, the QIV-HD vaccine compositions comprise an amount of HA per dose of about 45 μg per strain in about a 0.5 mL or 0.52 mL volume. In some embodiments, the QIV-HD vaccine compositions comprise an amount of HA per dose of about 45 μg per strain in about a 0.5 mL or 0.52 mL volume, wherein the vaccine composition comprises an HA from a Yamagata lineage of influenza B virus strain, an HA from a Victoria lineage of influenza B virus strain, an HA from an HIN1 influenza A virus strain, and an HA from an H3N2 influenza A virus strain. In some embodiments, the QIV-HD vaccine compositions comprise an amount of HA per dose of about 45 μg per strain in about a 0.5 mL or 0.52 mL volume in a pre-filled syringe. In some embodiments, the QIV-HD vaccine compositions comprise an amount of HA per dose of about 45 μg per strain in about a 0.5 mL or 0.52 mL volume in a pre-filled syringe, wherein the vaccine composition comprises an HA from a Yamagata lineage of influenza B virus strain, an HA from a Victoria lineage of influenza B virus strain, an HA from an HIN1 influenza A virus strain, and an HA from an H3N2 influenza A virus strain.

In some embodiments, the QIV-HD vaccine compositions comprise an amount of HA per dose of about 60 μg per strain in about a 0.4, 0.45, 0.5, 0.55, 0.65, 0.7, 0.75, 0.8, 0.85, 0.9 mL volume. In some embodiments, the QIV-HD vaccine compositions comprise an amount of HA per dose of about 60 μg per strain in about a 0.7 mL volume. In some embodiments, the QIV-HD vaccine compositions comprise an amount of HA per dose of about 60 μg per strain in about a 0.7 mL volume, wherein the vaccine composition comprises an HA from a Yamagata lineage of influenza B virus strain, an HA from a Victoria lineage of influenza B virus strain, an HA from an HIN1 influenza A virus strain, and an HA from an H3N2 influenza A virus strain. In some embodiments, the QIV-HD vaccine compositions comprise an amount of HA per dose of about 60 μg per strain in about a 0.7 mL volume in a pre-filled syringe. In some embodiments, the QIV-HD vaccine compositions comprise an amount of HA per dose of about 60 μg per strain in about a 0.7 mL volume in a pre-filled syringe, wherein the vaccine composition comprises an HA from a Yamagata lineage of influenza B virus strain, an HA from a Victoria lineage of influenza B virus strain, an HA from an HIN1 influenza A virus strain, and an HA from an H3N2 influenza A virus strain.

Typically, the concentration of HA is the same for each strain in a composition. In some embodiments, however, the concentrations for each strain the composition differ so long as the composition remains “high dose” (e.g., greater than 15 μg HA per dose).

### Pharmaceutical Compositions

In some embodiments, the QIV-HD vaccine comprises components in addition to the influenza antigens, such as, e.g., one or more pharmaceutically acceptable carrier(s), diluent(s), and/or excipient(s). In some embodiments, the vaccine composition comprises a pharmaceutically acceptable carrier, diluent, and/or excipient. In some embodiments, the vaccine composition comprises a pharmaceutically acceptable carrier, diluent, and/or excipient and an adjuvant. In some embodiments, the carrier, diluent, and/or excipient comprises buffered saline. In some embodiments, the carrier, diluent, and/or excipient comprises octylphenol ethoxylate (Triton X-100). In some embodiments, the carrier, diluent, and/or excipient comprises buffered saline and octylphenol ethoxylate (Triton X-100). In some embodiments, the carrier, diluent, and/or excipient comprises sodium chloride. In some embodiments, the carrier, diluent, and/or excipient comprises sodium phosphate. In some embodiments, the carrier, diluent, and/or excipient comprises dibasic sodium phosphate. In some embodiments, the carrier, diluent, and/or excipient comprises water. In some embodiments, the carrier, diluent, and/or excipient comprises formaldehyde. In some embodiments, the carrier, diluent, and/or excipient comprises ovalbumin. In some embodiments, the carrier, diluent, and/or excipient comprises sodium chloride, sodium phosphate (monobasic, dibasic, or both), and water. In some embodiments, the carrier, diluent, and/or excipient comprises sodium chloride, sodium phosphate (monobasic, dibasic, or both), water, formaldehyde, ovalbumin, and Triton X-100.

Compositions will be in aqueous form when administered but may be stored in a solid form and resuspended prior to administration.

In some embodiments, the QIV-HD vaccine composition comprises a preservative (e.g., thiomersal or 2-phenoxyethanol). In some embodiments, however, the vaccine composition is substantially free from mercurial material e.g., is thiomersal-free. In some embodiments, the vaccine composition is preservative-free.

In some embodiments, the vaccine composition comprises a physiological salt, such as a sodium salt. In some embodiments, the vaccine composition comprises sodium chloride (NaCl). In some embodiments, the vaccine composition comprises NaCl between about 1 and 20 mg/ml. In some embodiments, the vaccine composition comprises NaCl of about 1, 2, 3, 4, 5, 6, 7, 8, 9, or 10 g/L. Other salts that may be present include sodium phosphate, potassium chloride, potassium dihydrogen phosphate, disodium phosphate, disodium phosphate dehydrate, magnesium chloride, magnesium chloride hexahydrade, calcium chloride dihydrate, or others known to those of skill in the art. In some embodiments, the vaccine composition comprises monobasic sodium phosphate of about 0.1, 0.2, 0.2, 0.4, or 0.5 g/L. In some embodiments, the vaccine composition comprises dibasic sodium phosphate of about 1, 2, 3, 4, or 5 g/L. In some embodiments, the vaccine composition comprises monobasic sodium phosphate of about 0.1, 0.2, 0.2, 0.4, or 0.5 g/L, and dibasic sodium phosphate of about 1, 2, 3, 4, or 5 g/L. Where adjuvant is in a separate container from antigens, a salt, e.g., sodium chloride may be present in both containers. In some embodiments, the vaccine composition comprises NaCl of about 1, 2, 3, 4, 5, 6, 7, 8, 9, or 10 g/L, monobasic sodium phosphate of about 0.1, 0.2, 0.2, 0.4, or 0.5 g/L, and dibasic sodium phosphate of about 1, 2, 3, 4, or 5 g/L. Where adjuvant is in a separate container from antigens, a salt, e.g., sodium chloride may be present in both containers.

In some embodiments, the QIV-HD vaccine composition comprises one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range. The pH of a composition will generally be between 5.0 and 8.1, and more typically between 6.0 and 8.0, e.g., 6.5 and 7.5, or between 7.0 and 7.8.

In some embodiments, the vaccine composition is sterile. The composition is preferably non-pyrogenic e.g., containing less than 1 EU (endotoxin unit, a standard measure) per dose, and preferably <0.1 EU per dose. The composition is preferably gluten free.

Compositions of the invention may include detergent e.g., a polyoxyethylene sorbitan ester surfactant (known as “Tweens”), an octoxynol (such as octoxynol-9 (Triton X-100) or t-octylphenoxypolyethoxyethanol), a cetyl trimethyl ammonium bromide (“CTAB”), or sodium deoxycholate, particularly for a split or surface antigen vaccine. The detergent may be present only at trace amounts. In some embodiments, the vaccine composition for use in the methods disclosed herein comprise other residual components in trace amounts such as antibiotics (e.g., neomycin, kanamycin, or polymyxin B). Where adjuvant is in a separate container from antigens, this detergent will usually be present in the antigen-containing container.

In some embodiments, the vaccine composition comprises a unit dosage volume of about 0.3, 0.35, 0.4, 0.45, 0.5, 0.55, 0.6, 0.65, or 0.7 mL and a pharmaceutically acceptable carrier(s), diluent(s), and/or excipient(s).

### Adjuvants

In some embodiments, the QIV-HD vaccine composition comprises one or more adjuvants, which can function to enhance the immune responses (humoral and/or cellular) elicited in a patient who receives the composition. In some embodiments, the vaccine composition comprises an oil-in-water emulsion adjuvant. In some embodiments, the vaccine composition comprises squalene.

In some embodiments, the vaccine composition comprises oil-in-water emulsions and at least one surfactant.

In some embodiments, the vaccine composition comprises tocopherols.

In some embodiments, the vaccine composition comprises tocopherols and squalene. In some embodiment, the oil content is in the range of 2-20% (by volume).

In some embodiments, the vaccine composition comprises an adjuvant comprising a mineral-containing composition, including calcium salts and aluminum salts (or mixtures thereof). Calcium salts include calcium phosphate. Aluminum salts include hydroxides, phosphates, sulfates, etc., with the salts taking any suitable form (e.g. gel, crystalline, amorphous, etc.). The mineral containing compositions may also be formulated as a particle of metal salt.

In some embodiments, the vaccine composition comprises an adjuvant comprising one or more saponins, which are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculate (brides veil), and Saponaria officianalis.

Saponin adjuvant formulations include purified formulations, such as QS21, as well as lipid formulations, such as ISCOMs. QS21 is marketed as Stimulon®. Combinations of saponins and cholesterols can be used to form unique particles called immunostimulating complexes (ISCOMs). In some embodiments, an ISCOM includes a phospholipid such as phosphatidylethanolamine or phosphatidylcholine. Any known saponin can be used in ISCOMs. Preferably, the ISCOM includes one or more of: QuilA, QHA & QHC.

In some embodiments, the vaccine composition comprises an adjuvant comprising fatty adjuvants.

In some embodiments, the vaccine composition comprises an adjuvant comprising bacterial ADP-ribosylating toxins (e.g. the E. coli heat labile enterotoxin “LT”, cholera toxin “CT”, or pertussis toxin “PT”) and detoxified derivatives thereof, such as the mutant toxins known as LT-K63 and LT-R72.

In some embodiments, the vaccine composition comprises an adjuvant comprising bioadhesives and mucoadhesives, such as esterified hyaluronic acid microspheres or chitosan and its derivatives.

In some embodiments, the vaccine composition comprises an adjuvant comprising cytokine-inducing agents.

In some embodiments, the vaccine composition comprises an adjuvant comprising liposomes.

In some embodiments, the vaccine composition comprises an adjuvant comprising polyoxyethylene ethers and/or polyoxyethylene esters. Such formulations further include polyoxyethylene sorbitan ester surfactants in combination with an octoxynol as well as polyoxyethylene alkyl ethers or ester surfactants in combination with at least one additional non-ionic surfactant such as an octoxynol. Exemplary polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

In some embodiments, the vaccine composition comprises an adjuvant comprising muramyl peptides, such as N-acetylmuramyl-L-threonyl-D-isoglutamine (“thr-MDP”), N-acetyl-normuramyl-L-alanyl-D-isoglutamine (nor-MDP), N-acetylglucsaminyl-N-acetylmuramyl-L-Al-D-isoglu-L-Ala-dipalmitoxy propylamide (“DTP-DPP”, or Theramide™), N-acetylrnuramyl-L-alanyl-D-isoglutaminyl-L-alanine-2-(1′-2′dipalmitoyl-sn-glycero-3-hydroxyphosphoryloxy)-ethylamine (“MTP-PE”).

Compositions may include one or more adjuvant, e.g., 2, 3, 4, or more adjuvants. For example, they may advantageously include both an oil-in-water emulsion and a cytokine-inducing agent.

Antigens and adjuvants in a composition will typically be in admixture.

### Manufacture of Vaccines

In some embodiments, the QIV-HD vaccine compositions are prepared in eggs. In some embodiments, the eggs are chicken, e.g., hen eggs. In some embodiments, the vaccine compositions are prepared in cell lines which support influenza virus replication. The cell line will typically be of mammalian origin. Suitable mammalian cells include hamster, cattle, primate (including humans and monkeys) and dog cells. Various cell types may be used, such as kidney cells, fibroblasts, retinal cells, lung cells, etc. Examples of suitable hamster cells are the cell lines having the names BHK21 or HKCC. Suitable monkey cells are e.g., African green monkey cells, such as kidney cells as in the Vero cell line. Suitable dog cells are e.g., kidney cells, as in the CLDK and MDCK cell lines.

In some embodiments, the vaccine compositions are prepared in cell lines with mammalian-type glycosylation. In some embodiments, the vaccine compositions are prepared in avian cell lines, including cell lines derived from ducks or hens.

In some embodiments, the vaccine compositions are prepared in MDCK cell lines, derived from Madin-Darby canine kidney. The original MDCK cell line is available from the ATCC as CCL-34, but derivatives of this cell line and other MDCK cell lines may also be used.

In some embodiments, the vaccine compositions are grown on cells in adherent culture or in suspension. Microcarrier cultures can also be used. In some embodiments, the cells may thus be adapted for growth in suspension.

In some embodiments, the vaccine compositions are prepared in cell lines, optionally wherein the cells are grown in serum-free culture media and/or protein free media. A medium is referred to as a serum-free medium in the context of this disclosure if there are no additives from serum of human or animal origin. The cells growing in such cultures naturally contain proteins themselves, but a protein-free medium is understood to mean one in which multiplication of the cells (e.g., prior to infection) occurs with exclusion of proteins, growth factors, other protein additives and non-serum proteins, but can optionally include proteins such as trypsin or other proteases that may be necessary for viral growth.

In some embodiments, the vaccine compositions are prepared in cell lines, wherein the cell line supports influenza virus replication, and wherein they are grown below 37° C. (e.g., 30-36° C., or at about 30° C., 31° C., 32° C., 33° C., 34° C., 35° C., 36° C.) during viral replication.

Methods for propagating influenza virus in cultured cells generally includes the steps of inoculating a culture of cells with an inoculum of the strain to be grown, cultivating the infected cells for a desired time period for virus propagation, such as for example as determined by virus titer or antigen expression (e.g., between 24 and 168 hours after inoculation) and collecting the propagated virus.

The viral inoculum and the viral culture are preferably free from (i.e., will have been tested for and given a negative result for contamination by) herpes simplex virus, respiratory syncytial virus, parainfluenza virus 3, SARS coronavirus, adenovirus, rhinovirus, reoviruses, polyomaviruses, bimaviruses, circoviruses, and/or parvoviruses.

### Exemplary Methods and Uses

A method for immunizing a pediatric subject against influenza virus comprising administering to the pediatric subject a QIV-HD vaccine is provided. In some embodiments, a QIV-HD vaccine is provided for use in immunizing a pediatric subject. A QIV-HD vaccine composition is provided for use in the manufacture of a medicament for immunizing a pediatric subject against influenza. In some embodiments, the pediatric subject is aged 6 months to less than 18 years.

Methods and uses for immunizing a pediatric subject against influenza virus comprising administering to the pediatric subject a QIV-HD vaccine comprising: about 30 μg, about 45 μg, or about 60 μg of hemagglutinin from an H1N1 influenza A virus strain per dose; about 30 μg, about 45 μg, or about 60 μg of hemagglutinin from an H3N2 influenza A virus strain per dose; about 30 μg, about 45 μg, or about 60 μg of hemagglutinin from a Yamagata lineage of influenza B virus strain per dose; and about 30 μg, about 45 μg, or about 60 μg of hemagglutinin from a Victoria lineage of influenza B virus strain per dose; and wherein the pediatric subject is aged 6 months to less than 18 years are provided. The subject may be human.

Disclosed methods and uses will generally be used to generate an antibody response including, but not limited to, a protective antibody response. Methods for assessing antibody responses, neutralizing capability and protection after influenza virus vaccination are well known in the art. Human studies have shown that antibody titers against hemagglutinin of human influenza virus are correlated with protection (α serum sample hemagglutination-inhibition titer of about 30-40 gives around 50% protection from infection by a homologous virus). Antibody responses are typically measured by hemagglutination inhibition (HAI), by microneutralization, by single radial immunodiffusion (SRID), and/or by single radial hemolysis (SRH). These assay techniques are well known in the art.

The hemagglutination inhibition (HAI) assay is a common method for determining quantitative antibody titers for influenza virus and is widely used both for licensure of vaccines and for seroepidemiologic studies examining protection in populations. The assay relies on the ability of the hemagglutinin protein on the surface of influenza virus to bind to sialic acids on the surface of red blood cells (RBCs). If the patient's serum contains antibodies that block viral attachment, this interaction is inhibited.

In some embodiments, a method for immunizing a pediatric subject against influenza virus is provided comprising administering to the pediatric subject a QIV-HD vaccine composition. In some embodiments, the QIV-HD vaccine composition comprises: i) about 30 μg, about 45 μg, or about 60 μg of hemagglutinin from an HIN1 influenza A virus strain per dose; ii) about 30 μg, about 45 μg, or about 60 μg of hemagglutinin from an H3N2 influenza A virus strain per dose; iii) about 30 μg, about 45 μg, or about 60 μg of hemagglutinin from a Yamagata lineage of influenza B virus strain per dose; and iv) about 30 μg, about 45 μg, or about 60 μg of hemagglutinin from a Victoria lineage of influenza B virus strain per dose. In some embodiments, the pediatric subject is aged 6 months to less than 18 years.

In some embodiments, a method for eliciting a protective immune response against influenza virus in a pediatric subject is provided comprising administering to the pediatric subject a QIV-HD vaccine composition comprising about i) 30 μg, about 45 μg, or about 60 μg of hemagglutinin from an HIN1 influenza A virus strain per dose; ii) about 30 μg, about 45 μg, or about 60 μg of hemagglutinin from an H3N2 influenza A virus strain per dose; iii) about 30 μg, about 45 μg, or about 60 μg of hemagglutinin from a Yamagata lineage of influenza B virus strain per dose; and iv) about 30 μg, about 45 μg, or about 60 μg of hemagglutinin from a Victoria lineage of influenza B virus strain per dose, wherein the pediatric subject is aged 6 months to less than 18 years.

In some embodiments, a method for preventing influenza in a pediatric subject is provided comprising administering to the pediatric subject a QIV-HD vaccine composition comprising about i) 30 μg, about 45 μg, or about 60 μg of hemagglutinin from an HIN1 influenza A virus strain per dose; ii) about 30 μg, about 45 μg, or about 60 μg of hemagglutinin from an H3N2 influenza A virus strain per dose; iii) about 30 μg, about 45 μg, or about 60 μg of hemagglutinin from a Yamagata lineage of influenza B virus strain per dose; and iv) about 30 μg, about 45 μg, or about 60 μg of hemagglutinin from a Victoria lineage of influenza B virus strain per dose, wherein the pediatric subject is aged 6 months to less than 18 years.

In some embodiments, a method for generating antibodies against influenza virus in a pediatric subject is provided comprising administering to the pediatric subject a QIV-HD vaccine composition comprising about i) 30 μg, about 45 μg, or about 60 μg of hemagglutinin from an HIN1 influenza A virus strain per dose; ii) about 30 μg, about 45 μg, or about 60 μg of hemagglutinin from an H3N2 influenza A virus strain per dose; iii) about 30 μg, about 45 μg, or about 60 μg of hemagglutinin from a Yamagata lineage of influenza B virus strain per dose; and iv) about 30 μg, about 45 μg, or about 60 μg of hemagglutinin from a Victoria lineage of influenza B virus strain per dose, wherein the pediatric subject is aged 6 months to less than 18 years.

In some embodiments, a method for immunizing a pediatric subject against influenza virus is provided comprising administering to the pediatric subject a high dose influenza vaccine composition comprising about i) 30 μg of hemagglutinin from an HIN1 influenza A virus strain per dose; ii) about 30 μg of hemagglutinin from an H3N2 influenza A virus strain per dose; iii) about 30 μg of hemagglutinin from a Yamagata lineage of influenza B virus strain per dose; and iv) about 30 μg of hemagglutinin from a Victoria lineage of influenza B virus strain per dose, wherein the pediatric subject is aged 6 months to less than 18 years.

In some embodiments, a method for immunizing a pediatric subject against influenza virus is provided comprising administering to the pediatric subject a high dose influenza vaccine composition comprising about i) 45 μg of hemagglutinin from an HIN1 influenza A virus strain per dose; ii) about 45 μg of hemagglutinin from an H3N2 influenza A virus strain per dose; iii) about 45 μg of hemagglutinin from a Yamagata lineage of influenza B virus strain per dose; and iv) about 45 μg of hemagglutinin from a Victoria lineage of influenza B virus strain per dose, wherein the pediatric subject is aged 6 months to less than 18 years.

In some embodiments, a method for immunizing a pediatric subject against influenza virus is provided comprising administering to the pediatric subject a high dose influenza vaccine composition comprising about i) 60 μg of hemagglutinin from an HIN1 influenza A virus strain per dose; ii) about 60 μg of hemagglutinin from an H3N2 influenza A virus strain per dose; iii) about 60 μg of hemagglutinin from a Yamagata lineage of influenza B virus strain per dose; and iv) about 60 μg of hemagglutinin from a Victoria lineage of influenza B virus strain per dose, wherein the pediatric subject is aged 6 months to less than 18 years.

In some embodiments, the pediatric subject is aged 6 months to less than 18 years. In some embodiments, the pediatric subject is aged 9 to 17 years. In some embodiments, the pediatric subject is aged 5 to 8 years. In some embodiments, the pediatric subject is aged 36 months to less than 5 years. In some embodiments, the pediatric subject is aged 6 months to less than 36 months. In some embodiments, the pediatric subject is aged 6 months to less than 24 months. In some embodiments, the pediatric subject is aged 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In some embodiments, the pediatric subject is 1, 2, 3, 4, 5, 6, 7, 8, 9, 10, 11, 12, 13, 14, 15, 16, or 17 years old. In some embodiments, the pediatric subject is previously unvaccinated. In some embodiments, the pediatric subject is previously vaccinated. In some embodiments, the pediatric subject previously had influenza infection. In some embodiments, the pediatric subject previously did not have influenza infection.

In some embodiments, the pediatric subject is aged 6 months to less than 18 years and previously vaccinated or previously unvaccinated. In some embodiments, the pediatric subject is aged 9 to 17 years previously vaccinated or previously unvaccinated. In some embodiments, the pediatric subject is aged 5 to 8 years and previously vaccinated or previously unvaccinated. In some embodiments, the pediatric subject is aged 36 months to less than 5 years and previously vaccinated or previously unvaccinated. In some embodiments, the pediatric subject is aged 6 months to less than 36 months and previously vaccinated or previously unvaccinated. In some embodiments, the pediatric subject is aged 6 months to less than 24 months and previously vaccinated or previously unvaccinated.

In some embodiments, the pediatric subject is about 1, 2, 3, 4, 5, 6, 7, 8, 9, 10, 11, or 12 months old. In some embodiments, the pediatric subject is about 6, 7, 8, 9, 10, 11, or 12 months old. In some embodiments, the pediatric subject is about 13, 14, 15, 16, 17, 18, 19, 20, 21, 22, 23 or 24 months old. In some embodiments, the pediatric subject is about 1, 2, 3, 4, 5, 6, 7, 8, 9, 10, 11, 12, 13, 14, 15, 16, or 17 years old. In some embodiments, the pediatric subject is between 6 months and 9 years old. In some embodiments, the pediatric subject is between 6 months and 9 years old and is provided 2 doses of the QIV-HD vaccine. In some embodiments, the pediatric subject is between 6 months and 9 years old and is provided 2 doses of the QIV-HD vaccine at least 4 weeks apart. In some embodiments, the pediatric subject is between 6 months and 9 years old, is previously unvaccinated for influenza virus, and is provided 2 doses of the QIV-HD vaccine. In some embodiments, the pediatric subject is between 6 months and 9 years old, is previously vaccinated for influenza virus, and is provided 1 dose of the QIV-HD vaccine.

In an exemplary embodiment, previously influenza unvaccinated children 6 months to less than 9 years of age are administered 2 doses of the QIV-HD vaccine at least 28 days apart during an influenza season.

In an exemplary embodiment, previously influenza vaccinated children 6 months to less than 9 years of age are administered with 1 dose of the QIV-HD vaccine.

In an exemplary embodiment, children 9 to less than 18 years of age are administered with 1 dose of the QIV-HD vaccine regardless of their prior influenza vaccination history.

In some embodiments, previously unvaccinated pediatric subjects are subjects who have not received at least 2 doses of seasonal influenza vaccine in a prior influenza season, who have received only one dose of any influenza vaccine in the past, or whose vaccination history is unknown. In some embodiments, previously influenza vaccinated pediatric subjects are subjects who have received at least 2 doses of seasonal influenza vaccine in prior influenza seasons.

In some embodiments, a QIV-HD vaccine is provided for use in a method for immunizing, raising a protective immune response in, or generating antibodies against influenza in a pediatric subject, wherein the method prevents influenza virus infection in the pediatric subject. In some embodiments, the QIV-HD vaccine comprises any one of the compositions described herein at any one of the doses described herein. In some embodiments, the QIV-HD vaccine is administered at a dose of about 30 μg, about 45 μg, or about 60 μg of hemagglutinin per strain and has any one or more of the functional effects of preventing influenza, raising a protective immune response against influenza, or generating an antibody response against influenza.

In some embodiments, the method or use raises a protective immune response in the pediatric subject. In some embodiments, the immune response is an antibody response.

The QIV-HD vaccines may be administered to a selected pediatric subject using any of a number of conventional methodologies, including for example, parenteral, intravenous, intraperitoneal, subcutaneous, transcutaneous, intradermal, subdermal, transdermal, intramuscular, topical, intranasal, or other suitable route, including, administration, by injection, inhalation, insufflation, or ingestion. In some embodiments, the vaccine is administered intramuscularly.

Administration can be by a single dose schedule or a multiple dose schedule. Multiple doses may be used in a primary immunization schedule and/or in a booster immunization schedule. In a multiple dose schedule the various doses may be given by the same or different routes e.g., a parenteral prime and mucosal boost, a mucosal prime and parenteral boost, etc. Administration of more than one dose (typically two doses) is particularly useful in previously unvaccinated subjects, e.g., immunologically naive patients e.g., for people who have never received an influenza vaccine before, or for those who were never infected with influenza vaccine, or for vaccines including a new HA subtype. Multiple doses will typically be administered at least 1 week apart (e.g., about 2 weeks, about 3 weeks, about 4 weeks, about 6 weeks, about 8 weeks, about 12 weeks, about 16 weeks, etc. In some embodiments, the vaccine is administered once or twice to a single subject. In some embodiments, the vaccine is administered in a single dose. In some embodiments, two doses are provided, optionally about 15, 16, 17, 18, 19, 20, 21, 22, 23, 24, 25, 26, 27, 28, 29, or 30 days apart. In some embodiments, two doses are provided, optionally about 28 days apart.

In some embodiments, two doses of the vaccine are administered to subjects who are unvaccinated for influenza, and wherein administration of the two doses of the vaccine results in higher geometric mean titers (GMTs) against each of the strains used to vaccinate as compared to vaccination with QIV-SD. In some embodiments, two doses of the vaccine are administered to subjects 6 months to less than 3 years who are unvaccinated for influenza, and wherein administration of the two doses of the vaccine results in higher geometric mean titers (GMTs) against each of the strains used to vaccinate as compared to vaccination with QIV-SD. In some embodiments, the two doses of the vaccine are administered to a pediatric subject who is about 1, 2, 3, 4, 5, 6, 7, 8, 9, 10, 11, or 12 months old. In some embodiments, the pediatric subject is about 6, 7, 8, 9, 10, 11, or 12 months old. In some embodiments, the pediatric subject is about 1, 2, 3, 4, 5, 6, 7, 8, 9, 10, 11, 12, 13, 14, 15, 16, or 17 years old. In some embodiments, the vaccine is administered in a prefilled syringe. In some embodiments, the prefilled syringe comprises only the volume necessary to comprise the QIV-HD vaccine, the volume generally increasing as dose increases.

In some embodiments, administration reduces the incidence of laboratory confirmed influenza-like illness. In some embodiments, administration reduces the incidence of laboratory confirmed influenza-like illness as compared to vaccination of a similar-aged subject with a standard dose vaccine (e.g., TIV-SD or QIV-SD). In some embodiments, the laboratory confirmed influenza-like illness is the occurrence of fever greater than or equal to 38° C. for at least 24 hours. In some embodiments, the laboratory confirmed influenza-like illness is at least one of cough, sputum production, wheezing, difficulty breathing, nasal congestion, rhinorrhea, pharyngitis, otitis, vomiting, diarrhea, sore throat, chills (shivering), tiredness (fatigue), headache, and myalgia (muscle aches). In some embodiments, the laboratory confirmed influenza-like illness is the occurrence of fever greater than or equal to 38° C. for at least 24 hours and at least one of cough, sputum production, wheezing, difficulty breathing, nasal congestion, rhinorrhea, pharyngitis, otitis, vomiting, diarrhea, sore throat, chills (shivering), tiredness (fatigue), headache, and myalgia (muscle aches). In some embodiments, administration of the QIV-HD reduces the occurrence of laboratory-confirmed influenza-like illness caused by viral types/subtypes antigenically similar to those contained in the vaccine composition.

In some embodiments, administration reduces the occurrence of at least one of acute otitis media (AOM), acute lower respiratory tract infection (ALRI, e.g., pneumonia), hospitalization, and/or medication use.

In some embodiments, administration of the QIV-HD vaccine results in higher geometric mean titers (GMTs) against each of the strains used to vaccinate as compared to vaccination with QIV-SD, TIV-SD, and/or TIV-HD.

In some embodiments, administration of the QIV-HD vaccine results in higher seroneutralization against each of the strains used to vaccinate as compared to vaccination with QIV-SD, TIV-SD, and/or TIV-HD. In some embodiments, administration of the QIV-HD vaccine results in higher seroneutralization against each of the strains used to vaccinate as compared to vaccination with QIV-SD, TIV-SD, and/or TIV-HD, wherein the subject is 6 months to less than 3 years. In some embodiments, the pediatric subject is about 1, 2, 3, 4, 5, 6, 7, 8, 9, 10, 11, or 12 months old. In some embodiments, the pediatric subject is about 6, 7, 8, 9, 10, 11, or 12 months old. In some embodiments, the pediatric subject is about 1, 2, 3, 4, 5, 6, 7, 8, 9, 10, 11, 12, 13, 14, 15, 16, or 17 years old.

In some embodiments, administration of the QIV-HD vaccine to a pediatric subject results in a higher geometric mean HI antibody titer (GMT) ratio (QIV-HD/QIV-SD) than the GMT ratios of TIV-HD/TIV-SD in adults aged 65 or older who received QIV-SD, TIV-SD, or TIV-HD. In some embodiments, administration of the QIV-HD vaccine to a subject that is 6 months to less than 3 years results in a higher geometric mean HI antibody titer (GMT) ratio (QIV-HD/QIV-SD) than the GMT ratios of TIV-HD/TIV-SD in adults aged 65 or older who received QIV-SD, TIV-SD, or TIV-HD.

In some embodiments, the pediatric subject is immune compromised. In some embodiments, the pediatric subject is high risk. In some embodiments, a subject is deemed high risk if they have one or more of the following conditions: asthma, neurologic and/or neurodevelopment conditions (including disorders of the brain, spinal cord, peripheral nerve and muscle, such as cerebral palsy, epilepsy (seizure disorders), stroke, intellectual disability (mental retardation), moderate to severe developmental delay, muscular dystrophy, or spinal cord injury), chronic lung disease (such as cystic fibrosis), heart disease (such as congenital heart disease, congestive heart failure and coronary artery disease), blood disorders (such as sickle cell disease), endocrine disorders (such as diabetes mellitus), kidney disorders, liver disorders, metabolic disorders (such as inherited metabolic disorders and mitochondrial disorders), weakened immune system due to disease or medication (such as children or adolescents with HIV or AIDS, cancer, or those on chronic steroids), children who are taking aspirin or salicylate-containing medicines, extreme obesity, which has been associated with severe influenza illness in some studies of adults, may also be a risk factor for children, childhood obesity is defined as a body mass index (BMI) at or above the 95th percentile, for age and sex.

In some embodiments, the high-risk subject is immunocompromised, or has one or more of diabetes (type I or II), heart disease, asthma, lung condition, liver disease, renal/kidney disease, HIV, AIDS, or cancer.

In some embodiments, the vaccine compositions described herein are administered at the same time as other routine vaccines. In some embodiments, the routine vaccines include, for example, Pentacel® (DTaP5-IPV/Hib), Prevnar® (PCV7), Prevnar 13® (PCV13), RotaTeq® (RV5), ROTARIX® (RV1), ENGERIX-B® (HepB), RECOMBIVAX HB® (HepB), M-M-R® (MMR), M-M-R®_{II} (MMR), and VARIVAX® (V) vaccines. In some embodiments, the routine vaccines include, for example, Adacel® (Tdap5) and Gardasil® (HPV4). In some embodiments, the routine vaccines include DTaP5-IPV/HibHepB, Other routine vaccines are known in the art and may be provided to the subject at the same time, before, or after, the vaccine compositions described herein.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include,” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### Example 1—Safety and Immunogenicity of Different Dosages of High-Dose Quadrivalent Influenza Vaccine (QIV-HD) in Children 6 Months to Less Than 18 Years of Age

A randomized, dose-escalating, modified double-blind, active-controlled, multicenter Phase II trial in children aged 6 months through <18 years was conducted. The primary objectives were to assess the safety of QIV-HD in the studied ages, administered as one or two doses, at three dose levels (30, 45, and 60 μg HA/strain); to compare the antibody response induced by each test dosage of QIV-HD versus QIV-SD; and to compare the antibody response induced by the highest tolerable dosage of QIV-HD versus adjuvanted TIV.

Participants were enrolled in four stages using a stepwise age de-escalation and dose escalation design. In the US cohort, participants were randomized to receive one of the dose levels of QIV-HD or QIV-SD. In the Canadian cohort, the highest tolerated QIV-HD dosage was randomized with the adjuvanted TIV. For participants 8 years of age or younger, a blinded review of the 7-day safety data for each tested QIV-HD dose was performed to determine whether to proceed with the next planned dose.

All subjects enrolled in the QHD04 study (also referred to as QHD4) study received an influenza vaccine which was either 1 of the 3 investigational QIV-HD, which differ by the amount of HA per strain, or one of the 2 licensed comparator vaccines, QIV-SD or adjuvanted TIV (Fluad®). Subjects were vaccinated against the influenza viruses recommended by the WHO (Vaccines and Related Biological Products Advisory Committee [VRBPAC] in the US) for the 2018-2019 Northern Hemisphere (NH) influenza season.

The study design, vaccine content, and baseline characteristics of US and CA cohorts is shown in Tables 1A, 1B, 1C, and 1D, respectively.

Executive Summary of the Study:

Participants:

Participants had to be 6 months through <18 years of age. Participants <24 months of age had to have been born at full term (≥37 weeks) or have a birth weight of ≥2.5 kg. Participants who had received any vaccine in the 30 days before study vaccination were excluded. Participants previously vaccinated against influenza could not have been vaccinated against influenza in the previous 6 months. Unvaccinated participants could not have been vaccinated against influenza or have had a laboratory-confirmed influenza infection. Participants were excluded if they had a condition that, in the opinion of the Investigator, might compromise the participant's health or evaluation of the study vaccine. Other exclusion criteria are listed in Table 5A.

Vaccines and Administration:

QIV-HD was a split-virion inactivated influenza vaccine containing 30, 45, or 60 μg HA/strain of the A/H1N1, A/H3N2, B/Victoria, and B/Yamagata virus strains recommended by the World Health Organization (WHO)/Vaccines and Related Biological Products Advisory Committee of the US for the Northern Hemisphere 2018 2019 influenza season. QIV-SD (Fluarix Quadrivalent; GlaxoSmithKline Biologicals, Dresden, Germany) was a split-virion inactivated influenza vaccine containing 15 μg HA/strain of the A/H1N1, A/H3N2, B/Victoria, and B/Yamagata virus strains recommended by the WHO/Vaccines and Related Biological Products Advisory Committee of the US for the Northern Hemisphere 2018 2019 influenza season. MF59-adjuvanted TIV (FLUAD Pediatric; Seqirus UK Limited, Maidenhead, UK) contained 7.5 μg per strain of the A/H1N1, A/H3N2, and B/Victoria virus strains recommended by the WHO/National Advisory Committee on Immunization of Canada for the Northern Hemisphere 2018 2019 influenza season.

Participants 9 through <18 years of age and participants previously vaccinated against influenza 6 months through <9 years of age received one dose of QIV-HD or comparator vaccine. Participants aged 6 months through <9 years who had not previously been vaccinated against influenza or who had received only one previous dose in a prior influenza season received two doses of QIV-HD or comparator vaccine 28 days apart. All vaccines were administered by intramuscular injection. Vaccine received by study group and vaccinated group are shown in Table 20A. Table 1B summarizes the antigen content and injection volume of each tested QIV-HD dose.

Randomization:

The study was divided into 13 study groups. For each group, randomization was stratified by previous influenza vaccination status (previously vaccinated or unvaccinated). Interactive response technology was used to randomize eligible participants according to the randomization schedule shown in Table 16A.

Blinding:

The study was modified double-blind: an unblinded staff member at each site administered vaccine, the identity of which was not disclosed to the investigators in charge of safety assessments, the trial staff who collected the safety data, the laboratory personnel who analyzed blood samples, the participants, or the participants' patients or guardians. The person who administered the vaccine was not involved in any of the blinded study assessments.

Measurement of Hemagglutination Inhibition (HAI) Antibody Titers:

Participants provided blood samples on the day of the first vaccine dose (baseline) and at 28-35 days after each vaccine dose. HAI titers were measured as described previously (Greenberg D P et al. (2013) Vaccine 31:770-6). Briefly, serum samples isolated from collected blood and control sera were incubated with Type III neuraminidase from Vibrio cholerae to eliminate non-specific inhibitors. They were next incubated with an erythrocyte suspension to adsorb spontaneous anti-species agglutinins. The mixtures were then centrifuged and the resulting supernatants were used to prepare 10 two-fold dilutions (range 1:10 to 1:10,240), which were incubated with previously titrated influenza antigen (4 hemagglutination units/25 mL). An erythrocyte suspension was added to the mixtures, which were incubated. Titers were then recorded as the highest serum dilution in which complete HAI occurred. If the 1:10 dilution did not result in complete inhibition of hemagglutination, the HAI titer was reported as <10. If the 1:10,240 dilution gave complete inhibition of hemagglutination, the serum HAI titer was reported as ≥10,240. Endpoints based on HAI antibody titers included the titer 28 days after each vaccine dose, the ratio of titers post-versus pre-vaccination, and the seroconversion rate (defined as the percentage of participants with (i) a titer <10 before vaccination and a titer ≥40 after vaccination or (ii) a titer ≥10 before vaccination and a ≥4-fold increase in titer after vaccination).

Measurement of Seroneutralization Antibody Titers:

Neutralizing activity was analyzed using a microneutralization assay based on the methods of the influenza reference laboratories of the Centers for Disease Control and Prevention and the Health Protection Agency (Stephenson I et al. (2004) Virsus Res 103:91-5; Rowe T et al. (1999) J Clin Microbiol 37:937-43). Serially diluted, heat-inactivated human serum samples were pre-incubated with a fixed amount of challenge virus. They were next incubated overnight with Madin-Darby canine kidney cells, after which viral nucleoprotein production in infected cells was measured by enzyme linked immunosorbent assay using a monoclonal antibody specific to influenza A or influenza B nucleoprotein. The neutralizing antibody titer was defined as the reciprocal of the highest dilution that gives an optical density equal to the 50% reduction in the detection of influenza virus NP. The lower limit of quantitation was the reciprocal of the lowest dilution used (1:10). Titers less than this were reported as <10. Titers >10240 were pre-diluted, retested, and end point titers were reported.

Solicited Reactions:

On the day of each vaccine dose and for 7 days after vaccination, each participant (or their parent or guardian) used a diary card to record, on a daily basis, body temperature (and the route by which it was measured) and other solicited injection site and systemic reactions (including the intensity grade). For each event, any action taken by the participant or their parent or guardian (e.g. discontinuation of study vaccination) was to be recorded. Injection-site reactions comprised pain (children aged 3 through <18 years) or tenderness (children aged 6 months through <3 years), erythema, swelling, induration, and bruising. Systemic reactions for children aged 3 through <18 years were fever, headache, malaise, myalgia, and shivering. For children aged 6 months through <3 years, systemic reactions comprised fever, vomiting, abnormal crying, drowsiness, appetite loss, and irritability. Solicited reactions were graded as 3 for severe, 2 for moderate, and 1 for mild (see Table 16B for grading of solicited injection-site reactions and Table 16C for grading of solicited systemic reaction)

Unsolicited Adverse Events:

Unsolicited AEs and SAEs were defined as described in the International Council for Harmonisation E2A Guideline for Clinical Safety Data Management: Definitions and Standards for Expedited Reporting (ICH. E2A.: (1995) Clinical Safety Data Management: Definitions and Standards for Expedited Reporting. International Council for Harmonisation of Technical Requirements for Pharmaceuticals for Human Use) and were collected during the 28 days following each vaccine dose. Unsolicited systemic AEs occurring during the first 30 min after vaccination were recorded as immediate unsolicited systemic AEs.

SAEs (including AEs of special interest) were collected until 6 months after the last dose of vaccine. AEs of special interest included new onset of Guillain-Barré syndrome, encephalitis/myelitis (including transverse myelitis), Bell's palsy, convulsions (including febrile seizure), optic neuritis, and brachial neuritis. All AEs were assessed for intensity, seriousness, relationship to study vaccine, and action taken.

Statistical Analysis:

The immunogenicity endpoints for HAI antibody titer (titer, ratio of titers post-versus pre-vaccination, and seroconversion rate) and seroneutralization antibody titer were analyzed in the immunogenicity analysis set, defined as the subset of randomized subjects who received one dose of study vaccine (participants aged 9 through <18 years and previously vaccinated participants aged 6 months through <9 years) or two doses of the same study vaccine (previously unvaccinated participants aged 6 months through <9 years) and who provided a post vaccination blood sample.

The geometric mean titer (GMT), geometric mean of individual titer ratios (post-/pre-vaccination), and ratios of GMTs between vaccine groups were calculated, and their 95% confidence intervals (CIs) were calculated using the normal approximation of log-transformed titers. Seroconversion rate was calculated and the 95% CIs were calculated based on the Clopper-Pearson method (Newcombe RG (1998) Stat Med 17:857-72).

Solicited reactions and AEs were analyzed according to the vaccine received in the safety analysis set, defined as all participants who received at least one dose of study vaccine. Descriptive statistics were calculated.

Missing data were not imputed and there was no search for outliers. No statistical adjustments based on the outcomes of the blinded safety reviews were made, because no hypotheses were tested. Statistical analyses were performed using SAS version 9.4 or later (SAS Institute, Cary, N.C., USA).

Sample Size Estimation:

The study aimed to enroll approximately 700 participants divided into 13 groups (see Table 16A) with the purpose of having enough participants to observe trends in safety and immunogenicity; however, because no hypothesis was tested, no study size calculation was made.

TABLE 1A
Description of study design

| Stage | Age Group | Study | | | Vaccine Dosage |
|---|---|---|---|---|---|
| (Country) | (Vaccination Status*) | Group | N | Vaccine | (μg HA/strain/dose)† |
| 1 | 9 to 17 years | 12 | 30 | QIV-HD | 30 |
| (US) | (previously vaccinated | | 30 | QIV-HD | 45 |
| | and unvaccinated) | | 10 | unadjuvanted QIV-SD | 15 |
| | 9 to 17 years | 13 | 30 | QIV-HD | 60 |
| | (previously vaccinated | | 30 | unadjuvanted QIV-SD | 15 |
| | and unvaccinated) | | | | |
| | 5 to 8 years | 9 | 30 | QIV-HD | 30 |
| | (previously vaccinated | | 10 | unadjuvanted QIV-SD | 15 |
| | and unvaccinated) | | | | |
| | 36 months to <5 years | 1 | 30 | QIV-HD | 30 |
| | (previously vaccinated | | 10 | unadjuvanted QIV-SD | 15 |
| | and unvaccinated) | | | | |
| 2 | 5 to 8 years | 10 | 30 | QIV-HD | 45 |
| (US) | (previously vaccinated | | 10 | unadjuvanted QIV-SD | 15 |
| | and unvaccinated) | | | | |
| | 36 months to <5 years | 2 | 30 | QIV-HD | 45 |
| | (previously vaccinated | | 10 | unadjuvanted QIV-SD | 15 |
| | and unvaccinated) | | | | |
| | 6 to <36 months | 3 | 30 | QIV-HD | 30 |
| | (previously vaccinated | | 10 | unadjuvanted QIV-SD | 15 |
| | and unvaccinated) | | | | |
| 3 | 5 to 8 years | 11 | 30 | QIV-HD | 60 |
| (US) | (previously vaccinated | | 30 | unadjuvanted QIV-SD | 15 |
| | and unvaccinated) | | | | |
| | 36 months to <5 years | 4 | 45 | QIV-HD | 60 |
| | (previously vaccinated | | 45 | unadjuvanted QIV-SD | 15 |
| | and unvaccinated) | | | | |
| | 6 to <36 months | 5 | 30 | QIV-HD | 45 |
| | (previously vaccinated | | 10 | unadjuvanted QIV-SD | 15 |
| | and unvaccinated) | | | | |
| 4 | 6 to <36 months | 6 | 30 | QIV-HD | 30, 45, or 60† |
| (US) | (previously unvaccinated) | | 30 | unadjuvanted QIV-SD | 15 |
| | 6 to <36 months | 7 | 30 | QIV-HD | 30, 45, or 60† |
| | (previously vaccinated) | | 30 | unadjuvanted QIV-SD | 15 |
| 4 | 6 to <24 months | 8 | 30 | QIV-HD | 30, 45, or 60† |
| (Canada) | (previously unvaccinated) | | 30 | adjuvanted TIV | 7.5 |

Abbreviations: HA, hemagglutinin; QIV-SD, standard-dose quadrivalent influenza vaccine; TIV, trivalent influenza vaccine; μg, microgram.
*Vaccination status is determined by information provided by the parent/guardian.
†Dosage selection will be based on the ESDR of the prior Stages. The highest dosage with an acceptable safety review from prior Stages will determine the dose evaluated in Stage 4.

TABLE 1B
Influenza vaccine antigen contents and injection volumes

| | QIV-HD | | | QIV-SD | Adjuvanted TIV |
|---|---|---|---|---|---|
| Amount of | 30 | 45 | 60 | 15 | 7.5 |
| HA/strain | | | | | |
| (μg) | | | | | |
| Amount of | 120 | 180 | 240 | 60 | 22.5 |
| HA/dose | | | | | |
| (μg) | | | | | |
| A/H1N1 | A/Michigan/45/2015 | | | A/Singapore/GP1908/ | A/Singapore/GP1908/ |
| strain | X-275 | | | 2015 IVR-180 | IVR-180 |
| A/H3N2 | A/Singapore/INFIMH- | | | A/Singapore/INFIMH- | A/Singapore/INFIMH- |
| strain | 16-0019/2016 IVR-186 | | | 16-0019/2016 NIB-104 | 16-0019/2016 IVR-186 |
| B/Victoria | B/Maryland/15/2016 | | | B/Maryland/15/2016 | B/Maryland/15/2016 |
| strain | BX-69A | | | NYMC BX-69A | |
| B/Yamagata | B/Phuket/3073/2013 | | | B/Phuket/3073/2013 | — |
| strain | | | | | |
| Injection | 0.35 | 0.52 | 0.7 | 0.5 | 0.25 |
| volume | | | | | |
| (mL) | | | | | |

TABLE 1C
Baseline characteristics of the US cohort

| | QIV-SD | | QIV-HD | | QIV-HD | | QIV-HD | |
|---|---|---|---|---|---|---|---|---|
| | (15 μg) | | 30 μg | | 45 μg | | 60 μg | |
| Characteristic | N = 234 | | N = 122 | | N = 121 | | N = 158 | |
| Sex, n (%) | | | | | | | | |
| Male | 120 | (51.3) | 72 | (59.0) | 60 | (49.6) | 76 | (48.1) |
| Female | 114 | (48.7) | 50 | (41.0) | 61 | (50.4) | 82 | (51.9) |
| Age (years), mean (SD) | 5.1 | (4.0) | 5.8 | (3.9) | 6.1 | (4.3) | 5.2 | (4.2) |
| Racial origin | | | | | | | | |
| Black or African American | 58 | (24.8) | 22 | (18.0) | 28 | (23.1) | 40 | (25.3) |
| White | 157 | (67.1) | 90 | (73.8) | 80 | (66.1) | 108 | (68.4) |
| Multiple | 7 | (3.0) | 4 | (3.3) | 3 | (2.5) | 7 | (4.4) |
| Not Reported | 2 | (0.8) | 3 | (2.5) | 5 | (4.1) | 1 | (0.6) |
| Other | 10 | (4.3) | 3 | (2.4) | 5 | (4.2) | 2 | (1.3) |
| Ethnicity, n (%) | | | | | | | | |
| Hispanic or Latino | 68 | (29.1) | 31 | (25.4) | 21 | (17.4) | 40 | (25.3) |
| Not Hispanic or Latino | 164 | (70.1) | 90 | (73.8) | 96 | (79.3) | 117 | (74.1) |
| Unknown/Not reported | 2 | (0.8) | 1 | (0.8) | 4 | (3.3) | 1 | (0.6) |

TABLE 1D
Baseline characteristics of the Canadian cohort

| | Adjuvanted | | QIV-HD | | | |
|---|---|---|---|---|---|---|
| | TIV | | 60 μg | | Overall | |
| | N = 13 | | N = 13 | | N = 26 | |
| Sex | | | | | | |
| Male, n (%) | 7 | (53.8%) | 9 | (69.2%) | 16 | (61.5%) |
| Female, n (%) | 6 | (46.2%) | 4 | (30.8%) | 10 | (38.5%) |
| Age (years) | | | | | | |
| Mean (SD) | 1.0 | (0.28) | 0.8 | (0.19) | 0.9 | (0.26) |
| Race, n (%) | | | | | | |
| Black or African American | 0 | | 2 | (15.4%) | 2 | (7.7%) |
| White | 11 | (84.6%) | 9 | (69.2%) | 20 | (76.9%) |
| Other | 2 | (15.4%) | 2 | (15.4%) | 4 | (15.4%) |
| Ethnicity, n (%) | | | | | | |
| Hispanic or Latino | 0 | | 2 | (15.4%) | 2 | (7.7%) |
| Not Hispanic or Latino | 13 | (100%) | 11 | (84.6%) | 24 | (92.3%) |

TABLE 2
Table of Study Procedures 1 (subjects 9 to less than 18 years of age)

| | Visit/Contact | | | |
|---|---|---|---|---|
| | | Telephone | V03 | Telephone |
| | V01 | Call | (Second Visit)* | Call |
| | Study timelines (days) | | | |
| | | | | 180 days |
| | D 0 | D 8 | D 28 | after V01 |
| | Time windows (days) | | | |
| | | +2 | +7 | +14 |
| Informed consent form/assent form | X | | | |
| signed and dated | | | | |
| Inclusion/exclusion criteria | X | | | |
| Collection of demographic data | X | | | |
| Urine pregnancy test (if applicable)† | X | | | |
| History of seasonal influenza | X | | | |
| vaccination | | | | |
| Medical history | X | | | |
| Collection of reportable concomitant | D 0-D 28 | | | |
| medications | | | | |
| Physical examination‡ | X | | | |
| Randomization/allocation of | X | | | |
| subject number and unique dose | | | | |
| number§ | | | | |
| Blood sampling (BL), 5 mL | BL0001** | | BL0002 | |
| Vaccination | X | | | |
| Immediate surveillance (30 min) | X | | | |
| DC provided†† | DC1 | | | |
| DC reviewed and collected | | | DC1 | |
| Reporting of solicited injection site | D 0-D 7 | | | |
| and systemic reactions | | | | |
| Collection of unsolicited adverse | D 0-D 28 | | | |
| events | | | | |
| MA provided‡‡ | | | X | |
| Study active phase termination | | | X | |
| record | | | | |
| Follow-up telephone call | | X§§ | | X*** |

| | |
|---|---|
| Reporting of SAEs (including | Reported at any time during the study |
| AESIs)††† | |

Abbreviations: AESI, adverse event of special interest; BL, blood sampling; D, day; DC, diary card; MA, memory aid; SAE, serious adverse event; V, visit.
*Visit 2 is designated as Visit 3 to be consistent with the visits nomenclature of the 6 months to 8 years age groups.
†For females of childbearing potential. The urine pregnancy test was performed before vaccination. The pregnancy test must be negative for enrollment.
‡Targeted physical examination based on medical history was performed at V01. Targeted physical examination was also performed at the second visit (designated as V03), if necessary.
§Before blood sampling and vaccine injection.
**Collection of the blood sample before vaccination.
††Subjects/parents/guardians use the diary card to record information about solicited reactions, unsolicited AEs, SAEs, and AESIs from D 0 to D 7 after vaccination and continue to record information about unsolicited AEs, SAEs, and AESIs from D 8 to the second visit (designated as V03).
‡‡Subjects/parents/guardians use this MA to collect information on SAEs and AESIs from V03 (D 28 [+7 days]) to the end of the 6- month safety follow-up period.
§§During this telephone call, staff record relevant information concerning the subject's health status, find out whether the subject experienced any SAEs and AESIs not yet reported, and remind the subjects to bring the completed diary card to the second visit (designated as V03).
***During this telephone call, staff reviewed the MA to identify the occurrence of any SAEs and AESIs that have not yet been reported.
†††AESIs are captured as SAEs. These include new onset of Guillain-Barré syndrome, encephalitis/myelitis (including transverse myelitis), Bell's palsy, convulsions, optic neuritis, and brachial neuritis.

TABLE 3
Table of Study Procedures 2 (previously influenza
vaccinated subjects 6 months to 8 years of age)

| | Visit/Contact | | | |
|---|---|---|---|---|
| | | | | Telephone |
| | V01 | V02 | V03 | Call |
| | Study timelines (days) | | | |
| | | | | 180 days |
| | D 0 | D 8 | D 28 | after V01 |
| | Time windows (days) | | | |
| | | +3 | +7 | +14 |
| Informed consent form/assent form | X | | | |
| (if applicable*) signed and dated | | | | |
| Inclusion/exclusion criteria | X | | | |
| Collection of demographic data | X | | | |
| History of seasonal influenza | X | | | |
| vaccination | | | | |
| Medical history | X | | | |
| Collection of reportable concomitant | D 0-D 28 | | | |
| medications | | | | |
| Physical examination† | X | | | |
| Randomization/allocation of | X | | | |
| subject number and unique dose | | | | |
| number‡ | | | | |
| Blood sampling (BL), 5 mL | BL0001§ | | BL0002 | |
| Vaccination | X | | | |
| Immediate surveillance (30 min) | X | | | |
| DC provided** | DC1 | | | |
| DC reviewed and recorded†† | | DC1 | | |
| DC collected | | | DC1 | |
| Reporting of solicited injection site | D 0-D 7 | | | |
| and systemic reactions | | | | |
| Collection of unsolicited adverse | D 0-D 28 | | | |
| events | | | | |
| MA provided‡‡ | | | X | |
| Study active phase termination | | | X | |
| record | | | | |
| Follow-up telephone call§§ | | | | X |

| | |
|---|---|
| Reporting of SAEs (including | Reported at any time during the study |
| AESIs)*** | |

Abbreviations: AESI, adverse event of special interest; BL, blood sampling; D, day; DC, diary card; MA, memory aid; SAE, serious adverse event; V, visit.
*For children 7 to 8 years of age.
†Targeted physical examination based on medical history is performed at V01. Targeted physical examination may also be performed at V03, if necessary.
‡Before blood sampling and vaccine injection.
§Collection of the blood sample before vaccination.
**Subjects/parents/guardians use the diary card to record information about solicited reactions, unsolicited AEs, SAEs, and AESIs from D 0 to D 7 after vaccination and continue to record information about unsolicited AEs, SAEs, and AESIs from D 8 to V03.
††During V02, staff review and record D 0 to D 7 data in the CRB, find out whether the subject experienced any SAEs and AESIs not yet reported, and remind the subjects to bring the completed diary card to V03.
‡‡Subjects/parents/guardians use this MA to collect information on SAEs and AESIs from V03 to the end of the 6-month safety follow-up period.
§§During this telephone call, staff review the MA to identify the occurrence of any SAEs and AESIs that have not yet been reported.
***AESIs are captured as SAEs. These include new onset of Guillain-Barré syndrome, encephalitis/myelitis (including transverse myelitis), Bell's palsy, convulsions, optic neuritis, and brachial neuritis.

TABLE 4
Table of Study Procedures 3 (previously influenza unvaccinated subjects 6 months to 8 years of age)

| | Visit/Contact | | | | | |
|---|---|---|---|---|---|---|
| | V01 | V02 | V03 | V04 | V05 | Telephone Call |
| | Study timelines (days) | | | | | |
| | | | | 8 days | 28 days | 180 days |
| | D 0 | D 8 | D 28 | after V03 | after V03 | after V03 |
| | Time windows (days) | | | | | |
| | | +3 | +7 | +3 | +7 | +14 |
| Informed consent form/assent form | X | | | | | |
| (if applicable*) signed and dated | | | | | | |
| Inclusion/exclusion criteria | X | | | | | |
| Collection of demographic data | X | | | | | |
| History of seasonal influenza | X | | | | | |
| vaccination | | | | | | |
| Medical history | X | | | | | |
| Collection of reportable concomitant | D 0-D 56 | | | | | |
| medications | | | | | | |
| Physical examination† | X | | X | | | |
| Randomization/allocation of | X | | | | | |
| subject number and unique dose | | | | | | |
| number‡ | | | | | | |
| Allocation of unique dose number | | | X | | | |
| Temporary and definitive | | | X | | | |
| contraindications | | | | | | |
| Blood sampling (BL), 5 mL | BL0001§ | | BL0002§ | | BL0003 | |
| Vaccination | X | | X | | | |
| Immediate surveillance (30 min) | X | | X | | | |
| DC provided§** | DC1 | | DC2 | | | |
| DC reviewed and recorded†† | | DC1 | | DC2 | | |
| DC collected | | | DC1 | | DC2 | |
| Recording of solicited injection site | D 0-D 7 | | D 28-D 35 | | | |
| and systemic reactions | | | | | | |
| Collection of unsolicited adverse | D 0-D 28 | | | D 28-D 56 | | |
| events | | | | | | |
| MA provided‡‡ | | | | | X | |
| Study active phase termination | | | | | X | |
| record | | | | | | |
| Follow-up telephone call§§ | | | | | | X |

| | |
|---|---|
| Reporting of serious adverse events | Reported at any time during the study |
| (SAEs) (including AESIs)*** | |

Abbreviations: AESI, adverse event of special interest; BL, blood sampling; D, day; DC, diary card; SAE, serious adverse event; V, visit.
*For children 7 to 8 years of age.
†Targeted physical examination based on medical history is performed at V01. Targeted physical examination may also be performed at V03 or V05, if necessary.
‡Before blood sampling and vaccine injection
§Collection of the blood sample before vaccination.
**Subjects/parents/guardians use the diary cards to record information about solicited reactions, unsolicited AEs, SAEs, and AESIs after each vaccination (from D 0 to D 7 and from D 28 to D 35) and continue to record information about unsolicited AEs, SAEs, and AESIs from D 8 to V03 and D 36 to V05.
††During V02 and V04, staff review and record D 0 to D 7 and D 28 to D 35 data in the CRB, find out whether the subject experienced any SAEs and AESIs not yet reported, and remind the subjects to bring the completed diary card to their next scheduled visit.
‡‡Subjects/parents/guardians use this MA to collect information on SAEs and AESIs from V05 to the end of the 6-month safety follow-up period.
§§During this telephone call, staff review the MA to identify the occurrence of any SAEs and AESIs that have not yet been reported.
***AESIs are captured as SAEs. These include new onset of Guillain-Barré syndrome, encephalitis/myelitis (including transverse myelitis), Bell's palsy, convulsions, optic neuritis, and brachial neuritis.

TABLE 5
List of Abbreviations used in the Examples

| | |
|---|---|
| μg | microgram |
| Ab | antibody |
| ACIP | Advisory Committee on Immunization Practices |
| AE | adverse event |
| AESI | adverse event of special interest |
| ALRI | acute lower respiratory infections |
| AR | adverse reaction |
| BL | blood sampling |
| CDC | Centers for Disease Control and Prevention |
| CI | confidence interval |
| CRB | (electronic) case report book [all the case report |
| | forms for a subject] |
| CRF | (electronic) case report form |
| CRO | contract research organization |
| D | day |
| DC | diary card |
| dil | dilution |
| DP | drug product |
| EDC | electronic data capture |
| ELISA | enzyme-linked immunosorbent assay |
| ESDR | early safety data review |
| FVFS | first visit, first subject |
| FVLS | first visit, last subject |
| GMT | geometric mean titer |
| GBS | Guillain-Barré syndrome |
| GCI | Global Clinical Immunology |
| GCP | Good Clinical Practice |
| GPV | Global Pharmacovigilance |
| GSK | GlaxoSmithKline |
| HA | hemagglutinin |
| HAI | hemagglutination inhibition |
| HAU | hemagglutination unit |
| IAS | immunogenicity analysis set |
| ICF | informed consent form |
| ICH | International Council for Harmonization |
| IEC | Independent Ethics Committee |
| IM | intramuscular |
| IME | important medical event |
| IRB | Institutional Review Board |
| IRT | interactive response technology |
| LCLS | last contact, last subject |
| LLOQ | lower limit of quantitation |
| LLT | lowest level term |
| MedDRA | Medical Dictionary for Regulatory Activities |
| MDCK | Madin-Darby canine kidney |
| MA | memory aid |
| mg | milligram |
| mL | milliliter |
| NACI | National Advisory Committee on Immunization |
| NT | neutralization test |
| NH | Northern Hemisphere |
| NMT | not more than |
| NSAID | non-steroidal anti-inflammatory drug |
| PT | preferred term |
| QIV | quadrivalent influenza vaccine |
| QIV-HD | high dose quadrivalent influenza vaccine |
| QIV-SD | standard dose quadrivalent influenza vaccine |
| qs | quantity sufficient |
| RBC | red blood cell |
| SAE | serious adverse event |
| SafAS | safety analysis set |
| SAP | statistical analysis plan |
| SN | seroneutralization |
| SMT | Safety Management Team |
| SOC | system organ class |
| TIV-HD | high-dose trivalent influenza vaccine |
| TIV-SD | standard-dose trivalent influenza vaccine |
| ULOQ | upper limit of quantitation |
| US | United States |
| V | visit |
| VRBPAC | Vaccines and Related Biological Products Advisory |
| | Committee |
| WHO | World Health Organization |

### Details:

### 1.1—Rationale for Study

While influenza affects all age groups, infants and young children remain at increased risk for influenza because of their maturing immune system and lack of prior exposure and thus lack of immunity. Therefore, following a similar rationale that has been applied for adults 65 years of age and older, children may benefit from an increased antigen dose. Thus, a Phase II study evaluated if an increased antigen dose in the QIV-HD investigational product was safe and would improve immune responses in the pediatric population aged 6 months to less than 18 years compared to currently licensed standard-dose quadrivalent influenza vaccines (QIV-SD) (15 μg HA/strain). The goal of QHD04 was to select the appropriate vaccine dosage for advancement to Phase III clinical development.

QHD04 was conducted during the 2018-2019 NH influenza season in approximately 661 children 6 months to less than 18 years of age and evaluated 3 different dosages of QIV-HD in this pediatric population. A comparator vaccine was Fluarix Quadrivalent which is a unadjuvanted QIV-SD manufactured by GlaxoSmithKline (GSK). Another comparator vaccine, FLUAD® Pediatric, which is an adjuvanted TIV manufactured by Seqirus, was also evaluated since FLUAD® Pediatric is the only licensed pediatric vaccine which has been evaluated in relative efficacy studies. FLUAD® Pediatric is only licensed for the pediatric indication (6 months to less than 2 years of age) in Canada.

### 1.2—Study Objectives

Safety

To describe the safety of each dosage of QIV-HD used in the study during the 28 days following each vaccination, and serious adverse events (SAEs) (including adverse events of special interest [AESIs]) throughout the study.

Immunogenicity
To describe the antibody response induced by each dosage of QIV-HD used in the study compared with unadjuvanted QIV-SD by hemagglutination inhibition (HAI) measurement method.
To describe the antibody response induced by each dosage of QIV-HD used in the study compared with unadjuvanted QIV-SD by virus seroneutralization (SN) measurement method.
To describe the antibody response induced by the highest acceptable dosage of QIV-HD compared with adjuvanted TIV by HAI and virus SN measurement methods.

### 1.3—Investigators and Study Organization

This study was conducted in 16 centers in the US and Canada. The Principal Investigators and any sub-investigators at the individual sites are coordinated by 1 Coordinating Investigator.

Safety Management Team

An internal safety management team (SMT) performs an analysis of safety data during the conduct of the study after the first vaccination (Early Safety Data Review [ESDR]).

Monitoring, Data Management, and Statistical Analysis

Biostatistics, data management, monitoring, and medical writing are subcontracted to a Contract Research Organization (CRO) or performed in-house by the Sponsor.

Laboratory Analysis

Testing was performed at Sanofi Pasteur's Global Clinical Immunology (GCI) department or at an outsourced laboratory under the supervision of GCI.

### 1.4—Independent Ethics Committee/Institutional Review Board

Before the investigational product is shipped to the investigational site and before the inclusion of the first subject, the protocol, the informed consent form (ICF), the assent form, subject recruitment procedures, and any other written information to be provided to subjects was approved by, and/or received favorable opinion from, the appropriate Independent Ethics Committees (IECs) or Institutional Review Boards (IRBs).

In accordance with Good Clinical Practice (GCP) and local regulations, each Investigator and/or the Sponsor was responsible for obtaining this approval and/or favorable opinion before the start of the study.

### 1.4.1—Investigational Plan

Description of the Overall Study Design and Plan

Study Design

QHD04 is a Phase II, randomized, staged, modified double-blind, active-controlled, multi-center study conducted in 665 children 6 months to 17 years of age to evaluate the safety and immunogenicity of 3 dosages of QIV-HD administered by IM route versus QIV-SD or adjuvanted TIV.

The study was divided into 13 groups and enrolled in 4 stages. The study used a stepwise age de-escalation and dose ascension design for children 6 months to less than 5 years of age. Children 5 to 8 years of age also underwent a dose ascension design and began enrollment in Stage 1. Children 9 to 17 years of age were enrolled in Stage 1 and randomized to receive all three dose formulations (i.e., 30 μg, 45 μg, and 60 μg HA/strain/dose). An ESDR was conducted after Visit (V) 02 (at Day [D] 8 post-vaccination) of Stages 1, 2, and 3 for children 6 months to less than 5 years of age and Stages 1 and 2 for children 5 to 8 years of age. The ESDR for children 6 months to less than 5 years of age was independent of the ESDR for children 5 to 8 years of age.

The 13 study groups were divided according to:
Age (9 to 17 years (i.e., from the 9th birthday to the day before the 18th birthday), 5 to 8 years (i.e., from the 5th birthday to the day before the 9th birthday), 36 months to less than 5 years, 6 to less than 36 months, or 6 to less than 24 months)
Influenza vaccination history (previously influenza vaccinated, previously influenza unvaccinated, or both)
Administered vaccines (QIV-HD dosages [30 μg, 45 μg, or 60 μg HA/strain/dose], unadjuvanted QIV-SD, and adjuvanted TIV).

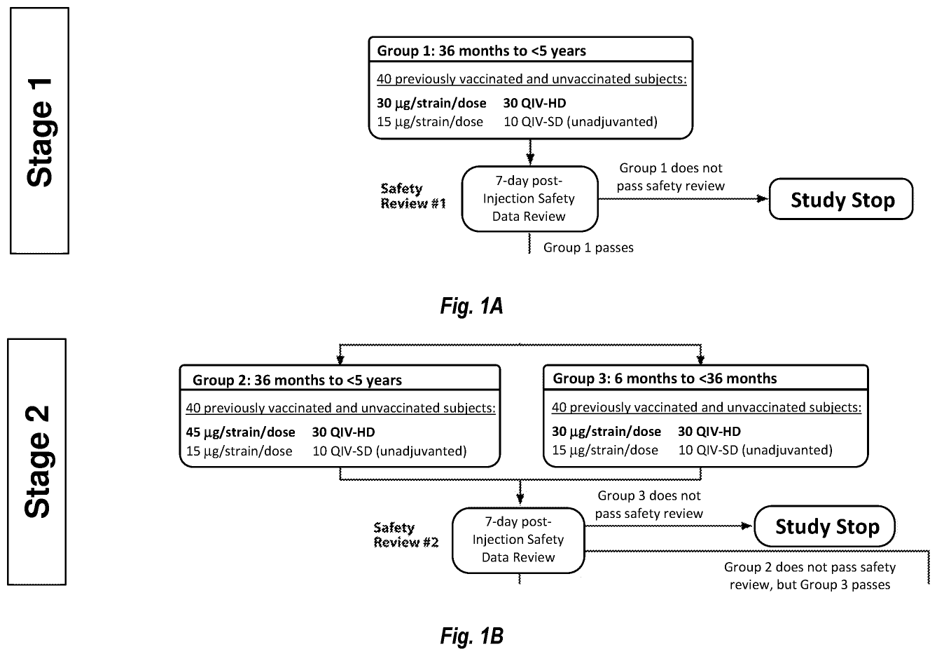

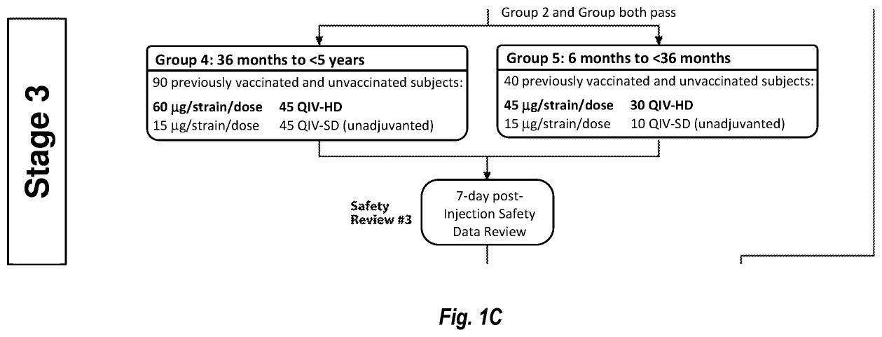

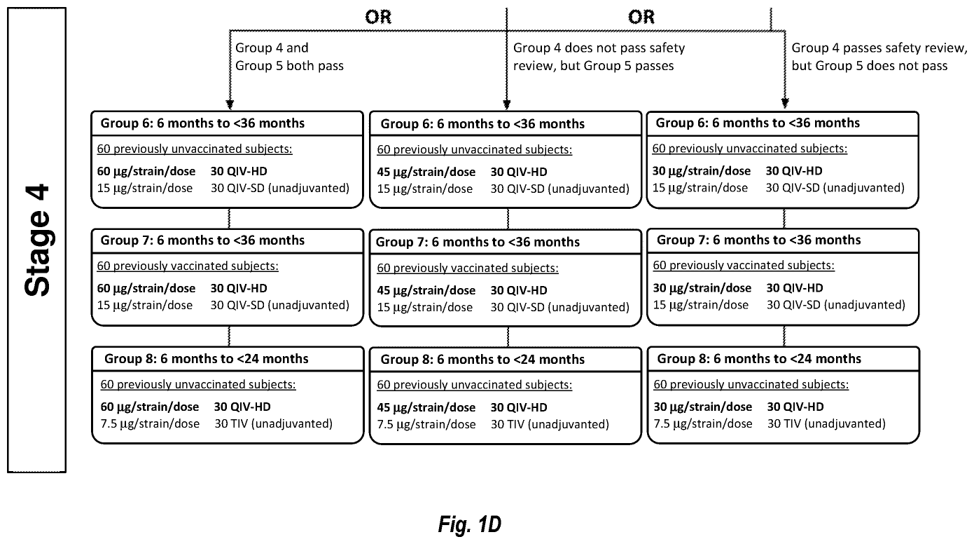

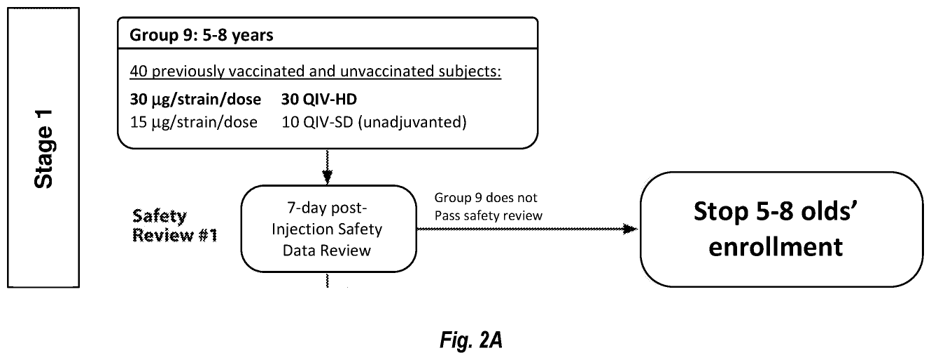

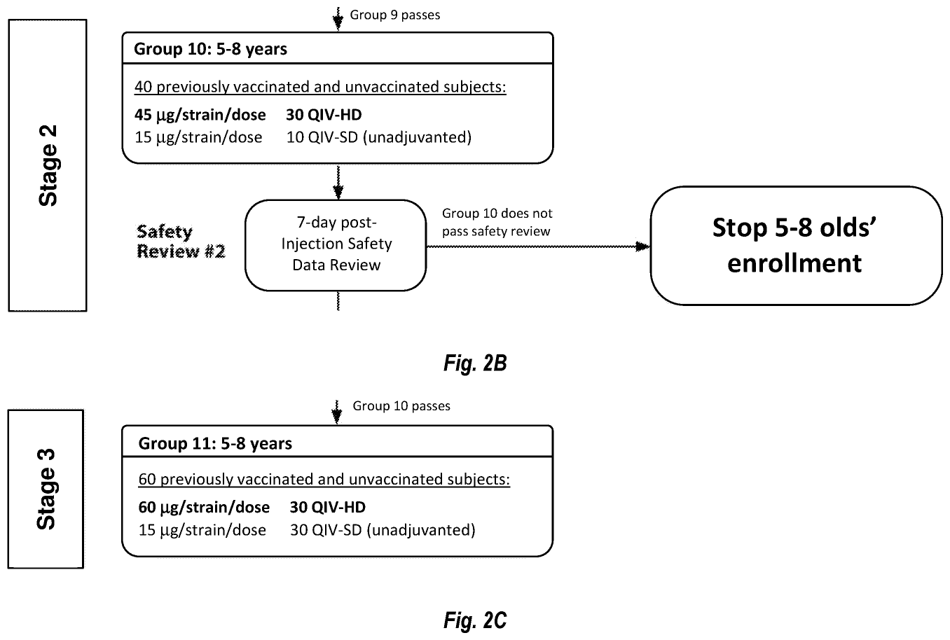

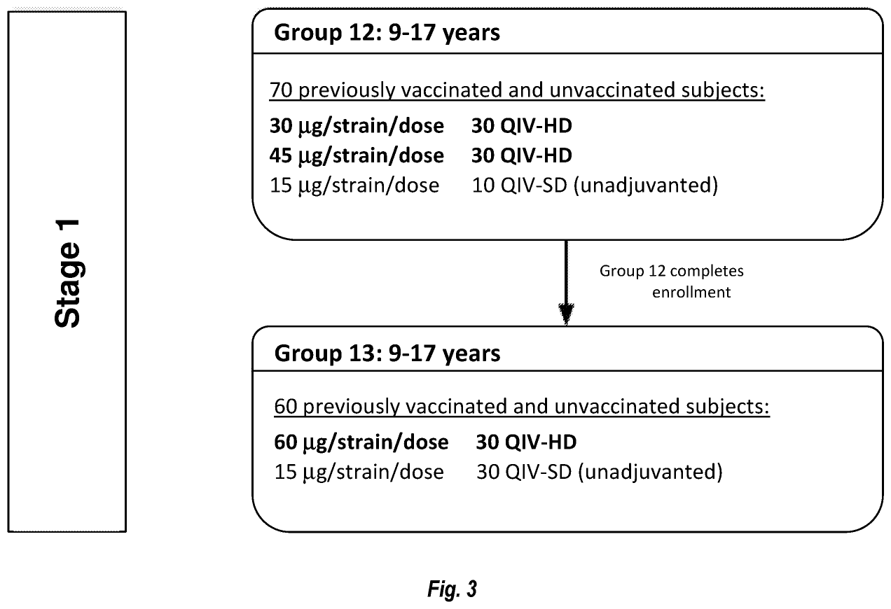

The study design is shown in Table 1A and FIGS. 1A-1D, 2A-2C and 3.

Stage 1 included 3 age groups (36 months to less than 5 years, 5 to 8 years, and 9 to 17 years) and was conducted in the US:
Previously influenza vaccinated and unvaccinated subjects 36 months to less than 5 years of age (Group 1) were randomized to receive either QIV-HD at 30 μg HA/strain/dose or the unadjuvanted QIV-SD. Based on the early safety data review, the study either stops (Group 1 does not pass safety review) or continues into Stage 2.
Previously influenza vaccinated and unvaccinated subjects 5 to 8 years of age (Group 9), were randomized to receive either QIV-HD at 30 μg HA/strain/dose or the unadjuvanted QIV-SD. Based on the early safety data review, the enrollment of this age group either stops (Group 9 does not pass safety review) or continues into Stage 2.
Previously influenza vaccinated and unvaccinated subjects 9 to 17 years of age (hereafter will be referred to as subjects 9 to 17 years of age) were divided into 2 groups (Group 12 and Group 13). Group 12 was enrolled first and was randomized to receive either QIV-HD at 30 μg, or 45 μg HA/strain/dose or the unadjuvanted QIV-SD. Once the enrollment of Group 12 was complete, subjects in Group 13 were randomized to receive either QIV-HD at 60 μg or the unadjuvanted QIV-SD. Subjects 9 to 17 years of age did not undergo an ESDR.

Stage 2 included 3 age groups (6 to less than 36 months, 36 months to less than 5 years, and 5 to 8 years) and was conducted in the US:
Previously influenza vaccinated and unvaccinated subjects 36 months to less than 5 years of age (Group 2) were randomized to receive either QIV-HD at 45 μg HA/strain/dose or the unadjuvanted QIV-SD and previously influenza vaccinated and unvaccinated subjects 6 to less than 36 months of age (Group 3) were randomized to receive either QIV-HD at 30 μg HA/strain/dose or the unadjuvanted QIV-SD. Based on the early safety data review, the study either stops (Group 3 does not pass safety review), continues and skip to Stage 4 (Group 2 does not pass safety review), or continues to Stage 3 (Group 2 and Group 3 both pass the safety review).
Previously influenza vaccinated and unvaccinated subjects 5 to 8 years of age (Group 10), was randomized to receive either QIV-HD at 45 μg HA/strain/dose or the unadjuvanted QIV-SD. Based on the early safety data review, the enrollment of this age group either stops (Group 10 does not pass safety review) or continues into Stage 3.

Stage 3 includes 3 age groups (6 to less than 36 months, 36 months to less than 5 years, and 5 to 8 years) and was conducted in the US:
Previously influenza vaccinated and unvaccinated subjects 36 months to less than 5 years of age (Group 4) were randomized to receive either QIV-HD at 60 μg HA/strain/dose or the unadjuvanted QIV-SD; and previously influenza vaccinated and unvaccinated subjects 6 to less than 36 months of age (Group 5) were randomized to receive either QIV-HD at 45 μg HA/strain/dose or the unadjuvanted QIV-SD. Based on the early safety data review, the highest dosage (QIV-HD at 60 μg HA/strain/dose) with an acceptable safety review was determined and used in Stage 4.
Previously influenza vaccinated and unvaccinated subjects 5 to 8 years of age (Group 11) were randomized to receive either QIV-HD at 60 μg HA/strain/dose or the unadjuvanted QIV-SD.

Stage 4 includes 2 age groups (6 to less than 36 months and 6 to less than 24 months) and was conducted in the US (subjects 6 to less than 36 months) and Canada (subjects 6 to less than 24 months):
Previously influenza unvaccinated subjects 6 to less than 36 months of age (Group 6) and previously influenza vaccinated subjects 6 to less than 36 months of age (Group 7) were randomized to receive either the highest dosage of QIV-HD (QIV-HD at 60 μg HA/strain/dose) with an acceptable safety review or the unadjuvanted QIV-SD.
Previously influenza unvaccinated subjects 6 to less than 24 months of age (Group 8) received either the highest dosage of QIV-HD (QIV-HD at 60 μg HA/strain/dose) with an acceptable safety review or the adjuvanted TIV.

### 1.4.1—Justification of the Study Design

QHD04 study was designed to describe the safety of 3 different dosages of QIV-HD in children 6 months to 17 years of age. The study was also designed to describe the immunogenicity of the different dosages of QIV-HD compared to a unadjuvanted QIV-SD (Fluarix® Quadrivalent, licensed in the US) in all stages of the study and compared to an adjuvanted TIV (FLUAD® Pediatric, licensed in Canada) during Stage 4 after the highest acceptable dosage of QIV-HD was established.

Given the different volumes in the 3 different dosages of the QIV-HD and the comparator vaccines, QHD04 was a staged, modified double-blind study with an unblinded administrator used at each study site. The administrator was not involved in any of the blinded study assessments (e.g., safety).

Since this was the first time that the QIV-HD was administered to the pediatric population, an ESDR was performed for subjects 6 months to 8 years of age prior to dosage ascension (30, 45, and 60 μg HA/strain/dose).

Given the matured immune system in children 9 to 17 years of age, no ESDR was needed for subjects 9 to 17 years of age prior to dosage ascension. Subjects 9 to 17 years of age were randomized to receive the 30 and 45 μg HA/strain/dose vaccines separately from subjects 9 to 17 years of age who receive the 60 μg HA/strain/dose formulation in order to maintain the randomization ratio across all age groups and thus allow for pooling of the 30, 45, and 60 μg HA/strain/dose formulations across all age groups as well.

The comparison of the QIV-HD that was used in Stage 4 (highest dosage with acceptable safety) to the adjuvanted TIV was an indicator in terms of expected vaccine efficacy since FLUAD Pediatric is the only licensed pediatric vaccine with studies evaluating both HAI immunogenicity and relative efficacy data.

### 1.4.2—Study Plan

The study plan is summarized in the Tables of Study Procedures (Tables 2-4).

Vaccination

All eligible subjects were randomized to receive 1 or 2 doses of either QIV-HD, Fluarix® Quadrivalent (unadjuvanted QIV-SD), or FLUAD Pediatric® (adjuvanted TIV). The dosage of QIV-HD that is administered will depend on both the age of the subject and the stage at which he/she is enrolled.
Subjects 9 to 17 years of age received 1 dose of the QIV-HD or the comparator vaccine on D0.
Previously influenza vaccinated subjects 6 months to 8 years of age received 1 dose of the QIV-HD or the comparator vaccine on DO.
Previously influenza unvaccinated subjects 6 months to 8 years of age received 2 doses of the QIV-HD or the comparator vaccine. Each dose was administered 28 days apart (at DO and D28).

Of note, the highest dosage with an acceptable safety review in prior stages determined the dose evaluated in Stage 4.

An unblinded administrator at each site administered the vaccine.

Blood Sampling

Subjects were to provide 2 or 3 blood samples, regardless of the stage of enrollment:
Subjects 9 to 17 years of age provide a pre-vaccination (baseline) blood sample at V01 (DO) and a post-vaccination blood sample at V03 (D28 [+7 days]) for HAI and SN testing. Note: Subjects 9 to 17 years of age were scheduled for 2 site visits. However, the second visit is designated as V03 to be consistent with the visits nomenclature of the 6 months to 8 years age groups.
Previously influenza vaccinated subjects 6 months to 8 years of age provided a pre-vaccination (baseline) blood sample at V01 (DO) and a post-vaccination blood sample at V03 (D28 [+7 days]) for HAI and SN testing.
Previously influenza unvaccinated subjects 6 months to 8 years of age provided a pre-vaccination (baseline) blood sample at V01 (DO) and a post-vaccination blood sample at V03 (D28 [+7 days]) and V05 (28 days after V03 [+7 days]) for HAI and SN testing.

Note: The blood sample was taken prior to vaccination during any visit when a subject receives a vaccination.

Collection of Safety Data

All subjects were observed for 30 minutes after vaccination, and any unsolicited systemic adverse events (AEs) occurring during that time were recorded as immediate unsolicited systemic AEs in the case report book (CRB).

Solicited reactions were collected up to 7 days after each vaccination, and unsolicited AEs were collected up to D28 (V03) for subjects receiving 1 dose and up to D56 (V05) for subjects receiving 2 doses. Serious adverse events (SAEs) and adverse events of special interest (AESIs) were collected throughout the study (D0 through approximately 6 months after the last vaccination). AESIs were captured as SAEs. These include new onset of Guillain-Barré syndrome (GBS), encephalitis/myelitis (including transverse myelitis), Bell's palsy, convulsions, optic neuritis, and brachial neuritis.

Subjects/parents/guardians were asked to notify the site immediately about any potential SAEs (including AESIs) at any time during the study.

The study staff contacted subjects 9 to 17 years of age or the subjects' parents/guardians by phone at D8 (+2 days) post-vaccination to identify whether the subject experienced any SAEs not yet reported and reminded the subjects/subjects' parents/guardians to bring the completed diary card with them to V03 (D28 [+7 days]). The study staff reviewed the D0 to V03 safety data with subjects at V03.

Subjects 6 months to 8 years of age returned to the site at D8 (+3 days) after each vaccination. Staff reviewed the recorded solicited reactions and unsolicited AEs, and determined whether the subject experienced any SAEs and AESIs not yet reported. Staff reviewed the safety data with subjects/subjects' parents/guardians at each visit. Staff also reminded the subjects/subjects' parents/guardians to bring the completed diary card with them to the subsequent visit.

The interactive response technology (IRT) system was used to randomly assign subjects to a study product and to assign subject numbers in each of the groups.

Electronic data capture (EDC) was used for the collection of data.

Review of Safety Data

The study was divided into 4 stages (Section 1.5). An ESDR was conducted for subjects 6 months to 8 years of age. The ESDR occurred following the collection of the safety data at V02 (approximately 8 days post-vaccination) of Stages 1, 2, and 3 by the SMT. Enrollment of subjects in the next stage was initiated if the safety review results were satisfactory.

1.4.3—Visit Procedures

Visit Procedures

Visit 1 (Day 0): Inclusion, Randomization, Blood sample, and Vaccination—for all Subjects

The Investigator or delegate:
1) Gives the subject and/or subject's parent/guardian information about the study.
2) Obtains informed consent and assent (for subjects 7 to 17 years of age) and answers any questions to ensure that the subjects and/or subject's parent/guardian have been informed of all aspects of the study that are relevant to their decision to participate.
3) Dates and signs the ICF (and assent form for subjects 7 to 17 years of age) after it has been signed and dated by the subject or subject's parent/guardian. Retains the original and give a signed copy to the subject or subject's parent/guardian.
4) Checks all inclusion and exclusion criteria through physical examination and medical interview of the subject and/or subject's parent/guardian. If the subject is not eligible, only the specific form entitled “Recruitment log” would state the subject identification, no CRB would be completed.
5) Collects relevant demographic information (e.g., date of birth, sex, ethnicity, and race).
6) For females of childbearing potential, conducts a urine pregnancy test.
7) Obtains verbal information on medical history.
8) Obtains information on seasonal influenza vaccination and any influenza diagnosis as follows:
   For previously influenza unvaccinated subjects, confirms that the subject has not been vaccinated with any seasonal influenza vaccine since birth and has never had a known influenza diagnosis.
      i. For the US sites, in following the Advisory Committee on Immunization Practices (ACIP) Guidelines, this study considers subjects 6 months to 8 years of age who have never been vaccinated against influenza or who have not received at least 2 doses of seasonal influenza vaccine in a prior influenza season as “previously influenza unvaccinated subjects” when enrolling and thus these subjects receive 2 doses of study vaccine at least 28 days apart. Subjects whose vaccination history is unknown are also to be considered as “previously influenza unvaccinated subjects” when enrolling and would also receive 2 doses of study vaccine at least 28 days apart.
      ii. For the Canadian sites, in following the National Advisory Committee on Immunization (NACI) recommendations, this study considers subjects 6 months to less than 24 months of age who have never been vaccinated against influenza in a prior influenza season as “previously influenza unvaccinated subjects” when enrolling and thus these subjects would receive 2 doses of study vaccine at least 28 days apart. Subjects who have received at least one dose of any influenza vaccine in the past would not be considered “previously influenza unvaccinated subjects”.
   For previously influenza vaccinated subjects, obtains the date (month and year) when the subject was last vaccinated with a seasonal influenza vaccination.
      i. For the US sites, in following the ACIP Guidelines, this study considers subjects 6 months to 8 years of age who have received at least 2 doses of seasonal influenza vaccine in prior influenza seasons as “previously influenza vaccinated subjects” when enrolling and thus these subjects would receive only 1 dose of study vaccine.
9) For subjects 6 to 12 months of age, obtains information on influenza vaccination during the mother's pregnancy and breastfeeding routines, if any.
10) Collects any reportable concomitant medications.
11) Performs and documents a targeted physical examination per standard site-specific immunization practices and record temperature (Tympanic and temporal artery thermometer should not be used) in the source documents.
12) Calls the IRT for assignment of the 12-digit subject number, randomization, and allocation of a dose number.
13) Draws approximately 5 mL of blood sample (the blood sampling should be performed before vaccination) for processing.
   Note: If 5 mL of blood sample cannot be drawn, a volume less than 5 mL can be obtained.
   Note: If the subject's parent/guardian withdraws consent before blood sampling (before any invasive procedure has been performed), the subject was not to be vaccinated. The subject was to be terminated from the study.
   Note: If the attempt(s) to collect blood are unsuccessful (3 attempts), then the subject was to be included in the study and vaccinated.

The unblinded qualified study staff member:
14) Administers the appropriate vaccine intramuscularly into the anterolateral muscle of the thigh or the deltoid muscle of the upper arm, as appropriate (the vaccine must be administered on the side opposite to that of the blood sampling).
15) Records the injection site/side/route/dose number and affixes the detachable corresponding label in the source documents.

The Investigator or delegate:
16) Keeps the subject under medical surveillance for at least 30 minutes after the injection and reports the occurrence or non-occurrence of any AE in the source documents.
17) Gives the subject or subject's parent/guardian the diary card (DC) 1 to record any injection site reactions and systemic AEs, together with instructions for its completion, including explanations on the definition and use of intensity scales for collection of AEs.
18) Gives the subject or subject's parent/guardian a ruler to measure the size of any injection site reaction, a thermometer for temperature measurement, and instructions on how to use them.
19) Reminds the subject or subject's parent/guardian to bring back the DC1 when they return for V02. For subjects 9 to 17 years of age, schedules the D8 telephone call.
20) Reminds the subject or subject's parent/guardian to promptly notify the site in case of an SAE/AESI that may occur at any time during the study.
21) Completes the relevant CRB forms for this visit.

Telephone Call (8 [+2] Days after Visit 1): Collection of Safety Information—for Subjects 9 to 17 Years of Age

If the telephone call falls on a weekend or a holiday, the telephone call is scheduled on the next business day.

The Investigator or delegate:
1) Records relevant information concerning the subject's health status on the telephone contact form. If an SAE occurred, will follow the instructions for reporting it.
2) Reminds the subject or subject's parent/guardian to do the following:
   Complete the remaining pages of the DC1 and bring the completed diary to V03 (D28 [+7 days]).
   Notify the site in case of an SAE.

Visit 2 (8 [+3] Days after Visit 1): Collection of Safety Information—for Subjects 6 Months to 8 Years of Age

If Visit 2 falls on a weekend or a holiday, the visit may be scheduled on the next business day.

The Investigator or delegate:
1) Reviews (for clarity, content, and completeness) the information in the DC1 with the subject or subject's parent/guardian and clarify with the subject or subject's parent/guardian, if required, any AEs, medications, or SAE that occurred since V01. If an SAE occurred, follows the instructions herein for reporting it.
2) Records the D0 to D7 safety data in the CRB.
3) Reminds the subject or subject's parent/guardian to do the following:
   Completes the remaining pages of the DC1 and bring the completed diary to V03
   Notifies the site in case of an SAE.

Visit 3 (28 [+7] Days after Visit 1)—for Subjects 9 to 17 Years of Age and for Previously Influenza Vaccinated Subjects 6 Months to 8 Years of Age

Subjects 9 to 17 years of age were scheduled for 2 site visits. However, the second visit was designated as V03 to be consistent with the visit nomenclature of the 6 months to 8 years age groups.

The Investigator or delegate:
1) Reviews (for clarity, content, and completeness) and collects the DC1 information with the parent/guardian and clarify with the parent/guardian if required any AEs, medications, or SAE that occurred since the telephone call (for subjects 9 to 17 years of age) or V02 (for previously vaccinated subjects 6 months to 8 years of age).
2) Draws approximately 5 mL of blood sample for processing.
   i. If 5 mL of blood sample cannot be drawn, a volume less than 5 mL can be obtained.
   ii. If the attempt(s) to collect blood are unsuccessful (3 attempts), the parent/guardian was given the opportunity to bring his/her child to the study site for another attempt within the visit window. If a blood sample cannot be obtained, the reason is recorded in the blood sampling page of the CRB. In this case, the subject was followed for safety in the study.
3) Provides the subject/parent/guardian with a memory aid (MA) and reviews the directions for its use.
4) Reminds the subject/parent/guardian to promptly notify the site in case of an SAE that may occur at any time during the study.
5) Schedules the 6-month safety follow-up phone call
6) Records all applicable information obtained into the CRB
7) Completes the termination record of the CRB.

Visit 3 (28 [+7] Days after Visit 1)—for Previously Influenza Unvaccinated Subjects 6 Months to 8 Years of Age

The Investigator or delegate:
1) Reviews (for clarity, content, and completeness) and collects the DC1 information with the parent/guardian and clarify with the parent/guardian if required any AEs, medications, or SAE that occurred since V02.
2) Conducts a targeted physical examination, if necessary.
3) Reviews temporary and definitive contraindications to vaccination.
4) Draws approximately 5 mL of blood sample (The blood sampling should be performed before vaccination).
   i. If 5 mL of blood sample cannot be drawn, a volume less than 5 mL can be obtained.
   ii. If the attempt(s) to collect blood is (are) unsuccessful (3 attempts), the parent/guardian is given the opportunity to bring his/her child to the study site for another attempt within the visit window. If a blood sample cannot be obtained, the reason will be recorded in the blood sampling page of the CRB. In this case, the subject remains in the study and is vaccinated after all the attempts to collect blood are complete.

The unblinded qualified study staff member:
5) Calls the IRT to obtain a unique dose number
6) Administers the appropriate vaccine intramuscularly into the anterolateral muscle of the thigh or the deltoid muscle of the upper arm, as appropriate (the vaccine must be administered on the side opposite to that of the blood sampling).
7) Records the injection site/side/route/dose number in the source documents and affixes the detachable corresponding label in the source document.

The Investigator or delegate:
8) Keeps the subject under medical surveillance for at least 30 minutes after the injection and reports the occurrence or non-occurrence of any AE in the source documents.
9) Gives the subject/parent/guardian the DC2 to record any injection site reactions and systemic AEs, together with instructions for its completion, including explanations on the definition and use of intensity scales for collection of AEs.
10) Reminds the subject/parent/guardian to bring back the DC2 when they return for V04.
11) Reminds the subject/parent/guardian to promptly notify the site in case of an SAE that may occur at any time during the study.
12) Completes the relevant CRB forms for this visit.

Visit 4 (8 [+3] Days after Visit 3)—for Previously Influenza Unvaccinated Subjects 6 Months to 8 Years of Age

The Investigator or delegate:
1) Reviews (for clarity, content, and completeness) the information in the DC2 with the subject/parent/guardian and clarify with the parent/guardian, if required, any AEs, medications, or SAE that occurred since V03.
2) Records the D28-D35 safety data in the CRB.
3) Reminds the subject/parent/guardian to do the following:
   Complete the remaining pages of the DC2 and bring them to V05
   Notify the site in case of an SAE.

Visit 5 (28 [+7] Days after Visit 3)—for Previously Influenza Unvaccinated Subjects 6 Months to 8 Years of Age

The Investigator or delegate:
1) Reviews (for clarity, content, and completeness) and collects the DC2 information with the parent/guardian and clarify with the subject/parent/guardian if required any AEs, medications, or SAE that occurred since V04. If an SAE occurred, follow the instructions herein for reporting it.
2) Draws approximately 5 mL of blood sample for processing.
   i. If 5 mL of blood sample cannot be drawn, a volume less than 5 mL can be obtained.
   ii. If the attempt(s) to collect blood is (are) unsuccessful (3 attempts), the parent/guardian is given the opportunity to bring his/her child to the study site for another attempt within the visit window. If a blood sample cannot be obtained, the reason is recorded in the blood sampling page of the CRB. In this case, the subject remains in the study.
3) Provides the subject/parent/guardian with a MA and reviews the directions for its use.
4) Reminds the subject/parent/guardian to promptly notify the site in case of an SAE that may occur at any time during the study.
5) Schedules the 6-month safety follow-up phone call
6) Records all applicable information obtained into the CRB
7) Completes the termination record of the CRB.

Safety Follow-Up Telephone Call—Approximately 6 Months after the Last Vaccination: Collection of SAEs
1) Inquiries about new occurrences of SAEs and perform follow-up on SAEs. If an SAE has occurred, inquires if the subject has received any vaccinations or other medications since the last contact.
2) Explains that this will be the last contact with the site for this study (except for subjects with SAEs that need further follow-up, as stated below).

If the first contact attempt to complete the last telephone call is unsuccessful, at least 2 separate additional attempts, conducted on different days, are made to contact these subjects. All attempts are documented in the subject's source notes. If, after at least 3 documented attempts, contact cannot be established, the subject is classified as Lost to Follow-Up.

The exceptions for the final phone calls are:
Subjects who voluntarily withdraw
Subjects who have been previously classified as lost to follow-up

Follow-Up of Subjects with Related AEs or with AEs that LED to Study/Vaccination Discontinuation:

A subject who experiences an AE (whether serious or non-serious) during the study is followed until the condition resolves, becomes stable, or becomes chronic (even after the end of the subject's participation in the study) if either of the following is true:
The AE is considered by the Investigator to be related to the product administered.
The AE caused the discontinuation of the subject from the study or from vaccination.

### 1.4.4—Early Safety Data Review

The safety of the investigational product was continuously monitored by the Sponsor. ESDRs were performed, the goal of which is to allow for a cautious, stepwise approach to vaccine administration. A stepwise dosage ascension approach was applied to subjects in age group 5 to 8 years (30 μg, 45 μg, and 60 μg HA/strain/dose). A stepwise age de-escalation vaccination approach was to be taken for subjects in age group 36 months to less than 5 years of age, with an ESDR prior to vaccinating subjects in the age group 6 to less than 36 months of age as well as stepwise dosage ascension. The ESDR was performed following V02 in subjects 6 months to 8 years of age.

The ESDRs for this study are planned after the 6 months to 8 years age groups in each stage have been vaccinated and have provided safety data for Days 0 to 7 post-vaccination, using the data collection methods described in the protocol. ESDR was not performed for subjects 9 to 17 years of age.

During each ESDR, enrollment of subjects was paused. However, already enrolled subjects 6 months to 8 years of age who required 2 vaccinations because of their previous vaccination status would receive their second dose at D28 as scheduled, which may be prior to the ESDR. Following a satisfactory safety review, enrollment of subjects would resume for the subsequent or appropriate Stage. The study visits according to the Table of Study Procedures continued to be followed for enrolled subjects within each active Stage (i.e., subjects complete their scheduled visits).

The safety data collected were entered into the CRBs and summarized by the Sponsor in a blinded manner. A review was performed by the Sponsor during the SMT meetings. It is understood that this review is based on preliminary data that have not been subject to validation and database lock. (The usual and ongoing process of monitoring safety signals outside of those specified in the protocol-defined early interim safety analysis continued unchanged.)

The following safety parameters were assessed as part of the early safety review:
Immediate reactions
Solicited injection site and systemic reactions
Unsolicited AEs
SAEs (including AESIs)

Enrollment was paused during the review, and the data was examined for the following occurrences:
An SAE (including AESIs) considered as related to the vaccination by the Investigator and Sponsor
more than 10% of subjects experiencing Grade 3 fever within 7 days after vaccination

If any of the above criteria are met, a decision was made as to whether enrollment in the study was allowed to resume.

Case unblinding may be performed if necessary.

### 1.5—Enrollment and Retention of Study Population

### 1.5.1—Recruitment Procedures

Subjects are recruited from the general population.

Documentation of the consent process should be recorded in the source documents.

### 1.5.2—Screening Criteria

There are no screening criteria other than the inclusion and exclusion criteria.

### 1.5.3—Inclusion Criteria

An individual had to fulfill all of the following criteria to be eligible for study enrollment:
1) Aged 6 months to 17 years on the day of inclusion (“6 months to 17 years” means from the 6th month after birth to the day before the 18th birthday).
2) Assent form has been signed and dated by the subject (7 to 17 years of age) and informed consent form has been signed and dated by the parent(s) or guardian(s) and by an independent witness, if required by local regulations.
3) Subject and parent/guardian are able to attend all scheduled visits and to comply with all study procedures.
4) For subjects aged less than 24 months: Born at full term of pregnancy (greater than or equal to 37 weeks) and/or with a birth weight greater than or equal to 2.5 kg.

### 1.5.4—Exclusion Criteria

An individual fulfilling any of the following criteria was to be excluded from study enrollment:

TABLE 5A
Exclusion criteria

| Applicable participants | Exclusion criterion |
|---|---|
| All participants | Participating (at the time of study enrollment or in the 4 |
| | weeks before the first dose of vaccine) or planning to |
| | participate (during the study period) in another clinical |
| | study investigating a vaccine, drug, medical device, or |
| | medical procedure |
| | Receipt of any vaccine in the 30 days before study |
| | vaccination (or before the first vaccine dose for participants |
| | who received two doses of influenza vaccine) or in the 28 |
| | days after study vaccination (or after the second dose of |
| | study vaccine for participants who received two doses of |
| | influenza vaccine) |
| | Receipt of immune globulins, blood, or blood-derived |
| | products in the previous 3 months |
| | Known or suspected congenital or acquired |
| | immunodeficiency; or receipt of immunosuppressive |
| | therapy in the previous 6 months; or long-term systemic |
| | corticosteroid therapy (prednisone or equivalent for more |
| | than 2 consecutive weeks in the previous 3 months) |
| | Known systemic hypersensitivity to any of the vaccine |
| | components or history of life-threatening reaction to any of |
| | the vaccines used in the study or to a vaccine containing any |
| | of the same substances |
| | Thrombocytopenia or bleeding disorder contraindicating |
| | intramuscular vaccination, in the investigator's judgement |
| | Deprived of freedom by an administrative or court order, or |
| | in an emergency setting, or hospitalized involuntarily |
| | Current alcohol abuse or drug addiction |
| | Chronic illness that, in the opinion of the investigator, might |
| | interfere with study conduct or completion^{a} |
| | Moderate or severe acute illness/infection (in the |
| | investigator's judgment) or febrile illness |
| | (temperature ≥38.0° C. or ≥100.4° F.) on the day of |
| | vaccination. A prospective participant was not to be included |
| | in the study until the condition had resolved or the febrile |
| | event had subsided |
| | Immediate family member of the investigator or an |
| | employee with direct involvement in the proposed study |
| | History of Guillain-Barré syndrome |
| | Any condition that, in the opinion of the Investigator, would |
| | pose a health risk to the participant if enrolled or could |
| | interfere with the evaluation of the vaccine |
| | History of clinically significant development delay (at the |
| | discretion of the Investigator), neurologic disorder, or |
| | seizure disorder |
| | Seropositivity for hepatitis B or hepatitis C |
| Previously vaccinated | Vaccinated against influenza with any vaccine in the |
| participants* | previous 6 months |
| Unvaccinated participants⁺ | History of vaccination against influenza with any vaccine |
| | History of laboratory confirmed influenza infection |
| Female participants of | Pregnant, or lactating, or sexually active and not using an |
| childbearing potential | effective method of contraception from at least 4 weeks |
| | prior to vaccination until at least 4 weeks after vaccination. |
| | To be considered of non-childbearing potential, a female |
| | must be pre-menarche. |

*For US, in alignment with ACIP guidelines participants 6 mo-8 y who had received at least 2 doses of seasonal influenza vaccine in prior influenza seasons;
⁺For US: following ACIP guidelines participants 6 mo to 8 y who had never been vaccinated against influenza or had not received at least 2 doses of seasonal influenza vaccine in a prior influenza season. Participants whose vaccination history was unknown were also considered unvaccinated; For Canada: following NACI recommendations, participants 6 mo to <24 mo who had never been vaccinated against influenza in a prior season.
^{a}Chronic illness may include, but is not limited to, cardiac disorders, renal disorders, auto-immune disorders, diabetes, psychiatric disorders or chronic infection.
^{b} Pre-menarche females will declare by themselves that they have not yet started menstruation. If a young female subject reaches menarche during the study, then she is to be considered as a woman of childbearing potential from that time forward.

### 1.5.6—Medical History

Prior to enrollment, subjects are assessed for pre-existing conditions and illnesses, both past and ongoing. Significant (clinically relevant) medical history (reported as diagnosis) including conditions/illnesses for which the subject is or has been followed by a physician or conditions/illnesses that could resume during the course of the study or lead to an SAE or to a repetitive outpatient care was to be collected in the CRB. The significant medical history section of the CRB contains a core list of body systems and disorders that could be used to prompt comprehensive reporting, as well as space for the reporting of specific conditions and illnesses.

For each condition, the data collected were limited to:
Diagnosis (this is preferable to reporting signs and symptoms)
Presence or absence of the condition at enrollment

Dates, medications, and body systems is not be recorded, and the information collected is not be coded. Its purpose is to assist in the later interpretation of safety data collected during the study.

### 1.5.7—Contraindications for Subsequent Vaccination

The contraindications apply only to previously influenza unvaccinated subjects who are going to receive 2 vaccine doses, 28 (+7) days apart.

### 1.5.7.1—Temporary Contraindications

If a subject experienced one of the conditions listed below, the Investigator postponed further vaccination until the condition was resolved. Postponement must still be within the timeframe for vaccination indicated in the Table of Study Procedures (Tables 2-4).
Febrile illness (temperature ≥38.0° C. [≥100.4° F.]) or moderate or severe acute illness/infection on the day of vaccination, according to Investigator judgment.

### 1.5.7.2—Definitive Contraindications

The following criteria are applicable to subjects who had never received any influenza vaccine before the start of the study and who were receiving 2 influenza vaccine injections during the study (1 at V01 and 1 at V03). If a subject experienced 1 of the conditions listed below, the Investigator discontinued vaccination.
D01: An anaphylactic or other significant allergic reaction to the previous dose of vaccine
D02: Receipt of any immune globulins, blood, or blood-derived products between V01 and V03.
D03: Known or suspected congenital or acquired immunodeficiency; or receipt of immunosuppressive therapy, such as anti-cancer chemotherapy or radiation therapy, since the preceding visit; or long-term systemic corticosteroid therapy (prednisone or equivalent for more than 2 consecutive weeks since the preceding visit).
D04: Thrombocytopenia or bleeding disorder, which may be a contraindication for IM vaccination, based on Investigator's judgement.
D05: Chronic illness that, in the opinion of the investigator, is at a stage where it might interfere with study conduct or completion.
D06: Development of any condition that in the opinion of the Investigator would pose a health risk to the subject or could interfere with the evaluation of the study vaccine (including GBS, clinically significant developmental delay, neurologic disorder, seizure disorder, hepatitis B, or hepatitis C).
D07: Any SAE related to the study vaccines following the previous study vaccination, based on investigator's judgement.

Subjects with a definitive contraindication (if any) were not vaccinated at V03, but continued to be followed up for the study-defined safety and immunogenicity assessments, as applicable.

In the event of a local or national immunization program with a pandemic influenza vaccine or other vaccine, subjects who received pandemic influenza vaccine or other vaccine at any time during the study were not withdrawn from the study.

### 1.5.8—Conditions for Withdrawal

Subjects/parents/guardians were informed that they have the right to withdraw from the study at any time. A subject may be withdrawn from the study:
At the discretion of the Investigator or Sponsor due to safety concerns or significant non-compliance with the protocol (based on the Investigator's judgment), without the subject's permission (withdrawal)
At the request of the subject/parent/guardian (dropout)

The Investigator determined whether voluntary withdrawal (if any) was due to safety concerns (in which case, the reason for discontinuation will be noted as “Adverse Event”) or for another reason.

Withdrawn subjects were not replaced.

### 1.5.9—Lost to Follow-Up Procedures

In the case of subjects who fail to return for a follow-up examination, documented reasonable effort (i.e., documented telephone calls and certified mail) are undertaken to locate or recall them, or at least to determine their health status while fully respecting their rights.

### 1.5.10—Classification of Subjects Who Discontinue the Study

For any subject who discontinues the study prior to completion, the most significant reason for early termination is documented in the CRB. Reasons are listed below from the most significant to the least significant:

TABLE 6
Classification of Subjects Who Discontinue the Study

| | |
|---|---|
| Adverse | To be used when the subject is permanently terminated from the study |
| Event | because of an AE (including an SAE). |
| | This category also applies if the subject experiences a definitive |
| | contraindication that is an SAE or AE. |
| Lost to | To be used when the subject cannot be found or contacted in spite of |
| Follow-up | efforts to locate him/her before the date of his/her planned last visit. |
| | The certified letter was sent by the investigator and returned unsigned, |
| | and the subject or parent/guardian did not give any other news and did |
| | not come to any following visit. |
| Protocol | To be used: |
| Deviation | In case of significant noncompliance with the protocol (e.g., |
| | deviation of the Inclusion/Exclusion criteria, non-compliance |
| | with time windows, blood sampling or vaccination refusal, |
| | missed injection/treatment, or error in the vaccine/treatment |
| | administration). |
| | If the subject experiences a definitive contraindication that is a |
| | protocol deviation. |
| | The subject or the parent/guardian signed the certified letter |
| | sent by the investigator but did not give any other news and did |
| | not come to any following visit. |
| Withdrawal by | To be used: |
| Subject or | When the subject or parent/guardian indicated unwillingness to |
| Parent/ | continue in the study |
| Guardian | When the subject or parent/guardian made the decision to |
| | discontinue participation in the study for any personal reason |
| | other than an SAE/AE (e.g., subject is relocating, inform |
| | consent withdrawal, etc.) |

### 1.5.11—Follow-Up of Discontinuations

The site completes all scheduled safety follow-ups and contacts any subject or parent/guardian of any subject who has prematurely terminated the study because of an AE, a protocol deviation, or loss of eligibility, including definitive contraindications. The follow-up duration in the event of discontinuation is 6 months after the last vaccination.

For subjects where the reason for early termination was lost to follow-up or if the subject/parent/guardian withdrew informed consent and specified that they do not want to be contacted again and it is documented in the source document, the site would not attempt to obtain further safety information.

If the subject's status at the end of the study is “Withdrawal by Subject or Parent/Guardian”, the site attempts to contact them for the 6-month follow-up except if they specified that they do not want to be contacted again and it is documented in the source document.

### 1.5.14—Follow-Up and Reporting of Pregnancies

Pregnancy is an exclusion criterion for enrollment in this study, but a subject could potentially become pregnant during her participation. In case of pregnancy, and if at least 1 dose of the study vaccine(s) has been administered, the subject was not discontinued from the study. However, the subject was followed for safety assessment (and may be followed for immunogenicity assessment, if applicable).

All pregnancy cases are reported if they occurred during the study and during the 6-month follow up period.

Pregnancy itself is not considered an AE, but any complications during pregnancy are considered as AEs, and in some cases could be considered SAEs. Spontaneous abortions, blighted ovum, fetal death, stillbirth, and congenital anomalies reported in the baby are always considered as SAEs, and the information should be provided to the Global Pharmacovigilance (GPV) Department regardless of when the SAE occurs (e.g., even after the end of the study).

### 1.6—Identity of the Investigational Products

### 1.6.1.—Identity of Study Product 1

The investigational QIV-HD is a split virion quadrivalent influenza vaccine (30 μg HA/strain) containing virus strains chosen by the WHO (VRBPAC in the US) for the NH 2018-2019 influenza season. The vaccine contains 2 antigens of type A (H1N1 and H3N2) and 2 antigens of type B (one each from Yamagata and Victoria lineages). Each pre-filled syringe contains a total of 120 μg HA antigen per 0.35 mL dose provided in sterile suspension for IM injection.

QIV-HD vaccine is thimerosal-free and prepared from influenza viruses propagated in embryonated chicken eggs.

1.6.1.1—Composition of Study Product 1

Each 0.35 mL dose of vaccine contains the following components: (Strains are based on WHO [VRBPAC in the US] recommendations for the 2018-2019 NH influenza season):

TABLE 7
Active substances of Study Product 1:

| | |
|---|---|
| A/Michigan/45/2015 X-275 (H1N1) | 30 μg HA |
| A/Singapore/INFIMH-16-0019/2016 IVR-186 (H3N2) | 30 μg HA |
| B/Maryland/15/2016 BX-69A (B/Colorado/6/2017-like | 30 μg HA |
| virus, Victoria lineage) | |
| B/Phuket/3073/2013 (Yamagata lineage) | 30 μg HA |

TABLE 8
Excipients of Study Product 1:

| | |
|---|---|
| Buffered saline solution | quantity sufficient (qs) |
| | to appropriate volume |
| Octylphenol Ethoxylate (Triton X-100 ®) | not more than (NMT) 175 μg |

Preservative is not used in the manufacture of QIV-HD.

1.6.1.2—Preparation and Administration of Study Product 1

The vaccine is provided in a pre-filled single-dose syringe and should be shaken before use. The vaccine is administered intramuscularly into the anterolateral muscle of the thigh or the deltoid muscle of the upper arm, as appropriate. If the vaccine is injected in the arm, it should be on the opposite arm from which blood was drawn before vaccination.

Prior to administration, all study products are inspected visually for cracks, broken seals, correct label content, and extraneous particulate matter and/or discoloration, whenever solution and container permit. If any of these conditions exists, the vaccine is not administered. A replacement dose is to be used.

Subjects are kept under observation for 30 minutes after each vaccination to ensure their safety, and any reactions during this period were documented in the CRB. Appropriate medical equipment and emergency medications, including epinephrine (1:1000), are available on site in the event of an anaphylactic, vasovagal, or other immediate allergic reaction.

1.6.1.3—Dose Selection and Timing of Study Product 1

The vaccination schedule is per standard practice for receipt of annual influenza vaccination for previously influenza vaccinated and influenza unvaccinated subjects and subjects 9 to 17 years of age.

### 1.6.2—Identity of Study Product 2

The investigational QIV-HD is a split virion quadrivalent influenza vaccine (45 μg HA/strain) containing virus strains chosen by the WHO (VRBPAC in the US) for the NH 2018-2019 influenza season. The vaccine contains 2 antigens of type A (H1N1 and H3N2) and 2 antigens of type B (one each from Yamagata and Victoria lineages). Each pre-filled syringe contains a total of 180 μg HA antigen per 0.52 mL dose provided in sterile suspension for IM injection.

QIV-HD vaccine is thimerosal-free and prepared from influenza viruses propagated in embryonated chicken eggs.

1.6.2.1—Composition of Study Product 2

Each 0.52 mL dose of vaccine contains the following components:

### (Strains are Based on WHO [VRBPAC in the US] Recommendations for the 2018-2019 NH Influenza Season):

TABLE 9
Active substances of Study Product 2:

| | |
|---|---|
| A/Michigan/45/2015 X-275 (H1N1) | 45 μg HA |
| A/Singapore/INFIMH-16-0019/2016 IVR-186 (H3N2) | 45 μg HA |
| B/Maryland/15/2016 BX-69A (B/Colorado/6/2017-like | 45 μg HA |
| virus, Victoria lineage) | |
| B/Phuket/3073/2013 (Yamagata lineage) | 45 μg HA |

TABLE 10
Excipients of Study Product 2:

| | |
|---|---|
| Buffered saline solution | qs to appropriate volume |
| Octylphenol Ethoxylate (Triton X-100) | NMT 260 μg |

Preservative is not used in the manufacture of QIV-HD.

1.6.2.2—Preparation and Administration of Study Product 2

The procedures for preparing and administering study product 2 are the same as those described for study product 1.

1.6.2.3—Dose Selection and Timing of Study Product 2

The dose selection and timing for the investigational product 2 are the same as those described for the investigational product 1.

1.6.3—Identity of Study Product 3

The investigational QIV-HD is a split virion quadrivalent influenza vaccine (60 μg HA/strain) containing virus strains chosen by the WHO (VRBPAC in the US) for the NH 2018-2019 influenza season. The vaccine contains 2 antigens of type A (H1N1 and H3N2) and 2 antigens of type B (one each from Yamagata and Victoria lineages). Each pre-filled syringe contains a total of 240 μg HA antigen per 0.7 mL dose provided in sterile suspension for IM injection.

QIV-HD vaccine is thimerosal-free and prepared from influenza viruses propagated in embryonated chicken eggs.

1.6.3.1—Composition of Study Product 3

Each 0.7 mL dose of vaccine contains the following components:

### (Strains are Based on WHO [VRBPAC in the US] Recommendations for the 2018-2019 NH Influenza Season):

TABLE 11
Active substances of Study Product 3:

| | |
|---|---|
| A/Michigan/45/2015 X-275 (H1N1) | 60 μg HA |
| A/Singapore/INFIMH-16-0019/2016 IVR-186 (H3N2) | 60 μg HA |
| B/Maryland/15/2016 BX-69A (B/Colorado/6/2017-like | 60 μg HA |
| virus (Victoria lineage) | |
| B/Phuket/3073/2013 (Yamagata lineage) | 60 μg HA |

TABLE 12
Excipients of Study Product 3:

| | |
|---|---|
| Buffered saline solution | qs to appropriate volume |
| Octylphenol Ethoxylate (Triton X-100) | NMT 350 μg |

Preservative is not used in the manufacture of QIV-HD.

1.6.3.2—Preparation and Administration

The procedures for preparing and administering study product 3 are the same as those described for study product 1.

1.6.3.3—Dose Selection and Timing

The dose selection and timing for the investigational product 3 are the same as those described for the investigational product 1.

1.6.4—Identity of Control Product 1

Fluarix® Quadrivalent: Influenza vaccine, Inactivated (GlaxoSmithKline Biologicals, Dresden, Germany)
Form: Liquid solution
Dose: 0.5 mL
Route: IM

1.6.4.1—Composition of Control Product 1

Each 0.5 mL dose contains 15 μg of HA for each of the following strains:

### Strains are Based on WHO (VRBPAC in the US) Recommendations for the 2018-2019 NH Influenza Season.

TABLE 13
Active Substances of Control Product 1:

| | |
|---|---|
| A/Singapore/GP1908/2015 IVR-180 (H1N1) | 15 μg HA |
| (A/Michigan/45/2015 [H1N1] pdm09-like virus) | |
| A/Singapore/INFIMH-16-0019/2016 NIB-104 (H3N2) | 15 μg HA |
| B/Maryland/15/2016 NYMC BX-69A | 15 μg HA |
| (B/Colorado/06/2017-like virus, Victoria lineage) | |
| B/Phuket/3073/2013 (Yamagata lineage) | 15 μg HA |

TABLE 14
Excipients of Control Product 1:

| | |
|---|---|
| Octylphenol-10 (Triton X-100) | ≤0.115 milligram (mg) |
| α-Tocopheryl hydrogen succinate | ≤0.135 mg |
| Polysorbate 80 (Tween 80) | ≤0.550 mg |

Fluarix Quadrivalent does not contain a preservative.

### 1.6.4.2—Preparation and Administration of Control Product 1

Fluarix® Quadrivalent was to be prepared and administered according to manufacturer's package insert (See, e.g., Fluarix® Quadrivalent [package insert]. GlaxoSmithKline Biologicals (Dresden, Germany).

### 1.6.4.3—Dose Selection and Timing of Control Product 1

Fluarix® Quadrivalent was to be administered to a randomized set of subjects in Groups 1 through 7 and Groups 9 through 13 as a single 0.5 mL dose at V01 for subjects 9 to 17 years of age and previously influenza vaccinated subjects 6 months to 8 years of age and at V01 and V03 for previously influenza unvaccinated subjects 6 months to 8 years of age.

### 1.6.5—Identity of Control Product 2

FLUAD® Pediatric: Influenza vaccine, Surface Antigen, Inactivated, Adjuvanted with MF59C.1 (Seqirus UK Limited, Maidenhead, UK)
Form: Liquid solution
Dose: 0.25 mL
Route: IM

### 1.6.5.1—Composition of Control Product 2

Each 0.25 mL dose contains 7.5 μg of HA for each of the following strains:

### Strains are Based on WHO (NACI in Canada) Recommendations for the 2018-2019 NH Influenza Season.

TABLE 15
Active substances of Control Product 2:

| | |
|---|---|
| A/Singapore/GP1908/2015 IVR-180 (H1N1) | 7.5 μg HA |
| (A/Michigan/45/2015 [H1N1] pdm09-like virus) | |
| A/Singapore/INFIMH-16-0019/2016, IVR-186 (H3N2) | 7.5 μg HA |
| B/Maryland/15/2016 (B/Colorado/06/2017-like virus, | 7.5 μg HA |
| Victoria lineage) | |

TABLE 16
Excipients of Control Product 2

| | |
|---|---|
| Sodium chloride | 2.00 mg |
| Potassium chloride | 0.05 mg |
| Potassium dihydrogen phosphate | 0.05 mg |
| Disodium phosphate dehydrate | 0.335 mg |
| Magnesium chloride hexahydrate | 0.025 mg |
| Calcium chloride dihydrate | 0.03 mg |
| Water for injection | qs to appropriate volume |

FLUAD® Pediatric is formulated with the adjuvant MF59, an oil-in-water emulsion of squalene oil.

FLUAD® Pediatric does not contain a preservative.

1.6.5.2—Preparation and Administration of Control Product 2

FLUAD® Pediatric was to be prepared and administered according to manufacturer's product monograph (FLUAD Pediatric® [product Monograph]. Seqirus UK Limited (Maidenhead, UK)).

1.6.5.3—Dose Selection and Timing of Control Product 2

FLUAD® Pediatric is administered to a randomized set of subjects in Group 8 as a single 0.25 mL dose at V01 and V03.

### 1.6.6—Product Storage

At the site, products are kept in a secure place with restricted access. Vaccines are stored in a refrigerator at a temperature ranging from +2° C. to +8° C., and not to be frozen. The temperature is monitored and documented for the entire time that the vaccine is at the study site. In case of accidental freezing or disruption of the cold chain, vaccines are not be administered and must be quarantined.

### 1.6.7—Blinding and Code-breaking Procedures

The study is designed as a staged, modified double-blind study with the following measures to ensure to ensure the integrity of the data:
The unblinded qualified study staff member, independent of the safety evaluation and other study evaluations, administers the vaccine
The Investigators (or delegates) in charge of safety assessment, the study staff who collect the safety data, and the laboratory personnel who analyze the blood samples do not know which product was administered
The subject/parent/guardian does not know which product was administered. To maintain the blinding of the subject/parent/guardian, the vaccine syringe label is covered with appropriate materials prior to administration.

The Investigator responsible for safety assessment does not attend the vaccination session but is available in case of emergency (e.g., anaphylactic shock).

Dose numbers are used to identify each vaccine syringe for the purpose of randomization, vaccination, and the recording of vaccine administered. Dose numbers are randomly assigned to QIV-HD and the commercial vaccines syringes. The IRT vendor is responsible for providing the subject identification and dose number to be received by the enrolled subject. The subject/parent/guardian, the Investigator, and study staff members who collect the safety data and laboratory personnel who analyze the blood samples are blinded to the group assignment. The individual responsible for preparing/administering vaccine is not be authorized to collect any safety/serology data.

The code may be broken in the event of an AE only when the identification of the vaccine received could influence the treatment of the subject. Code-breaking is limited to the subject(s) experiencing the AE.

### 1.6.8—Randomization and Allocation Procedures

For the randomization of dose numbers, the Sponsor or designee supplies a computer-generated randomization list, which is used for the labeling and packaging.

The study is randomized and modified double-blinded for all study groups. On the day of enrollment, subjects who meet all of the inclusion criteria and do not fulfill any of exclusion criteria and complete the informed consent process are randomly assigned to receive:
either QIV-HD at 30 μg or 45 μg HA/strain/dose or Fluarix® Quadrivalent in a 3:3:1 ratio for previously influenza vaccinated and unvaccinated subjects 9 to 17 years of age enrolled during Stage 1 in the US.
either QIV-HD at 60 μg HA/strain/dose or Fluarix® Quadrivalent in a 1:1 ratio for previously influenza vaccinated and unvaccinated subjects 9 to 17 years of age enrolled during Stage 1 in the US.
either QIV-HD at 30 μg HA/strain/dose or Fluarix® Quadrivalent in a 3:1 ratio for previously influenza vaccinated and unvaccinated subjects 36 months to 8 years of age enrolled during Stage 1 in the US.
either QIV-HD at 45 μg HA/strain/dose or Fluarix® Quadrivalent in a 3:1 ratio for previously influenza vaccinated and unvaccinated subjects 36 months to 8 years of age enrolled during Stage 2 in the US.
either QIV-HD at 30 μg HA/strain/dose or Fluarix® Quadrivalent in a 3:1 ratio for previously influenza vaccinated and unvaccinated subjects 6 to less than 36 months of age enrolled during Stage 2 in the US.
either QIV-HD at 60 μg HA/strain/dose (highest dosage of QIV-HD) or Fluarix® Quadrivalent in a 1:1 ratio for previously influenza vaccinated and unvaccinated subjects 36 months to 8 years of age enrolled during Stage 3 in the US.
either QIV-HD at the highest dosage with acceptable safety review or Fluarix® Quadrivalent in a 1:1 ratio for previously vaccinated subjects 6 to 36 months of age enrolled in Stage 4 in the US.
either QIV-HD at the highest dosage with acceptable safety review or Fluarix® Quadrivalent in a 1:1 ratio for previously unvaccinated subjects 6 to 36 months of age enrolled in Stage 4 in the US.
either QIV-HD at the highest dosage with acceptable safety review or FLUAD Pediatric in a 1:1 ratio for previously unvaccinated subjects 6 to less than 24 months of age enrolled in Stage 4 in Canada.

Site staff connects to the IRT, enters the identification and security information, and confirms a minimal amount of data in response to IRT prompts. The IRT provides the group assignment and have the site staff confirm it. Stratified randomization is applied for all subjects enrolled in each group stratified by previous influenza vaccination status and country. Site is not a strata for randomization due to the small sample size in each group. The allocation ratios between investigational HD vaccines to the comparators are: 3:1 ratio in groups 1, 2, 3, 5, 9, and 10; 3:3:1 ratio in group 12; and 1:1 ratio in groups 4, 6, 7, 8, 11, and 13. If the subject is not eligible to participate in the study, then the information is only recorded on the subject recruitment log.

TABLE 16A
Randomization schedule

| | | | | Number of | |
|---|---|---|---|---|---|
| | | | Previous | participants | |
| | | | vaccination | to be | |
| Stage | Age group | Country | status | randomized | Randomization |
| 1 | 9 | US | V/U | 70 | 3:3:1 to QIV-HD 30 μg, QIV- |
| | through <18 y | | | | HD 45 μg, or QIV-SD |
| | 9 | US | V/U | 60 | 1:1 to QIV-HD 60 μg or QIV- |
| | through <18 y | | | | SD |
| | 5 | US | V/U | 40 | 3:1 to QIV-HD 30 μg or QIV - |
| | through <9 y | | | | SD |
| | 3 | US | V/U | 40 | 3:1 to QIV-HD 30 μg or QIV- |
| | through <5 y | | | | SD |
| 2 | 5 | US | V/U | 40 | 3:1 to QIV-HD 45 μg or QIV- |
| | through <9 y | | | | SD |
| | 3 | US | V/U | 40 | 3:1 to QIV-HD 45 μg or QIV- |
| | through <5 y | | | | SD |
| | 6 mo | US | V/U | 40 | 3:1 to QIV-HD 30 μg or QIV- |
| | through <3 y | | | | SD |
| 3 | 5 | US | V/U | 60 | 1:1 to QIV-HD 60 μg or QIV- |
| | through <9 y | | | | SD |
| | 3 | US | V/U | 90 | 1:1 to QIV-HD 60 μg or QIV- |
| | through <5 y | | | | SD |
| | 6 mo | US | V/U | 40 | 3:1 to QIV-HD 45 μg or QIV- |
| | through <3 y | | | | SD |
| 4 | 6 mo | US | U | 60 | 1:1 to QIV-HD at the highest |
| | through <3 y | | | | dose with acceptable safety or |
| | | | | | QIV-SD |
| | 6 mo | US | V | 60 | 1:1 to QIV-HD at the highest |
| | through <3 y | | | | dose with acceptable safety or |
| | | | | | QIV-SD |
| | 6 mo | Canada | U | 60 | 1:1 to QIV-HD at the highest |
| | through <2 y | | | | dose with acceptable safety or |
| | | | | | adjuvanted TIV |

U, previously unvaccinated;
V, previously vaccinated

### 1.6.9—Concomitant Medications and Other Therapies

At the time of enrollment, ongoing medications and other therapies (e.g., blood products) are recorded in the source document as well as new medications prescribed for new medical conditions/AEs during study participation.

Reportable medications are collected in the CRB from the day of first vaccination to the end of the solicited and unsolicited follow-up period (from D0 to D28 for subjects 9 to 17 years of age and previously influenza vaccinated subjects 6 months to 8 years of age and from D0 to D56 for previously influenza unvaccinated subjects 6 months to 8 years of age).

Reportable medications include medications that impact or may impact the consistency of the safety information collected after any vaccination and/or the immune response to vaccination. Three standard categories of reportable medications are defined:
Category 1: medications impacting or that may have an impact on the evaluation of the safety (e.g., antipyretics, analgesics, and non-steroidal anti-inflammatory drugs [NSAIDs]).
Category 2: medications impacting or that may have an impact on the immune response (e.g., other vaccines, blood products, immune-suppressors, immune-modulators with immunosuppressive properties, anti-proliferative drugs such as DNA synthesis inhibitors).
Category 3: medications impacting or that may have an impact on both the safety and the immune response (e.g., steroids/corticosteroids)

### 1.7—Management of Samples

Blood samples for the assessment of antibody responses are collected at V01 and V03 (D28 [+7 days]) for subjects 9 to 17 years of age and previously influenza vaccinated subjects 6 months to 8 years of age, and at V01, V03, and V05 for the previously influenza unvaccinated subjects 6 months to 8 years of age.

1.7.1—Sample Collection

At V01 and V03 (D28 [+7 days]) for subject 9 to 17 years of age and previously influenza vaccinated subjects 6 months to 8 years of age, and at V01, V03, and V05 for the previously influenza unvaccinated subjects 6 months to 8 years of age, 5 mL of blood is collected in tubes provided by or recommended by the Sponsor. Immediately prior to the blood draw, the staff member performing the procedure verifies the subject's identity; verifies the assigned subject's number on the pre-printed label that contains that subject's number and the sampling stage; and attaches the label to the tube. Blood is taken from the limb opposite to the one that will be used for vaccination.

Note: if 5 mL of blood sample cannot be drawn, a volume less than 5 mL can be obtained.

1.7.2—Sample Preparation

Following the blood draw, the tubes are left undisturbed, positioned vertically and not shaken, for a minimum of 1 hour and a maximum of 24 hours to allow the blood to clot. Samples can be stored at room temperature for up to 2 hours; beyond 2 hours, they must be refrigerated at a temperature of +2° C. to +8° C. after the period of clotting at room temperature and must be centrifuged within a maximum of 24 hours.

After centrifugation, the serum is transferred to the appropriate number of aliquoting tubes. These tubes are pre-labeled with adhesive labels that identify the study code, the subject's number and the sampling stage or visit number.

1.7.3—Sample Storage and Shipment

During storage, serum tubes are kept in a freezer whose temperature is set and maintained at −20° C. or below. The temperature is monitored and documented on the appropriate form during the entire study. Sera are shipped frozen, using dry ice to maintain them in a frozen state, in the packaging container provided by the carrier.

### 1.8—Endpoints and Assessment Methods

### 1.8.1—Safety Definitions

The following definitions are taken from the ICH E2A Guideline for Clinical Safety Data Management: Definitions and Standards for Expedited Reporting.

Adverse Event (AE):

An AE is any untoward medical occurrence in a patient or in a clinical investigation subject administered a medicinal product and which does not necessarily have a causal relationship with this treatment. An AE can therefore be any unfavorable and unintended sign (including an abnormal laboratory finding, for example), symptom or disease temporally associated with the use of a medicinal product, whether or not considered related to the medicinal product.

Therefore, an AE may be:
A new illness
The worsening of a pre-existing condition
An effect of the vaccination, including the comparator
A combination of the above

All AEs include serious and non-serious AEs.

Surgical procedures are not AEs; they are the actions taken to treat a medical condition. It is the condition leading to the action taken that is the AE (if it occurs during the study period).

Pre-existing medical conditions are not to be reported as AEs. However, if a pre-existing medical condition worsens following study interventions in frequency or intensity, or if according to the Investigator there is a change in its clinical significance, this change is reported as an AE (exacerbation). This applies equally to recurring episodes of pre-existing conditions (e.g., asthma) if the frequency or intensity increases post-vaccination.

Serious Adverse Event (SAE):

Serious and severe are not synonymous. The term severe is often used to describe the intensity of a specific event as corresponding to Grade 3. This is not the same as serious which is based on subject/event outcome or action criteria usually associated with events that pose a threat to a subject's life or functioning. Seriousness, not severity, serves as a guide for defining regulatory reporting obligations.

An SAE is any untoward medical occurrence that at any dose
Results in death
Is life-threatening (The term “life-threatening” refers to an event in which the subject was at risk of death at the time of the event; it does not refer to an event which hypothetically might have caused death if it were more severe)
Requires inpatient hospitalization or prolongation of existing hospitalization (All medical events leading to hospitalizations will be recorded and reported as SAEs, with the exception of: hospitalization planned before inclusion into the study or outpatient treatment with no hospitalization)
Results in persistent or significant disability/incapacity (“Persistent or significant disability or incapacity” means that there is a substantial disruption of a person's ability to carry out normal life functions)
Is a congenital anomaly/birth defect
Is an important medical event (IME).

Medical and scientific judgment should be exercised in deciding whether expedited reporting is appropriate in other situations, such as IMEs that may not be immediately life-threatening or result in death or hospitalization but may jeopardize the health of the subject or may require intervention to prevent one of the other outcomes listed in the definition above. These IMEs should also usually be considered serious. Examples of such events include allergic bronchospasm requiring intensive treatment in an emergency room or at home, blood dyscrasias or convulsions that do not result in inpatient hospitalization, or the development of drug dependency or drug abuse, new-onset diabetes, or autoimmune disease.

Adverse Reaction:

All noxious and unintended responses to a medicinal product related to any dose should be considered adverse reactions (AR).

(The phrase “responses to a medicinal product” means that a causal relationship between a medicinal product and an AE is at least a reasonable possibility)

The following additional definitions are used:

Immediate Event/Reaction:

Immediate events are recorded to capture medically relevant unsolicited systemic AEs (including those related to the product administered) that occur within the first 30 minutes after vaccination.

Solicited Reaction:

A solicited reaction is an “expected” adverse reaction (sign or symptom) observed and reported under the conditions (nature and onset) prelisted in the protocol and CRB (e.g., injection site pain or headache occurring between D0 and D7 post-vaccination).

By definition, solicited reactions are to be considered as being related to the product administered.

For injectable vaccines, solicited reactions can either be solicited injection site reactions or solicited systemic reactions.

Unsolicited AE/AR:

An unsolicited AE is an observed AE that does not fulfill the conditions prelisted in the CRB in terms of diagnosis and/or onset window post-vaccination. For example, if headache between D0 and D7 is a solicited reaction (i.e., prelisted in the protocol and CRB), then a headache starting on D7 is a solicited reaction, whereas headache starting on D8 post-vaccination is an unsolicited AE. Unsolicited AEs includes both serious (SAEs) and non-serious unsolicited AEs.

Injection Site Reaction:

An injection site reaction is an AR at and around the injection site. Injection site reactions are commonly inflammatory reactions. They are considered to be related to the product administered.

TABLE 16B
Grading of solicited injection-site reactions

| Age group | Reaction | Grade 1 | Grade 2 | Grade 3 |
|---|---|---|---|---|
| 6 months | Tenderness | Minor reaction | Crying or | Crying when |
| through <3 | | when injection | protesting when | injected limb is |
| years | | site is touched | injection site is | moved or reduced |
| | | | touched | mobility of the |
| | | | | injected limb |
| | Erythema | >0 to <25 mm | 25 to <50 mm | ≥50 mm |
| | Swelling | | | |
| | Induration | | | |
| | Bruising | | | |
| 3 | Pain | Easily tolerated | Interferes with | Makes it |
| through <12 | | | normal behavior | impossible to |
| years | | | or activities | perform usual |
| | | | | activities |
| | Erythema | >0 to <25 mm | 25 to <50 mm | ≥50 mm |
| | Swelling | | | |
| | Induration | | | |
| | Bruising | | | |
| 12 | Pain | Transient | Alleviated with | Interrupts usual |
| through <18 | | | additional | activities of |
| years | | | therapeutic | daily living, or |
| | | | intervention | significantly |
| | | | | affects clinical |
| | | | | status, or requires |
| | | | | intensive therapeutic |
| | | | | intervention |
| | Erythema | 25 to 50 mm | 51 to 100 mm | >100 mm |
| | Swelling | | | |
| | Induration | | | |
| | Bruising | | | |

Systemic AE:

Systemic AEs are all AEs that are not injection or administration site reactions. They therefore include systemic manifestations such as headache, fever, as well as localized or topical manifestations that are not associated with the vaccination or administration site (e.g., erythema that is localized but that is not occurring at the injection site).

TABLE 16C
Grading of solicited systemic reactions

| Age group | Reaction | Grade 1 | Grade 2 | Grade 3 |
|---|---|---|---|---|
| 6 months | Fever | 38.0° C. | >38.5° C. | >39.5° C. |
| through <3 | | to 38.5° C. | to 39.5° C. | or >103.1° F. |
| years | | or 100.4° F. | or >101.3° F. | |
| | | to 101.3° F. | to 103.1° F. | |
| | Vomiting | 1 episode | 2 to 5 episodes | ≥6 episodes |
| | | per 24 hours | per 24 hours | per 24 hours |
| | | | | or requiring |
| | | | | parenteral |
| | | | | hydration |
| | Abnormal | <1 hour | 1 to 3 hours | >3 hours |
| | crying | | | |
| | Drowsiness | Sleepier than | Not interested in | Sleeping most |
| | | usual or less | surroundings or | of the time or |
| | | interested in | did not wake up | difficult to |
| | | surroundings | for a feed/meal | wake up |
| | Appetite loss | Eating less than | Missing 1 or 2 | Refusing ≥3 |
| | | normal | feeds/meals | feeds/meals |
| | | | completely | |
| | Irritability | Easily consoled | Requiring | Inconsolable |
| | | | increased | |
| | | | attention | |
| 3 | Fever | 38.0° C. | 38.5° C. | ≥39.0° C. |
| through <18 | | to 38.4° C. | to 38.9° C. | or ≥102.1° F. |
| years | | or 100.4° F. | or 101.2° F. | |
| | | to 101.1° F. | to 102.0° F. | |
| | Headache | Transient and | Alleviated with | Interrupts usual |
| | Malaise | requires only | additional | activities of |
| | Myalgia | minimal | therapeutic | daily living, or |
| | Shivering | treatment or | intervention | significantly |
| | | therapeutic | | affects clinical |
| | | intervention | | status, or |
| | | | | requires intensive |
| | | | | therapeutic |
| | | | | intervention |

Adverse Event of Special Interest (AESI):

An adverse event of special interest is one of scientific and medical concern specific to the Sponsor's product or program, for which ongoing monitoring and rapid communication by the investigator to the sponsor can be appropriate. Such an event might warrant further investigation in order to characterize and understand it. Depending on the nature of the event, rapid communication by the study Sponsor to other parties (e.g., regulators) might also be warranted.

1.8.2—Safety Endpoints

The endpoints for the evaluation of safety are:
Occurrence, nature (Medical Dictionary for Regulatory Activities [MedDRA] preferred term [PT]), duration, intensity, and relationship to vaccination of any unsolicited systemic AEs reported in the 30 minutes after each vaccination.
Occurrence, time to onset, number of days of occurrence, intensity, action taken, and whether the reaction led to early termination from the study, of solicited (prelisted in the subject's diary card and CRB) injection site reactions and systemic reactions occurring up to 7 days after each vaccination.
Occurrence, nature (MedDRA system organ class [SOC] and PT), time to onset, duration, intensity, relationship to vaccination (for systemic AEs only), and whether the event led to early termination from the study, of unsolicited AEs up to 28 days after each vaccination.
Occurrence, nature (MedDRA SOC and PT), time to onset, seriousness criteria, relationship to vaccination, outcome, and whether the event led to early termination from the study, of SAEs (including AESIs) throughout the study.
Occurrence, nature (MedDRA PT), time to onset, and relationship to vaccination of AESIs throughout the study.

1.8.3—Safety Assessment Methods

At V01, the Investigator or a delegate performs a clinical or medically-driven physical examination.

At V03 (D28 [+7 days]), and/or V05, as applicable, the Investigator or a delegate performs a targeted clinical or medically-driven physical examination, as necessary, and asks the subject/parent/guardian about any solicited reactions and unsolicited AEs recorded in the diary card, as well as about any other AEs that may have occurred since the previous visit.

1.8.3.1—Immediate Post-vaccination Observation Period

Subjects are kept under observation for 30 minutes after each vaccination to ensure their safety. Any AE that occurs during this period is noted on the source document and recorded in the CRB, as follows:
Unsolicited systemic AEs are recorded as immediate AEs in the CRB (presence marked as “yes” and details collected).
Solicited and unsolicited injection site reactions and solicited systemic reactions are recorded in the CRB in the same way as any reactions starting on the day of vaccination.
SAEs are recorded in the CRB and reported to the Sponsor in the same way as any other SAEs.

1.8.3.2—Reactogenicity (Solicited Reactions From Day 0 to Day 7 After Each Vaccination)

After each vaccination, subjects/parents/guardians are provided with a diary card, a digital thermometer, and a flexible ruler, and are instructed how to use them. The following items are recorded by the subjects in the diary card on the day of vaccination and for the next 7 days (i.e., D0 to D7) until resolution:
Daily temperature, with the route by which it was taken
Daily measurement or intensity grade of all other solicited injection site and systemic reactions
Action taken for each event (e.g., medication)

The action(s) taken by the subject/parent/guardian to treat and/or manage any solicited reactions is classified in the CRB using the following list (all applicable items should be checked):
None
Medication
Health care provider contact
Hospitalized
Discontinuation of study vaccination

The below tables present, respectively, the injection site reactions and systemic reactions that are prelisted in the diary cards and CRB, together with the intensity scales.

TABLE 17A
Solicited injection site reactions for infants and toddlers aged <36
months: terminology, definitions, and intensity scales

| CRB term | Injection site | Injection site | Injection site | Injection site | Injection site |
|---|---|---|---|---|---|
| (MedDRA LLT) | tenderness | erythema | swelling | induration | bruising |
| MedDRA PT | Injection site | Injection site | Injection site | Injection site | Injection site |
| | pain | erythema | swelling | induration | bruising |
| Diary card | Tenderness | Redness | Swelling | Hardening | Bruising |
| term | | | | | |
| Definition | Pain when | Presence of | Swelling at or | Hardening at or | Bruising is |
| | the injection | a redness | near the | near the | the result of |
| | site is | including the | injection site | injection site. | the diffusion |
| | touched or | approximate | Swelling or | Hardening | of blood in |
| | injected limb | point of | edema is caused | is caused | the skin from |
| | mobilized | needle entry | by a fluid | by a slow | ruptured |
| | | | infiltration in | diffusion of | blood vessels |
| | | | tissue or cavity | the product | that forms a |
| | | | and, depending | in the tissue | purple or black |
| | | | on the space | leading to | and blue spot |
| | | | available for the | a thick or | on the skin. |
| | | | fluid to disperse, | hard area | It can be |
| | | | swelling may be | to touch at | best described |
| | | | either soft | or near the | by looking at |
| | | | (typically) or | injection | its size. |
| | | | firm (less | site and thus | |
| | | | typical) to touch | can be best | |
| | | | and thus can be | described | |
| | | | best described | by looking at | |
| | | | by looking at | the size of the | |
| | | | the size of the | hardening. | |
| | | | swelling | | |
| Intensity | Grade 1: | Grade 1: >0 | Grade 1: >0 | Grade 1: >0 | Grade 1: >0 |
| scale* | Minor reaction | to <25 mm | to <25 mm | to <25 mm | to <25 mm |
| | when injection | Grade 2: ≥25 | Grade 2: ≥25 | Grade 2: ≥25 | Grade 2: ≥25 |
| | site is touched | to <50 mm | to <50 mm | to <50 mm | to <50 mm |
| | Grade 2: | Grade 3: ≥50 | Grade 3: ≥50 | Grade 3: ≥50 | Grade 3: ≥50 |
| | Cries or protests | mm | mm | mm | mm |
| | when injection | | | | |
| | site is touched | | | | |
| | Grade 3: | | | | |
| | Cries when | | | | |
| | injected limb | | | | |
| | is mobilized, | | | | |
| | or the movement | | | | |
| | of the injected | | | | |
| | limb is reduced | | | | |

*For the subjective reaction of tenderness, parents/guardians record the intensity level (Grade 1, 2, or 3) in the diary card. For the measurable reactions of redness, swelling, hardening, and bruising, they record just the size of the reaction, and the classification as Grade 1, 2, or 3 is assigned at the time of the statistical analysis.

TABLE 17B
Solicited injection site reactions for children aged 3 to
11 years: terminology, definitions, and intensity scales

| CRB term | Injection site | Injection site | Injection site | Injection site | Injection site |
|---|---|---|---|---|---|
| (MedDRA LLT) | pain | erythema | swelling | induration | bruising |
| MedDRA PT | Injection site | Injection site | Injection site | Injection site | Injection site |
| | pain | erythema | swelling | induration | bruising |
| Diary card | Pain | Redness | Swelling | Hardening | Bruising |
| term | | | | | |
| Definition | Pain either | Presence of | Swelling at or | Hardening at or | Bruising is |
| | present | a redness | near the | near the | the result of |
| | spontaneously | including the | injection site | injection site. | the diffusion |
| | or when the | approximate | Swelling or | Hardening | of blood in |
| | injection site | point of | edema is caused | is caused | the skin from |
| | is touched or | needle entry | by a fluid | by a slow | ruptured |
| | injected limb | | infiltration in | diffusion of | blood vessels |
| | is mobilized | | tissue or cavity | the product | that forms a |
| | | | and, depending | in the tissue | purple or black |
| | | | on the space | leading to | and blue spot |
| | | | available for the | a thick or | on the skin. |
| | | | fluid to disperse, | hard area | It can be |
| | | | swelling may be | to touch at | best described |
| | | | either soft | or near the | by looking at |
| | | | (typically) or | injection | its size. |
| | | | firm (less | site and thus | |
| | | | typical) to touch | can be best | |
| | | | and thus can be | described | |
| | | | best described | by looking at | |
| | | | by looking at | the size of the | |
| | | | the size of the | hardening. | |
| | | | swelling | | |
| Intensity | Grade 1: | Grade 1: >0 | Grade 1: >0 | Grade 1: >0 | Grade 1: >0 |
| scale* | Easily | to <25 mm | to <25 mm | to <25 mm | to <25 mm |
| | tolerated | Grade 2: ≥25 | Grade 2: ≥25 | Grade 2: ≥25 | Grade 2: ≥25 |
| | Grade 2: | to <50 mm | to <50 mm | to <50 mm | to <50 mm |
| | Sufficiently | Grade 3: ≥50 | Grade 3: ≥50 | Grade 3: ≥50 | Grade 3: ≥50 |
| | discomforting | mm | mm | mm | mm |
| | to interfere | | | | |
| | with normal | | | | |
| | behavior or | | | | |
| | activities | | | | |
| | Grade 3: | | | | |
| | Incapacitating, | | | | |
| | unable to | | | | |
| | perform usual | | | | |
| | activities | | | | |

*For the subjective reaction of pain, parents/guardians record the intensity level (Grade 1, 2, or 3) in the diary card. For the measurable reactions of redness, swelling, hardening, and bruising, they record just the size of the reaction, and the classification as Grade 1, 2, or 3 is assigned at the time of the statistical analysis.

TABLE 17C
Solicited injection site reactions for children aged 12 to
17 years: terminology, definitions, and intensity scales

| CRB term | Injection site | Injection site | Injection site | Injection site | Injection site |
|---|---|---|---|---|---|
| (MedDRA LLT) | pain | erythema | swelling | induration | bruising |
| MedDRA PT | Injection site | Injection site | Injection site | Injection site | Injection site |
| | pain | erythema | swelling | induration | bruising |
| Diary card | Pain | Redness | Swelling | Hardening | Bruising |
| term | | | | | |
| Definition | Pain either | Presence of | Swelling at or | Hardening at or | Bruising is |
| | present | a redness | near the | near the | the result of |
| | spontaneously | including the | injection site | injection site. | the diffusion |
| | or when the | approximate | Swelling or | Hardening | of blood in |
| | injection site | point of | edema is caused | is caused | the skin from |
| | is touched or | needle entry | by a fluid | by a slow | ruptured |
| | injected limb | | infiltration in | diffusion of | blood vessels |
| | is mobilized | | tissue or cavity | the product | that forms a |
| | | | and, depending | in the tissue | purple or black |
| | | | on the space | leading to | and blue spot |
| | | | available for the | a thick or | on the skin. |
| | | | fluid to disperse, | hard area | It can be |
| | | | swelling may be | to touch at | best described |
| | | | either soft | or near the | by looking at |
| | | | (typically) or | injection | its size. |
| | | | firm (less | site and thus | |
| | | | typical) to touch | can be best | |
| | | | and thus can be | described | |
| | | | best described | by looking at | |
| | | | by looking at | the size of the | |
| | | | the size of the | hardening. | |
| | | | swelling | | |
| Intensity | Grade 1: A type of | Grade 1: ≥25 | Grade 1: ≥25 | Grade 1: ≥25 | Grade 1: ≥25 |
| scale* | adverse event that | to ≤50 mm | to ≤50 mm | to ≤50 mm | to ≤50 mm |
| | is usually transient | Grade 2: ≥51 | Grade 2: ≥51 | Grade 2: ≥51 | Grade 2: ≥51 |
| | and may require | to ≤100 mm | to ≤100 mm | to ≤100 mm | to ≤100 mm |
| | only minimal | Grade 3: >100 | Grade 3: >100 | Grade 3: >100 | Grade 3: >100 |
| | treatment or | mm | mm | mm | mm |
| | therapeutic | | | | |
| | intervention. The | | | | |
| | event does not | | | | |
| | generally interfere | | | | |
| | with usual | | | | |
| | activities of daily | | | | |
| | living. | | | | |
| | Grade 2: A type of | | | | |
| | adverse event that | | | | |
| | is usually | | | | |
| | alleviated with | | | | |
| | additional | | | | |
| | therapeutic | | | | |
| | intervention. The | | | | |
| | event interferes | | | | |
| | with usual | | | | |
| | activities of daily | | | | |
| | living, causing | | | | |
| | discomfort but | | | | |
| | poses no | | | | |
| | significant or | | | | |
| | permanent risk of | | | | |
| | harm to the | | | | |
| | research | | | | |
| | participant. | | | | |
| | Grade 3: A type of | | | | |
| | adverse event that | | | | |
| | interrupts usual | | | | |
| | activities of daily | | | | |
| | living, or | | | | |
| | significantly | | | | |
| | affects clinical | | | | |
| | status, or may | | | | |
| | require intensive | | | | |
| | therapeutic | | | | |
| | intervention. | | | | |

*For the subjective reaction of pain, subjects/parents/guardians record the intensity level (Grade 1, 2, or 3) in the diary card. For the measurable reactions of redness, swelling, hardening, and bruising they record just the size of the reaction, and the classification as Grade 1, 2, or 3 is assigned at the time of the statistical analysis.

TABLE 18
Solicited systemic reactions for infants and toddlers aged <36
months: terminology, definitions, and intensity scales

| CRB term | | | Crying | | Appetite | |
|---|---|---|---|---|---|---|
| (MedDRA LLT) | Fever | Vomiting | abnormal | Drowsiness | lost | Irritability |
| MedDRA PT | Pyrexia | Vomiting | Crying | Somnolence | Decreased | Irritability |
| | | | | | appetite | |
| Diary card | Temperature | Vomiting | Abnormal | Drowsiness | Loss of | Irritability |
| term | | | crying | | appetite | |
| Definition | Elevation of | Vomiting | Inconsolable | Reduced | See | An excessive |
| | temperature | does not | crying | interest in | intensity | response to |
| | to ≥°38.0° C. | include | without a | surroundings, | scale | stimuli: |
| | (≥100.4° F.) | spitting up | determined | or increased | | increased |
| | | | reason | sleeping | | fussiness, |
| | | | | | | whining, and |
| | | | | | | fretfulness |
| | | | | | | despite |
| | | | | | | attempts to |
| | | | | | | comfort the |
| | | | | | | infant and |
| | | | | | | despite |
| | | | | | | caregiver |
| | | | | | | responses |
| | | | | | | that would |
| | | | | | | normally be |
| | | | | | | soothing |
| Intensity | Grade 1: ≥38.0° C. | Grade 1: 1 | Grade 1: <1 | Grade 1: | Grade 1: | Grade 1: |
| scale* | to ≤38.5° C. | episode per | hour | Sleepier than | Eating less | Easily |
| | or ≥100.4° F. | 24 hours | Grade 2: 1-3 | usual or less | than normal | consolable |
| | to ≤101.3° F. | Grade 2: 2-5 | hours | interested in | Grade 2: | Grade 2: |
| | Grade 2: >38.5° C. | episodes per | Grade 3: >3 | surroundings | Missed 1 | Requiring |
| | to ≤39.5° C. | 24 hours | hours | Grade 2: Not | or 2 feeds/ | increased |
| | or >101.3° F. | Grade 3: ≥6 | | interested in | meals | attention |
| | to ≤103.1° F. | episodes per | | surroundings | completely | Grade 3: |
| | Grade 3: >39.5° C. | 24 hours or | | or did not | Grade 3: | Inconsolable |
| | or >103.1° F. | requiring | | wake up for a | Refuses ≥3 | |
| | | parenteral | | feed/meal | feeds/meals | |
| | | hydration | | Grade 3: | or refuses | |
| | | | | Sleeping | most feeds/ | |
| | | | | most of the | meals | |
| | | | | time or | | |
| | | | | difficult to | | |
| | | | | wake up | | |

*For all reactions but fever, parents/guardians record the intensity level (Grade 1, 2, or 3) in the diary card. For fever, they record the body temperature, and the classification as Grade 1, 2, or 3 is assigned at the time of the statistical analysis based on the unit used to measure the temperature and the intensity scale.

TABLE 19
Solicited systemic reactions for children aged 3 to 17 years: terminology, definitions, and intensity scales

| CRB term | | | | | |
|---|---|---|---|---|---|
| (MedDRA LLT) | Fever | Headache | Malaise | Myalgia | Shivering |
| MedDRA PT | Pyrexia | Headache | Malaise | Myalgia | Chills |
| Diary card | Temperature | Headache | Feeling | Muscle aches | Chills |
| term | | | unwell | and pains | |
| Definition | Elevation of | Pain or | General ill | Muscle aches | Cold feeling. |
| | temperature | discomfort in | feeling. | and pains are | |
| | to ≥°38.0° C. | the head or | Malaise is a | common and | |
| | (≥100.4° F.) | scalp. Does | generalized | can involve | |
| | | not include | feeling of | more than | |
| | | migraine. | discomfort, | one muscle at | |
| | | | illness, or | the same | |
| | | | lack of well- | time. Muscle | |
| | | | being that can | pain can also | |
| | | | be associated | involve the | |
| | | | with a disease | soft tissues | |
| | | | state. It can | that surround | |
| | | | be accompanied | muscles. | |
| | | | by a sensation | These structures, | |
| | | | of exhaustion | which are often | |
| | | | or inadequate | referred to as | |
| | | | energy to | connective | |
| | | | accomplish | tissues, include | |
| | | | usual | ligaments, | |
| | | | activities. | tendons, and | |
| | | | | fascia (thick | |
| | | | | bands of tendons). | |
| | | | | Does not apply | |
| | | | | to muscle pain | |
| | | | | at the | |
| | | | | injection site | |
| | | | | which should | |
| | | | | be reported as | |
| | | | | injection site | |
| | | | | pain. | |
| Intensity | Grade 1: ≥38.0° C. | Grade 1: A | Grade 1: A | Grade 1: A | Grade 1: A |
| scale* | to ≤38.4° C., | type of AE | type of AE | type of AE | type of AE |
| | or ≥100.4° F. | that is usually | that is usually | that is usually | that is usually |
| | to ≤101.1° F. | transient and | transient and | transient and | transient and |
| | | may require | may require | may require | may require |
| | | only minimal | only minimal | only minimal | only minimal |
| | | treatment or | treatment or | treatment or | treatment or |
| | | therapeutic | therapeutic | therapeutic | therapeutic |
| | | intervention. | intervention. | intervention. | intervention. |
| | | The event | The event | The event | The event |
| | | does not | does not | does not | does not |
| | | generally | generally | generally | generally |
| | | interfere with | interfere with | interfere with | interfere with |
| | | usual | usual | usual | usual |
| | | activities of | activities of | activities of | activities of |
| | | daily living. | daily living. | daily living. | daily living. |
| | Grade 2: ≥38.5° C. | Grade 2: A | Grade 2: A | Grade 2: A | Grade 2: A |
| | to ≤38.9° C., | type of AE | type of AE | type of AE | type of AE |
| | or ≥101.2° F. | that is usually | that is usually | that is usually | that is usually |
| | to ≤102.0° F. | alleviated with | alleviated with | alleviated with | alleviated with |
| | | additional | additional | additional | additional |
| | | therapeutic | therapeutic | therapeutic | therapeutic |
| | | intervention. | intervention. | intervention. | intervention. |
| | | The event | The event | The event | The event |
| | | interferes | interferes | interferes | interferes |
| | | with usual | with usual | with usual | with usual |
| | | activities of | activities of | activities of | activities of |
| | | daily living, | daily living, | daily living, | daily living, |
| | | causing | causing | causing | causing |
| | | discomfort | discomfort | discomfort | discomfort |
| | | but poses no | but poses no | but poses no | but poses no |
| | | significant or | significant or | significant or | significant or |
| | | permanent | permanent | permanent | permanent |
| | | risk of harm | risk of harm | risk of harm | risk of harm |
| | | to the | to the | to the | to the |
| | | research | research | research | research |
| | | participant. | participant. | participant. | participant. |
| | Grade 3: | Grade 3: A | Grade 3: A | Grade 3: A | Grade 3: A |
| | ≥39.0° C. | type of AE | type of AE | type of AE | type of AE |
| | or ≥102.1° F. | that interrupts | that interrupts | that interrupts | that interrupts |
| | | usual activities | usual activities | usual activities | usual activities |
| | | of daily | of daily | of daily | of daily |
| | | living, or | living, or | living, or | living, or |
| | | significantly | significantly | significantly | significantly |
| | | affects | affects | affects | affects |
| | | clinical | clinical | clinical | clinical |
| | | status, or may | status, or may | status, or may | status, or may |
| | | require | require | require | require |
| | | intensive | intensive | intensive | intensive |
| | | therapeutic | therapeutic | therapeutic | therapeutic |
| | | intervention. | intervention. | intervention. | intervention. |

*For all reactions but fever, subjects/parents/guardians record the intensity level (Grade 1, 2, or 3) in the diary card. For fever, they record the body temperature, and the classification as Grade 1, 2, or 3 is assigned at the time of the statistical analysis based on the unit used to measure the temperature and the intensity scale.

Important notes for the accurate assessment of temperature:

Subjects/parents/guardians measure body temperature once per day, preferably always at the same time. The optimal time for measurement is the evening, when body temperature is the highest. Temperature is also to be measured at the time of any apparent fever. The observed daily temperature and the route of measurement are to be recorded in the diary card, and the highest temperature is recorded by the site in the CRB. The preferred route for this study is rectal for subjects 6 months to <4 years of age and oral for subjects 4 years of age and older. However, axillary is also acceptable for subjects 6 months of age and older if unable to obtain the preferred route of measurement. Pre-vaccination temperature is also systematically collected by the investigator on the source document. Tympanic thermometers must not be used.

1.8.3.3—Unsolicited Adverse Events

In addition to recording solicited reactions, subjects/parents/guardians are instructed to record any other medical events that may occur during the 28-day period after each vaccination. Space is provided in the diary card for this purpose.

Information on SAEs is collected and assessed throughout the study, from inclusion until 6 months after the last vaccination. Any SAE occurring at any time during the study is reported by the Investigator in the CRB according to the completion instructions provided by the Sponsor; this includes checking the “Serious” box on the AE CRF and completing the appropriate Death/Safety Complementary Information CRFs. All information concerning the SAE is reported either as part of the initial reporting or during follow-up reporting if relevant information became available later (e.g., outcome, medical history, results of investigations, copy of hospitalization reports). In case a subject experiences febrile convulsion (neurological event associating fever and seizure), the assessment is performed according to the “Guideline for definition and collection of cases of febrile convulsion”, and this event will be considered an SAE.

For each unsolicited AE (whether serious or non-serious), the following information is to be recorded:
Start and stop dates (The stop date of all related AEs is actively solicited. For other events, the investigator provides the stop date when it becomes available. AEs for which no stop date was obtained during the course of the study is considered as ongoing at the end of the study.)
Intensity of the event:
1. For measurable unsolicited AEs that are part of the list of solicited reactions, the size of the AE as well as the temperature for fever is collected and analyzed based on the corresponding scale used for solicited reactions.
2. All other unsolicited AEs are classified according to the following intensity scale:
   a. Grade 1: A type of AE that is usually transient and may require only minimal treatment or therapeutic intervention. The event does not generally interfere with usual activities of daily living.
   b. Grade 2: A type of AE that is usually alleviated with additional therapeutic intervention. The event interferes with usual activities of daily living, causing discomfort but poses no significant or permanent risk of harm to the research participant.
   c. Grade 3: A type of AE that interrupts usual activities of daily living, or significantly affects clinical status, or may require intensive therapeutic intervention.
Whether the AE was related to the investigational product (for unsolicited systemic AEs).
The Investigator assesses the causal relationship between the AE and the investigational product as either “Not related” or “Related”.
Action taken for each AE (e.g., medication)
The action(s) taken by the subject/parent/guardian to treat and/or manage any unsolicited AEs is classified in the CRB using the following list (all applicable items should be checked):
   None
   Medication
   Health care provider contact
   Hospitalized
   Discontinuation of study vaccination
Whether the AE was serious
For each SAE, the investigator completes all seriousness criteria that apply (outcome, elapsed time, and relationship to study procedures)
Whether the AE caused study discontinuation

1.8.3.4—Adverse Events of Special Interest

AESIs are captured as SAEs and are collected throughout the study. These include new onset of GBS, encephalitis/myelitis (including transverse myelitis), Bell's palsy, convulsions, optic neuritis, and brachial neuritis (Sejvar J J et al. (2011) Vaccine; 29(3):599-612; Bonhoeffer J et al. (2004) Vaccine 22(5-6):557-62; Sejvar J J et al. (2007) Vaccine 25(31):5771-92).

1.8.3.5—Assessment of Causality

The Investigator assesses the causal relationship between each unsolicited systemic AE and the product administered as either not related or related, based on the following definitions:
Not related—The AE is clearly/most probably caused by other etiologies such as an underlying condition, therapeutic intervention, or concomitant therapy; or the delay between vaccination and the onset of the AE is incompatible with a causal relationship; or the AE started before the first vaccination (screening phase, if applicable)
Related—There is a “reasonable possibility” that the AE was caused by the product administered, meaning that there is evidence or arguments to suggest a causal relationship.

Note: By convention, all AEs reported at the injection site (whether solicited or unsolicited) and all solicited systemic AEs are considered to be related to the administered product and therefore are referred to as reactions and do not require the Investigator's opinion on relatedness.

Adverse events likely to be related to the product, whether serious or not, that persist at the end of the study is followed up by the Investigator until their complete disappearance or the stabilization of the subject's condition. The Investigator informs the Sponsor of the date of final disappearance of the event or the date of “chronicity” establishment.

### 1.9 Immunogenicity

### 1.9.1 Immunogenicity Endpoints

### 1.9.1.1 Immunogenicity by HAI Method

The endpoints for the evaluation of immunogenicity by HAI method are:

For Subjects 9 to 17 Years of Age and Previously Influenza Vaccinated Subjects 6 Months to 8 Years of Age:
HAI antibody (Ab) titers obtained on D0 and D28
Individual HAI titers ratio D28/DO
Seroconversion (titer <10 [1/dilution {dil}] at D0 and post-injection titer ≥40 [1/dil] at D28, or titer ≥10 [1/dil] at D0 and a ≥4-fold increase in titer [1/dil] at D28)
Percentage of subjects with titers ≥40 (1/dil) at D0 and D28

For Previously Influenza Unvaccinated Subjects 6 Months to 8 Years of Age:
HAI Ab titers obtained on D0, D28, and D56
Individual HAI titers ratio D28/D0 and D56/DO
Seroconversion (titer <10 [1/dil] at D0 and post-injection titer ≥40 [1/dil] at D28 and D56, or titer ≥10 [1/dil] at D0 and a ≥4-fold increase in titer [1/dil] at D28 and D56)
Percentage of subjects with titers ≥40 (1/dil) at D0, D28, and D56

### 1.9.1.2 Immunogenicity by Virus SN Method

The endpoints for the evaluation of immunogenicity by virus SN method are:

For Subjects 9 to 17 Years of Age and Previously Influenza Vaccinated Subjects 6 Months to 8 Years of Age:
Individual neutralization test (NT) Ab titer on D0 and D28
Individual NT Ab titer ratio (fold increase in serum NT post-vaccination relative to D0) at D28
Subjects with NT Ab titers ≥20 (1/dil), ≥40 (1/dil), ≥80 (1/dil) at D28
Fold-increase in NT Ab titer [post/pre]≥2 and ≥4 at D28
Detectable NT (NT Ab titer ≥10 [1/dil]) at D0 and D28

For previously influenza unvaccinated subjects 6 months to 8 years of age:
Individual NT Ab titer on D0, D28, and D56
Individual NT Ab titer ratio (fold increase in serum NT post-vaccination relative to DO) at D28 and D56
Subjects with NT Ab titers ≥20 (1/dil), ≥40 (1/dil), ≥80 (1/dil) at D28 and D56
Fold-increase in NT Ab titer [post/pre]≥2 and ≥4 at D28 and D56
Detectable NT (NT Ab titer ≥10 [1/dil]) at D0, D28, and D56

### 1.9.2—Immunogenicity Assessment Methods

Anti-Influenza Virus Ab Titration by Inhibition of Hemagglutination

Test serum samples and quality control sera (sheep, ferret, and/or human sera) are incubated with Sigma Type III neuraminidase from Vibrio cholerae to eliminate non-specific inhibitors. Adsorption of spontaneous anti-species agglutinins is then performed by incubating the test serum samples and quality control sera with a red blood cell (RBC) suspension. Following this, the mixtures are centrifuged and the supernatants containing the treated sera are collected for testing. Ten two-fold dilutions (starting at 1:10) of the treated test serum samples and quality control sera are incubated with a previously titrated influenza antigen at a concentration of 4 hemagglutination unit (HAU)/25 μL. Influenza antigen is not added to the serum control wells containing only serum and RBCs. The mixture is then incubated and an RBC suspension is added. Following incubation, the results are read. The endpoint of the assay is the highest serum dilution in which complete inhibition of hemagglutination occurred. Each serum sample is titrated in two independent assay runs, and the 2 values, which cannot differ by more than 1 two-fold dilution, are reported. The GMT between the 2 values is calculated at the time of statistical analysis. The lower limit of quantitation (LLOQ) is set at the lowest dilution used in the assay, 1:10. Titers below this level are reported as <10 (1/dil). If the lowest/first serum dilution used in the assay exhibits complete inhibition of hemagglutination, the serum Ab titer is reported as <10 (1/dil). If the highest/last serum dilution used in the assay exhibits complete inhibition of hemagglutination, the serum Ab titer is reported as ≥10240 (1/dil).

Influenza Virus Neutralization Test

This NT measures Abs directed against the viral neutralization epitopes of the influenza virus, which may be different from the hemagglutination epitopes, therefore, the NT titers may be different from the HAI titers.

To measure NT, serially diluted, heat-inactivated human serum samples are pre-incubated with a fixed amount of challenge virus prior to the addition of Madin-Darby canine kidney (MDCK) cells. After overnight incubation, the viral nucleoprotein production in infected MDCK cells is measured by enzyme-linked immunosorbent assay (ELISA), using monoclonal Ab specific to either influenza A nucleoprotein or influenza B nucleoprotein. Since serum neutralizing Abs to the influenza virus inhibits the viral infection of MDCK cells, the ELISA optical density results are inversely proportional to the titers of neutralizing Ab present in the serum. The LLOQ is set at the reciprocal of the lowest dilution used in the assay, i.e., 10 (1/dil). Titers below this level are reported as <10 (1/dil). The highest titer that would be reported is 10239 (1/dil). Anything >10240 (1/dil) is pre-diluted, retested, and end point titers are reported.

### 1.10—Statistical Methods and Determination of Sample Size

1.10.1—Statistical Methods

All statistical analyses are performed under the responsibility of the Sponsor's Biostatistics Platform using the SAS® software, Version 9.4 or above (SAS Institute, Cary, N.C., USA).

Hypotheses and Statistical Methods for the Objectives

i. Hypotheses

There are no hypotheses for safety and immunogenicity. All analyses are descriptive.

ii. Statistical Methods

For the cohorts controlled by the non-adjuvanted QIV-SD, results are described per stage, according to vaccine received, and per age group. Age groups are also pooled within the same vaccine group for the main endpoints. The descriptive results may also be presented by the pooled QIV-HD group (60 μg) in the two countries. Subgroup immunogenicity analyses per previous vaccination status and/or baseline serostatus are presented when appropriate. If any of the stages are not completed, analyses for the higher dose or next stage were not performed.

For the cohort controlled by the adjuvanted TIV, results are described according to vaccine received.

Safety

Safety endpoints are analyzed descriptively for subjects in SafAS who received the QIV-HD. Solicited reactions (solicited injection site and systemic reactions), unsolicited AEs, SAEs, and AESIs are summarized. The main parameters are described by single proportions with the 95% CI (Clopper-Pearson method) (Newcombe RG (1998) Stat Med.

17:857-72).

Immunogenicity

Immunogenicity endpoints are summarized with 95% CIs. The 95% CIs for the GMTs and GMT ratios (GMTRs) are calculated using normal approximation of log-transformed titers. The 95% CIs for the proportions are based on the Clopper-Pearson method. The ratios of GMTs are obtained between groups with the 95% CIs calculated using normal approximation of log-transformed titers. The differences in the seroconversion rates between groups are computed along with the 2-sided 95% CIs by the Wilson-Score method without continuity correction (Newcombe RG (1998) Stat Med. 1998; 17:873-90). Additional parameters may be displayed as appropriate.

Reverse cumulative distribution curves against each strain are performed for baseline (V01) and post-vaccination immunogenicity (D28 or D56 as appropriate).

The immunogenicity analysis set (IAS) is used for the main immunogenicity analyses.

1.10.2—Analysis Sets

Two main analysis sets are used: the IAS and the Safety Analysis Set (SafAS).

i. Immunogenicity Analysis Set

The IAS is defined as the subset of randomized subjects who received 1 dose of a study vaccine (for subjects 9 to 17 years of age and for previously influenza vaccinated subjects 6 months to 8 years of age) or 2 doses of a same study vaccine (for previously influenza unvaccinated subjects 6 months to 8 years of age) and had a post-vaccination blood sample. Subjects are analyzed as treated.

ii. Safety Analysis Set

The SafAS is defined as those subjects who have received at least one dose of the study vaccines (for which safety data are scheduled to be collected).

All subjects have their safety analyzed as following:
After each dose, according to the vaccine they actually received at the dose considered
After any dose, according to the vaccine received at the first dose. In case the second dose received is different, safety data recorded at V04 and V05 will be excluded from this analysis and listed separately.

Safety data recorded for a vaccine received out of the protocol design is excluded from the analysis (and listed separately).

iii. Populations Used in Analyses

All randomized subjects with data in the CRB are taken into account in the description of the population (e.g., the disposition, the demographic, or baseline characteristics).

The safety analyses are performed on the SafAS.

The immunogenicity analyses from HAI assay and SN assay are both performed on the IAS.

1.10.3—Handling of Missing Data and Outliers

i. Safety

No replacement is done. Nevertheless, missing relationship is considered as related at the time of the statistical analysis. No search for outliers is performed. In all subject listings, partial and missing data are clearly indicated as missing.

ii. Immunogenicity

In order to appropriately manage replicate values for analysis purposes, the individual geometric mean of all values is computed for each blood sample after managing extreme values as described. The computed value is then considered the titer for that particular blood sample.
If a titer is <LLOQ, then the computed value, LLOQ/2, is used.
If a titer is ≥LLOQ and <upper limit of quantitation (ULOQ) (or ≤ULOQ), then the titer itself is used.
If a titer is ≥ULOQ (or >ULOQ), then computed value, ULOQ, is used.

Any other replacement to be applied to specific endpoints is described in the statistical analysis plan (SAP).

Missing data is not be imputed. No test or search for outliers was performed.

1.10.4—Interim/Preliminary Analysis

For Stages 1, 2, and 3, there is an early blinded look at the 7-day safety data for subjects 6 months to 8 years of age to determine whether the next group is enrolled. No statistical adjustment is necessary because no hypotheses is tested.

A limited statistical analysis of the unblinded safety and immunogenicity data obtained up to D28 or D56 may be conducted. A final analysis is conducted once the 6-month safety data have been collected and the final database lock has occurred.

No statistical adjustment is necessary because no hypotheses is tested.

1.10.5—Determination of Sample Size and Power Calculation

QHD04 is a Phase II study to describe the safety and immunogenicity of 3 different dosages of QIV-HD. The sample size is not powered. The study includes a total of approximately 700 subjects that are divided into 13 groups as follows:
Groups 1, 2, 3, 5, 9, and 10 each include 40 subjects (previously influenza vaccinated or previously influenza unvaccinated) in a 3:1 ratio (QIV-HD: QIV-SD).
Group 4 includes 90 subjects (previously influenza vaccinated or previously influenza unvaccinated) in a 1:1 ratio (QIV-HD: QIV-SD).
Groups 7 includes 60 subjects who are previously influenza vaccinated in a 1:1 ratio (QIV-HD: QIV-SD).
Groups 6 and 8 each include 60 subjects who are previously influenza unvaccinated in a 1:1 ratio (QIV-HD: QIV-SD or TIV).
Group 11 and 13 each include 60 subjects (previously influenza vaccinated or previously influenza unvaccinated) in a 1:1 ratio (QIV-HD: QIV-SD).
Group 12 includes 70 subjects (previously influenza vaccinated or previously influenza unvaccinated) in a 3:3:1 ratio (QIV-HD: QIV-HD: QIV-SD).

### Example 2—Results

### 2.1—Serological Assays

### 2.1.A—Influenza Virus Hemagglutination Inhibition Test

The influenza virus HAI assay is based on the ability of specific anti-influenza antibodies to inhibit agglutination of RBCs induced by influenza virus Hemagglutinin (HA). The serum to be tested are pretreated with neuraminidase to eliminate the non-specific inhibitors and the anti-species hemagglutinins, which may interfere with the test results.

The influenza virus HA will cause agglutination of RBCs from various species. The serum of individuals that have been successfully vaccinated against influenza, or have suffered from the infection, will contain high levels of antibodies against these HA antigens. Serial dilutions of serum are incubated with a fixed amount of influenza antigen. Antibodies present in the serum sample will inhibit the agglutination of RBCs when influenza virus antigen is added to the assay. The endpoint of the assay is the highest serum dilution in which complete inhibition of hemagglutination occurs.

Justification/Rationale

A variety of serological techniques have been developed for assessment of influenza virus vaccine responses in clinical trials or for disease detection. Methods include HAI, single radial hemolysis (SRH), virus neutralization test (NT), and enzyme linked Immunosorbent assay (ELISA). HAI antibody titers are a relevant surrogate marker of protection in vaccinated populations, therefore, the influenza virus HAI assay is an appropriate serological method due to its strain-specificity and simplicity to perform.

### 2.1.B—Influenza Virus Neutralization Test

The influenza virus NT (also sometimes identified as the Seroneutralization Assay) is an in vitro functional assay that measures the level of influenza virus neutralizing antibodies in human sera. The NT is based on the ability of neutralizing antibodies against influenza virus to inhibit the infection of Madin-Darby Canine Kidney (MDCK) cells with influenza virus. Two-fold serially diluted sera are pre-incubated with 100 Tissue Culture Infectious Dose (TCID) 50/50 μL of influenza virus prior to addition of MDCK cells. Following overnight incubation, the cells are fixed and the presence of influenza virus nucleoprotein (NP) in infected cells is detected by ELISA. The absence of infectivity constitutes a positive neutralization reaction and indicates the presence of influenza virus-specific neutralizing antibodies in human sera.

Justification/Rationale

The NT detects neutralizing antibodies to influenza virus (Rowe T et al. (1999) J Clin Microbiol. 37(4):937-43) and is a sensitive and specific assay for detecting virus-specific antibody to influenza virus of all types. The NT can detect antibodies in human serum at levels that would not be detected using the HAI assay (Id.). For some influenza virus strains, the NT assay has been shown to yield higher titers than the HAI assay (Hancock K et al. (2009) N Engl J Med. 361(20):1945-52).

### 2.2—Subject Disposition

Presented herein in Example 2 are certain results of the study outlined in Example 1, which included 665 randomized subjects from 13 sites in the United States and 3 sites in Canada. A total of 661 (99.4%) subjects were vaccinated and included in the safety analysis set (SafAS). Table 20A lists the number of doses actually received as per study design. A total of 645 (97%) subjects completed the study as per protocol. A total of 642 (96.5%) subjects with a post-vaccination blood sample were included in the immunogenicity analysis set (IAS). Only safety data from the Canadian arm of the study is reported herein as results continue to be analyzed.

TABLE 20A
Vaccine received by study group and vaccinated group - SafAS

| | | | QIV-HD | | | QIV-SD | | | Adjuvanted TIV | | |
|---|---|---|---|---|---|---|---|---|---|---|---|
| | Previous | | (N = 414) | | | (N = 234) | | | (N = 13) | | |
| | influenza | Investigational | Only 1 | 2 | | Only 1 | 2 | | Only 1 | 2 | |
| | vaccination | vaccine | Dose | Doses | Total | Dose | Doses | Total | Dose | Doses | Total |
| Study group | status* | dosage† | n | n | n | n | n | n | n | n | n |
| #1: 36 months-<5 | Both | 30 μg | 23 | 9 | 32 | 10 | 2 | 12 | — | — | — |
| years | | | | | | | | | | | |
| #2: 36 months-<5 | Both | 45 μg | 24 | 6 | 30 | 8 | 2 | 10 | — | — | — |
| years | | | | | | | | | | | |
| #3: 6-<36 months | Both | 30 μg | 14 | 15 | 29 | 5 | 6 | 11 | — | — | — |
| #4: 36 months-<5 | Both | 60 μg | 38 | 6 | 44 | 38 | 8 | 46 | — | — | — |
| years | | | | | | | | | | | |
| #5: 6-<36 months | Both | 45 μg | 12 | 18 | 30 | 3 | 7 | 10 | — | — | — |
| #6: 6-<36 months | Unvaccinated | 60 μg | 0 | 40 | 40 | 2 | 37 | 39 | — | — | — |
| #7: 6-<36 months | Vaccinated | 60 μg | 14 | 0 | 14 | 15 | 0 | 15 | — | — | — |
| #8: 6-<24 months | Unvaccinated | 60 μg | 0 | 13 | 13 | — | — | — | 0 | 13 | 13 |
| #9: 5-8 years | Both | 30 μg | 27 | 5 | 32 | 8 | 1 | 9 | — | — | — |
| #10: 5-8 years | Both | 45 μg | 26 | 5 | 31 | 9 | 1 | 10 | — | — | — |
| #11: 5-8 years | Both | 60 μg | 26 | 4 | 30 | 27 | 3 | 30 | — | — | — |
| #12: 9-17 years | Both | 30 or | 59 | 0 | 59 | 11 | 0 | 11 | — | — | — |
| | | 45 μg | | | | | | | | | |
| #13: 9-17 years | Both | 60 μg | 30 | 0 | 30 | 31 | 0 | 31 | — | — | — |

n: number of subjects fulfilling the item listed
*Both: the subjects are either previously influenza vaccinated or unvaccinated.
†X μg: X μg of hemagglutinin (HA) per strain per dose.
QIV-HD: high-dose quadrivalent influenza vaccine (Investigational vaccine).
QIV-SD: standard-dose quadrivalent influenza vaccine (Fluarix Quadrivalent).
Adjuvanted TIV: adjuvanted trivalent influenza vaccine (FLUAD Pediatric).
Notation ‘—’ is displayed in all non-applicable cells.

Table 20B presents the baseline demographics among vaccine groups based on the immunogenicity analysis set. In the US, when considering the full age group (6 months to less than 18 years), each of the QIV-HD doses was primarily compared with its QIV-SD control cohort from the relevant study groups as per the study design (according to the randomization schedule to ensure a similar distribution in age and stage in the comparison and also separately compared to the QIV-SD pooled group (i.e., all subjects who received QIV-SD, regardless of the study stage). The overall distribution of each of the demographics (sex, age, and racial origin) was similar which indicates relatively balanced characteristics among vaccine groups.

TABLE 20B
Baseline demographics by vaccinated group - IAS

| | US (6 months-17 years) | | | | | | | | | | | | | | | |
|---|---|---|---|---|---|---|---|---|---|---|---|---|---|---|---|---|
| | QIV-HD | | QIV-HD | | QIV-HD | | | | | | | | Pooled | | | |
| | (30 μg) | | (45 μg) | | (60 μg) | | QIV-SD* | | QIV-SD† | | QIV-SD‡ | | QIV-SD | | All | |
| Overall | (N = 118) | | (N = 115) | | (N = 156) | | (N = 40) | | (N = 41) | | (N = 157) | | (N = 227) | | (N = 616) | |
| Sex: n (%) | | | | | | | | | | | | | | | | |
| Male | 70 | (59.3) | 58 | (50.4) | 76 | (48.7) | 19 | (47.5) | 15 | (36.6) | 88 | (56.1) | 118 | (52.0) | 322 | (52.3) |
| Female | 48 | (40.7) | 57 | (49.6) | 80 | (51.3) | 21 | (52.5) | 26 | (63.4) | 69 | (43.9) | 109 | (48.0) | 294 | (47.7) |
| Missing | 0 | | 0 | | 0 | | 0 | | 0 | | 0 | | 0 | | 0 | |
| Sex ratio: | 1.46 | | 1.02 | | 0.95 | | 0.90 | | 0.58 | | 1.28 | | 1.08 | | 1.10 | |
| Male/Female | | | | | | | | | | | | | | | | |
| Age (year) | | | | | | | | | | | | | | | | |
| M | 118 | | 115 | | 156 | | 40 | | 41 | | 157 | | 227 | | 616 | |
| Mean (SD) | 5.9 | (3.94) | 6.2 | (4.30) | 5.2 | (4.25) | 6.0 | (4.50) | 6.2 | (4.40) | 5.2 | (4.16) | 5.2 | (4.05) | 5.5 | (4.14) |
| Min; Max | 0.7; 15.0 | | 0.8; 17.0 | | 0.6; 17.0 | | 0.7; 16.0 | | 0.8; 16.0 | | 0.5; 17.0 | | 0.5; 17.0 | | 0.5; 17.0 | |
| Median | 5.0 | | 5.0 | | 4.0 | | 4.0 | | 5.0 | | 4.0 | | 4.0 | | 4.0 | |
| Q1; Q3 | 3.0; 8.0 | | 3.0; 9.0 | | 2.0; 7.0 | | 2.5; 9.5 | | 3.0; 9.0 | | 2.0; 7.0 | | 2.0; 7.0 | | 2.0; 8.0 | |
| Age subgroup: n (%) | | | | | | | | | | | | | | | | |
| 6-<36 months | 26 | (22.0) | 27 | (23.5) | 54 | (34.6) | 10 | (25.0) | 10 | (24.4) | 51 | (32.5) | 71 | (31.3) | 178 | (28.9) |
| 36 months-<5 years | 32 | (27.1) | 28 | (24.3) | 42 | (26.9) | 11 | (27.5) | 10 | (24.4) | 45 | (28.7) | 66 | (29.1) | 168 | (27.3) |
| 5-8 years | 31 | (26.3) | 31 | (27.0) | 30 | (19.2) | 8 | (20.0) | 10 | (24.4) | 30 | (19.1) | 48 | (21.1) | 140 | (22.7) |
| 9-17 years | 29 | (24.6) | 29 | (25.2) | 30 | (19.2) | 11 | (27.5) | 11 | (26.8) | 31 | (19.7) | 42 | (18.5) | 130 | (21.1) |
| Racial origin: n (%) | | | | | | | | | | | | | | | | |
| American Indian or | 2 | (1.7) | 0 | | 0 | | 0 | | 0 | | 1 | (0.6) | 1 | (0.4) | 3 | (0.5) |
| Alaska Native | | | | | | | | | | | | | | | | |
| Asian | 1 | (0.8) | 2 | (1.7) | 0 | | 0 | | 0 | | 3 | (1.9) | 3 | (1.3) | 6 | (1.0) |
| Black or African | 22 | (18.6) | 26 | (22.6) | 40 | (25.6) | 7 | (17.5) | 5 | (12.2) | 46 | (29.3) | 57 | (25.1) | 145 | (23.5) |
| American | | | | | | | | | | | | | | | | |
| Native Hawaiian or | 0 | | 3 | (2.6) | 2 | (1.3) | 0 | | 1 | (2.4) | 3 | (1.9) | 4 | (1.8) | 9 | (1.5) |
| Other Pacific | | | | | | | | | | | | | | | | |
| Islander | | | | | | | | | | | | | | | | |
| White | 87 | (73.7) | 76 | (66.1) | 106 | (67.9) | 30 | (75.0) | 34 | (82.9) | 99 | (63.1) | 153 | (67.4) | 422 | (68.5) |
| Multiple | 4 | (3.4) | 3 | (2.6) | 7 | (4.5) | 3 | (7.5) | 1 | (2.4) | 3 | (1.9) | 7 | (3.1) | 21 | (3.4) |
| Unknown | 0 | | 0 | | 0 | | 0 | | 0 | | 0 | | 0 | | 0 | |
| Not Reported | 2 | (1.7) | 5 | (4.3) | 1 | (0.6) | 0 | | 0 | | 2 | (1.3) | 2 | (0.9) | 10 | (1.6) |
| Ethnicity: n (%) | | | | | | | | | | | | | | | | |
| Hispanic or Latino | 31 | (26.3) | 20 | (17.4) | 39 | (25.0) | 16 | (40.0) | 10 | (24.4) | 44 | (28.0) | 65 | (28.6) | 155 | (25.2) |
| Not Hispanic or | 86 | (72.9) | 91 | (79.1) | 116 | (74.4) | 24 | (60.0) | 31 | (75.6) | 111 | (70.7) | 160 | (70.5) | 453 | (73.5) |
| Latino | | | | | | | | | | | | | | | | |

| Unknown | 0 | | 1 | (0.9) | 0 | | 0 | 0 | 0 | | 0 | | 1 | (0.2) |
|---|---|---|---|---|---|---|---|---|---|---|---|---|---|---|
| Not Reported | 1 | (0.8) | 3 | (2.6) | 1 | (0.6) | 0 | 0 | 2 | (1.3) | 2 | (0.9) | 7 | (1.1) |

| | Canada (6-<24 months) | | | |
|---|---|---|---|---|
| | | QIV-HD (60 μg) | Adjuvanted TIV | All |
| Overall | | (N = 13) | (N = 13) | (N = 26) |

| | | | | | | |
|---|---|---|---|---|---|---|
| Sex: n (%) | | | | | | |
| Male | 9 | (69.2) | 7 | (53.8) | 16 | (61.5) |
| Female | 4 | (30.8) | 6 | (46.2) | 10 | (38.5) |

| | | | |
|---|---|---|---|
| Missing | 0 | 0 | 0 |
| Sex ratio: | 2.25 | 1.17 | 1.60 |
| Male/Female | | | |
| Age (year) | | | |
| M | 13 | 13 | 26 |

| | | | | | | |
|---|---|---|---|---|---|---|
| Mean (SD) | 0.8 | (0.19) | 1.0 | (0.28) | 0.9 | (0.26) |

| | | | |
|---|---|---|---|
| Min; Max | 0.6; 1.2 | 0.8; 1.8 | 0.6; 1.8 |
| Median | 0.8 | 1.0 | 0.9 |
| Q1; Q3 | 0.8; 0.9 | 0.8; 1.1 | 0.8; 1.1 |
| Racial origin: n (%) | | | |
| American Indian or | 0 | 0 | 0 |
| Alaska Native | | | |
| Asian | 0 | 0 | 0 |

| Black or African | 2 | (15.4) | 0 | | 2 | (7.7) |
|---|---|---|---|---|---|---|
| American | | | | | | |
| Native Hawaiian or | 0 | | 1 | (7.7) | 1 | (3.8) |
| Other Pacific | | | | | | |
| Islander | | | | | | |
| White | 9 | (69.2) | 11 | (84.6) | 20 | (76.9) |
| Multiple | 2 | (15.4) | 1 | (7.7) | 3 | (11.5) |

| | | | |
|---|---|---|---|
| Unknown | 0 | 0 | 0 |
| Not Reported | 0 | 0 | 0 |

| Ethnicity: n (%) | | | | | | |
|---|---|---|---|---|---|---|
| Hispanic or Latino | 2 | (15.4) | 0 | | 2 | (7.7) |
| Not Hispanic or | 11 | (84.6) | 13 | (100) | 24 | (92.3) |
| Latino | | | | | | |

| | | | |
|---|---|---|---|
| Unknown | 0 | 0 | 0 |
| Not Reported | 0 | 0 | 0 |

n: number of subjects fulfilling the item listed
M: number of subjects with available data for the relevant endpoint
Q1; Q3: first quartile; third quartile
*QIV-SD: from QIV-SD control group restricted to relevant study groups for comparison to QIV-HD 30 μg
†QIV-SD: from QIV-SD control group restricted to relevant study groups for comparison to QIV-HD 45 μg
‡QIV-SD: from QIV-SD control group restricted to relevant study groups for comparison to QIV-HD 60 μg

### 2.3—Safety Results

Executive Summary of Safety Results:

A. US Cohort

Participants:

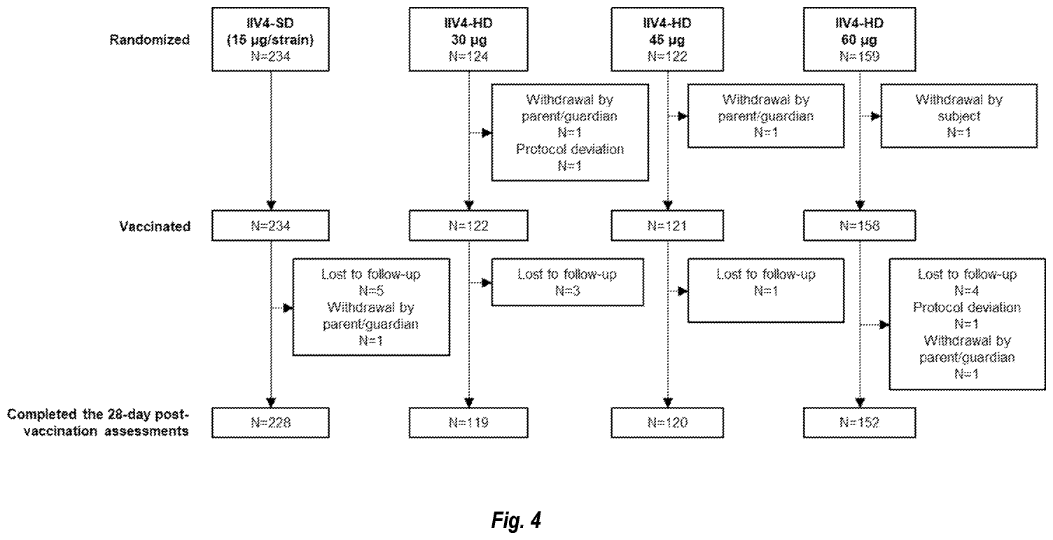

Among 639 randomized US subjects, 635 were vaccinated, all as randomized (122 with QIV-HD 30 μg, 121 with QIV-HD 45 μg, 158 with QIV-HD 60 μg, and 234 with QIV-SD) (FIG. 4). Within the QIV-SD group, 228 participants completed the study. Five participants in the QIV-HD 30 μg group, one in the QIV-HD 45 μg group, and six in the QIV-HD 60 μg group did not complete the 28-day post-vaccination visit.

Most participants were White and non-Hispanic. The overall distribution of each of the demographics (sex, age, and racial origin) was relatively similar among vaccine groups (Table 1C).

Safety and Solicited Reactions:

Rates of unsolicited AEs were similar irrespective of antigen dose (Table 20D). This was also observed when limited to participants aged 6 months through <3 years (Table 20F). No related SAEs, AEs leading to study discontinuation, or deaths were reported. Two SAEs occurred within 28 days post-vaccination in participants who received QIV-HD 60 μg that were considered unrelated to study vaccine: febrile seizure (also considered an AE of special interest) 22 days after the first vaccine dose in a participant 16 months of age; and respiratory syncytial virus infection 12 days after the first vaccine dose in a participant 2 years of age. No other AESIs occurred in the study. One immediate (<30 min) unsolicited AE considered related to the study vaccine was reported: mild worsening of chronic pain in the right shoulder in a participant aged 14 years vaccinated with QIV-HD 60 μg.

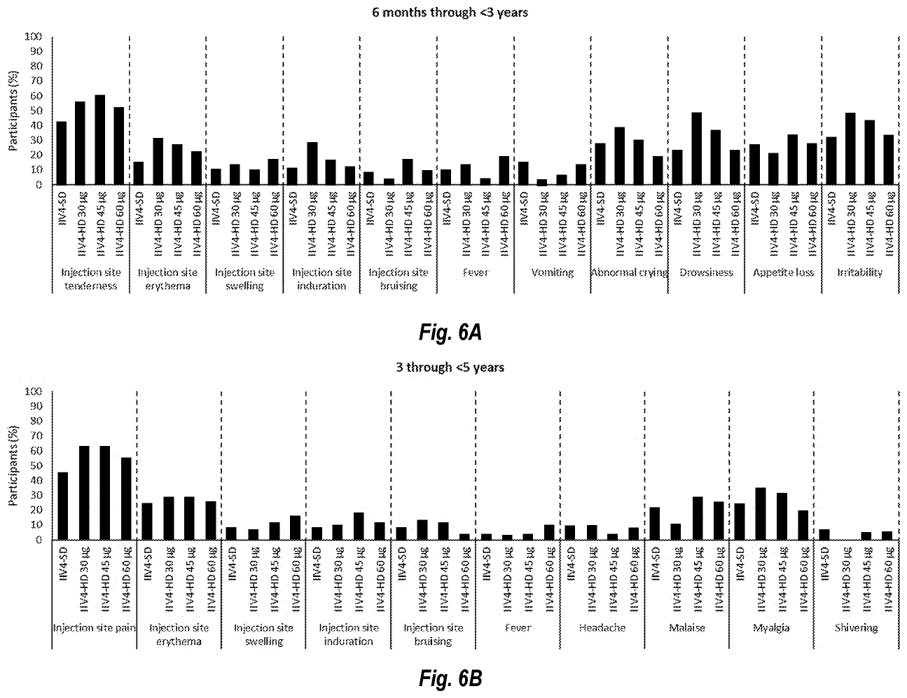

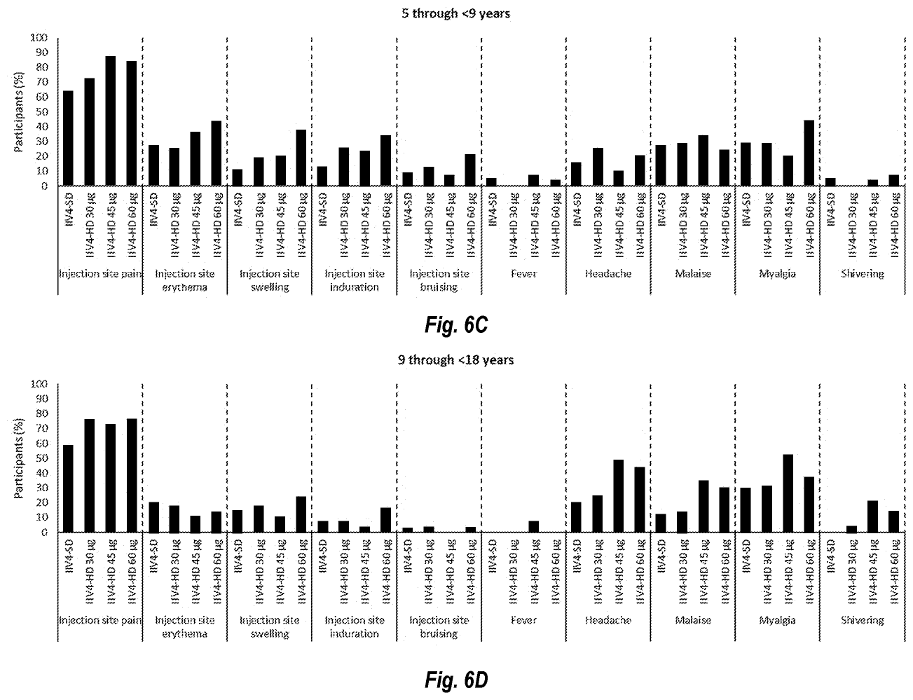

Reactogenicity (solicited injection site reactions and solicited systemic reactions) was slightly higher for QIV-HD than for QIV-SD (Table 20D). The most frequent injection-site reactions were injection-site tenderness (participants aged 6 months through <3 years) and injection-site pain (participants aged 3 through <18 years) (FIGS. 6A-6D). In participants aged 6 months through <3 years, the most frequent systemic reactions were abnormal crying, drowsiness, appetite loss, and irritability. In participants aged 3 through <5 years and 5 through <9 years, the most frequent systemic reactions included malaise and myalgia. In participants aged 9 through <18 years, the most frequent systemic reactions included headache and myalgia (FIG. 6D).

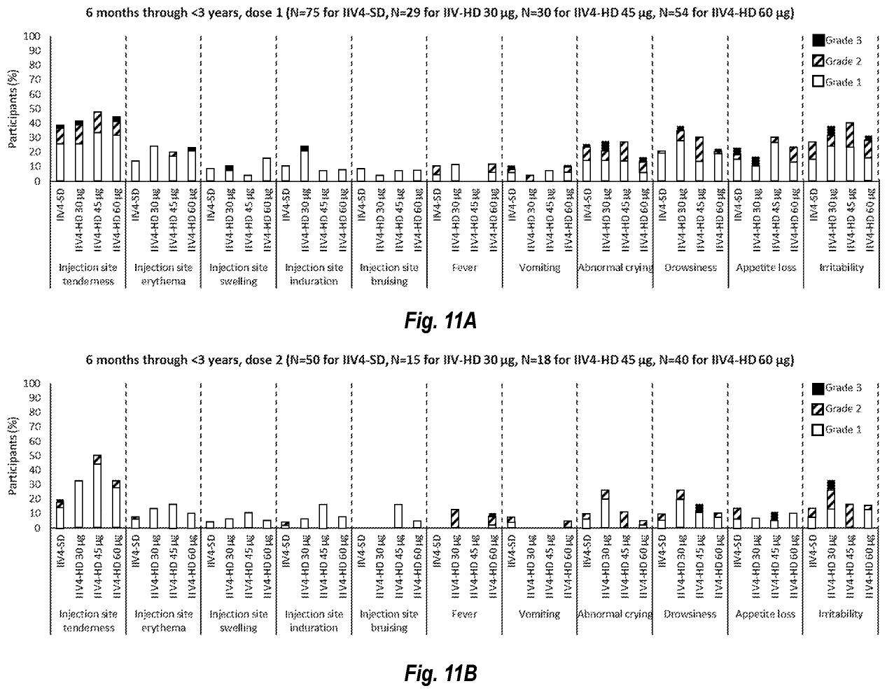

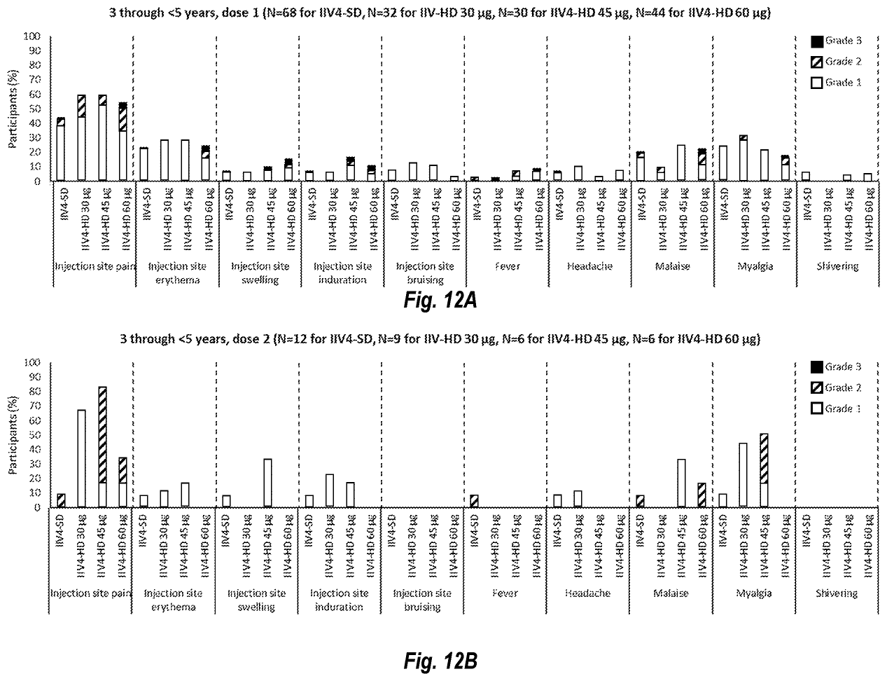

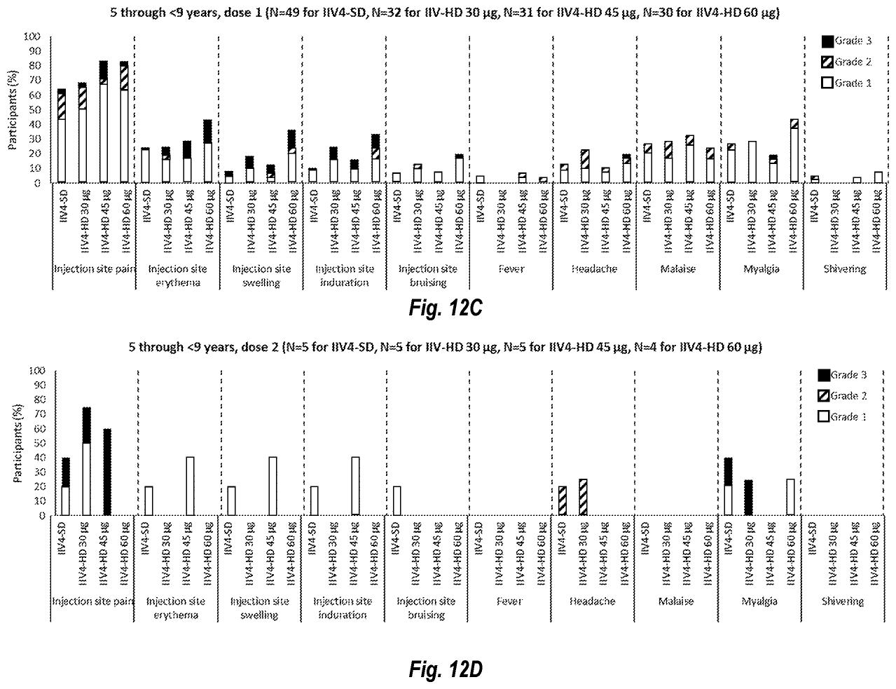

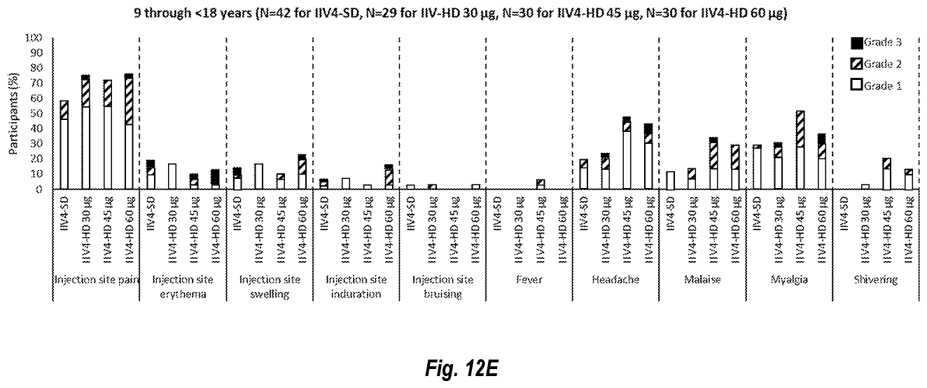

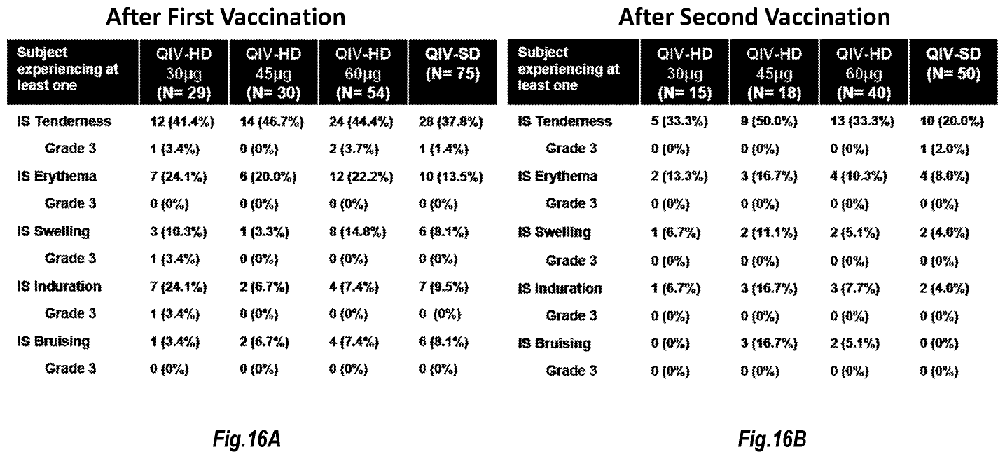

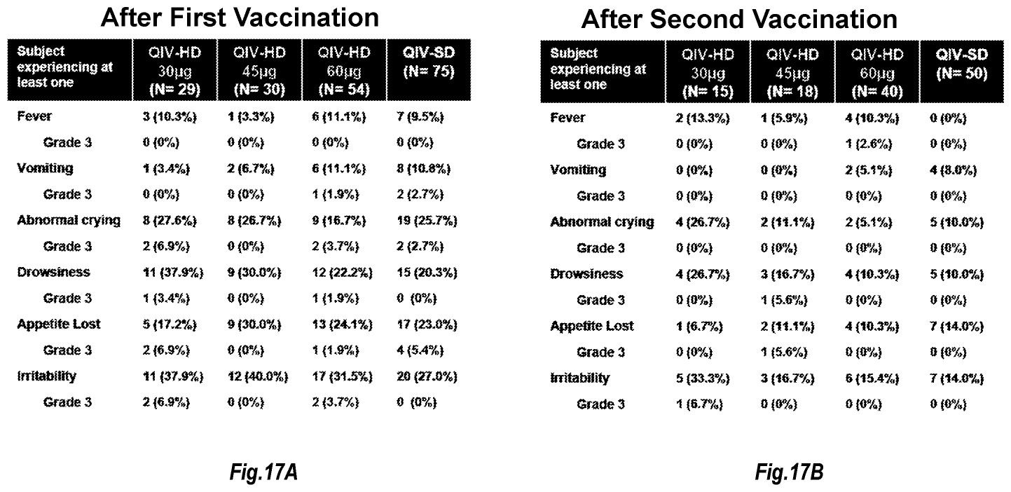

Most solicited reactions were of grade 1 or grade 2 (FIGS. 11A-11B and FIGS. 12A-12B). The rate of grade 3 solicited injection-site reactions tended to increase with increasing vaccine dose (Table 20D). Otherwise, there was no evidence of substantial dose-related adverse events. See, also, FIGS. 16A-16B and FIGS. 17A-17B.

B. Canadian Cohort:

Participants:

All participants were vaccinated as randomized: 13 with QIV-HD 60 μg and 13 with adjuvanted TIV (7.5 μg HA/strain). All participants completed the study. Demographic data for the Canadian cohort are shown in Table 1D.

Safety and solicited reactions:

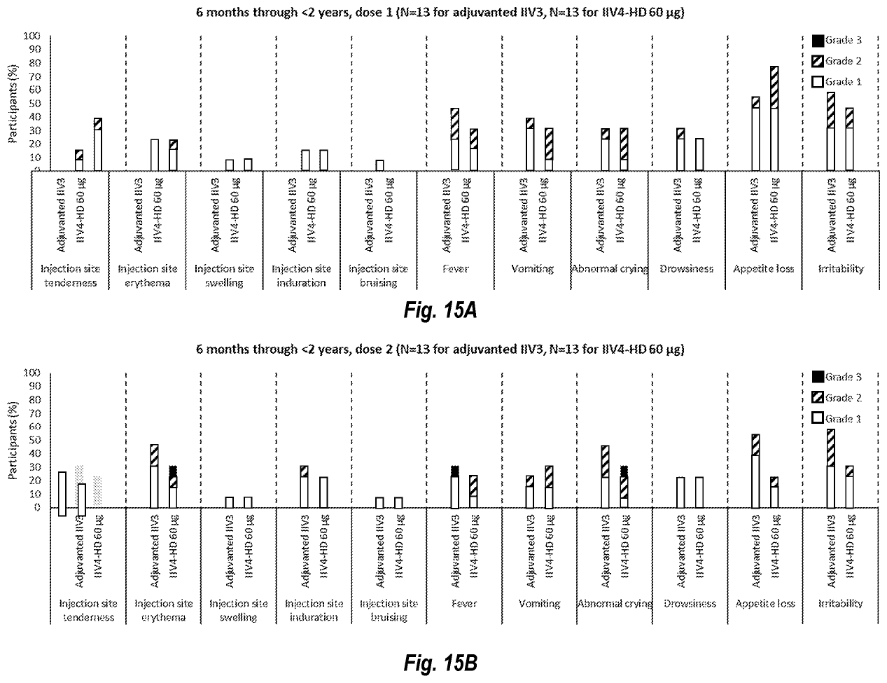

Summary data for unsolicited AEs in the Canadian cohort are shown in Table 20E. One participant experienced an SAE of severe otitis media during the 6-month safety follow-up after the last dose of vaccine. The SAE was assessed as not related to study vaccine by the investigator. Rates of individual injection-site and systemic solicited reactions are shown by vaccine and grade in FIGS. 15A-15B.

### Details of Safety Results:

2.3.1—Overview of Safety Findings for Subjects 6 Months to 17 Years of Age

Table 20C presents an overview of safety findings throughout the study for the subjects in all age groups: immediate AE information (within 30 minutes after vaccination), solicited injection site and systemic reactions (up to 7 days after vaccination), unsolicited AEs/ARs and AEs/ARs leading to withdrawal from the study (up to 28 days after vaccination), and SAEs including AESIs (throughout the study).

TABLE 20C
Safety overview after any vaccination - SafAS

| | US (6 months-17 years) | | | | | | | | | | | |
|---|---|---|---|---|---|---|---|---|---|---|---|---|
| Subjects | QIV-HD (30 μg) | | | QIV-SD* | | | QIV-HD (45 μg) | | | QIV-SD† | | |
| experiencing | (N = 122) | | | (N = 43) | | | (N = 121) | | | (N = 41) | | |
| at least one: | n/M | % | (95% CI) | n/M | % | (95% CI) | n/M | % | (95% CI) | n/M | % | (95% CI) |
| Within 28 days | | | | | | | | | | | | |
| after any | | | | | | | | | | | | |
| vaccination | | | | | | | | | | | | |
| Immediate | 0/122 | 0 | (0; 3.0) | 0/43 | 0 | (0; 8.2) | 0/121 | 0 | (0; 3.0) | 0/41 | 0 | (0; 8.6) |
| unsolicited AE | | | | | | | | | | | | |
| Immediate | 0/122 | 0 | (0; 3.0) | 0/43 | 0 | (0; 8.2) | 0/121 | 0 | (0; 3.0) | 0/41 | 0 | (0; 8.6) |
| unsolicited AR | | | | | | | | | | | | |
| Solicited | 95/122 | 77.9 | (69.5; 84.9) | 26/43 | 60.5 | (44.4; 75.0) | 94/120 | 78.3 | (69.9; 85.3) | 26/41 | 63.4 | (46.9; 77.9) |
| reaction | | | | | | | | | | | | |
| Solicited | 89/122 | 73.0 | (64.2; 80.6) | 22/43 | 51.2 | (35.5; 66.7) | 89/119 | 74.8 | (66.0; 82.3) | 24/41 | 58.5 | (42.1; 73.7) |
| injection | | | | | | | | | | | | |
| site reaction | | | | | | | | | | | | |
| Solicited | 62/122 | 50.8 | (41.6; 60.0) | 17/43 | 39.5 | (25.0; 55.6) | 62/120 | 51.7 | (42.4; 60.9) | 19/41 | 46.3 | (30.7; 62.6) |
| systemic | | | | | | | | | | | | |
| reaction | | | | | | | | | | | | |
| Unsolicited AE | 46/122 | 37.7 | (29.1; 46.9) | 13/43 | 30.2 | (17.2; 46.1) | 46/121 | 38.0 | (29.3; 47.3) | 13/41 | 31.7 | (18.1; 48.1) |
| Unsolicited | 46/122 | 37.7 | (29.1; 46.9) | 13/43 | 30.2 | (17.2; 46.1) | 46/121 | 38.0 | (29.3; 47.3) | 13/41 | 31.7 | (18.1; 48.1) |
| non-serious AE | | | | | | | | | | | | |
| Unsolicited | 46/122 | 37.7 | (29.1; 46.9) | 13/43 | 30.2 | (17.2; 46.1) | 45/121 | 37.2 | (28.6; 46.4) | 13/41 | 31.7 | (18.1; 48.1) |
| non-serious | | | | | | | | | | | | |
| systemic AE | | | | | | | | | | | | |
| Unsolicited AR | 4/122 | 3.3 | (0.9; 8.2) | 3/43 | 7.0 | (1.5; 19.1) | 8/121 | 6.6 | (2.9; 12.6) | 1/41 | 2.4 | (0.1; 12.9) |
| Unsolicited | 4/122 | 3.3 | (0.9; 8.2) | 3/43 | 7.0 | (1.5; 19.1) | 8/121 | 6.6 | (2.9; 12.6) | 1/41 | 2.4 | (0.1; 12.9) |
| non-serious AR | | | | | | | | | | | | |
| Unsolicited | 0/122 | 0 | (0; 3.0) | 0/43 | 0 | (0; 8.2) | 3/121 | 2.5 | (0.5; 7.1) | 0/41 | 0 | (0; 8.6) |
| non-serious | | | | | | | | | | | | |
| injection | | | | | | | | | | | | |
| site AR | | | | | | | | | | | | |
| Unsolicited | 4/122 | 3.3 | (0.9; 8.2) | 3/43 | 7.0 | (1.5; 19.1) | 5/121 | 4.1 | (1.4; 9.4) | 1/41 | 2.4 | (0.1; 12.9) |
| non-serious | | | | | | | | | | | | |
| systemic AR | | | | | | | | | | | | |
| AE leading to | 0/122 | 0 | (0; 3.0) | 0/43 | 0 | (0; 8.2) | 0/121 | 0 | (0; 3.0) | 0/41 | 0 | (0; 8.6) |
| study | | | | | | | | | | | | |
| discontinuation | | | | | | | | | | | | |
| SAE within 28 | 0/122 | 0 | (0; 3.0) | 0/43 | 0 | (0; 8.2) | 0/121 | 0 | (0; 3.0) | 0/41 | 0 | (0; 8.6) |
| days after any | | | | | | | | | | | | |
| vaccination | | | | | | | | | | | | |
| Fatal | 0/122 | 0 | (0; 3.0) | 0/43 | 0 | (0; 8.2) | 0/121 | 0 | (0; 3.0) | 0/41 | 0 | (0; 8.6) |
| AE of special | 0/122 | 0 | (0; 3.0) | 0/43 | 0 | (0; 8.2) | 0/121 | 0 | (0; 3.0) | 0/41 | 0 | (0; 8.6) |
| interest | | | | | | | | | | | | |
| From 29 days | | | | | | | | | | | | |
| after the last | | | | | | | | | | | | |
| vaccination | | | | | | | | | | | | |
| SAE | 0/122 | 0 | (0; 3.0) | 0/43 | 0 | (0; 8.2) | 0/121 | 0 | (0; 3.0) | 0/41 | 0 | (0; 8.6) |
| Fatal | 0/122 | 0 | (0; 3.0) | 0/43 | 0 | (0; 8.2) | 0/121 | 0 | (0; 3.0) | 0/41 | 0 | (0; 8.6) |
| AE of special | 0/122 | 0 | (0; 3.0) | 0/43 | 0 | (0; 8.2) | 0/121 | 0 | (0; 3.0) | 0/41 | 0 | (0; 8.6) |
| interest (AESI) | | | | | | | | | | | | |
| During the | | | | | | | | | | | | |
| entire study | | | | | | | | | | | | |
| SAE | 0/122 | 0 | (0; 3.0) | 0/43 | 0 | (0; 8.2) | 0/121 | 0 | (0; 3.0) | 0/41 | 0 | (0; 8.6) |
| Fatal | 0/122 | 0 | (0; 3.0) | 0/43 | 0 | (0; 8.2) | 0/121 | 0 | (0; 3.0) | 0/41 | 0 | (0; 8.6) |
| AE of special | 0/122 | 0 | (0; 3.0) | 0/43 | 0 | (0; 8.2) | 0/121 | 0 | (0; 3.0) | 0/41 | 0 | (0; 8.6) |
| interest (AESI) | | | | | | | | | | | | |

| | US (6 months-17 years) | | | | | | | | |
|---|---|---|---|---|---|---|---|---|---|
| Subjects | QIV-HD (60 μg) | | | QIV-SD‡ | | | Pooled QIV-SD | | |
| experiencing | (N = 158) | | | (N = 161) | | | (N = 234) | | |
| at least one: | n/M | % | (95% CI) | n/M | % | (95% CI) | n/M | % | (95% CI) |
| Within 28 days | | | | | | | | | |
| after any | | | | | | | | | |
| vaccination | | | | | | | | | |
| Immediate | 1/158 | 0.6 | (0; 3.5) | 0/161 | 0 | (0; 2.3) | 0/234 | 0 | (0; 1.6) |
| unsolicited AE | | | | | | | | | |
| Immediate | 1/158 | 0.6 | (0; 3.5) | 0/161 | 0 | (0; 2.3) | 0/234 | 0 | (0; 1.6) |
| unsolicited AR | | | | | | | | | |
| Solicited | 111/158 | 70.3 | (62.5; 77.3) | 101/159 | 63.5 | (55.5; 71.0) | 146/232 | 62.9 | (56.4; 69.2) |
| reaction | | | | | | | | | |
| Solicited | 101/158 | 63.9 | (55.9; 71.4) | 88/159 | 55.3 | (47.3; 63.2) | 128/232 | 55.2 | (48.5; 61.7) |
| injection | | | | | | | | | |
| site reaction | | | | | | | | | |
| Solicited | 71/158 | 44.9 | (37.0; 53.0) | 66/159 | 41.5 | (33.8; 49.6) | 97/232 | 41.8 | (35.4; 48.4) |
| systemic | | | | | | | | | |
| reaction | | | | | | | | | |
| Unsolicited AE | 53/158 | 33.5 | (26.2; 41.5) | 56/161 | 34.8 | (27.5; 42.7) | 80/234 | 34.2 | (28.1; 40.7) |
| Unsolicited | 52/158 | 32.9 | (25.7; 40.8) | 56/161 | 34.8 | (27.5; 42.7) | 80/234 | 34.2 | (28.1; 40.7) |
| non-serious AE | | | | | | | | | |
| Unsolicited | 52/158 | 32.9 | (25.7; 40.8) | 56/161 | 34.8 | (27.5; 42.7) | 80/234 | 34.2 | (28.1; 40.7) |
| non-serious | | | | | | | | | |
| systemic AE | | | | | | | | | |
| Unsolicited AR | 8/158 | 5.1 | (2.2; 9.7) | 6/161 | 3.7 | (1.4; 7.9) | 10/234 | 4.3 | (2.1; 7.7) |
| Unsolicited | 8/158 | 5.1 | (2.2; 9.7) | 6/161 | 3.7 | (1.4; 7.9) | 10/234 | 4.3 | (2.1; 7.7) |
| non-serious AR | | | | | | | | | |
| Unsolicited | 0/158 | 0 | (0; 2.3) | 0/161 | 0 | (0; 2.3) | 0/234 | 0 | (0; 1.6) |
| non-serious | | | | | | | | | |
| injection | | | | | | | | | |
| site AR | | | | | | | | | |
| Unsolicited | 8/158 | 5.1 | (2.2; 9.7) | 6/161 | 3.7 | (1.4; 7.9) | 10/234 | 4.3 | (2.1; 7.7) |
| non-serious | | | | | | | | | |
| systemic AR | | | | | | | | | |
| AE leading to | 0/158 | 0 | (0; 2.3) | 0/161 | 0 | (0; 2.3) | 0/234 | 0 | (0; 1.6) |
| study | | | | | | | | | |
| discontinuation | | | | | | | | | |
| SAE within 28 | 2/158 | 1.3 | (0.2; 4.5) | 0/161 | 0 | (0; 2.3) | 0/234 | 0 | (0; 1.6) |
| days after any | | | | | | | | | |
| vaccination | | | | | | | | | |
| Fatal | 0/158 | 0 | (0; 2.3) | 0/161 | 0 | (0; 2.3) | 0/234 | 0 | (0; 1.6) |
| AE of special | 1/158 | 0.6 | (0; 3.5) | 0/161 | 0 | (0; 2.3) | 0/234 | 0 | (0; 1.6) |
| interest | | | | | | | | | |
| From 29 days | | | | | | | | | |
| after the last | | | | | | | | | |
| vaccination | | | | | | | | | |
| SAE | 0/158 | 0 | (0; 2.3) | 0/161 | 0 | (0; 2.3) | 0/234 | 0 | (0; 1.6) |
| Fatal | 0/158 | 0 | (0; 2.3) | 0/161 | 0 | (0; 2.3) | 0/234 | 0 | (0; 1.6) |
| AE of special | 0/158 | 0 | (0; 2.3) | 0/161 | 0 | (0; 2.3) | 0/234 | 0 | (0; 1.6) |
| interest (AESI) | | | | | | | | | |
| During the | | | | | | | | | |
| entire study | | | | | | | | | |
| SAE | 2/158 | 1.3 | (0.2; 4.5) | 0/161 | 0 | (0; 2.3) | 0/234 | 0 | (0; 1.6) |
| Fatal | 0/158 | 0 | (0; 2.3) | 0/161 | 0 | (0; 2.3) | 0/234 | 0 | (0; 1.6) |
| AE of special | 1/158 | 0.6 | (0; 3.5) | 0/161 | 0 | (0; 2.3) | 0/234 | 0 | (0; 1.6) |
| interest (AESI) | | | | | | | | | |

Canada (6-less than 24 months)

| Subjects | QIV-HD (60 μg) | | | Adjuvanted TIV | | |
|---|---|---|---|---|---|---|
| experiencing | (N = 13) | | | (N = 13) | | |
| at least one: | n/M | % | (95% CI) | n/M | % | (95% CI) |
| Within 28 days | | | | | | |
| after any | | | | | | |
| vaccination | | | | | | |
| Immediate | 0/13 | 0 | (0; 24.7) | 0/13 | 0 | (0; 24.7) |
| unsolicited AE | | | | | | |
| Immediate | 0/13 | 0 | (0; 24.7) | 0/13 | 0 | (0; 24.7) |
| unsolicited AR | | | | | | |
| Solicited | 13/13 | 100 | (75.3; 100) | 13/13 | 100 | (75.3; 100) |
| reaction | | | | | | |
| Solicited | 9/13 | 69.2 | (38.6; 90.9) | 9/13 | 69.2 | (38.6; 90.9) |
| injection | | | | | | |
| site reaction | | | | | | |
| Solicited | 13/13 | 100 | (75.3; 100) | 13/13 | 100 | (75.3; 100) |
| systemic | | | | | | |
| reaction | | | | | | |
| Unsolicited AE | 11/13 | 84.6 | (54.6; 98.1) | 12/13 | 92.3 | (64.0; 99.8) |
| Unsolicited | 11/13 | 84.6 | (54.6; 98.1) | 12/13 | 92.3 | (64.0; 99.8) |
| non-serious AE | | | | | | |
| Unsolicited | 11/13 | 84.6 | (54.6; 98.1) | 12/13 | 92.3 | (64.0; 99.8) |
| non-serious | | | | | | |
| systemic AE | | | | | | |
| Unsolicited AR | 1/13 | 7.7 | (0.2; 36.0) | 3/13 | 23.1 | (5.0; 53.8) |
| Unsolicited | 1/13 | 7.7 | (0.2; 36.0) | 3/13 | 23.1 | (5.0; 53.8) |
| non-serious AR | | | | | | |
| Unsolicited | 0/13 | 0 | (0; 24.7) | 1/13 | 7.7 | (0.2; 36.0) |
| non-serious | | | | | | |
| injection | | | | | | |
| site AR | | | | | | |
| Unsolicited | 1/13 | 7.7 | (0.2; 36.0) | 2/13 | 15.4 | (1.9; 45.4) |
| non-serious | | | | | | |
| systemic AR | | | | | | |
| AE leading to | 0/13 | 0 | (0; 24.7) | 0/13 | 0 | (0; 24.7) |
| study | | | | | | |
| discontinuation | | | | | | |
| SAE within 28 | 0/13 | 0 | (0; 24.7) | 0/13 | 0 | (0; 24.7) |
| days after any | | | | | | |
| vaccination | | | | | | |
| Fatal | 0/13 | 0 | (0; 24.7) | 0/13 | 0 | (0; 24.7) |
| AE of special | 0/13 | 0 | (0; 24.7) | 0/13 | 0 | (0; 24.7) |
| interest | | | | | | |
| From 29 days | | | | | | |
| after the last | | | | | | |
| vaccination | | | | | | |
| SAE | 0/13 | 0 | (0; 24.7) | 1/13 | 7.7 | (0.2; 36.0) |
| Fatal | 0/13 | 0 | (0; 24.7) | 0/13 | 0 | (0; 24.7) |
| AE of special | 0/13 | 0 | (0; 24.7) | 0/13 | 0 | (0; 24.7) |
| interest (AESI) | | | | | | |
| During the | | | | | | |
| entire study | | | | | | |
| SAE | 0/13 | 0 | (0; 24.7) | 1/13 | 7.7 | (0.2; 36.0) |
| Fatal | 0/13 | 0 | (0; 24.7) | 0/13 | 0 | (0; 24.7) |
| AE of special | 0/13 | 0 | (0; 24.7) | 0/13 | 0 | (0; 24.7) |
| interest (AESI) | | | | | | |

n: number of subjects experiencing the endpoint listed in the first column
M: number of subjects with available data for the relevant endpoint
*QIV-SD: from QIV-SD control group restricted to relevant study groups for comparison to QIV-HD 30 μg
†QIV-SD: from QIV-SD control group restricted to relevant study groups for comparison to QIV-HD 45 μg
‡QIV-SD: from QIV-SD control group restricted to relevant study groups for comparison to QIV-HD 60 μg

TABLE 20D
Consolidated Results - Solicited reactions and
unsolicited adverse events in the US cohort

| | QIV-SD | QIV-HD | QIV-HD | QIV-HD |
|---|---|---|---|---|
| | (15 μg) | 30 μg | 45 μg | 60 μg |
| Event | (N = 234) | (N = 122) | (N = 121) | (N = 158) |
| Any solicited reaction (within 7 | 146 (62.9% | 95 (77.9% | 94 (78.3% | 111 (70.3% |
| days of vaccination)^{a} | [56.4; 69.2]) | [96.5; 84.9]) | [69.9; 85.3]) | [62.5; 77.3]) |
| Injection-site | 128 (55.2% | 89 (73.0% | 89 (74.8% | 101 (63.9% |
| | [48.5; 61.7]) | [64.2; 80.6]) | [66.0; 82.3]) | [55.9; 71.4]) |
| Systemic | 97 (41.8% | 62 (50.8% | 62 (51.7% | 71 (44.9% |
| | [35.4; 48.4]) | [41.6; 60.0]) | [42.2; 62.9]) | [37; 53]) |
| Grade 3 solicited reaction (within 7 | 17 (7.3%) | 11 (9.0%) | 12 (10.0%) | 25 (15.8%) |
| days of vaccination)^{a} | | | | |
| Injection-site | 10 (4.3%) | 7 (5.7%) | 10 (8.3%) | 16 (10.1%) |
| Systemic | 8 (3.4%) | 8 (6.6%) | 3 (2.5%) | 13 (8.2%) |
| Any unsolicited AE (within 28 | 80 (34.2% | 46 (37.7% | 46 (38.0% | 53 (33.5% |
| days of the last vaccination) | [28.1; 40.7]) | [29.1; 46.9]) | [29.2; 47.3]) | [26.2; 41.5]) |
| Immediate (<30 min) | 0 (0% | 0 (0% | 0 (0% | 1 (0.6% |
| | [0; 1.6]) | [0; 3.0]) | [0; 3.0]) | [0; 3.5]) |
| Treatment-related | 0 (0% | 0 (0% | 0 (0% | 1 (0.6% |
| | [0; 1.6]) | [0; 3.0]) | [0; 3.0]) | [0; 3.5]) |
| Leading to study discontinuation | 0 (0% | 0 (0% | 0 (0% | 0 (0% |
| | [0; 1.6]) | [0; 3.0]) | [0; 3.0]) | [0; 2.3]) |
| SAE (within 6 months of the last | 0 (0% | 0 (0% | 0 (0% | 2 (1.3% |
| vaccination) | [0; 1.6]) | [0; 3.0]) | [0; 3.0]) | [0.2; 4.5]) |
| Treatment-related | 0 (0% | 0 (0% | 0 (0% | 0 (0% |
| | [0; 1.6]) | [0; 3.0]) | [0; 3.0]) | [0; 2.3]) |
| Deaths | 0 (0% | 0 (0% | 0 (0% | 0 (0% |
| | [0; 1.6]) | [0; 3.0]) | [0; 3.0]) | [0; 2.3]) |
| AE of special interest | 0 (0% | 0 (0% | 0 (0% | 1 (0.6% |
| | [0; 1.6]) | [0; 3.0]) | [0; 3.0]) | [0; 3.5])b |

^{a}Complete data for solicited reactions were not available for two participants who received QIV-HD 45 μg and two participants who received QIV-SD;
bfebrile seizure.

TABLE 20E
Solicited reactions and unsolicited adverse events within
28 days after any vaccine dosing in the Canadian cohort

| | Adjuvanted TIV | | QIV-HD | |
|---|---|---|---|---|
| | (7.5 μg) | | 60 μg | |
| Event | (N = 13) | | (N = 13) | |
| Any solicited reaction (within 7 | 13 | (100%) | 13 | (100%) |
| days of vaccination) | | | | |
| Injection-site reaction | 9 | (69.2%) | 9 | (69.2%) |
| Systemic reaction | 13 | (100%) | 13 | (100%) |
| Grade 3 | 1 | (7.7%) | 2 | (15.4%) |
| Injection-site reaction | 0 | (0%) | 1 | (7.7%) |
| Systemic reaction | 1 | (7.7%) | 1 | (7.7%) |
| Any unsolicited AE (within 28 | 12 | (92.3%) | 11 | (84.6%) |
| days of the last vaccination) | | | | |
| Immediate unsolicited AE (<30 | 0 | (0%) | 0 | (0%) |
| min) | | | | |
| Treatment-related | 0 | (0%) | 0 | (0%) |
| Leading to study discontinuation | 0 | (0%) | 0 | (0%) |
| SAE (within 6 months of the last | 1 | (7.7%)* | 0 | (0%) |
| vaccination) | | | | |
| Treatment-related | 0 | (0%) | 0 | (0%) |
| Death | 0 | (0%) | 0 | (0%) |
| AE of special interest | 0 | (0%) | 0 | (0%) |

*Severe Otitis Media
Results are for the safety analysis set.
AE, adverse event;
SAE, serious adverse event

TABLE 20F
Solicited reactions and unsolicited adverse events
in US participants aged 6 months through <3 years

| | QIV-SD | | QIV-HD | | QIV-HD | | QIV-HD | |
|---|---|---|---|---|---|---|---|---|
| | (15 μg) | | 30 μg | | 45 μg | | 60 μg | |
| Event | (N = 75) | | (N = 29) | | (N = 30) | | (N = 54) | |
| Any solicited reaction (within 7 | 42 | (56.8%) | 20 | (69.0%) | 21 | (70.0%) | 36 | (66.7%) |
| days of vaccination) ^{a} | | | | | | | | |
| Injection-site reaction | 32 | (43.2%) | 18 | (62.1%) | 19 | (63.3%) | 28 | (51.9%) |
| Systemic reaction | 37 | (50.0%) | 18 | (62.1%) | 16 | (53.3%) | 24 | (44.4%) |
| Grade 3 | 8 | (10.8%) | 4 | (13.8%) | 1 | (3.3%) | 7 | (13.0%) |
| Injection-site reaction | 2 | (2.7%) | 2 | (6.9%) | 0 | (0%) | 2 | (3.7%) |
| Systemic reaction | 6 | (8.1%) | 4 | (13.8%) | 1 | (3.3%) | 6 | (11.1%) |
| Any unsolicited AE (within 28 | 38 | (50.7%) | 16 | (55.2%) | 13 | (43.3%) | 23 | (42.6%) |
| days of the last vaccination) | | | | | | | | |
| Immediate unsolicited AE (<30 | 0 | (0%) | 0 | (0%) | 0 | (0%) | 0 | (0%) |
| min) | | | | | | | | |
| Treatment-related | 0 | (0%) | 0 | (0%) | 0 | (0%) | 0 | (0%) |
| Leading to study discontinuation | 0 | (0%) | 0 | (0%) | 0 | (0%) | 0 | (0%) |
| SAE (within 6 months of the last | 0 | (0%) | 0 | (0%) | 0 | (0%) | 2 | (3.7%) |
| vaccination) | | | | | | | | |
| Treatment-related | 0 | (0%) | 0 | (0%) | 0 | (0%) | 0 | (0%) |
| Death | 0 | (0%) | 0 | (0%) | 0 | (0%) | 0 | (0%) |
| AE of special interest | 0 | (0%) | 0 | (0%) | 0 | (0%) | 1 | (1.9%)* |

*febrile seizure
Results are for the safety analysis set.
^{a} Data for solicited reactions were not available for one participant who received QIV-SD.
AE, adverse event;
SAE, serious adverse event

The safety data pertaining to children 6 months to less than 18 years of age are summarized in the subsequent sections.

The safety analyses were performed on the SafAS, defined as those subjects who have received at least one dose of the study vaccines. The SafAS consisted of a total of 661 subjects (635 subjects in the US and 26 subjects in Canada).

2.3.2—Subjects 9—Less than 18 Years of Age (Groups 12 and 13 [Stage 1]—US)

Solicited Reactions Between D0 and D7

Within 7 days of vaccination, the percentages of subjects who reported at least 1 solicited reaction were similar among the 3 QIV-HD groups (30 μg, 45 μg, and 60 μg) and slightly higher for each QIV-HD group compared to QIV-SD group: 82.8% (24/29), 80.0% (24/30), 80.0% (24/30), and 65.9% (27/41), respectively (Table 21).

Solicited injection site reactions:

The percentages of subjects who reported at least 1 solicited injection site reaction were 79.3% (23/29), 79.3% (23/29), 76.7% (23/30), and 61.0% (25/41) subjects in the QIV-HD groups (30 μg, 45 μg, and 60 μg) and QIV-SD group, respectively (Table 21).

Solicited systemic reactions:

The percentages of subjects who reported at least 1 solicited systemic reaction were 51.7% (15/29), 70.0% (21/30), 63.3% (19/30), and 39.0% (16/41) subjects in the QIV-HD (30 μg, 45 μg, and 60 μg) groups and QIV-SD group, respectively (Table 21).

Unsolicited non-serious AEs between D0 and D28 and SAEs/AESIs throughout the study

Within 30 minutes after vaccination, 1 subject in the QIV-HD (60 μg) group experienced 1 unsolicited AE which was also considered an AR (Table 21).

Within 28 days after vaccination, the percentages of subjects who experienced at least 1 unsolicited non-serious AE in the QIV-HD (30 μg, 45 μg, and 60 μg) and QIV-SD group were 20.7% (6/29), 26.7% (8/30), 13.3% (4/30), and 16.7% (7/42), respectively. The percentages of subjects who experienced at least 1 unsolicited non-serious AR in the QIV-HD (30 μg, 45 μg, and 60 μg) and QIV-SD group was 3.4% (1/29), 6.7% (2/30), 10.0% (3/30), and 4.8% (2/42), respectively (Table 21).

TABLE 21
Safety overview after vaccination (US subjects 9-17 years) - SafAS

| | US (9-17 years) | | | | | | | | | | | |
|---|---|---|---|---|---|---|---|---|---|---|---|---|
| Subjects | QIV-HD (30 μg) | | | QIV-HD (45 μg) | | | QIV-HD (60 μg) | | | QIV-SD | | |
| experiencing | (N = 29) | | | (N = 30) | | | (N = 30) | | | (N = 42) | | |
| at least one: | n/M | % | (95% CI) | n/M | % | (95% CI) | n/M | % | (95% CI) | n/M | % | (95% CI) |
| Within 28 days | | | | | | | | | | | | |
| after any | | | | | | | | | | | | |
| vaccination | | | | | | | | | | | | |
| Immediate | 0/29 | 0 | (0; 11.9) | 0/30 | 0 | (0; 11.6) | 1/30 | 3.3 | (0.1; 17.2) | 0/42 | 0 | (0; 8.4) |
| unsolicited AE | | | | | | | | | | | | |
| Immediate | 0/29 | 0 | (0; 11.9) | 0/30 | 0 | (0; 11.6) | 1/30 | 3.3 | (0.1; 17.2) | 0/42 | 0 | (0; 8.4) |
| unsolicited AR | | | | | | | | | | | | |
| Solicited | 24/29 | 82.8 | (64.2; 94.2) | 24/30 | 80.0 | (61.4; 92.3) | 24/30 | 80.0 | (61.4; 92.3) | 27/41 | 65.9 | (49.4; 79.9) |
| reaction | | | | | | | | | | | | |
| Solicited | 23/29 | 79.3 | (60.3; 92.0) | 23/29 | 79.3 | (60.3; 92.0) | 23/30 | 76.7 | (57.7; 90.1) | 25/41 | 61.0 | (44.5; 75.8) |
| injection | | | | | | | | | | | | |
| site reaction | | | | | | | | | | | | |
| Solicited | 15/29 | 51.7 | (32.5; 70.6) | 21/30 | 70.0 | (50.6; 85.3) | 19/30 | 63.3 | (43.9; 80.1) | 16/41 | 39.0 | (24.2; 55.5) |
| systemic | | | | | | | | | | | | |
| reaction | | | | | | | | | | | | |
| Unsolicited AE | 6/29 | 20.7 | (8.0; 39.7) | 8/30 | 26.7 | (12.3; 45.9) | 4/30 | 13.3 | (3.8; 30.7) | 7/42 | 16.7 | (7.0; 31.4) |
| Unsolicited | 6/29 | 20.7 | (8.0; 39.7) | 8/30 | 26.7 | (12.3; 45.9) | 4/30 | 13.3 | (3.8; 30.7) | 7/42 | 16.7 | (7.0; 31.4) |
| non-serious AE | | | | | | | | | | | | |
| Unsolicited | 6/29 | 20.7 | (8.0; 39.7) | 7/30 | 23.3 | (9.9; 42.3) | 4/30 | 13.3 | (3.8; 30.7) | 7/42 | 16.7 | (7.0; 31.4) |
| non-serious | | | | | | | | | | | | |
| systemic AE | | | | | | | | | | | | |
| Unsolicited AR | 1/29 | 3.4 | (0.1; 17.8) | 2/30 | 6.7 | (0.8; 22.1) | 3/30 | 10.0 | (2.1; 26.5) | 2/42 | 4.8 | (0.6; 16.2) |
| Unsolicited | 1/29 | 3.4 | (0.1; 17.8) | 2/30 | 6.7 | (0.8; 22.1) | 3/30 | 10.0 | (2.1; 26.5) | 2/42 | 4.8 | (0.6; 16.2) |
| non-serious AR | | | | | | | | | | | | |
| Unsolicited | 0/29 | 0 | (0; 11.9) | 1/30 | 3.3 | (0.1; 17.2) | 0/30 | 0 | (0; 11.6) | 0/42 | 0 | (0; 8.4) |
| non-serious | | | | | | | | | | | | |
| injection | | | | | | | | | | | | |
| site AR | | | | | | | | | | | | |
| Unsolicited | 1/29 | 3.4 | (0.1; 17.8) | 1/30 | 3.3 | (0.1; 17.2) | 3/30 | 10.0 | (2.1; 26.5) | 2/42 | 4.8 | (0.6; 16.2) |
| non-serious | | | | | | | | | | | | |
| systemic AR | | | | | | | | | | | | |
| AE leading to | 0/29 | 0 | (0; 11.9) | 0/30 | 0 | (0; 11.6) | 0/30 | 0 | (0; 11.6) | 0/42 | 0 | (0; 8.4) |
| study | | | | | | | | | | | | |
| discontinuation | | | | | | | | | | | | |
| SAE within 28 | 0/29 | 0 | (0; 11.9) | 0/30 | 0 | (0; 11.6) | 0/30 | 0 | (0; 11.6) | 0/42 | 0 | (0; 8.4) |
| days after any | | | | | | | | | | | | |
| vaccination | | | | | | | | | | | | |
| Fatal | 0/29 | 0 | (0; 11.9) | 0/30 | 0 | (0; 11.6) | 0/30 | 0 | (0; 11.6) | 0/42 | 0 | (0; 8.4) |
| AE of special | 0/29 | 0 | (0; 11.9) | 0/30 | 0 | (0; 11.6) | 0/30 | 0 | (0; 11.6) | 0/42 | 0 | (0; 8.4) |
| interest | | | | | | | | | | | | |
| From 29 days | | | | | | | | | | | | |
| after the last | | | | | | | | | | | | |
| vaccination | | | | | | | | | | | | |
| SAE | 0/29 | 0 | (0; 11.9) | 0/30 | 0 | (0; 11.6) | 0/30 | 0 | (0; 11.6) | 0/42 | 0 | (0; 8.4) |
| Fatal | 0/29 | 0 | (0; 11.9) | 0/30 | 0 | (0; 11.6) | 0/30 | 0 | (0; 11.6) | 0/42 | 0 | (0; 8.4) |
| AE of special | 0/29 | 0 | (0; 11.9) | 0/30 | 0 | (0; 11.6) | 0/30 | 0 | (0; 11.6) | 0/42 | 0 | (0; 8.4) |
| interest (AESI) | | | | | | | | | | | | |
| During the | | | | | | | | | | | | |
| entire study | | | | | | | | | | | | |
| SAE | 0/29 | 0 | (0; 11.9) | 0/30 | 0 | (0; 11.6) | 0/30 | 0 | (0; 11.6) | 0/42 | 0 | (0; 8.4) |
| Fatal | 0/29 | 0 | (0; 11.9) | 0/30 | 0 | (0; 11.6) | 0/30 | 0 | (0; 11.6) | 0/42 | 0 | (0; 8.4) |
| AE of special | 0/29 | 0 | (0; 11.9) | 0/30 | 0 | (0; 11.6) | 0/30 | 0 | (0; 11.6) | 0/42 | 0 | (0; 8.4) |
| interest (AESI) | | | | | | | | | | | | |

n: number of subjects experiencing the endpoint listed in the first column
M: number of subjects with available data for the relevant endpoint

The SOC with the highest incidence of unsolicited non-serious AEs was “Gastrointestinal disorders” with 3 AEs reported by 10.3% (3/29) of subjects in the QIV-HD (30 μg) group; 3 AEs reported by 7.6% (2/30) of subjects in the QIV-HD (45 μg) group; 3 AEs reported by 6.7% (2/30) of subjects in the QIV-HD (60 μg) group; and 4 AEs reported by 7.1% (3/42) of subjects in the QIV-SD group. The most frequently reported PT in this SOC for the QIV-HD (30 μg, 45 μg, 60 μg) groups and QIV-SD group was nausea.

None of the subjects experienced any AEs leading to study discontinuation, SAEs, fatal SAEs, or AESIs throughout the study (Table 21).

2.3.3—Subjects 5-8 Years of Age (Groups 9 [Stage 1], 10 [Stage 2], and 11 [Stage 3]—US)

Solicited Reactions Between D0 and D7

Within 7 days of any vaccination, the percentages of subjects who reported at least 1 solicited reaction were similar among the QIV-HD groups (30 μg, 45 μg, and 60 μg) and slightly higher for each QIV-HD group compared to QIV-SD group: 87.5% (28/32), 90.3% (28/31), 86.7% (26/30), and 73.5% (36/49), respectively (Table 22).

Solicited Injection Site Reactions:

The percentages of subjects who reported at least 1 solicited injection site reaction were 78.1% (25/32), 87.1% (27/31), 86.7% (26/30), and 71.4% (35/49) subjects in the QIV-HD groups (30 μg, 45 μg, 60 μg) and QIV-SD group, respectively (Table 22).

Few Grade 3 reactions were reported. There was no increase in incidence of solicited injection site reactions after the 2nd vaccination compared to the 1st vaccination.

Solicited Systemic Reactions:

The percentages of subjects who reported at least 1 solicited systemic reaction were 50.0% (16/32), 38.7% (12/31), 50.0% (15/30), and 40.8% (20/49) subjects in the QIV-HD (30 μg, 45 μg, 60 μg) groups and QIV-SD group, respectively (Table 22).

Few Grade 3 reactions were reported. There was no increase in the incidence of solicited systemic reactions after the 2nd vaccination compared to the 1st vaccination.

Unsolicited Non-Serious AEs Between D0 and D28 and SAEs/AESIs Throughout the Study

Within 30 minutes after any vaccination, none of the subjects experienced any unsolicited AE or AR.

Within 28 days after any vaccination, The percentages of subjects who experienced at least 1 unsolicited non-serious AE in the QIV-HD (30 μg, 45 μg, 60 μg) and QIV-SD group was 37.5% (12/32), 35.5% (11/31), 26.7% (8/30), and 28.6% (14/49), respectively. The percentages of subjects who experienced at least 1 unsolicited non-serious AR in the QIV-HD (30 μg, 45 μg, and 60 μg) and QIV-SD group were 3.1% (1/32), 3.2% (1/31), 3.3% (1/30), and 2.0% (1/49), respectively (Table 22).

TABLE 22
Safety overview after any vaccination (US subjects 5-8 years) - SafAS

| | US (5-8 years) | | | | | | | | | | | |
|---|---|---|---|---|---|---|---|---|---|---|---|---|
| Subjects | QIV-HD (30 μg) | | | QIV-HD (45 μg) | | | QIV-HD (60 μg) | | | QIV-SD | | |
| experiencing | (N = 32) | | | (N = 31) | | | (N = 30) | | | (N = 49) | | |
| at least one: | n/M | % | (95% CI) | n/M | % | (95% CI) | n/M | % | (95% CI) | n/M | % | (95% CI) |
| Within 28 days | | | | | | | | | | | | |
| after any | | | | | | | | | | | | |
| vaccination | | | | | | | | | | | | |
| Immediate | 0/32 | 0 | (0; 10.9) | 0/31 | 0 | (0; 11.2) | 0/30 | 0 | (0; 11.6) | 0/49 | 0 | (0; 7.3) |
| unsolicited AE | | | | | | | | | | | | |
| Immediate | 0/32 | 0 | (0; 10.9) | 0/31 | 0 | (0; 11.2) | 0/30 | 0 | (0; 11.6) | 0/49 | 0 | (0; 7.3) |
| unsolicited AR | | | | | | | | | | | | |
| Solicited | 28/32 | 87.5 | (71.0; 96.5) | 28/31 | 90.3 | (74.2; 98.0) | 26/30 | 86.7 | (69.3; 96.2) | 36/49 | 73.5 | (58.985.1) |
| reaction | | | | | | | | | | | | |
| Solicited | 25/32 | 78.1 | (60.0; 90.7) | 27/31 | 87.1 | (70.2; 96.4) | 26/30 | 86.7 | (69.3; 96.2) | 35/49 | 71.4 | (56.783.4) |
| injection | | | | | | | | | | | | |
| site reaction | | | | | | | | | | | | |
| Solicited | 16/32 | 50.0 | (31.9; 68.1) | 12/31 | 38.7 | (21.8; 57.8) | 15/30 | 50.0 | (31.3; 68.7) | 20/49 | 40.8 | (27.055.8) |
| systemic | | | | | | | | | | | | |
| reaction | | | | | | | | | | | | |
| Unsolicited AE | 12/32 | 37.5 | (21.1; 56.3) | 11/31 | 35.5 | (19.2; 54.6) | 8/30 | 26.7 | (12.3; 45.9) | 14/49 | 28.6 | (16.6433) |
| Unsolicited | 12/32 | 37.5 | (21.1; 56.3) | 11/31 | 35.5 | (19.2; 54.6) | 8/30 | 26.7 | (12.3; 45.9) | 14/49 | 28.6 | (16.6433) |
| non-serious AE | | | | | | | | | | | | |
| Unsolicited | 12/32 | 37.5 | (21.1; 56.3) | 11/31 | 35.5 | (19.2; 54.6) | 8/30 | 26.7 | (12.3; 45.9) | 14/49 | 28.6 | (16.6433) |
| non-serious | | | | | | | | | | | | |
| systemic AE | | | | | | | | | | | | |
| Unsolicited AR | 1/32 | 3.1 | (0.1; 16.2) | 1/31 | 3.2 | (0.1; 16.7) | 1/30 | 3.3 | (0.1; 17.2) | 1/49 | 2.0 | (0.1; 10.9) |
| Unsolicited | 1/32 | 3.1 | (0.1; 16.2) | 1/31 | 3.2 | (0.1; 16.7) | 1/30 | 3.3 | (0.1; 17.2) | 1/49 | 2.0 | (0.1; 10.9) |
| non-serious AR | | | | | | | | | | | | |
| Unsolicited | 0/32 | 0 | (0; 10.9) | 1/31 | 3.2 | (0.1; 16.7) | 0/30 | 0 | (0; 11.6) | 0/49 | 0 | (0; 7.3) |
| non-serious | | | | | | | | | | | | |
| injection | | | | | | | | | | | | |
| site AR | | | | | | | | | | | | |
| Unsolicited | 1/32 | 3.1 | (0.1; 16.2) | 0/31 | 0 | (0; 11.2) | 1/30 | 3.3 | (0.1; 17.2) | 1/49 | 2.0 | (0.1; 10.9) |
| non-serious | | | | | | | | | | | | |
| systemic AR | | | | | | | | | | | | |
| AE leading to | 0/32 | 0 | (0; 10.9) | 0/31 | 0 | (0; 11.2) | 0/30 | 0 | (0; 11.6) | 0/49 | 0 | (0; 7.3) |
| study | | | | | | | | | | | | |
| discontinuation | | | | | | | | | | | | |
| SAE within 28 | 0/32 | 0 | (0; 10.9) | 0/31 | 0 | (0; 11.2) | 0/30 | 0 | (0; 11.6) | 0/49 | 0 | (0; 7.3) |
| days after any | | | | | | | | | | | | |
| vaccination | | | | | | | | | | | | |
| Fatal | 0/32 | 0 | (0; 10.9) | 0/31 | 0 | (0; 11.2) | 0/30 | 0 | (0; 11.6) | 0/49 | 0 | (0; 7.3) |
| AE of special | 0/32 | 0 | (0; 10.9) | 0/31 | 0 | (0; 11.2) | 0/30 | 0 | (0; 11.6) | 0/49 | 0 | (0; 7.3) |
| interest | | | | | | | | | | | | |
| From 29 days | | | | | | | | | | | | |
| after the last | | | | | | | | | | | | |
| vaccination | | | | | | | | | | | | |
| SAE | 0/32 | 0 | (0; 10.9) | 0/31 | 0 | (0; 11.2) | 0/30 | 0 | (0; 11.6) | 0/49 | 0 | (0; 7.3) |
| Fatal | 0/32 | 0 | (0; 10.9) | 0/31 | 0 | (0; 11.2) | 0/30 | 0 | (0; 11.6) | 0/49 | 0 | (0; 7.3) |
| AE of special | 0/32 | 0 | (0; 10.9) | 0/31 | 0 | (0; 11.2) | 0/30 | 0 | (0; 11.6) | 0/49 | 0 | (0; 7.3) |
| interest (AESI) | | | | | | | | | | | | |
| During the | | | | | | | | | | | | |
| entire study | | | | | | | | | | | | |
| SAE | 0/32 | 0 | (0; 10.9) | 0/31 | 0 | (0; 11.2) | 0/30 | 0 | (0; 11.6) | 0/49 | 0 | (0; 7.3) |
| Fatal | 0/32 | 0 | (0; 10.9) | 0/31 | 0 | (0; 11.2) | 0/30 | 0 | (0; 11.6) | 0/49 | 0 | (0; 7.3) |
| AE of special | 0/32 | 0 | (0; 10.9) | 0/31 | 0 | (0; 11.2) | 0/30 | 0 | (0; 11.6) | 0/49 | 0 | (0; 7.3) |
| interest (AESI) | | | | | | | | | | | | |

n: number of subjects experiencing the endpoint listed in the first column
M: number of subjects with available data for the relevant endpoint

The SOC with the highest incidence of unsolicited non-serious AEs was “Respiratory, thoracic and mediastinal disorders” with 7 AEs reported by 15.6% (5/32) of subjects in the QIV-HD (30 μg) group; 12 AEs reported by 25.8% (8/31) of subjects in the QIV-HD (45 μg) group; 3 AEs reported by 10.0% (3/30) of subjects in the QIV-HD (60 μg) group; and 10 AEs reported by 12.2% (6/49) of subjects in the QIV-SD group. The most frequently reported PT in the QIV-HD (30 μg, 45 μg, and 60 μg) groups and QIV-SD group was cough.

None of the subjects experienced any AEs leading to study discontinuation, SAEs, fatal SAEs, or AESIs throughout the study (Table 22).

2.3.4—Subjects 36 Months—Less than 5 Years of Age (Groups 1 [Stage 1], 2 [Stage 2], and 4 [Stage 3]—US)

Solicited Reactions Between D0 and D7

Within 7 days of any vaccination, the percentages of subjects who reported at least 1 solicited reaction were similar among 2 QIV-HD groups (30 μg and 45 μg) and slightly lower in the QIV-HD (60 μg) and QIV-SD groups: 71.9% (23/32), 72.4% (21/29), 56.8% (25/44), and 60.3% (41/68) in the QIV-HD (30 μg, 45 μg, and 60 μg) groups and QIV-SD group, respectively (Table 23).

Solicited Injection Site Reactions:

The percentages of subjects who reported at least 1 solicited injection site reaction were 71.9% (23/32), 69.0% (20/29), 54.5% (24/44), and 52.9% (36/68) subjects in the QIV-HD groups (30 μg, 45 μg, and 60 μg) and QIV-SD group, respectively (Table 23).

Few Grade 3 reactions were reported after vaccination 1. There was no increase in incidence of solicited injection site reactions after the 2nd vaccination compared to the 1st vaccination. No Grade 3 reactions were reported after vaccination 2.

Solicited Systemic Reactions:

The percentages of subjects who reported at least 1 solicited systemic reaction were 40.6% (13/32), 44.8% (13/29), 29.5% (13/44), and 35.3% (24/68) subjects in the QIV-HD (30 μg, 45 μg, 60 μg) groups and QIV-SD group, respectively (Table 23).

Few Grade 3 reactions were reported after vaccination 1. There was no increase in the incidence of solicited systemic reactions after the 2nd vaccination compared to the 1st vaccination. No Grade 3 reactions were reported after vaccination 2.

Unsolicited Non-Serious AEs Between D0 and D28 and SAEs/AESIs Throughout the Study

Within 30 minutes after any vaccination, none of the subjects experienced any unsolicited AE or AR.

Within 28 days after any vaccination, the percentages of subjects who experienced at least 1 unsolicited non-serious AE in the QIV-HD (30 μg, 45 μg, and 60 μg) and QIV-SD group was 37.5% (12/32), 46.7% (14/30), 40.9% (18/44), and 30.9% (21/68), respectively. The percentages of subjects who experienced at least 1 unsolicited non-serious AR in the QQV-HD (30 μg, 45 μg, and 60 Vg) and QIV-SD group was 0% (0/32), 3.3% (1/30), 2.3% (1/44), and 2.9% (2/68), respectively (Table 23).

The SOC with the highest incidence of unsolicited non-serious AEs was “Respiratory, thoracic and mediastinal disorders” with 6 AEs reported by 15.6% (5/32) of subjects in the QIV-HD (30 μg) group; 8 AEs reported by 23.3% (7/30) of subjects in the QIV-HD (45 μg) group; 15 AEs reported by 20.5% (9/44) of subjects in the QIV-HD (60 μg) group; and 19 AEs reported by 23.5% (16/68) of subjects in the QIV-SD group. The most frequently reported PT in the QIV-HD (30 tag, 45 tag, 60 μg) groups and QIV-SD group was cough.

None of the subjects experienced any AEs leading to study discontinuation, SAEs, fatal SAEs, or AESIs throughout the study (Table 23).

TABLE 23
Safety overview after any vaccination (US subjects 36 months-less than 5 years) - SafAS

| | US (36 months-less than 5 years) | | | | | | | | | | | |
|---|---|---|---|---|---|---|---|---|---|---|---|---|
| Subjects | QIV-HD (30 μg) | | | QIV-HD (45 μg) | | | QIV-HD (60 μg) | | | QIV-SD | | |
| experiencing | (N = 32) | | | (N = 30) | | | (N = 44) | | | (N = 68) | | |
| at least one: | n/M | % | (95% CI) | n/M | % | (95% CI) | n/M | % | (95% CI) | n/M | % | (95% CI) |
| Within 28 days | | | | | | | | | | | | |
| after any | | | | | | | | | | | | |
| vaccination | | | | | | | | | | | | |
| Immediate | 0/32 | 0 | (0; 10.9) | 0/30 | 0 | (0; 11.6) | 0/44 | 0 | (0; 8.0) | 0/68 | 0 | (0; 5.3) |
| unsolicited AE | | | | | | | | | | | | |
| Immediate | 0/32 | 0 | (0; 10.9) | 0/30 | 0 | (0; 11.6) | 0/44 | 0 | (0; 8.0) | 0/68 | 0 | (0; 5.3) |
| unsolicited AR | | | | | | | | | | | | |
| Solicited | 23/32 | 71.9 | (53.3; 86.3) | 21/29 | 72.4 | (52.8; 87.3) | 25/44 | 56.8 | (41.0; 71.7) | 41/68 | 60.3 | (47.7; 72.0) |
| reaction | | | | | | | | | | | | |
| Solicited | 23/32 | 71.9 | (53.3; 86.3) | 20/29 | 69.0 | (49.2; 84.7) | 24/44 | 54.5 | (38.8; 69.6) | 36/68 | 52.9 | (40.4; 65.2) |
| injection | | | | | | | | | | | | |
| site reaction | | | | | | | | | | | | |
| Solicited | 13/32 | 40.6 | (23.7; 59.4) | 13/29 | 44.8 | (26.4; 64.3) | 13/44 | 29.5 | (16.8; 45.2) | 24/68 | 35.3 | (24.1; 47.8) |
| systemic | | | | | | | | | | | | |
| reaction | | | | | | | | | | | | |
| Unsolicited AE | 12/32 | 37.5 | (21.1; 56.3) | 14/30 | 46.7 | (28.3; 65.7) | 18/44 | 40.9 | (26.3; 56.8) | 21/68 | 30.9 | (20.2; 43.3) |
| Unsolicited | 12/32 | 37.5 | (21.1; 56.3) | 14/30 | 46.7 | (28.3; 65.7) | 18/44 | 40.9 | (26.3; 56.8) | 21/68 | 30.9 | (20.2; 43.3) |
| non-serious AE | | | | | | | | | | | | |
| Unsolicited | 12/32 | 37.5 | (21.1; 56.3) | 14/30 | 46.7 | (28.3; 65.7) | 18/44 | 40.9 | (26.3; 56.8) | 21/68 | 30.9 | (20.2; 43.3) |
| non-serious | | | | | | | | | | | | |
| systemic AE | | | | | | | | | | | | |
| Unsolicited AR | 0/32 | 0 | (0; 10.9) | 1/30 | 3.3 | (0.1; 17.2) | 1/44 | 2.3 | (0.1; 12.0) | 2/68 | 2.9 | (0.4; 10.2) |
| Unsolicited | 0/32 | 0 | (0; 10.9) | 1/30 | 3.3 | (0.1; 17.2) | 1/44 | 2.3 | (0.1; 12.0) | 2/68 | 2.9 | (0.4; 10.2) |
| non-serious AR | | | | | | | | | | | | |
| Unsolicited | 0/32 | 0 | (0; 10.9) | 0/30 | 0 | (0; 11.6) | 0/44 | 0 | (0; 8.0) | 0/68 | 0 | (0; 5.3) |
| non-serious | | | | | | | | | | | | |
| injection | | | | | | | | | | | | |
| site AR | | | | | | | | | | | | |
| Unsolicited | 0/32 | 0 | (0; 10.9) | 1/30 | 3.3 | (0.1; 17.2) | 1/44 | 2.3 | (0.1; 12.0) | 2/68 | 2.9 | (0.4; 10.2) |
| non-serious | | | | | | | | | | | | |
| systemic AR | | | | | | | | | | | | |
| AE leading to | 0/32 | 0 | (0; 10.9) | 0/30 | 0 | (0; 11.6) | 0/44 | 0 | (0; 8.0) | 0/68 | 0 | (0; 5.3) |
| study | | | | | | | | | | | | |
| discontinuation | | | | | | | | | | | | |
| SAE within 28 | 0/32 | 0 | (0; 10.9) | 0/30 | 0 | (0; 11.6) | 0/44 | 0 | (0; 8.0) | 0/68 | 0 | (0; 5.3) |
| days after any | | | | | | | | | | | | |
| vaccination | | | | | | | | | | | | |
| Fatal | 0/32 | 0 | (0; 10.9) | 0/30 | 0 | (0; 11.6) | 0/44 | 0 | (0; 8.0) | 0/68 | 0 | (0; 5.3) |
| AE of special | 0/32 | 0 | (0; 10.9) | 0/30 | 0 | (0; 11.6) | 0/44 | 0 | (0; 8.0) | 0/68 | 0 | (0; 5.3) |
| interest | | | | | | | | | | | | |
| From 29 days | | | | | | | | | | | | |
| after the last | | | | | | | | | | | | |
| vaccination | | | | | | | | | | | | |
| SAE | 0/32 | 0 | (0; 10.9) | 0/30 | 0 | (0; 11.6) | 0/44 | 0 | (0; 8.0) | 0/68 | 0 | (0; 5.3) |
| Fatal | 0/32 | 0 | (0; 10.9) | 0/30 | 0 | (0; 11.6) | 0/44 | 0 | (0; 8.0) | 0/68 | 0 | (0; 5.3) |
| AE of special | 0/32 | 0 | (0; 10.9) | 0/30 | 0 | (0; 11.6) | 0/44 | 0 | (0; 8.0) | 0/68 | 0 | (0; 5.3) |
| interest (AESI) | | | | | | | | | | | | |
| During the | | | | | | | | | | | | |
| entire study | | | | | | | | | | | | |
| SAE | 0/32 | 0 | (0; 10.9) | 0/30 | 0 | (0; 11.6) | 0/44 | 0 | (0; 8.0) | 0/68 | 0 | (0; 5.3) |
| Fatal | 0/32 | 0 | (0; 10.9) | 0/30 | 0 | (0; 11.6) | 0/44 | 0 | (0; 8.0) | 0/68 | 0 | (0; 5.3) |
| AE of special | 0/32 | 0 | (0; 10.9) | 0/30 | 0 | (0; 11.6) | 0/44 | 0 | (0; 8.0) | 0/68 | 0 | (0; 5.3) |
| interest (AESI) | | | | | | | | | | | | |

n: number of subjects experiencing the endpoint listed in the first column
M: number of subjects with available data for the relevant endpoint

2.3.5—Subjects 6—Less than 36 Months of Age (Groups 3 [Stage 2], 5 [Stage 3], and 6-7 [Stage 4]—US)

Solicited Reactions Between D0 and D7

Within 7 days of any vaccination, the percentages of subjects who reported at least 1 solicited reaction were similar among the 3 QIV-HD groups (30 μg, 45 μg, and 60 μg) and slightly higher for each QIV-HD group compared to QIV-SD group: 69.0% (20/29), 70.0% (21/30), 66.7% (36/54), and 56.8% (42/74) in the QIV-HD (30 μg, 45 μg, 60 μg) groups and QIV-SD group, respectively (Table 24).

Solicited Injection Site Reactions:

The percentages of subjects who reported at least 1 solicited injection site reaction were 62.1% (18/29), 63.3% (19/30), 51.9% (28/54), and 43.2% (32/74) subjects in the QIV-HD groups (30 μg, 45 μg, 60 μg) and QIV-SD group, respectively (Table 24).

After vaccination 1, the most frequently reported solicited injection site reactions in the QIV-HD (30 μg, 45 μg, 60 μg) groups and QIV-SD group was tenderness, followed by erythema. Injection site reactions that were less frequently reported by subjects were swelling, induration, and bruising.

After vaccination 2, the most frequently reported solicited injection site reactions was tenderness. Swelling, erythema, induration, and bruising were less frequently reported. The frequency of injection site reactions reported by subjects in the QIV-HD (30 μg, 45 μg, 60 μg) groups and QIV-SD group did not increase after vaccination 2.

The majority of solicited injection site reactions in the 4 vaccine groups were of Grade 1 intensity.

After vaccination 1, there were few reports of Grade 3 tenderness, swelling, and induration. Grade 3 erythema and bruising were not reported in any vaccine group.

After vaccination 2, there were no reports of Grade 3 solicited injection site reactions in the 3 QIV-HD groups, and only 1 subject in the QIV-SD group experienced injection site tenderness of Grade 3 intensity.

Solicited Systemic Reactions:

Within 7 days of any vaccination, the percentages of subjects who experienced at least 1 solicited systemic reaction were similar between all study groups for each solicited systemic reaction with the overall percentages being: 62.1% (18/29), 53.3% (16/30), 44.4% (24/54), and 50.0% (37/74) subjects in the QIV-HD (30 μg, 45 μg, 60 μg) groups and QIV-SD group, respectively.

After vaccination 1, the most frequently reported solicited systemic reactions in the QIV-HD (30 μg, 45 μg, 60 μg) groups and QIV-SD group was irritability, followed by drowsiness, crying abnormal, and appetite lost. Systemic reactions that were less frequently reported by subjects in the QIV-HD (30 μg, 45 μg, 60 μg) groups and QIV-SD group were fever and vomiting.

After vaccination 2, the most frequently reported solicited systemic reactions in the QIV-HD (30 μg, 45 μg, 60 μg) groups and QIV-SD group was irritability, followed by drowsiness and crying abnormal. Systemic reactions that were less frequently reported by the subjects in the QIV-HD (30 μg, 45 μg, 60 μg) groups and QIV-SD group were appetite lost, fever, and vomiting.

The majority of solicited systemic reactions in the 4 vaccine groups were of Grade 1 or Grade 2 intensity.

Few Grade 3 reactions were reported among 4 vaccine groups after vaccination 1. After vaccination 2, Grade 3 drowsiness and Grade 3 appetite lost were reported by 1 subject each in the QIV-HD (45 μg) group, and Grade 3 fever was reported by 1 subject in the QIV-HD (60 μg) group. In addition, there was no increase in the incidence of solicited systemic reactions after vaccination 2 compared to vaccination 1.

Unsolicited Non-Serious AEs Between D0 and D28 and SAEs/AESI Throughout the Study

Within 30 minutes after any vaccination, none of the subjects experienced any unsolicited AE or AR.

Within 28 days after any vaccination, the percentages of subjects who experienced at least 1 unsolicited non-serious systemic AE in the QIV-HD (30 μg, 45 μg, 60 μg) and QIV-SD group were 55.2% (16/29), 43.3% (13/30), 40.7% (22/54), and 50.7% (38/75), respectively. The percentages of subjects who experienced at least 1 unsolicited non-serious AR in the QIV-HD (30 μg, 45 μg, 60 μg) and QIV-SD group were 6.9% (2/29), 13.3% (4/30), 5.6% (3/54), and 6.7% (5/75), respectively. One subject in the QIV-HD (45 μg) group experienced non-solicited injection site AR (Table 24).

TABLE 24
Safety overview after any vaccinations (US subjects 6-less than 36 months) - SafAS

| | US (6-less than 36 months) | | | | | | | | | | | |
|---|---|---|---|---|---|---|---|---|---|---|---|---|
| Subjects | QIV-HD (30 μg) | | | QIV-HD (45 μg) | | | QIV-HD (60 μg) | | | QIV-SD | | |
| experiencing | (N = 29) | | | (N = 30) | | | (N = 54) | | | (N = 75) | | |
| at least one: | n/M | % | (95% CI) | n/M | % | (95% CI) | n/M | % | (95% CI) | n/M | % | (95% CI) |
| Within 28 days | | | | | | | | | | | | |
| after any | | | | | | | | | | | | |
| vaccination | | | | | | | | | | | | |
| Immediate | 0/29 | 0 | (0; 11.9) | 0/30 | 0 | (0; 11.6) | 0/54 | 0 | (0; 6.6) | 0/75 | 0 | (0; 4.8) |
| unsolicited AE | | | | | | | | | | | | |
| Immediate | 0/29 | 0 | (0; 11.9) | 0/30 | 0 | (0; 11.6) | 0/54 | 0 | (0; 6.6) | 0/75 | 0 | (0; 4.8) |
| unsolicited AR | | | | | | | | | | | | |
| Solicited | 20/29 | 69.0 | (49.2; 84.7) | 21/30 | 70.0 | (50.6; 85.3) | 36/54 | 66.7 | (52.5; 78.9) | 42/74 | 56.8 | (44.7; 68.2) |
| reaction | | | | | | | | | | | | |
| Solicited | 18/29 | 62.1 | (42.3; 79.3) | 19/30 | 63.3 | (43.9; 80.1) | 28/54 | 51.9 | (37.8; 65.7) | 32/74 | 43.2 | (31.8; 55.3) |
| injection | | | | | | | | | | | | |
| site reaction | | | | | | | | | | | | |
| Solicited | 18/29 | 62.1 | (42.3; 79.3) | 16/30 | 53.3 | (34.3; 71.7) | 24/54 | 44.4 | (30.9; 58.6) | 37/74 | 50.0 | (38.1; 61.9) |
| systemic | | | | | | | | | | | | |
| reaction | | | | | | | | | | | | |
| Unsolicited AE | 16/29 | 55.2 | (35.7; 73.6) | 13/30 | 43.3 | (25.5; 62.6) | 23/54 | 42.6 | (29.2; 56.8) | 38/75 | 50.7 | (38.9; 62.4) |
| Unsolicited | 16/29 | 55.2 | (35.7; 73.6) | 13/30 | 43.3 | (25.5; 62.6) | 22/54 | 40.7 | (27.6; 55.0) | 38/75 | 50.7 | (38.9; 62.4) |
| non-serious AE | | | | | | | | | | | | |
| Unsolicited | 16/29 | 55.2 | (35.7; 73.6) | 13/30 | 43.3 | (25.5; 62.6) | 22/54 | 40.7 | (27.6; 55.0) | 38/75 | 50.7 | (38.9; 62.4) |
| non-serious | | | | | | | | | | | | |
| systemic AE | | | | | | | | | | | | |
| Unsolicited AR | 2/29 | 6.9 | (0.8; 22.8) | 4/30 | 13.3 | (3.8; 30.7) | 3/54 | 5.6 | (1.2; 15.4) | 5/75 | 6.7 | (2.2; 14.9) |
| Unsolicited | 2/29 | 6.9 | (0.8; 22.8) | 4/30 | 13.3 | (3.8; 30.7) | 3/54 | 5.6 | (1.2; 15.4) | 5/75 | 6.7 | (2.2; 14.9) |
| non-serious AR | | | | | | | | | | | | |
| Unsolicited | 0/29 | 0 | (0; 11.9) | 1/30 | 3.3 | (0.1; 17.2) | 0/54 | 0 | (0; 6.6) | 0/75 | 0 | (0; 4.8) |
| non-serious | | | | | | | | | | | | |
| injection | | | | | | | | | | | | |
| site AR | | | | | | | | | | | | |
| Unsolicited | 2/29 | 6.9 | (0.8; 22.8) | 3/30 | 10.0 | (2.1; 26.5) | 3/54 | 5.6 | (1.2; 15.4) | 5/75 | 6.7 | (2.2; 14.9) |
| non-serious | | | | | | | | | | | | |
| systemic AR | | | | | | | | | | | | |
| AE leading to | 0/29 | 0 | (0; 11.9) | 0/30 | 0 | (0; 11.6) | 0/54 | 0 | (0; 6.6) | 0/75 | 0 | (0; 4.8) |
| study | | | | | | | | | | | | |
| discontinuation | | | | | | | | | | | | |
| SAE within 28 | 0/29 | 0 | (0; 11.9) | 0/30 | 0 | (0; 11.6) | 2/54 | 3.7 | (0.5; 12.7) | 0/75 | 0 | (0; 4.8) |
| days after any | | | | | | | | | | | | |
| vaccination | | | | | | | | | | | | |
| Fatal | 0/29 | 0 | (0; 11.9) | 0/30 | 0 | (0; 11.6) | 0/54 | 0 | (0; 6.6) | 0/75 | 0 | (0; 4.8) |
| AE of special | 0/29 | 0 | (0; 11.9) | 0/30 | 0 | (0; 11.6) | 1/54 | 1.9 | (0; 9.9) | 0/75 | 0 | (0; 4.8) |
| interest | | | | | | | | | | | | |
| From 29 days | | | | | | | | | | | | |
| after the last | | | | | | | | | | | | |
| vaccination | | | | | | | | | | | | |
| SAE | 0/29 | 0 | (0; 11.9) | 0/30 | 0 | (0; 11.6) | 0/54 | 0 | (0; 6.6) | 0/75 | 0 | (0; 4.8) |
| Fatal | 0/29 | 0 | (0; 11.9) | 0/30 | 0 | (0; 11.6) | 0/54 | 0 | (0; 6.6) | 0/75 | 0 | (0; 4.8) |
| AE of special | 0/29 | 0 | (0; 11.9) | 0/30 | 0 | (0; 11.6) | 0/54 | 0 | (0; 6.6) | 0/75 | 0 | (0; 4.8) |
| interest (AESI) | | | | | | | | | | | | |
| During the | | | | | | | | | | | | |
| entire study | | | | | | | | | | | | |
| SAE | 0/29 | 0 | (0; 11.9) | 0/30 | 0 | (0; 11.6) | 2/54 | 3.7 | (0.5; 12.7) | 0/75 | 0 | (0; 4.8) |
| Fatal | 0/29 | 0 | (0; 11.9) | 0/30 | 0 | (0; 11.6) | 0/54 | 0 | (0; 6.6) | 0/75 | 0 | (0; 4.8) |
| AE of special | 0/29 | 0 | (0; 11.9) | 0/30 | 0 | (0; 11.6) | 1/54 | 1.9 | (0; 9.9) | 0/75 | 0 | (0; 4.8) |
| interest (AESI) | | | | | | | | | | | | |

n: number of subjects experiencing the endpoint listed in the first column
M: number of subjects with available data for the relevant endpoint

The SOC with the highest incidence of unsolicited non-serious AEs was “Respiratory, thoracic and mediastinal disorders” with 8 AEs reported by 17.2% (5/29) of subjects in the QIV-HD (30 μg) group; 17 AEs reported by 30% (9/30) of subjects in the QIV-HD (45 μg) group; 10 AEs reported by 7.4% (4/54) of subjects in the QIV-HD (60 μg) group; and 20 AEs reported by 18.7% (14/75) of subjects in the QIV-SD group. The most frequently reported PT in this SOC for the QIV-HD (30 μg, 45 μg, and 60 μg) groups and QIV-SD group was cough.

None of the subjects experienced any AEs leading to study discontinuation or fatal SAEs (Table 24).

Two SAEs were reported between D0 and D28 in the QIV-HD (60 μg) group: febrile seizure (considered an AESI) and RSV infection. Both SAEs were considered unrelated to the vaccine by the Investigator and the Sponsor.

The febrile seizure SAE occurred in a 16-month-old, Group 6 subject (previously influenza unvaccinated) who received two doses of the study vaccine. 22 days post-vaccination, the subject had a fever of 104° F. (axillary) and a sibling witnessed a possible seizure at home. The subject's seizure episode reportedly included symptoms of his eyes roll to the back of his head, his body becoming limp, shaking, and foaming at the mouth for approximately 3 minutes. The subject did not lose consciousness. The mother brought the subject to the Emergency Room where the outside hospital records reported nasal congestion and a possible upper respiratory tract infection. However, the mother denied any other symptoms. The subject was not hospitalized and did not have any further seizure episodes or new symptoms develop. The mother felt the fever was due to the subject's teething. The subject received the second dose of study vaccine after this febrile seizure episode because the mother did not report the subject's seizure episode until Visit 04 after the second dose of study vaccine had been administered. Prior to enrolling in the study, the subject had last received hepatitis A, MMR, PCV13, and varicella vaccines approximately one month prior to the first influenza vaccine dose in this study. The investigator did not consider the febrile seizure to be related to the study vaccine given the hospital records reporting an upper respiratory tract infection.

The RSV infection SAE occurred in a 2-year-old, Group 6 subject (previously influenza unvaccinated) who received two doses of the study vaccine. Symptoms of a cough and difficulty breathing began 12 days after the first dose, and a fever to 103° F. was recorded 14 days after the first dose. An RSV test was positive and an influenza test was negative. Due to worsening symptoms, the subject went to the Emergency Room and was hospitalized 16 days after the first dose. The subject was also diagnosed with otitis media and treated with amoxicillin during the hospitalization. Symptoms resolved 22 days after the first dose. The investigator did not consider the RSV infection to be related to the study vaccine given that there was RSV infection activity in the community during that time and the subject likely contracted RSV from a sick contact.

2.3.6—Subjects 6—Less than 24 Months of Age (Group 8 [Stage 4]—Canada)

Solicited Reactions Between D0 and D7

Within 7 days of any vaccination, all the subjects (100%) in the QIV-HD (60 μg) and adjuvanted TIV reported at least 1 solicited reaction (Table 20C).

Solicited Injection Site Reactions:

The percentages of subjects who reported at least 1 solicited injection site reaction were 69.2% (9/13) for both the QIV-HD (60 μg) group and adjuvanted TIV group (Table 20C).

The majority of solicited injection site reactions in the QIV-HD (60 μg) and TIV adjuvanted vaccine groups were of Grade 1 intensity.

After vaccination 1, there were no reports of Grade 3 solicited injection site reactions in the QIV-HD (60 μg) group and adjuvanted TIV group.

After vaccination 2, 1 subject experienced Grade 3 erythema in the QIV-HD (60 μg) group.

Solicited Systemic Reactions:

All the subjects (100%) in the QIV-HD (60 μg) and adjuvanted TIV reported at least 1 solicited systemic reaction (Table 20C).

The majority of solicited injection site reactions in the QIV-HD (60 μg) and TIV adjuvanted vaccine groups were of Grade 1 or Grade 2 intensity.

After vaccination 1, there were no reports of Grade 3 solicited systemic reactions in the QIV-HD (60 μg) group and adjuvanted TIV group.

After vaccination 2, 1 subject experienced Grade 3 crying abnormal in the QIV-HD (60 μg) group and 1 subject experienced Grade 3 fever in the adjuvanted TIV group.

Unsolicited non-serious AEs and SAEs between D0 and D28 and SAEs/AESIs throughout the study

Within 30 minutes after any vaccination, none of the subjects experienced any unsolicited AE or AR.

Within 28 days after any vaccination, the percentages of subjects who experienced at least 1 unsolicited non-serious systemic AE in the QIV-HD (60 μg) and adjuvanted-TIV groups was 84.6% (11/13) and 92.3% (12/13), respectively. The percentages of subjects who experienced at least 1 unsolicited non-serious systemic AR in the QIV-HD (60 μg) and adjuvanted-TIV groups was 7.7% (1/13) and 15.4% (2/13), respectively. One subject in the adjuvanted-TIV group experienced unsolicited non-serious injection site AR (Table 20C).

The SOC with the highest incidence of unsolicited non-serious AEs was “Infections and infestations” with 25 AEs reported by 76.9% (10/13) of subjects in the QIV-HD (60 μg) group and 21 AEs reported by 69.2% (9/13) of subjects in the adjuvanted TIV group. The most frequently reported PT in the QIV-HD (60 μg) groups and adjuvanted TIV group was Nasopharyngitis.

None of the subjects experienced any AEs leading to study discontinuation, SAEs, fatal SAEs, and AESIs.

2.3.7—Overall Safety Conclusions

Vaccination with the 30, 45, and 60 μg dose QIV-HD formulations was found to be safe and well-tolerated among children 6 months to less than 18 years of age, with no safety concerns identified. In general, QIV-HD showed slightly higher reactogenicity (solicited injection site reactions and solicited systemic reactions) compared with QIV-SD. However, reactogenicity did not increase with increasing QIV-HD dose (i.e., HA content). Moreover, reactogenicity did not increase after a second dose was administered, compared to the reactogenicity after the first dose was administered, in subjects who received 2 doses of study vaccine approximately 28 days apart.

QIV-HD and QIV-SD showed similar rates of unsolicited events, AEs leading to study discontinuation, SAEs, fatal SAEs, and AESIs within 28 days after any vaccination. No deaths or related SAEs were reported in any groups throughout the study. Within 28 days after any vaccination, 2 SAEs (febrile seizure [considered an AESI] and RSV infection, both in the QIV-HD 60 μg dose group) were reported as unrelated as assessed by the Investigator and the Sponsor. Between D29 and the end of the study, 1 SAE (severe bilateral otitis in the adjuvanted TIV group) was reported as unrelated as assessed by the Investigator and the Sponsor.

The safety profile of QIV-HD and adjuvanted TIV were similar in the Canadian cohort of the study.

### 2.4—Immunogenicity Results

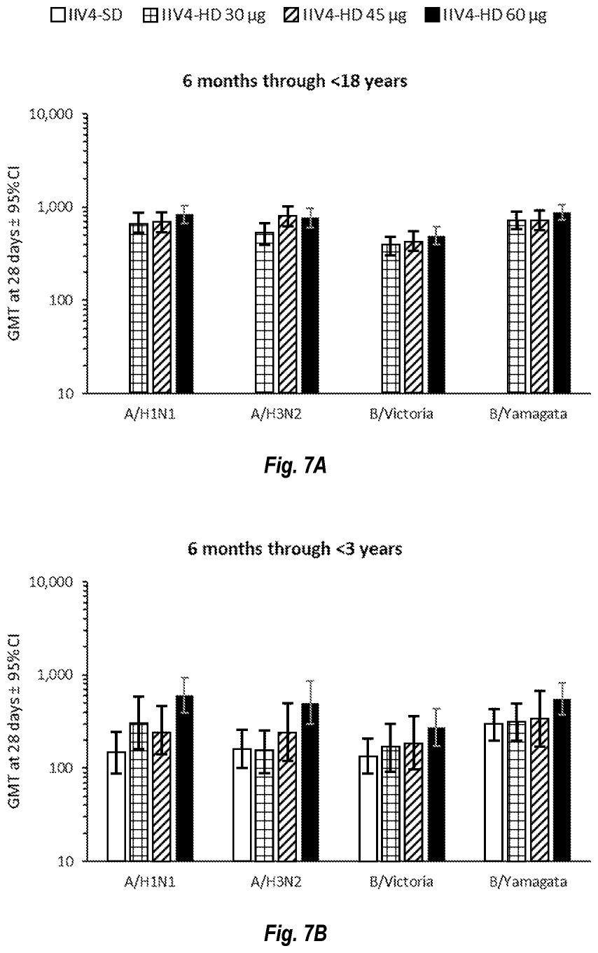

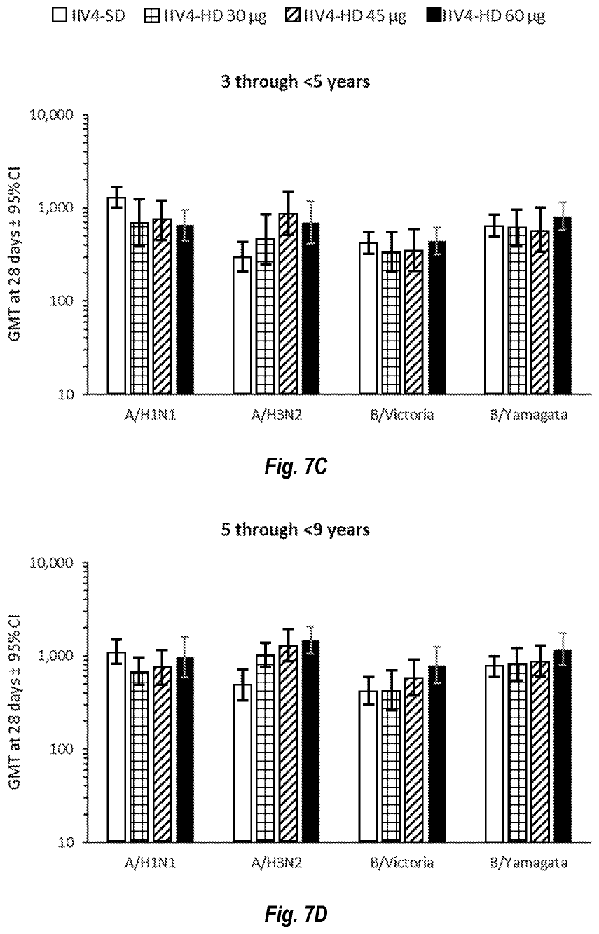

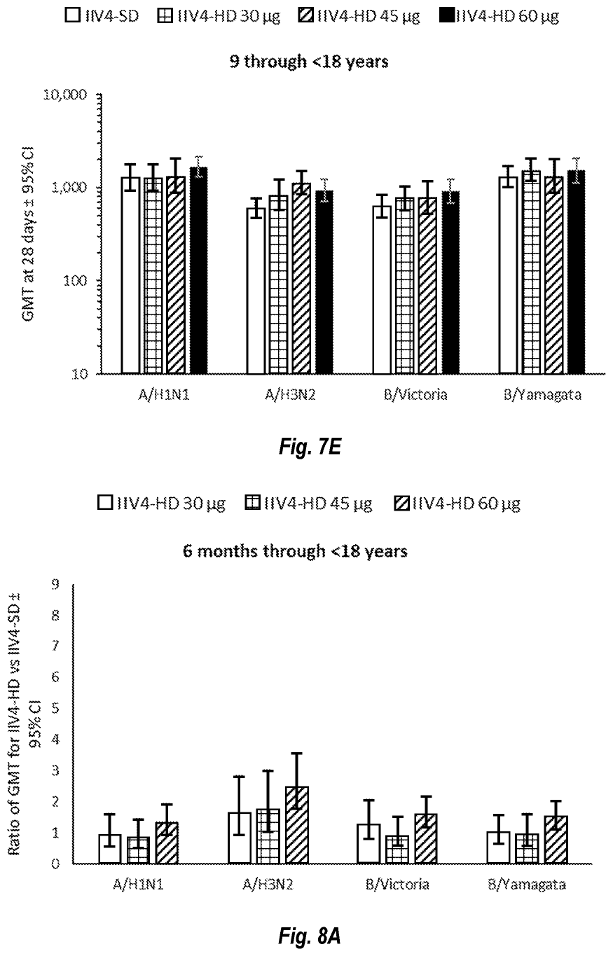

GMT at 28 days post-vaccination increased with increasing age (FIGS. 7A-7E and Table 25). GMT generally increased with increasing QIV-HD dose, especially in children aged 6 months through <3 years. This difference was not apparent in children aged 3 to <5 years. An analysis adjusting for the pre-vaccination titer also showed the same trends.

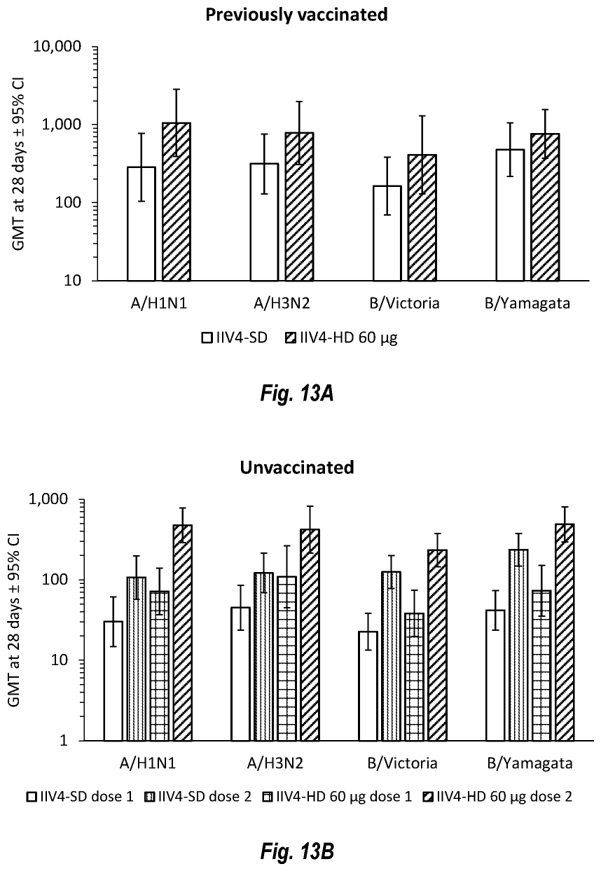

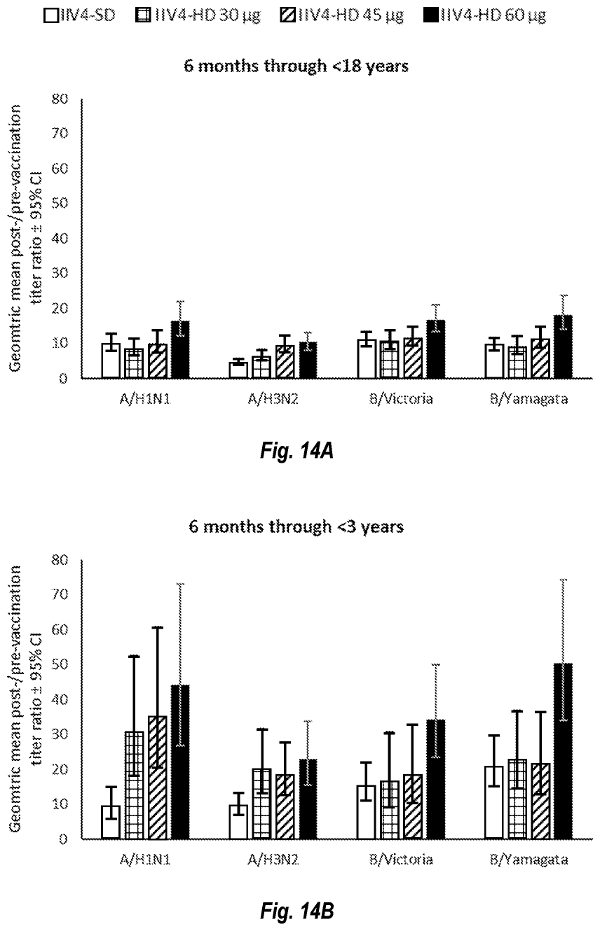

For previously unvaccinated participants aged 6 months through <3 years (who received two vaccine doses of QIV-HD 60 μg), GMT were higher after the second vaccine dose than after the first vaccine dose (FIGS. 13A-13B). However, GMTs were lower after the second vaccine dose in previously unvaccinated participants than after a single dose of vaccination in previously vaccinated participants.

After a single dose of QIV-HD in previously unvaccinated participants, similar GMTs were observed for H1N1 and H3N2 in comparison to 2 doses of QIV-SD, although B antibody levels were lower.

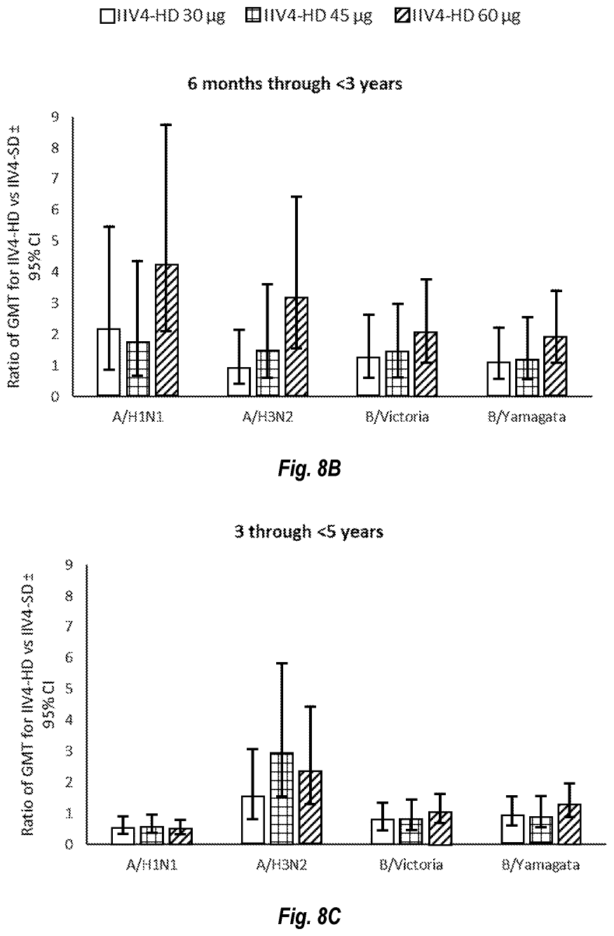

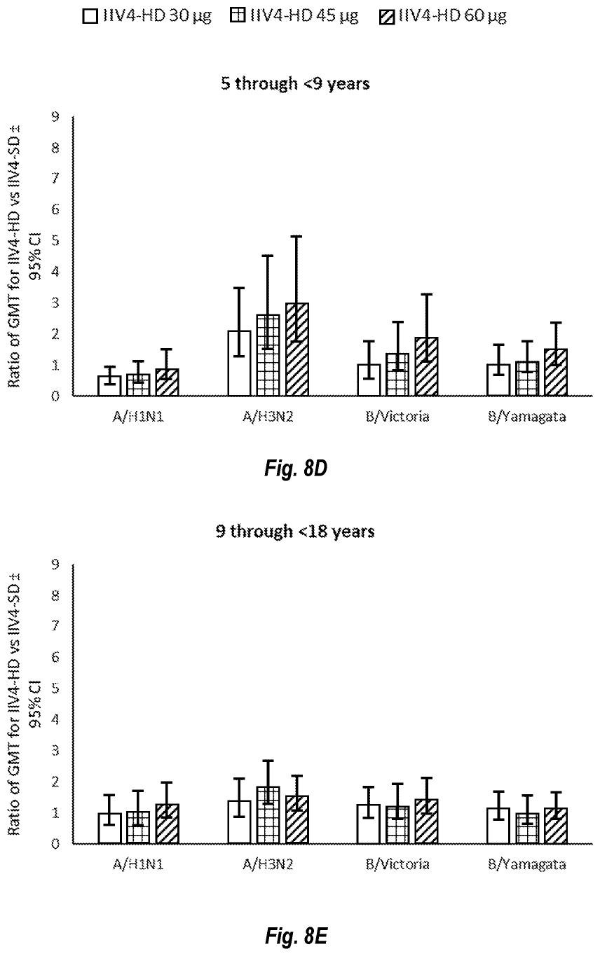

Overall, ratios of GMTs for QIV-HD versus QIV-SD were higher for QIV-HD 60 μg than for QIV-HD 30 μg or QIV-HD 45 μg (FIGS. 8A-8E). For QIV-HD 60 μg, the GMT ratio (95% CI) versus QIV-SD was 1.35 (0.94, 1.94) for A/H1N1, 2.51 (1.77, 3.55) for A/H3N2, 1.60 (1.17, 2.18) for B/Victoria, and 1.51 (1.13, 2.03) for B/Yamagata. The GMT ratio (95% CI) versus QIV-SD was highest for participants aged 6 months through <3 years: 4.24 (2.05, 8.76) for A/H1N1, 3.14 (1.53, 6.44) for A/H3N2, 2.04 (1.10, 3.77) for B/Victoria, and 1.92 (1.08, 3.41) for B/Yamagata.

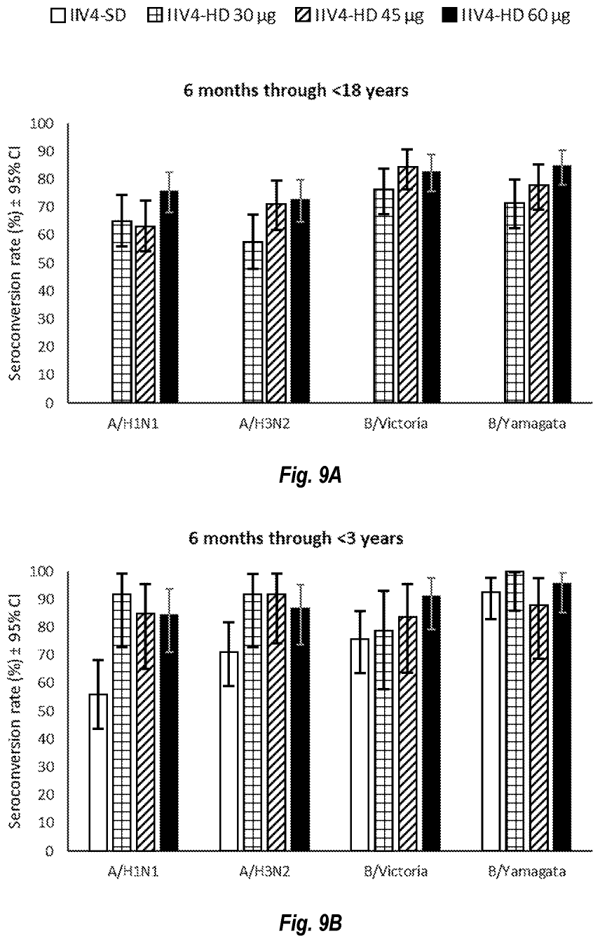

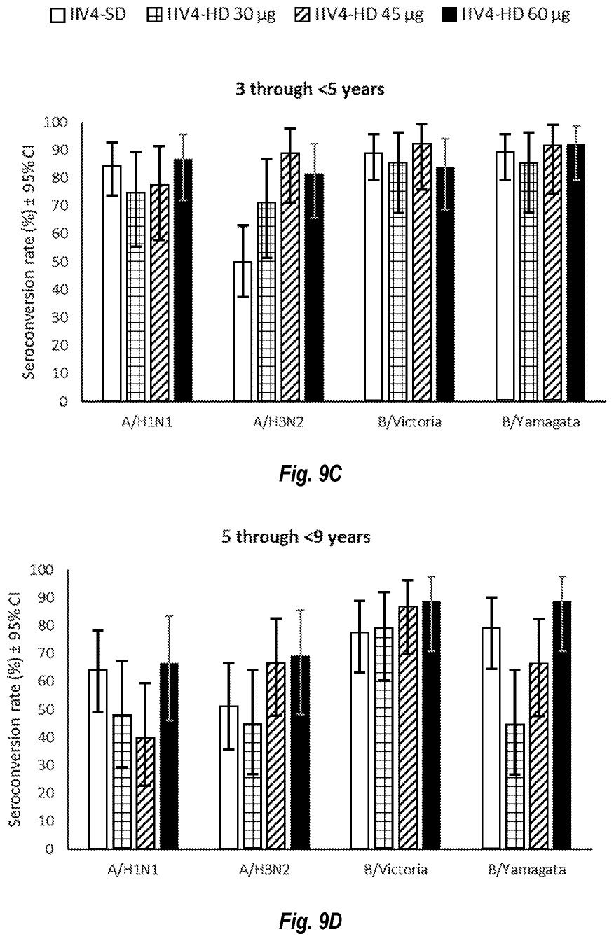

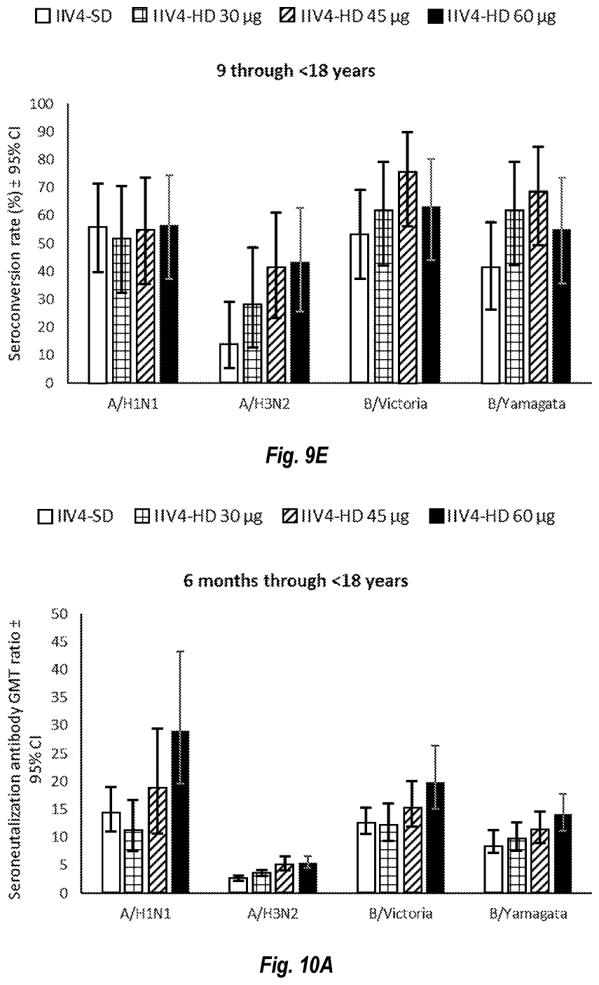

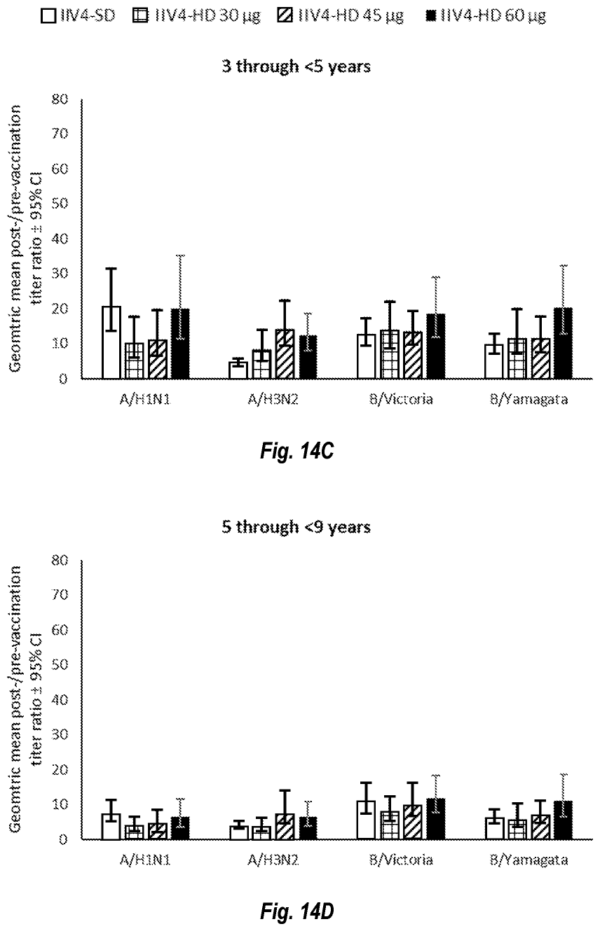

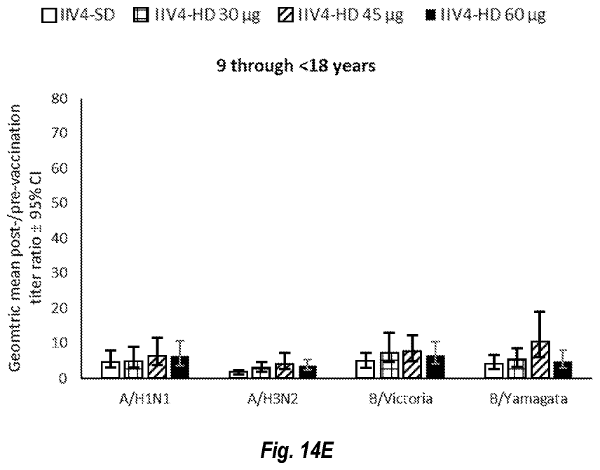

Ratios of Geometric mean titers of post- to pre-vaccination are shown in FIGS. 14A-14E. There was a trend of higher geometric mean post-/pre-vaccination titer ratios for QIV-HD 60 μg versus other QIV-HD doses, especially in participants aged 6 months through <3 years. Overall seroconversion rates were generally highest for QIV-HD 60 μg, although the differences between QIV-HD doses were modest (FIGS. 9A-9E). Seroconversion rates were generally higher for younger participants (6 months through <3 years and 3 through <5 years) than for older participants (5 through <9 years and 9 through <18 years).

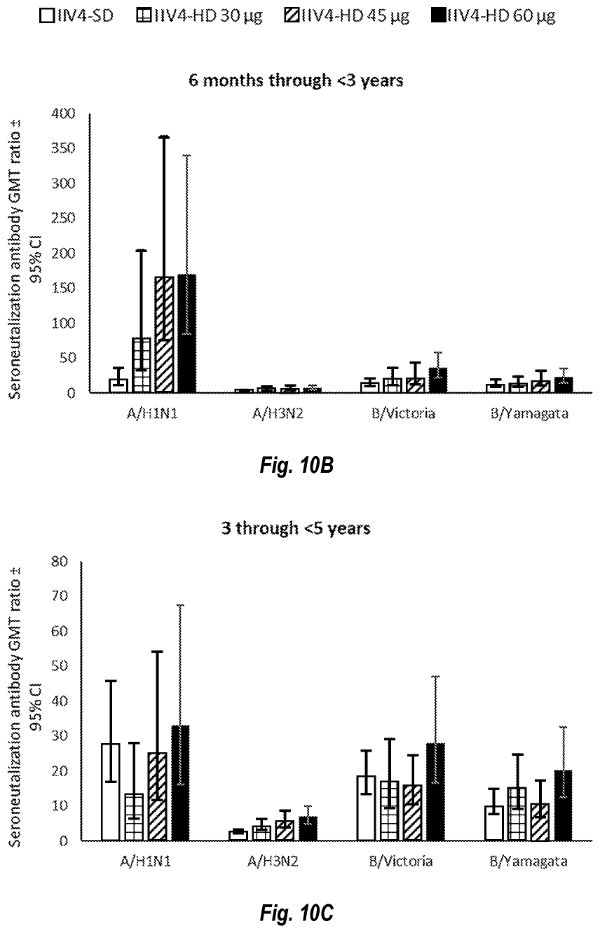

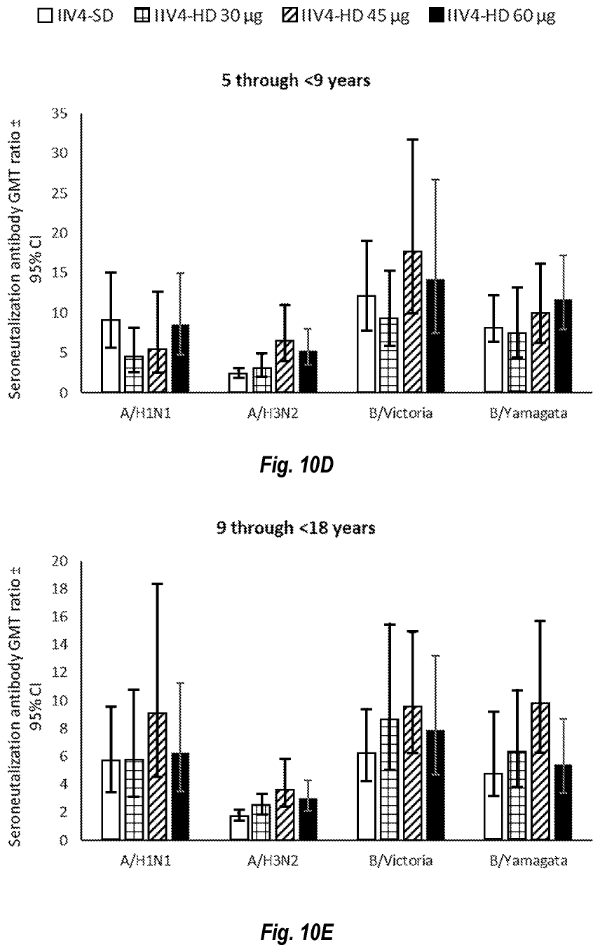

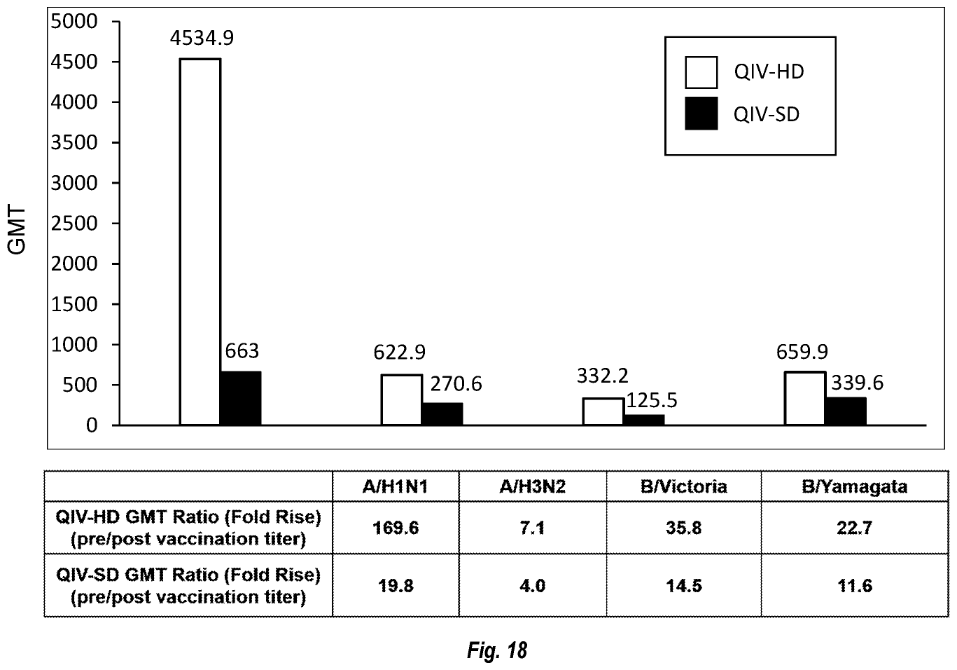

Overall, ratios of seroneutralization antibody GMTs for 28 days after the last vaccine dose versus day 0 were highest for QIV-HD 60 μg (FIGS. 10A-10E), For QIV-HD 60 μg, the seroneutralization antibody GMT ratio (95% CI) was 29.1 (19.6, 43.2) for A/H1N1, 5.5 (4.6, 6.7) for A/H3N2, 19.9 (15.1, 26.3) for B/Victoria, and 14.1 (11.1, 17.8) for B/Yamagata. Overall, the seroneutralization antibody GMT ratio (95% CI) was highest for A/H1N1 in participants aged 6 months through <3 years: 19.8 (11.2, 35.1) for IIV4 SD, 79.9 (31.1, 205.3) for IIV4-HD 30 μg, 164.8 (73.8, 367.9) for IIV4 HD 45 μg, and 169.6 (84.6, 339.7) for IIV4-HD 60 μg. See, also, FIG. 18.

TABLE 25
Geometric mean titer at 28 days post-last vaccination for the US cohort

| Age group | Strain* | QIV-SD (15 μg) | | QIV-HD 30 μg | | QIV-HD 45 μg | | QIV-HD 60 μg | |
|---|---|---|---|---|---|---|---|---|---|
| 6 months | A/H1N1 | 142.3 (84.2, | N = 68 | 302.7 (156.6, | N = 25 | 248.4 (135.1, | N = 26 | 603.3 (386.2, | N = 47 |
| through <3 | | 240.6) | | 585.2) | | 456.7) | | 942.7) | |
| years | A/H3N2 | 160.8 (100.0, | N = 68 | 149.3 (88.0, | N = 25 | 239.2(117.2, | N = 25 | 505.5 (295.6, | N = 47 |
| | | 258.5) | | 253.3) | | 488.3) | | 864.3) | |
| | B/Victoria | 135.2 (90.0, | N = 68 | 166.8 (92.6, | N = 25 | 186.4 (96.2, | N = 25 | 276.1 (174.5, | N = 47 |
| | | 203.2) | | 300.6) | | 360.9) | | 436.9) | |
| | B/Yamagata | 290.5 (195.2, | N = 68 | 320.0 (206.7, | N = 25 | 343.0 (171.2, | N = 25 | 556.3 (372.7, | N = 47 |
| | | 432.2) | | 495.4) | | 687.0) | | 830.5) | |
| 3 | A/H1N1 | 1300.6 (1009.6, | N = 65 | 697.9 (397.0, | N = 32 | 742.5 (454.9, | N = 28 | 651.2 (440.0, | N = 40 |
| through <5 | | 1675.6) | | 1227.0) | | 1211.9) | | 963.8) | |
| years | A/H3N2 | 297.0 (202.8, | N = 65 | 462.5 (253.0, | N = 32 | 883.0 (514.6, | N = 28 | 704.0 (418.1, | N = 40 |
| | | 434.9) | | 845.2) | | 1515.1) | | 1185.4) | |
| | B/Victoria | 420.0 (315.3, | N = 65 | 334.2 (206.3, | N = 32 | 353.3 (211.5, | N = 28 | 440.9 (317.7, | N = 40 |
| | | 559.4) | | 541.2) | | 590.2) | | 611.9) | |
| | B/Yamagata | 640.0 (489.0, | N = 65 | 612.9 (391.8, | N = 32 | 585.0 (337.8, | N = 27 | 815.7 (576.5, | N = 40 |
| | | 837.5) | | 958.7) | | 1013.1) | | 1154.2) | |
| 5 | A/H1N1 | 1109.3 (830.8, | N = 46 | 678.1 (485.9 | N = 30 | 765.4 (505.4, | N = 31 | 974.9(593.3, | N = 28 |
| through <9 | | 1481.2) | | 946.1) | | 1159.1) | | 1601.9) | |
| years | A/H3N2 | 492.6 (338.6, | N = 45 | 1027.7 (772.6, | N = 30 | 1280.0 (854.0, | N = 31 | 1474.1 (1057.6, | N = 27 |
| | | 716.6) | | 1367.1) | | 1918.5) | | 2054.7) | |
| | B/Victoria | 422.9 (301.6, | N = 46 | 427.1 (262.9, | N = 30 | 585.2 (375.0, | N = 31 | 799.7 (507.0, | N = 28 |
| | | 592.9) | | 694.1) | | 913.4) | | 1261.4) | |
| | B/Yamagata | 769.9 (600.4, | N = 45 | 815.7 (541.6, | N = 30 | 885.1 (600.3, | N = 31 | 1173.8 (784.2, | N = 28 |
| | | 987.4) | | 1228.5) | | 1305.0) | | 1757.0) | |
| 9 | A/H1N1 | 1301.3 (943.3, | N = 42 | 1280.0 (912.9, | N = 29 | 1326.7 (861.3, | N = 29 | 1669.6 (1301.6, | N = 30 |
| through <18 | | 1795.2) | | 1794.6) | | 2043.6) | | 2141.6) | |
| years | A/H3N2 | 609.1 (478.4, | N = 42 | 842.5 (581.8, | N = 29 | 1135.8 (848.9, | N = 29 | 937.0 (714.9, | N = 30 |
| | | 775.5) | | 1219.9) | | 1519.6) | | 1228.1) | |
| | B/Victoria | 640.0 (486.6, | N = 42 | 774.9 (575.1, | N = 29 | 784.2 (526.0, | N = 29 | 915.6 (688.3, | N = 30 |
| | | 841.8) | | 1044.0) | | 1169.2) | | 1217.9) | |
| | B/Yamagata | 1333.9 (1046.9, | N = 42 | 1549.7 (1165.0, | N = 29 | 1326.7 (876.6, | N = 29 | 1531.3 (1136.5, | N = 29 |
| | | 1699.7) | | 2061.4) | | 2008.0) | | 2063.3) | |

*A/Michigan/45/2015 (H1N1); A/Singapore/INFIMH-16-0019/2016 IVR-186 (H3N2); B/Maryland/15/2016 (Victoria lineage); B/Phuket/3073/2013 (Yamagata lineage)
Results are for the immunogenicity analysis set.
NC, not calculated

### Details:

The immunogenicity data in children 6 months to less than 18 years of age in the study of Example 1 are provided.

The immunogenicity analyses were performed on the IAS defined as the subset of randomized subjects who received 1 dose of a study vaccine (for subjects 9 to less than 18 years of age and for previously influenza vaccinated subjects 6 months to 8 years of age) or 2 doses of a same study vaccine (for previously influenza unvaccinated subjects 6 months to 8 years of age) and had a post-vaccination blood sample. Subjects were analyzed as treated.

2.4.1—Subjects 9 to less than 18 years of age (Groups 12 and 13 [Stage 1]—US)

HAI GMTs

At baseline (pre-vaccination), GMTs were similar between the QIV-HD (30 μg, 45 μg, and 60 μg) and QIV-SD group for the A/H1N1, A/H3N2, and B/Victoria strains. For the B/Yamagata strain, GMTs were similar between the QIV-HD (30 μg and 60 μg) and QIV-SD group and tended to be lower for the QIV-HD (45 μg) group (Table 26A).

At 28 days after vaccination, GMTs increased as compared to baseline (Table 26).

HAI GMTs (Comparison of QIV-HD to QIV-SD)

The GMT ratios for QIV-HD (30 μg, 45 μg, 60 μg) over the QIV-SD group were 0.98, 1.02, and 1.28, respectively, for the A/H1N1 strain; 1.38, 1.86, and 1.54, respectively, for the A/H3N2 strain; 1.21, 1.23, and 1.43, respectively, for the B/Victoria strain; and 1.16, 0.99, and 1.15, respectively, for the B/Yamagata strain (Table 26B).

HAI GMTRs (geometric mean of individual titer ratios of post-vaccination/pre-vaccination)

At 28 days after vaccination, GMTRs for the QIV-HD (30 μg, 45 μg, 60 μg) groups and QIV-SD group were 5.20, 6.45, 6.20, and 5.07, respectively, for the A/H1N1; 3.05, 4.30, 3.56, and 1.67, respectively, for the A/H3N2 strain; 7.63, 8.00, 6.57, and 5.07, respectively, for the B/Victoria strain; and 5.52, 10.7, 4.90, and 4.39, respectively, for the B/Yamagata strain (Table 26A). GMTRs for the QIV-HD (30 μg, 45 μg, and 60 μg) compared to QIV-SD group were similar for the A/H1N1, higher for the A/H3N2 and B/Victoria strains, and either similar or higher for the B/Yamagata strain.

HAI Seroconversion Rates

At 28 days after vaccination, the seroconversion rates for the QIV-HD (30 μg, 45 μg, 60 μg) groups and QIV-SD group were 51.7%, 55.2%, 56.7%, and 56.1%, respectively, for the A/H1N1 strain; 28.6%, 41.4%, 43.3%, and 14.6%, respectively, for the A/H3N2 strain; 62.1%, 75.9%, 63.3%, and 53.7%, respectively, for the B/Victoria strain; and 62.1%, 69.0%, 55.2%, and 41.5%, respectively, for the B/Yamagata strain (Table 27). In general, the seroconversion rates for the QIV-HD (30 μg, 45 μg, and 60 μg) compared to QIV-SD were higher for the A/H3N2, B/Victoria, and B/Yamagata strains, and similar for the A/H1N1 strain.

SN GMTs (Baseline and after Vaccination)

At baseline (pre-vaccination), GMTs were generally similar between the QIV-HD (30 μg, 45 μg, and 60 μg) compared to QIV-SD group against all 4 influenza strains (Table 28).

At 28 days after vaccination, all GMTs significantly increased compared to baseline (Table 28). Compared to QIV-SD, QIV-HD (60 μg) group had higher GMTs for the A/H3N2 and B/Victoria strains and similar GMTs for the A/H1N1 and B/Yamagata strains.

SN GMTR (Geometric Means of Individual Titer Ratios of Post-Vaccination/Pre-Vaccination)

GMTRs for the QIV-HD (30 μg, 45 μg, 60 μg) groups and QIV-SD group are shown in Table 28. QIV-HD (60 μg) group had higher GMTRs for the A/H3N2 and B/Victoria strains and similar GMTRs for the A/H1N1 and B/Yamagata strains compared to QIV-SD group.

SN: Subjects with 4-Fold Rise in Titer

At 28 days after vaccination, the number of subjects for the QIV-HD (30 μg, 45 μg, and 60 μg) groups and QIV-SD group with a 4-fold rise in titer is shown in Table 28. The number of subjects with a 4-fold rise for the QIV-HD (60 μg) compared to QIV-SD was similar for the A/H1N1, B/Victoria, and B/Yamagata strains and higher for the A/H3N2 strain.

TABLE 26A
Summary of HAI antibody response for each antigen (US subjects 9-17 years) - IAS

| | | US (9-17 years) | | | | | | | | | | | | | | | |
|---|---|---|---|---|---|---|---|---|---|---|---|---|---|---|---|---|---|
| | | QIV-HD (30 μg) | | | | | | | | QIV-HD (45 μg) | | | | | | | |
| | | (N = 29) | | | | | | | | (N = 29) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 29 | | 28 | | 29 | | 29 | | 29 | | 29 | | 29 | | 29 | |
| vaccination | GMT | 246 | | 276 | | 102 | | 281 | | 206 | | 264 | | 98.0 | | 124 | |
| (D 0) | (95% CI) | (137; 441) | | (200; 381) | | (58.7; 176) | | (199; 397) | | (127; 333) | | (163; 428) | | (56.3; 171) | | (72.9; 213) | |
| | Subject with | 2 | (6.9) | 0 | | 2 | (6.9) | 0 | | 1 | (3.4) | 1 | (3.4) | 3 | (10.3) | 1 | (3.4) |
| | titers <10 | | | | | | | | | | | | | | | | |
| | (1/dil): n (%) | | | | | | | | | | | | | | | | |
| | (95% CI) | (0.8; 22.8) | | (0; 12.3) | | (0.8; 22.8) | | (0; 11.9) | | (0.1; 17.8) | | (0.1; 17.8) | | (2.2; 27.4) | | (0.1; 17.8) | |
| | Subject with | 26 | (89.7) | 28 | (100) | 22 | (75.9) | 29 | (100) | 26 | (89.7) | 28 | (96.6) | 24 | (82.8) | 23 | (79.3) |
| | titers >=40 | | | | | | | | | | | | | | | | |
| | (1/dil): n (%) | | | | | | | | | | | | | | | | |
| | (95% CI) | (72.6; 97.8) | | (87.7; 100) | | (56.5; 89.7) | | (88.1; 100) | | (72.6; 97.8) | | (82.2; 99.9) | | (64.2; 94.2) | | (60.3; 92.0) | |
| Post- | M | 29 | | 29 | | 29 | | 29 | | 29 | | 29 | | 29 | | 29 | |
| vaccination | GMT | 1280 | | 842 | | 775 | | 1550 | | 1327 | | 1136 | | 784 | | 1327 | |
| (D 28) | (95% CI) | (913; 1795) | | (582; 1220) | | (575; 1044) | | (1165; 2061) | | (861; 2044) | | (849; 1520) | | (526; 1169) | | (877; 2008) | |
| | Subject with | 0 | | 0 | | 0 | | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 11.9) | | (0; 11.9) | | (0; 11.9) | | (0; 11.9) | | (0; 11.9) | | (0; 11.9) | | (0; 11.9) | | (0; 11.9) | |
| | Subject with | 29 | (100) | 29 | (100) | 29 | (100) | 29 | (100) | 29 | (100) | 29 | (100) | 29 | (100) | 29 | (100) |
| | titers >=40 | | | | | | | | | | | | | | | | |
| | (1/dil): n (%) | | | | | | | | | | | | | | | | |
| | (95% CI) | (88.1; 100) | | (88.1; 100) | | (88.1; 100) | | (88.1; 100) | | (88.1; 100) | | (88.1; 100) | | (88.1; 100) | | (88.1; 100) | |
| Post- | M | 29 | | 28 | | 29 | | 29 | | 29 | | 29 | | 29 | | 29 | |
| vaccination | GMTR | 5.20 | | 3.05 | | 7.63 | | 5.52 | | 6.45 | | 4.30 | | 8.00 | | 10.7 | |
| response | (95% CI) | (3.00; 9.02) | | (1.99; 4.67) | | (4.49; 13.0) | | (3.45; 8.85) | | (3.59; 11.6) | | (2.52; 7.34) | | (5.26; 12.2) | | (5.93; 19.1) | |
| based on | Seroconversion | 15 | (51.7) | 8 | (28.6) | 18 | (62.1) | 18 | (62.1) | 16 | (55.2) | 12 | (41.4) | 22 | (75.9) | 20 | (69.0) |
| pre- | rate: n (%) | | | | | | | | | | | | | | | | |
| vaccination | (95% CI) | (32.5; 70.6) | | (13.2; 48.7) | | (42.3; 79.3) | | (42.3; 79.3) | | (35.7; 73.6) | | (23.5; 61.1) | | (56.5; 89.7) | | (49.2; 84.7) | |
| (D 28/D 0) | | | | | | | | | | | | | | | | | |
| | | US (9-17 years) | | | | | | | | | | | | | | | |
| | | QIV-HD (60 μg) | | | | | | | | QIV-SD | | | | | | | |
| | | (N = 30) | | | | | | | | (N = 42) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 30 | | 30 | | 30 | | 30 | | 41 | | 41 | | 41 | | 41 | |
| vaccination | GMT | 269 | | 263 | | 139 | | 316 | | 257 | | 373 | | 127 | | 304 | |
| (D 0) | (95% CI) | (164; 441) | | (189; 367) | | (83.4; 233) | | (183; 548) | | (165; 401) | | (287; 484) | | (83.4; 194) | | (192; 482) | |
| | Subject with | 0 | | 0 | | 1 | (3.3) | 1 | (3.3) | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 11.6) | | (0; 11.6) | | (0.1; 17.2) | | (0.1; 17.2) | | (0; 8.6) | | (0; 8.6) | | (0; 8.6) | | (0; 8.6) | |
| | Subject with | 29 | (96.7) | 29 | (96.7) | 25 | (83.3) | 28 | (93.3) | 36 | (87.8) | 41 | (100) | 33 | (80.5) | 37 | (90.2) |
| | titers >=40 | | | | | | | | | | | | | | | | |
| | (1/dil): n (%) | | | | | | | | | | | | | | | | |
| | (95% CI) | (82.8; 99.9) | | (82.8; 99.9) | | (65.3; 94.4) | | (77.9; 99.2) | | (73.8; 95.9) | | (91.4; 100) | | (65.1; 91.2) | | (76.9; 97.3) | |
| Post- | M | 30 | | 30 | | 30 | | 29 | | 42 | | 42 | | 42 | | 42 | |
| vaccination | GMT | 1670 | | 937 | | 916 | | 1531 | | 1301 | | 609 | | 640 | | 1334 | |
| (D 28) | (95% CI) | (1302; 2142) | | (715; 1228) | | (688; 1218) | | (1136; 2063) | | (943; 1795) | | (478; 776) | | (487; 842) | | (1047; 1700) | |
| | Subject with | 0 | | 0 | | 0 | | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 11.6) | | (0; 11.6) | | (0; 11.6) | | (0; 11.9) | | (0; 8.4) | | (0; 8.4) | | (0; 8.4) | | (0; 8.4) | |
| | Subject with | 30 | (100) | 30 | (100) | 30 | (100) | 29 | (100) | 42 | (100) | 42 | (100) | 42 | (100) | 42 | (100) |
| | titers >=40 | | | | | | | | | | | | | | | | |
| | (1/dil): n (%) | | | | | | | | | | | | | | | | |
| | (95% CI) | (88.4; 100) | | (88.4; 100) | | (88.4; 100) | | (88.1; 100) | | (91.6; 100) | | (91.6; 100) | | (91.6; 100) | | (91.6; 100) | |
| Post- | M | 30 | | 30 | | 30 | | 29 | | 41 | | 41 | | 41 | | 41 | |
| vaccination | GMTR | 6.20 | | 3.56 | | 6.57 | | 4.90 | | 5.07 | | 1.67 | | 5.07 | | 4.39 | |
| response | (95% CI) | (3.63; 10.6) | | (2.36; 5.39) | | (4.15; 10.4) | | (3.05; 7.88) | | (3.16; 8.14) | | (1.27; 2.22) | | (3.52; 7.31) | | (2.82; 6.83) | |
| based on | Seroconversion | 17 | (56.7) | 13 | (43.3) | 19 | (63.3) | 16 | (55.2) | 23 | (56.1) | 6 | (14.6) | 22 | (53.7) | 17 | (41.5) |
| pre- | rate: n (%) | | | | | | | | | | | | | | | | |

| | | | | | | | | | |
|---|---|---|---|---|---|---|---|---|---|
| vaccination | (95% CI) | (37.4; 74.5) | (25.5; 62.6) | (43.9; 80.1) | (35.7; 73.6) | (39.7; 71.5) | (5.6; 29.2) | (37.4; 69.3) | (26.3; 57.9) |
| (D 28/D 0) | | | | | | | | | |

n: number of subjects experiencing the endpoint listed in the first two columns
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
GMTR (Geometric means of titer ratio): geometric mean of the individual titer ratios (post-vaccination/pre-vaccination).
Seroconversion: subjects with a pre-vaccination HAI titer <10 (1/dil) and a post-vaccination titer >=40 (1/dil) or a pre-vaccination titer >=10 (1/dil) and a >=4-fold increase in post-vaccination.

TABLE 26B
Comparison of QIV-HD to QIV-SD using GMTs by HAI method at
28 days after vaccination (US subjects 9-17 years) - IAS

| | | US (9-17 years) | | | | | | | |
|---|---|---|---|---|---|---|---|---|---|
| | | | | | | | | QIV-HD/QIV-SD | |
| Investigational | Antigen/ | QIV-HD | | | QIV-SD | | | GMT | |
| dosage | strain | M | GMT | (95% CI) | M | GMT | (95% CI) | ratio | (95% CI) |
| QIV-HD | A/H1N1 | 29 | 1280 | (913; 1795) | 42 | 1301 | (943; 1795) | 0.98 | (0.61; 1.57) |
| (30 μg) | A/H3N2 | 29 | 842 | (582; 1220) | 42 | 609 | (478; 776) | 1.38 | (0.91; 2.09) |
| | B/Victoria | 29 | 775 | (575; 1044) | 42 | 640 | (487; 842) | 1.21 | (0.81; 1.82) |
| | B/Yamagata | 29 | 1550 | (1165; 2061) | 42 | 1334 | (1047; 1700) | 1.16 | (0.80; 1.68) |
| QIV-HD | A/H1N1 | 29 | 1327 | (861; 2044) | 42 | 1301 | (943; 1795) | 1.02 | (0.61; 1.71) |
| (45 μg) | A/H3N2 | 29 | 1136 | (849; 1520) | 42 | 609 | (478; 776) | 1.86 | (1.29; 2.70) |
| | B/Victoria | 29 | 784 | (526; 1169) | 42 | 640 | (487; 842) | 1.23 | (0.77; 1.94) |
| | B/Yamagata | 29 | 1327 | (877; 2008) | 42 | 1334 | (1047; 1700) | 0.99 | (0.64; 1.55) |
| QIV-HD | A/H1N1 | 30 | 1670 | (1302; 2142) | 42 | 1301 | (943; 1795) | 1.28 | (0.84; 1.97) |
| (60 μg) | A/H3N2 | 30 | 937 | (715; 1228) | 42 | 609 | (478; 776) | 1.54 | (1.07; 2.20) |
| | B/Victoria | 30 | 916 | (688; 1218) | 42 | 640 | (487; 842) | 1.43 | (0.96; 2.13) |
| | B/Yamagata | 29 | 1531 | (1136; 2063) | 42 | 1334 | (1047; 1700) | 1.15 | (0.79; 1.67) |

M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.

TABLE 27
Comparison of QIV-HD to QIV-SD using seroconversion rate by HAI
method at 28 days after vaccination (US subjects 9-17 year) - IAS

| | | US (9-17 years) | | | | | | | |
|---|---|---|---|---|---|---|---|---|---|
| | | | | | | | | QIV-HD minus QIV-SD | |
| Investigational | Antigen/ | QIV-HD | | | QIV-SD | | | Difference | |
| dosage | strain | n/M | % | (95% CI) | n/M | % | (95% CI) | of % | (95% CI) |
| QIV-HD | A/H1N1 | 15/29 | 51.7 | (32.5; 70.6) | 23/41 | 56.1 | (39.7; 71.5) | −4.37 | (−26.63; 18.25) |
| (30 μg) | A/H3N2 | 8/28 | 28.6 | (13.2; 48.7) | 6/41 | 14.6 | (5.6; 29.2) | 13.94 | (−5.25; 33.98) |
| | B/Victoria | 18/29 | 62.1 | (42.3; 79.3) | 22/41 | 53.7 | (37.4; 69.3) | 8.41 | (−14.62; 29.73) |
| | B/Yamagata | 18/29 | 62.1 | (42.3; 79.3) | 17/41 | 41.5 | (26.3; 57.9) | 20.61 | (−2.98; 41.11) |
| QIV-HD | A/H1N1 | 16/29 | 55.2 | (35.7; 73.6) | 23/41 | 56.1 | (39.7; 71.5) | −0.93 | (−23.44; 21.35) |
| (45 μg) | A/H3N2 | 12/29 | 41.4 | (23.5; 61.1) | 6/41 | 14.6 | (5.6; 29.2) | 26.75 | (5.71; 46.24) |
| | B/Victoria | 22/29 | 75.9 | (56.5; 89.7) | 22/41 | 53.7 | (37.4; 69.3) | 22.20 | (−0.75; 41.29) |
| | B/Yamagata | 20/29 | 69.0 | (49.2; 84.7) | 17/41 | 41.5 | (26.3; 57.9) | 27.50 | (3.81; 46.92) |
| QIV-HD | A/H1N1 | 17/30 | 56.7 | (37.4; 74.5) | 23/41 | 56.1 | (39.7; 71.5) | 0.57 | (−21.83; 22.51) |
| (60 μg) | A/H3N2 | 13/30 | 43.3 | (25.5; 62.6) | 6/41 | 14.6 | (5.6; 29.2) | 28.70 | (7.60; 47.81) |
| | B/Victoria | 19/30 | 63.3 | (43.9; 80.1) | 22/41 | 53.7 | (37.4; 69.3) | 9.67 | (−13.16; 30.68) |
| | B/Yamagata | 16/29 | 55.2 | (35.7; 73.6) | 17/41 | 41.5 | (26.3; 57.9) | 13.71 | (−9.54; 35.09) |

n: number of subjects experiencing seroconversion.
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
Seroconversion: subjects with a pre-vaccination HAI titer <10 (1/dil) and a post-vaccination titer >=40 (1/dil) or a pre-vaccination titer >=10 (1/dil) and a >=4-fold increase in post-vaccination.

TABLE 28
Summary of SN antibody response for each antigen (US subjects 9-17 years) - IAS

| | | US (9-17 years) | | | | | | | | | | | | | | | |
|---|---|---|---|---|---|---|---|---|---|---|---|---|---|---|---|---|---|
| | | QIV-HD (30 ug) | | | | | | | | QIV-HD (45 μg) | | | | | | | |
| | | (N = 29) | | | | | | | | (N = 29) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 28 | | 28 | | 28 | | 28 | | 29 | | 29 | | 29 | | 29 | |
| vaccination | GMT | 2066 | | 343 | | 140 | | 339 | | 1506 | | 282 | | 120 | | 208 | |
| (D 0) | (95% CI) | (1075; 3971) | | (267; 440) | | (79.2; 246) | | (244; 470) | | (789; 2874) | | (194; 409) | | (68.3; 210) | | (130; 331) | |
| | Subject with | 0 | | 0 | | 0 | | 0 | | 1 | (3.4) | 0 | | 3 | (10.3) | 0 | |
| | titers <10 | | | | | | | | | | | | | | | | |
| | (1/dil): n (%) | | | | | | | | | | | | | | | | |
| | (95% CI) | (0; 12.3) | | (0; 12.3) | | (0; 12.3) | | (0; 12.3) | | (0.1; 17.8) | | (0; 11.9) | | (2.2; 27.4) | | (0; 11.9) | |
| | Subject with | 27 | (96.4) | 28 | (100) | 21 | (75.0) | 28 | (100) | 28 | (96.6) | 28 | (96.6) | 22 | (75.9) | 25 | (86.2) |
| | titers >=40 | | | | | | | | | | | | | | | | |
| | (1/dil): n (%) | | | | | | | | | | | | | | | | |
| | (95% CI) | (81.7; 99.9) | | (87.7; 100) | | (55.1; 89.3) | | (87.7; 100) | | (82.2; 99.9) | | (82.2; 99.9) | | (56.5; 89.7) | | (68.3; 96.1) | |
| Post- | M | 29 | | 29 | | 29 | | 29 | | 29 | | 29 | | 29 | | 29 | |
| vaccination | GMT | 11929 | | 901 | | 1235 | | 2148 | | 13667 | | 1032 | | 1156 | | 2051 | |
| (D 28) | (95% CI) | (7752; 18356) | | (627; 1294) | | (833; 1830) | | (1539; 2998) | | (8634; 21634) | | (777; 1372) | | (722; 1851) | | (1320; 3187) | |
| | Subject with | 0 | | 0 | | 0 | | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 11.9) | | (0; 11.9) | | (0; 11.9) | | (0; 11.9) | | (0; 11.9) | | (0; 11.9) | | (0; 11.9) | | (0; 11.9) | |
| | Subject with | 29 | (100) | 29 | (100) | 29 | (100) | 29 | (100) | 29 | (100) | 29 | (100) | 29 | (100) | 29 | (100) |
| | titers >=40 | | | | | | | | | | | | | | | | |
| | (1/dil): n (%) | | | | | | | | | | | | | | | | |
| | (95% CI) | (88.1; 100) | | (88.1; 100) | | (88.1; 100) | | (88.1; 100) | | (88.1; 100) | | (88.1; 100) | | (88.1; 100) | | (88.1; 100) | |
| Post- | M | 28 | | 28 | | 28 | | 28 | | 29 | | 29 | | 29 | | 29 | |
| vaccination | GMTR | 5.83 | | 2.50 | | 8.73 | | 6.37 | | 9.08 | | 3.66 | | 9.65 | | 9.87 | |
| response | (95% CI) | (3.14; 10.8) | | (1.87; 3.34) | | (4.96; 15.4) | | (3.80; 10.7) | | (4.50; 18.3) | | (2.30; 5.84) | | (6.24; 14.9) | | (6.21; 15.7) | |
| based on | Subjects with | 20 | (71.4) | 14 | (50.0) | 23 | (82.1) | 19 | (67.9) | 22 | (75.9) | 17 | (58.6) | 26 | (89.7) | 28 | (96.6) |
| pre- | 2-Fold | | | | | | | | | | | | | | | | |
| vaccination | rise*: n (%) | | | | | | | | | | | | | | | | |
| (D 28/D 0) | (95% CI) | (51.3; 86.8) | | (30.6; 69.4) | | (63.1; 93.9) | | (47.6; 84.1) | | (56.5; 89.7) | | (38.9; 76.5) | | (72.6; 97.8) | | (82.2; 99.9) | |
| | Subjects with | 13 | (46.4) | 7 | (25.0) | 18 | (64.3) | 17 | (60.7) | 15 | (51.7) | 11 | (37.9) | 22 | (75.9) | 19 | (65.5) |
| | 4-Fold | | | | | | | | | | | | | | | | |
| | rise*: n (%) | | | | | | | | | | | | | | | | |
| | (95% CI) | (27.5; 66.1) | | (10.7; 44.9) | | (44.1; 81.4) | | (40.6; 78.5) | | (32.5; 70.6) | | (20.7; 57.7) | | (56.5; 89.7) | | (45.7; 82.1) | |
| | | US (9-17 years) | | | | | | | | | | | | | | | |
| | | QIV-HD (60 μg) | | | | | | | | QIV-SD | | | | | | | |
| | | (N = 30) | | | | | | | | (N = 42) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 30 | | 30 | | 30 | | 30 | | 42 | | 42 | | 42 | | 42 | |
| vaccination | GMT | 2097 | | 284 | | 159 | | 405 | | 2148 | | 328 | | 146 | | 384 | |
| (D 0) | (95% CI) | (1243; 3536) | | (222; 363) | | (92.8; 273) | | (254; 645) | | (1355; 3407) | | (253; 425) | | (95.7; 223) | | (253; 583) | |
| | Subject with | 0 | | 0 | | 1 | (3.3) | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 11.6) | | (0; 11.6) | | (0.1; 17.2) | | (0; 11.6) | | (0; 8.4) | | (0; 8.4) | | (0; 8.4) | | (0; 8.4) | |
| | Subject with | 30 | (100) | 30 | (100) | 24 | (80.0) | 28 | (93.3) | 42 | (100) | 41 | (97.6) | 33 | (78.6) | 41 | (97.6) |
| | titers >=40 | | | | | | | | | | | | | | | | |
| | (1/dil): n (%) | | | | | | | | | | | | | | | | |
| | (95% CI) | (88.4; 100) | | (88.4; 100) | | (61.4; 92.3) | | (77.9; 99.2) | | (91.6; 100) | | (87.4; 99.9) | | (63.2; 89.7) | | (87.4; 99.9) | |
| Post- | M | 30 | | 29 | | 30 | | 30 | | 42 | | 42 | | 42 | | 42 | |
| vaccination | GMT | 13238 | | 851 | | 1250 | | 2206 | | 12239 | | 574 | | 915 | | 1848 | |
| (D 28) | (95% CI) | (10374; 16892) | | (660; 1098) | | (823; 1898) | | (1654; 2942) | | (8639; 17341) | | (463; 711) | | (641; 1306) | | (1474; 2317) | |
| | Subject with | 0 | | 0 | | 0 | | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 11.6) | | (0; 11.9) | | (0; 11.6) | | (0; 11.6) | | (0; 8.4) | | (0; 8.4) | | (0; 8.4) | | (0; 8.4) | |
| | Subject with | 30 | (100) | 29 | (100) | 30 | (100) | 30 | (100) | 42 | (100) | 42 | (100) | 41 | (97.6) | 42 | (100) |
| | titers >=40 | | | | | | | | | | | | | | | | |
| | (1/dil): n (%) | | | | | | | | | | | | | | | | |
| | (95% CI) | (88.4; 100) | | (88.1; 100) | | (88.4; 100) | | (88.4; 100) | | (91.6; 100) | | (91.6; 100) | | (87.4; 99.9) | | (91.6; 100) | |
| Post- | M | 30 | | 29 | | 30 | | 30 | | 42 | | 42 | | 42 | | 42 | |
| vaccination | GMTR | 6.31 | | 3.01 | | 7.86 | | 5.45 | | 5.70 | | 1.75 | | 6.26 | | 4.81 | |
| response | (95% CI) | (3.52; 11.3) | | (2.11; 4.30) | | (4.69; 13.2) | | (3.41; 8.70) | | (3.41; 9.52) | | (1.39; 2.20) | | (4.18; 9.38) | | (3.09; 7.49) | |
| based on | Subjects with | 23 | (76.7) | 18 | (62.1) | 26 | (86.7) | 23 | (76.7) | 26 | (61.9) | 16 | (38.1) | 33 | (78.6) | 32 | (76.2) |
| pre- | 2-Fold | | | | | | | | | | | | | | | | |
| vaccination | rise*: n (%) | | | | | | | | | | | | | | | | |
| (D 28/D 0) | (95% CI) | (57.7; 90.1) | | (42.3; 79.3) | | (69.3; 96.2) | | (57.7; 90.1) | | (45.6; 76.4) | | (23.6; 54.4) | | (63.2; 89.7) | | (60.5; 87.9) | |
| | Subjects with | 15 | (50.0) | 10 | (34.5) | 19 | (63.3) | 15 | (50.0) | 20 | (47.6) | 5 | (11.9) | 23 | (54.8) | 15 | (35.7) |
| | 4-Fold | | | | | | | | | | | | | | | | |
| | rise*: n (%) | | | | | | | | | | | | | | | | |

| | | | | | | | | |
|---|---|---|---|---|---|---|---|---|
| (95% CI) | (31.3; 68.7) | (17.9; 54.3) | (43.9; 80.1) | (31.3; 68.7) | (32.0; 63.6) | (4.0; 25.6) | (38.7; 70.2) | (21.6; 52.0) |

n: number of subjects experiencing the endpoint listed in the first two columns
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
GMTR (Geometric means of titer ratio): geometric mean of the individual titer ratios (post-vaccination/pre-vaccination).
*X-Fold rise: the computed value (=post-vaccination computed value/pre-vaccination computed value) >=X.

2.4.2—Subjects 5 to 8 Years of Age (Groups 9 [Stage 1], 10 [Stage 2], and 11 [Stage 3]—US)

HAI GMTs (Baseline and after Last Vaccination)

At baseline (pre-vaccination), GMTs were similar between the QIV-HD (30 μg, 45 μg, 60 μg) and QIV-SD group for the A/H1N1, A/H3N2, B/Victoria, and B/Yamagata strains (Table 29).

At 28 days after the last vaccination, all GMTs significantly increased compared to baseline (Table 30).

HAI GMTs (Comparison of QIV-HD/QIV-SD)

The GMT ratio for QIV-HD (30 μg, 45 μg, 60 μg) over QIV-SD group was 0.61, 0.69, and 0.88, respectively, for the A/H1N1 strain; 2.09, 2.60, and 2.99, respectively, for the A/H3N2 strain; 1.01, 1.38, and 1.89, respectively, for the B/Victoria strain; and 1.06, 1.15, and 1.52, respectively, for the B/Yamagata strain (Table 30).

HAI GMTRs (Geometric Mean of Individual Titer Ratios of Post-Vaccination/Pre-Vaccination)

At 28 days after the last vaccination, GMTRs for the QIV-HD (30 μg, 45 μg, 60 μg) groups and QIV-SD group were 4.10, 4.65, 6.51, and 7.58, respectively, for the A/H1N1; 3.95, 7.64, 6.55, and 3.90, respectively, for the A/H3N2 strain; 8.00, 9.96, 11.9, and 11.1, respectively, for the B/Victoria strain; and 5.86, 7.05, 11.0, and 6.42, respectively, for the B/Yamagata strain (Table 29). GMTRs for the QIV-HD (30 μg, 45 μg, and 60 μg) compared to QIV-SD group were similar or higher for the A/H3N2, B/Victoria, and B/Yamagata strains, but lower for the A/H1N1 strain.

HAI Seroconversion Rates (after Last Vaccination)

At 28 days after last vaccination, the seroconversion rates for the QIV-HD (30 μg, 45 μg, 60 μg) groups and QIV-SD group were 48.3%, 40.%0, 66.7%, and 64.4%, respectively, for the A/H1N1; 44.8%, 66.7%, 69.2%, and 51.2%, respectively, for the A/H3N2 strain; 79.3%, 86.7%, 88.9%, and 77.8%, respectively, for the B/Victoria strain; and 44.8%, 66.7%, 88.9%, and 79.5%, respectively, for the B/Yamagata strain. The seroconversion rates for QIV-HD (60 μg) group were higher or similar compared to the QIV-HD (30 μg and 45 μg) and QIV-SD group for the 4 influenza strains (Table 31).

SN GMTs (Baseline and after Last Vaccination)

At baseline (pre-vaccination), GMTs were similar between the QIV-HD (30 μg, 45 μg, 60 μg) compared to QIV-SD group against all 4 influenza strains (Table 32).

At 28 days after last vaccination, all GMTs significantly increased compared to baseline. QIV-HD (60 μg) group had higher GMTs for the A/H3N2 and B/Victoria strains and similar GMTs for the A/H1N1 and B/Yamagata strains compared to QIV-SD group (Table 32).

SN GMTR (Geometric Means of Individual Titer Ratios of Post-Vaccination/Pre-Vaccination)

GMTRs for the QIV-HD (30 tag, 45 tag, 60 μg) groups and QIV-SD group are shown in Table 32. QV-HD (60 2 g) group had higher GMTRs for the A/H3N2 strain and similar GMTRs for the A/H1N1, B/Victoria, and B/Yamagata strains compared to QIV-SD group.

SN: Subjects with 4-Fold Rise in Titer

At 28 days after last vaccination, the number of subjects for the QIV-HD (30 μg, 45 μg, 60 μg) groups and Q(V-SD group with a 4-fold rise in titer is shown in Table 32. The number of subjects with a 4-fold rise for the QIV-HD (60 μg) compared to QIV-SD was similar for the A/H1N1, B/Victoria, and B/Yamagata strains and higher for the A/H3N2 strain.

TABLE 29
Summary of HAI antibody response for each antigen (US subjects 5-8 years) - IAS

| | | US (5-8 years) | | | | | | | | | | | | | | | |
|---|---|---|---|---|---|---|---|---|---|---|---|---|---|---|---|---|---|
| | | QIV-HD (30 μg) | | | | | | | | QIV-HD (45 μg) | | | | | | | |
| | | (N = 31) | | | | | | | | (N = 31) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 30 | | 30 | | 30 | | 30 | | 30 | | 30 | | 30 | | 30 | |
| vaccination | GMT | 171 | | 243 | | 51.0 | | 120 | | 171 | | 168 | | 59.9 | | 131 | |
| (D 0) | (95% CI) | (112; 264) | | (155; 379) | | (28.1; 92.4) | | (62.1; 231) | | (87.1; 338) | | (93.9; 299) | | (34.7; 104) | | (74.2; 233) | |
| | Subject with | 1 | (3.3) | 0 | | 5 | (16.7) | 3 | (10.0) | 4 | (13.3) | 2 | (6.7) | 3 | (10.0) | 3 | (10.0) |
| | titers <10 | | | | | | | | | | | | | | | | |
| | (1/dil): n (%) | | | | | | | | | | | | | | | | |
| | (95% CI) | (0.1; 17.2) | | (0; 11.6) | | (5.6; 34.7) | | (2.1; 26.5) | | (3.8; 30.7) | | (0.8; 22.1) | | (2.1; 26.5) | | (2.1; 26.5) | |
| | Subject with | 27 | (90.0) | 28 | (93.3) | 17 | (56.7) | 23 | (76.7) | 25 | (83.3) | 26 | (86.7) | 19 | (63.3) | 24 | (80.0) |
| | titers >=40 | | | | | | | | | | | | | | | | |
| | (1/dil): n (%) | | | | | | | | | | | | | | | | |
| | (95% CI) | (73.5; 97.9) | | (77.9; 99.2) | | (37.4; 74.5) | | (57.7; 90.1) | | (65.3; 94.4) | | (69.3; 96.2) | | (43.9; 80.1) | | (61.4; 92.3) | |
| Post- | M | 30 | | 30 | | 30 | | 30 | | 31 | | 31 | | 31 | | 31 | |
| vaccination | GMT | 678 | | 1028 | | 427 | | 816 | | 765 | | 1280 | | 585 | | 885 | |
| (D 28 or | (95% CI) | (486; 946) | | (773; 1367) | | (263; 694) | | (542; 1228) | | (505; 1159) | | (854; 1918) | | (375; 913) | | (600; 1305) | |
| D 56 §) | Subject with | 0 | | 0 | | 0 | | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 11.6) | | (0; 11.6) | | (0; 11.6) | | (0; 11.6) | | (0; 11.2) | | (0; 11.2) | | (0; 11.2) | | (0; 11.2) | |
| | Subject with | 30 | (100) | 30 | (100) | 30 | (100) | 30 | (100) | 31 | (100) | 31 | (100) | 31 | (100) | 31 | (100) |
| | titers >=40 | | | | | | | | | | | | | | | | |
| | (1/dil): n (%) | | | | | | | | | | | | | | | | |
| | (95% CI) | (88.4; 100) | | (88.4; 100) | | (88.4; 100) | | (88.4; 100) | | (88.8; 100) | | (88.8; 100) | | (88.8; 100) | | (88.8; 100) | |
| Post- | M | 29 | | 29 | | 29 | | 29 | | 30 | | 30 | | 30 | | 30 | |
| vaccination | GMTR | 4.10 | | 3.95 | | 8.00 | | 5.86 | | 4.65 | | 7.64 | | 9.96 | | 7.05 | |
| response | (95% CI) | (2.54; 6.61) | | (2.50; 6.26) | | (5.26; 12.2) | | (3.35; 10.3) | | (2.53; 8.53) | | (4.18; 14.0) | | (6.12; 16.2) | | (4.43; 11.2) | |
| based on | Seroconversion | 14 | (48.3) | 13 | (44.8) | 23 | (79.3) | 13 | (44.8) | 12 | (40.0) | 20 | (66.7) | 26 | (86.7) | 20 | (66.7) |
| pre- | rate: n (%) | | | | | | | | | | | | | | | | |
| vaccination | (95% CI) | (29.4; 67.5) | | (26.4; 64.3) | | (60.3; 92.0) | | (26.4; 64.3) | | (22.7; 59.4) | | (47.2; 82.7) | | (69.3; 96.2) | | (47.2; 82.7) | |
| (D 28 or | | | | | | | | | | | | | | | | | |
| D 56 §/D 0) | | | | | | | | | | | | | | | | | |
| | | US (5-8 years) | | | | | | | | | | | | | | | |
| | | QIV-HD (60 μg) | | | | | | | | QIV-SD | | | | | | | |
| | | (N = 30) | | | | | | | | (N = 48) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 27 | | 27 | | 27 | | 27 | | 47 | | 46 | | 47 | | 47 | |
| vaccination | GMT | 158 | | 226 | | 68.6 | | 109 | | 162 | | 146 | | 39.7 | | 119 | |
| (D 0) | (95% CI) | (69.9; 357) | | (140; 365) | | (35.8; 131) | | (55.6; 213) | | (108; 244) | | (94.0; 227) | | (27.0; 58.5) | | (79.7; 178) | |
| | Subject with | 5 | (18.5) | 0 | | 3 | (11.1) | 3 | (11.1) | 3 | (6.4) | 2 | (4.3) | 5 | (10.6) | 3 | (6.4) |
| | titers <10 | | | | | | | | | | | | | | | | |
| | (1/dil): n (%) | | | | | | | | | | | | | | | | |
| | (95% CI) | (6.3; 38.1) | | (0; 12.8) | | (2.4; 29.2) | | (2.4; 29.2) | | (1.3; 17.5) | | (0.5; 14.8) | | (3.5; 23.1) | | (1.3; 17.5) | |
| | Subject with | 19 | (70.4) | 25 | (92.6) | 17 | (63.0) | 19 | (70.4) | 40 | (85.1) | 39 | (84.8) | 24 | (51.1) | 39 | (83.0) |
| | titers >=40 | | | | | | | | | | | | | | | | |
| | (1/dil): n (%) | | | | | | | | | | | | | | | | |
| | (95% CI) | (49.8; 86.2) | | (75.7; 99.1) | | (42.4; 80.6) | | (49.8; 86.2) | | (71.7; 93.8) | | (71.1; 93.7) | | (36.1; 65.9) | | (69.2; 92.4) | |
| Post- | M | 28 | | 27 | | 28 | | 28 | | 46 | | 45 | | 46 | | 45 | |
| vaccination | GMT | 975 | | 1474 | | 800 | | 1174 | | 1109 | | 493 | | 423 | | 770 | |
| (D 28 or | (95% CI) | (593; 1602) | | (1058; 2055) | | (507; 1261) | | (784; 1757) | | (831; 1481) | | (339; 717) | | (302; 593) | | (600; 987) | |
| D 56 §) | Subject with | 0 | | 0 | | 0 | | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 12.3) | | (0; 12.8) | | (0; 12.3) | | (0; 12.3) | | (0; 7.7) | | (0; 7.9) | | (0; 7.7) | | (0; 7.9) | |
| | Subject with | 28 | (100) | 27 | (100) | 28 | (100) | 28 | (100) | 46 | (100) | 44 | (97.8) | 44 | (95.7) | 45 | (100) |
| | titers >=40 | | | | | | | | | | | | | | | | |
| | (1/dil): n (%) | | | | | | | | | | | | | | | | |
| | (95% CI) | (87.7; 100) | | (87.2; 100) | | (87.7; 100) | | (87.7; 100) | | (92.3; 100) | | (88.2; 99.9) | | (85.2; 99.5) | | (92.1; 100) | |
| Post- | M | 27 | | 26 | | 27 | | 27 | | 45 | | 43 | | 45 | | 44 | |
| vaccination | GMTR | 6.51 | | 6.55 | | 11.9 | | 11.0 | | 7.58 | | 3.90 | | 11.1 | | 6.42 | |
| response | (95% CI) | (3.68; 11.5) | | (3.93; 10.9) | | (7.74; 18.3) | | (6.54; 18.6) | | (5.09; 11.3) | | (2.88; 5.29) | | (7.54; 16.2) | | (4.72; 8.73) | |
| based on | Seroconversion | 18 | (66.7) | 18 | (69.2) | 24 | (88.9) | 24 | (88.9) | 29 | (64.4) | 22 | (51.2) | 35 | (77.8) | 35 | (79.5) |
| pre- | rate: n (%) | | | | | | | | | | | | | | | | |

| | | | | | | | | | |
|---|---|---|---|---|---|---|---|---|---|
| vaccination | (95% CI) | (46.0; 83.5) | (48.2; 85.7) | (70.8; 97.6) | (70.8; 97.6) | (48.8; 78.1) | (35.5; 66.7) | (62.9; 88.8) | (64.7; 90.2) |
| (D 28 or | | | | | | | | | |
| D 56 §/D 0) | | | | | | | | | |

n: number of subjects experiencing the endpoint listed in the first two columns
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
* D 28 or D 56: at 28 days after the last vaccination.
GMTR (Geometric means of titer ratio): geometric mean of the individual titer ratios (post-vaccination/pre-vaccination).
Seroconversion: subjects with a pre-vaccination HAI titer <10 (1/dil) and a post-vaccination titer >=40 (1/dil) or a pre-vaccination titer >=10 (1/dil) and a >=4-fold increase in post-vaccination.

TABLE 30
Comparison of QIV-HD to QIV-SD using GMTs by HAI method at 28
days after the last vaccination (US subjects 5-8 year) - IAS

| | | US (5-8 years) | | | | | | | |
|---|---|---|---|---|---|---|---|---|---|
| | | | | | | | | QIV-HD/QIV-SD | |
| Investigational | Antigen/ | QIV-HD | | | QIV-SD | | | GMT | |
| dosage | strain | M | GMT | (95% CI) | M | GMT | (95% CI) | ratio | (95% CI) |
| QIV-HD | A/H1N1 | 30 | 678 | (486; 946) | 46 | 1109 | (831; 1481) | 0.61 | (0.39; 0.95) |
| (30 μg) | A/H3N2 | 30 | 1028 | (773; 1367) | 45 | 493 | (339; 717) | 2.09 | (1.26; 3.47) |
| | B/Victoria | 30 | 427 | (263; 694) | 46 | 423 | (302; 593) | 1.01 | (0.58; 1.77) |
| | B/Yamagata | 30 | 816 | (542; 1228) | 45 | 770 | (600; 987) | 1.06 | (0.68; 1.65) |
| QIV-HD | A/H1N1 | 31 | 765 | (505; 1159) | 46 | 1109 | (831; 1481) | 0.69 | (0.43; 1.12) |
| (45 μg) | A/H3N2 | 31 | 1280 | (854; 1918) | 45 | 493 | (339; 717) | 2.60 | (1.49; 4.52) |
| | B/Victoria | 31 | 585 | (375; 913) | 46 | 423 | (302; 593) | 1.38 | (0.81; 2.38) |
| | B/Yamagata | 31 | 885 | (600; 1305) | 45 | 770 | (600; 987) | 1.15 | (0.75; 1.77) |
| QIV-HD | A/H1N1 | 28 | 975 | (593; 1602) | 46 | 1109 | (831; 1481) | 0.88 | (0.52; 1.49) |
| (60 μg) | A/H3N2 | 27 | 1474 | (1058; 2055) | 45 | 493 | (339; 717) | 2.99 | (1.74; 5.14) |
| | B/Victoria | 28 | 800 | (507; 1261) | 46 | 423 | (302; 593) | 1.89 | (1.09; 3.28) |
| | B/Yamagata | 28 | 1174 | (784; 1757) | 45 | 770 | (600; 987) | 1.52 | (0.98; 2.36) |

M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.

TABLE 31
Comparison of QIV-HD to QIV-SD using seroconversion rate by HAI method
at 28 days after the last vaccination (US subjects 5-8 years) - IAS

| | | US (5-8 years) | | | | | | | |
|---|---|---|---|---|---|---|---|---|---|
| | | | | | | | | QIV-HD minus QIV-SD | |
| Investigational | Antigen/ | QIV-HD | | | QIV-SD | | | Difference | |
| dosage | strain | n/M | % | (95% CI) | n/M | % | (95% CI) | of % | (95% CI) |
| QIV-HD | A/H1N1 | 14/29 | 48.3 | (29.4; 67.5) | 29/45 | 64.4 | (48.8; 78.1) | −16.17 | (−37.08; 6.47) |
| (30 μg) | A/H3N2 | 13/29 | 44.8 | (26.4; 64.3) | 22/43 | 51.2 | (35.5; 66.7) | −6.34 | (−28.05; 16.43) |
| | B/Victoria | 23/29 | 79.3 | (60.3; 92.0) | 35/45 | 77.8 | (62.9; 88.8) | 1.53 | (−18.64; 19.28) |
| | B/Yamagata | 13/29 | 44.8 | (26.4; 64.3) | 35/44 | 79.5 | (64.7; 90.2) | −34.72 | (−53.58; −12.18) |
| QIV-HD | A/H1N1 | 12/30 | 40.0 | (22.7; 59.4) | 29/45 | 64.4 | (48.8; 78.1) | −24.44 | (−44.18; −1.51) |
| (45 μg) | A/H3N2 | 20/30 | 66.7 | (47.2; 82.7) | 22/43 | 51.2 | (35.5; 66.7) | 15.50 | (−7.35; 35.67) |
| | B/Victoria | 26/30 | 86.7 | (69.3; 96.2) | 35/45 | 77.8 | (62.9; 88.8) | 8.89 | (−10.11; 25.07) |
| | B/Yamagata | 20/30 | 66.7 | (47.2; 82.7) | 35/44 | 79.5 | (64.7; 90.2) | −12.88 | (−33.04; 7.03) |
| QIV-HD | A/H1N1 | 18/27 | 66.7 | (46.0; 83.5) | 29/45 | 64.4 | (48.8; 78.1) | 2.22 | (−20.30; 22.94) |
| (60 μg) | A/H3N2 | 18/26 | 69.2 | (48.2; 85.7) | 22/43 | 51.2 | (35.5; 66.7) | 18.07 | (−5.84; 38.35) |
| | B/Victoria | 24/27 | 88.9 | (70.8; 97.6) | 35/45 | 77.8 | (62.9; 88.8) | 11.11 | (−8.40; 26.92) |
| | B/Yamagata | 24/27 | 88.9 | (70.8; 97.6) | 35/44 | 79.5 | (64.7; 90.2) | 9.34 | (−9.99; 25.15) |

n: number of subjects experiencing seroconversion.
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
Seroconversion: subjects with a pre-vaccination HAI titer <10 (1/dil) and a post-vaccination titer >=40 (1/dil) or a pre-vaccination titer >=10 (1/dil) and a >=4-fold increase in post-vaccination.

TABLE 32
Summary of SN antibody response for each antigen (US subjects 5-8 years) - IAS

| | | US (5-8 years) | | | | | | | | | | | | | | | |
|---|---|---|---|---|---|---|---|---|---|---|---|---|---|---|---|---|---|
| | | QIV-HD (30 μg) | | | | | | | | QIV-HD (45 μg) | | | | | | | |
| | | (N = 31) | | | | | | | | (N = 31) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 30 | | 29 | | 30 | | 30 | | 29 | | 28 | | 29 | | 29 | |
| vaccination | GMT | 1598 | | 364 | | 53.0 | | 190 | | 1317 | | 240 | | 52.5 | | 158 | |
| (D 0) | (95% CI) | (968; 2639) | | (250; 531) | | (31.0; 90.6) | | (105; 344) | | (572; 3033) | | (146; 393) | | (29.0; 95.2) | | (94.7; 263) | |
| | Subject with | 0 | | 0 | | 2 | (6.7) | 1 | (3.3) | 1 | (3.4) | 0 | | 5 | (17.2) | 0 | |
| | titers <10 | | | | | | | | | | | | | | | | |
| | (1/dil): n (%) | | | | | | | | | | | | | | | | |
| | (95% CI) | (0; 11.6) | | (0; 11.9) | | (0.8; 22.1) | | (0.1; 17.2) | | (0.1; 17.8) | | (0; 12.3) | | (5.8; 35.8) | | (0; 11.9) | |
| | Subject with | 29 | (96.7) | 29 | (100) | 16 | (53.3) | 25 | (83.3) | 26 | (89.7) | 26 | (92.9) | 16 | (55.2) | 23 | (79.3) |
| | titers >=40 | | | | | | | | | | | | | | | | |
| | (1/dil): n (%) | | | | | | | | | | | | | | | | |
| | (95% CI) | (82.8; 99.9) | | (88.1; 100) | | (34.3; 71.7) | | (65.3; 94.4) | | (72.6; 97.8) | | (76.5; 99.1) | | (35.7; 73.6) | | (60.3; 92.0) | |
| Post- | M | 29 | | 29 | | 29 | | 29 | | 31 | | 31 | | 31 | | 31 | |
| vaccination | GMT | 6964 | | 1104 | | 527 | | 1651 | | 6766 | | 1529 | | 932 | | 1545 | |
| (D 28 or | (95% CI) | (4795; 10112) | | (829; 1471) | | (284; 976) | | (1051; 2592) | | (4333; 10565) | | (1033; 2262) | | (538; 1614) | | (911; 2621) | |
| D 56 *) | Subject with | 0 | | 0 | | 0 | | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 11.9) | | (0; 11.9) | | (0; 11.9) | | (0; 11.9) | | (0; 11.2) | | (0; 11.2) | | (0; 11.2) | | (0; 11.2) | |
| | Subject with | 29 | (100) | 29 | (100) | 29 | (100) | 29 | (100) | 31 | (100) | 31 | (100) | 29 | (93.5) | 30 | (96.8) |
| | titers >=40 | | | | | | | | | | | | | | | | |
| | (1/dil): n (%) | | | | | | | | | | | | | | | | |
| | (95% CI) | (88.1; 100) | | (88.1; 100) | | (88.1; 100) | | (88.1; 100) | | (88.8; 100) | | (88.8; 100) | | (78.6; 99.2) | | (83.3; 99.9) | |
| Post- | M | 28 | | 27 | | 28 | | 28 | | 29 | | 28 | | 29 | | 29 | |
| vaccination | GMTR | 4.68 | | 3.13 | | 9.41 | | 7.57 | | 5.55 | | 6.54 | | 17.7 | | 10.0 | |
| response | (95% CI) | (2.70; 8.09) | | (2.00; 4.88) | | (5.79; 15.3) | | (4.34; 13.2) | | (2.42; 12.7) | | (3.85; 11.1) | | (9.92; 31.7) | | (6.17; 16.2) | |
| based on | Subjects with | 17 | (60.7) | 16 | (59.3) | 27 | (96.4) | 22 | (78.6) | 16 | (55.2) | 21 | (75.0) | 28 | (96.6) | 25 | (86.2) |
| pre- | 2-Fold | | | | | | | | | | | | | | | | |
| vaccination | rise^{†}: n (%) | | | | | | | | | | | | | | | | |
| (D 28 or | (95% CI) | (40.6; 78.5) | | (38.8; 77.6) | | (81.7; 99.9) | | (59.0; 91.7) | | (35.7; 73.6) | | (55.1; 89.3) | | (82.2; 99.9) | | (68.3; 96.1) | |
| D 56 */D 0) | Subjects with | 14 | (50.0) | 11 | (40.7) | 22 | (78.6) | 15 | (53.6) | 12 | (41.4) | 16 | (57.1) | 25 | (86.2) | 22 | (75.9) |
| | 4-Fold | | | | | | | | | | | | | | | | |
| | rise^{†}: n (%) | | | | | | | | | | | | | | | | |
| | (95% CI) | (30.6; 69.4) | | (22.4; 61.2) | | (59.0; 91.7) | | (33.9; 72.5) | | (23.5; 61.1) | | (37.2; 75.5) | | (68.3; 96.1) | | (56.5; 89.7) | |
| | | US (5-8 years) | | | | | | | | | | | | | | | |
| | | QIV-HD (60 μg) | | | | | | | | QIV-SD | | | | | | | |
| | | (N = 30) | | | | | | | | (N = 48) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 28 | | 28 | | 28 | | 28 | | 45 | | 45 | | 45 | | 45 | |
| vaccination | GMT | 1163 | | 304 | | 63.4 | | 145 | | 1188 | | 250 | | 40.2 | | 170 | |
| (D 0) | (95% CI) | (527; 2567) | | (215; 429) | | (33.3; 121) | | (83.4; 251) | | (716; 1971) | | (180; 347) | | (26.9; 60.0) | | (114; 252) | |
| | Subject with | 0 | | 0 | | 4 | (14.3) | 1 | (3.6) | 2 | (4.4) | 0 | | 3 | (6.7) | 0 | |
| | titers <10 | | | | | | | | | | | | | | | | |
| | (1/dil): n (%) | | | | | | | | | | | | | | | | |
| | (95% CI) | (0; 12.3) | | (0; 12.3) | | (4.0; 32.7) | | (0.1; 18.3) | | (0.5; 15.1) | | (0; 7.9) | | (1.4; 18.3) | | (0; 7.9) | |
| | Subject with | 26 | (92.9) | 28 | (100) | 18 | (64.3) | 24 | (85.7) | 43 | (95.6) | 44 | (97.8) | 19 | (42.2) | 36 | (80.0) |
| | titers >=40 | | | | | | | | | | | | | | | | |
| | (1/dil): n (%) | | | | | | | | | | | | | | | | |
| | (95% CI) | (76.5; 99.1) | | (87.7; 100) | | (44.1; 81.4) | | (67.3; 96.0) | | (84.9; 99.5) | | (88.2; 99.9) | | (27.7; 57.8) | | (65.4; 90.4) | |
| Post- | M | 30 | | 30 | | 30 | | 30 | | 46 | | 45 | | 46 | | 45 | |
| vaccination | GMT | 9822 | | 1663 | | 889 | | 1647 | | 10565 | | 589 | | 489 | | 1382 | |
| (D 28 or | (95% CI) | (5726; 16849) | | (1236; 2236) | | (472; 1673) | | (1112; 2439) | | (7674; 14544) | | (435; 799) | | (316; 757) | | (984; 1940) | |
| D 56 *) | Subject with | 0 | | 0 | | 1 | (3.3) | 0 | | 0 | | 0 | | 1 | (2.2) | 0 | |
| | titers <10 | | | | | | | | | | | | | | | | |
| | (1/dil): n (%) | | | | | | | | | | | | | | | | |
| | (95% CI) | (0; 11.6) | | (0; 11.6) | | (0.1; 17.2) | | (0; 11.6) | | (0; 7.7) | | (0; 7.9) | | (0.1; 11.5) | | (0; 7.9) | |
| | Subject with | 30 | (100) | 30 | (100) | 28 | (93.3) | 30 | (100) | 46 | (100) | 45 | (100) | 44 | (95.7) | 45 | (100) |
| | titers >=40 | | | | | | | | | | | | | | | | |
| | (1/dil): n (%) | | | | | | | | | | | | | | | | |
| | (95% CI) | (88.4; 100) | | (88.4; 100) | | (77.9; 99.2) | | (88.4; 100) | | (92.3; 100) | | (92.1; 100) | | (85.2; 99.5) | | (92.1; 100) | |
| Post- | M | 28 | | 28 | | 28 | | 28 | | 43 | | 43 | | 43 | | 43 | |
| vaccination | GMTR | 8.48 | | 5.31 | | 14.2 | | 11.7 | | 9.23 | | 2.39 | | 12.2 | | 8.07 | |
| response | (95% CI) | (4.80; 15.0) | | (3.54; 7.98) | | (7.52; 26.7) | | (7.92; 17.2) | | (5.65; 15.1) | | (1.87; 3.05) | | (7.76; 19.0) | | (6.26; 10.4) | |
| based on | Subjects with | 22 | (78.6) | 23 | (82.1) | 27 | (96.4) | 28 | (100) | 35 | (81.4) | 19 | (44.2) | 40 | (93.0) | 43 | (100) |
| pre- | 2-Fold | | | | | | | | | | | | | | | | |
| vaccination | rise^{†}: n (%) | | | | | | | | | | | | | | | | |
| (D 28 or | (95% CI) | (59.0; 91.7) | | (63.1; 93.9) | | (81.7; 99.9) | | (87.7; 100) | | (66.6; 91.6) | | (29.1; 60.1) | | (80.9; 98.5) | | (91.8; 100) | |
| D 56 */D 0) | Subjects with | 18 | (64.3) | 12 | (42.9) | 25 | (89.3) | 25 | (89.3) | 27 | (62.8) | 10 | (23.3) | 35 | (81.4) | 33 | (76.7) |
| | 4-Fold | | | | | | | | | | | | | | | | |
| | rise^{†}: n (%) | | | | | | | | | | | | | | | | |

| | | | | | | | | |
|---|---|---|---|---|---|---|---|---|
| (95% CI) | (44.1; 81.4) | (24.5; 62.8) | (71.8; 97.7) | (71.8; 97.7) | (46.7; 77.0) | (11.8; 38.6) | (66.6; 91.6) | (61.4; 88.2) |

n: number of subjects experiencing the endpoint listed in the first two columns
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
* D 28 or D 56: at 28 days after the last vaccination.
^{†}X-Fold rise: the computed value (=post-vaccination computed value/pre-vaccination computed value) >=X.
GMTR (Geometric means of titer ratio): geometric mean of the individual titer ratios (post-vaccination/pre-vaccination).

2.4.3—Subjects 36 Months to Less than 5 Years of Age (Groups 1 [Stage 1], 2 [Stage 2], and 4 [Stage 3]—US)

HAI GMTs (Baseline and after Vaccination)

At baseline (pre-vaccination), GMTs were similar between the QIV-HD (30 μg, 45 μg, 60 μg) and QIV-SD group for the A/H1N1, A/H3N2, B/Victoria, and B/Yamagata strain (Table 33).

At 28 days after last vaccination, all GMTs significantly increased compared to baseline (Table 34).

HAI GMTs (Comparison of QIV-HD to QIV-SD)

The GMT ratio for QIV-HD (30 μg, 45 μg, 60 μg) over QIV-SD group was 0.54, 0.57, and 0.50, respectively, for the A/H1N1 strain; 1.56, 2.97, and 2.37, respectively, for the A/H3N2 strain; 0.80, 0.84, and 1.05, respectively, for the B/Victoria strain; and 0.96, 0.91, and 1.27, respectively, for the B/Yamagata strain (Table 34).

HAI GMTRs (Geometric Mean of Individual Titer Ratios of Post-Vaccination/Pre-Vaccination)

At 28 days after last vaccination, GMTRs for the QIV-HD (30 μg, 45 μg, 60 μg) groups and QIV-SD group were 10.2, 11.2, 20.1, and 20.8, respectively, for the A/H1N1; 8.41, 14.3, 12.3, and 4.58, respectively, for the A/H3N2 strain; 14.0, 13.5, 18.5, and 12.9, respectively, for the B/Victoria strain; and 11.7, 11.5, 20.3, and 9.83, respectively, for the B/Yamagata strain (Table 33). GMTRs for the QIV-HD (30 μg, 45 μg, and 60 μg) compared to QIV-SD group were higher for the A/H3N2, B/Victoria, and B/Yamagata strains and lower for the A/H1N1 strain.

HAI Seroconversion Rates (after Last Vaccination)

At 28 days after last vaccination, the seroconversion rates for the QIV-HD (30 μg, 45 μg, 60 μg) groups and QIV-SD group were 75.0%, 77.8%, 86.8%, and 84.6%, respectively, for the A/H1N1 strain; 71.4%, 88.9%, 81.6%, and 50.0%, respectively, for the A/H3N2 strain; 85.7%, 92.6%, 84.2%, and 89.2%, respectively, for the B/Victoria strain; and 85.7%, 92.0%, 92.3%, and 89.1%, respectively, for the B/Yamagata strain. The seroconversion rates for the QIV-HD (30 μg, 45 μg, 60 μg) groups were higher for the A/H3N2 and similar for the A/H1N1, B/Victoria, and B/Yamagata strain compared with QIV-SD (Table 35).

SN GMTs (Baseline and after Last Vaccination)

At baseline (pre-vaccination), GMTs were similar between the QIV-HD (30 μg, 45 μg, 60 μg) and QIV-SD group for the A/H3N2, B/Victoria, and B/Yamagata strains. For the A/H1N1 strain, GMTs were similar between the QIV-HD (45 μg and 60 μg) and QIV-SD groups and tended to have higher GMTs for the QIV-HD (30 μg) (Table 36).

At 28 days after last vaccination, all GMTs significantly increased compared to baseline. Compared to QIV-SD group, QIV-HD (60 μg) group had lower GMTs for the A/H1N1 strains, higher GMTs for the A/H3N2 and B/Yamagata strains, and similar GMTs for the B/Victoria strain (Table 36).

SN GMTR (Geometric Means of Individual Titer Ratios of Post-Vaccination/Pre-Vaccination)

GMTRs for the QIV-HD (30 μg, 45 μg, 60 μg) groups and QIV-SD groups are shown in Table 36. Compared to QIV-SD group, QIV-HD (60 μg) group had higher GMTRs for the A/H3N2, B/Victoria, and B/Yamagata strains and similar GMTRs for the A/H1N1 strain (Table 36).

SN: Subjects with 4-fold rise in titer

At 28 days after last vaccination, the number of subjects for the QIV-HD (30 μg, 45 μg, 60 μg) groups and QIV-SD group with a 4-fold rise in titer is shown in Table 36. The number of subjects with a 4-fold rise for the QIV-HD (60 μg) compared to QIV-SD was similar for the A/H1N1, B/Victoria, and B/Yamagata strains and higher for the A/H3N2 strain.

TABLE 33
Summary of HAI antibody response for each antigen (US subjects 36 months-less than 5 years) - IAS

| | | US (36 months-less than 5 years) | | | | | | | | | | | | | | | |
|---|---|---|---|---|---|---|---|---|---|---|---|---|---|---|---|---|---|
| | | QIV-HD (30 μg) | | | | | | | | QIV-HD (45 μg) | | | | | | | |
| | | (N = 32) | | | | | | | | (N = 28) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 28 | | 28 | | 28 | | 28 | | 27 | | 27 | | 27 | | 26 | |
| vaccination | GMT | 73.4 | | 53.2 | | 26.3 | | 53.8 | | 65.1 | | 61.1 | | 25.5 | | 50.8 | |
| (D 0) | (95% CI) | (36.4; 148) | | (26.4; 107) | | (16.0; 43.0) | | (29.2; 99.1) | | (29.7; 143) | | (33.1; 113) | | (14.1; 46.1) | | (24.8; 104) | |
| | Subject with | 6 | (21.4) | 7 | (25.0) | 5 | (17.9) | 4 | (14.3) | 8 | (29.6) | 5 | (18.5) | 8 | (29.6) | 6 | (23.1) |
| | titers <10 | | | | | | | | | | | | | | | | |
| | (1/dil): n (%) | | | | | | | | | | | | | | | | |
| | (95% CI) | (8.3; 41.0) | | (10.7; 44.9) | | (6.1; 36.9) | | (4.0; 32.7) | | (13.8; 50.2) | | (6.3; 38.1) | | (13.8; 50.2) | | (9.0; 43.6) | |
| | Subject with | 18 | (64.3) | 16 | (57.1) | 10 | (35.7) | 17 | (60.7) | 17 | (63.0) | 20 | (74.1) | 11 | (40.7) | 14 | (53.8) |
| | titers >=40 | | | | | | | | | | | | | | | | |
| | (1/dil): n (%) | | | | | | | | | | | | | | | | |
| | (95% CI) | (44.1; 81.4) | | (37.2; 75.5) | | (18.6; 55.9) | | (40.6; 78.5) | | (42.4; 80.6) | | (53.7; 88.9) | | (22.4; 61.2) | | (33.4; 73.4) | |
| Post- | M | 32 | | 32 | | 32 | | 32 | | 28 | | 28 | | 28 | | 27 | |
| vaccination | GMT | 698 | | 462 | | 334 | | 613 | | 742 | | 883 | | 353 | | 585 | |
| (D 28 or | (95% CI) | (397; 1227) | | (253; 845) | | (206; 541) | | (392; 959) | | (455; 1212) | | (515; 1515) | | (211; 590) | | (338; 1013) | |
| D 56 §) | Subject with | 0 | | 0 | | 0 | | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 10.9) | | (0; 10.9) | | (0; 10.9) | | (0; 10.9) | | (0; 12.3) | | (0; 12.3) | | (0; 12.3) | | (0; 12.8) | |
| | Subject with | 31 | (96.9) | 29 | (90.6) | 31 | (96.9) | 31 | (96.9) | 28 | (100) | 27 | (96.4) | 27 | (96.4) | 27 | (100) |
| | titers >=40 | | | | | | | | | | | | | | | | |
| | (1/dil): n (%) | | | | | | | | | | | | | | | | |
| | (95% CI) | (83.8; 99.9) | | (75.0; 98.0) | | (83.8; 99.9) | | (83.8; 99.9) | | (87.7; 100) | | (81.7; 99.9) | | (81.7; 99.9) | | (87.2; 100) | |
| Post- | M | 28 | | 28 | | 28 | | 28 | | 27 | | 27 | | 27 | | 25 | |
| vaccination | GMTR | 10.2 | | 8.41 | | 14.0 | | 11.7 | | 11.2 | | 14.3 | | 13.5 | | 11.5 | |
| response | (95% CI) | (5.88; 17.9) | | (5.01; 14.1) | | (8.80; 22.2) | | (6.88; 20.0) | | (6.26; 19.9) | | (9.02; 22.5) | | (9.47; 19.4) | | (7.37; 17.8) | |
| based on | Seroconversion | 21 | (75.0) | 20 | (71.4) | 24 | (85.7) | 24 | (85.7) | 21 | (77.8) | 24 | (88.9) | 25 | (92.6) | 23 | (92.0) |
| pre- | rate: n (%) | | | | | | | | | | | | | | | | |
| vaccination | (95% CI) | (55.1; 89.3) | | (51.3; 86.8) | | (67.3; 96.0) | | (67.3; 96.0) | | (57.7; 91.4) | | (70.8; 97.6) | | (75.7; 99.1) | | (74.0; 99.0) | |
| (D 28 or | | | | | | | | | | | | | | | | | |
| D 56 §/D 0) | | | | | | | | | | | | | | | | | |
| | | US (36 months-less than 5 years) | | | | | | | | | | | | | | | |
| | | QIV-HD (60 μg) | | | | | | | | QIV-SD | | | | | | | |
| | | (N = 42) | | | | | | | | (N = 66) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 39 | | 39 | | 39 | | 40 | | 66 | | 65 | | 66 | | 65 | |
| vaccination | GMT | 32.3 | | 57.1 | | 24.5 | | 40.7 | | 65.5 | | 64.3 | | 31.7 | | 61.9 | |
| (D 0) | (95% CI) | (18.3; 57.0) | | (32.3; 101) | | (15.3; 39.3) | | (26.1; 63.4) | | (42.1; 102) | | (42.3; 97.8) | | (22.4; 45.1) | | (43.2; 88.8) | |
| | Subject with | 13 | (33.3) | 8 | (20.5) | 12 | (30.8) | 7 | (17.5) | 9 | (13.6) | 12 | (18.5) | 17 | (25.8) | 7 | (10.8) |
| | titers <10 | | | | | | | | | | | | | | | | |
| | (1/dil): n (%) | | | | | | | | | | | | | | | | |
| | (95% CI) | (19.1; 50.2) | | (9.3; 36.5) | | (17.0; 47.6) | | (7.3; 32.8) | | (6.4; 24.3) | | (9.9; 30.0) | | (15.8; 38.0) | | (4.4; 20.9) | |
| | Subject with | 17 | (43.6) | 23 | (59.0) | 17 | (43.6) | 21 | (52.5) | 41 | (62.1) | 45 | (69.2) | 31 | (47.0) | 44 | (67.7) |
| | titers >=40 | | | | | | | | | | | | | | | | |
| | (1/dil): n (%) | | | | | | | | | | | | | | | | |
| | (95% CI) | (27.8; 60.4) | | (42.1; 74.4) | | (27.8; 60.4) | | (36.1; 68.5) | | (49.3; 73.8) | | (56.6; 80.1) | | (34.6; 59.7) | | (54.9; 78.8) | |
| Post- | M | 40 | | 40 | | 40 | | 40 | | 65 | | 65 | | 65 | | 65 | |
| vaccination | GMT | 651 | | 704 | | 441 | | 816 | | 1301 | | 297 | | 420 | | 640 | |
| (D 28 or | (95% CI) | (440; 964) | | (418; 1185) | | (318; 612) | | (577; 1154) | | (1010; 1676) | | (203; 435) | | (315; 559) | | (489; 838) | |
| D 56 §) | Subject with | 0 | | 0 | | 0 | | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 8.8) | | (0; 8.8) | | (0; 8.8) | | (0; 8.8) | | (0; 5.5) | | (0; 5.5) | | (0; 5.5) | | (0; 5.5) | |
| | Subject with | 40 | (100) | 38 | (95.0) | 39 | (97.5) | 40 | (100) | 65 | (100) | 58 | (89.2) | 63 | (96.9) | 64 | (98.5) |
| | titers >=40 | | | | | | | | | | | | | | | | |
| | (1/dil): n (%) | | | | | | | | | | | | | | | | |
| | (95% CI) | (91.2; 100) | | (83.1; 99.4) | | (86.8; 99.9) | | (91.2; 100) | | (94.5; 100) | | (79.1; 95.6) | | (89.3; 99.6) | | (91.7; 100) | |
| Post- | M | 38 | | 38 | | 38 | | 39 | | 65 | | 64 | | 65 | | 64 | |
| vaccination | GMTR | 20.1 | | 12.3 | | 18.5 | | 20.3 | | 20.8 | | 4.58 | | 12.9 | | 9.83 | |
| response | (95% CI) | (11.4; 35.3) | | (8.10; 18.6) | | (11.9; 28.9) | | (12.8; 32.4) | | (13.7; 31.5) | | (3.56; 5.89) | | (9.53; 17.4) | | (7.55; 12.8) | |
| based on | Seroconversion | 33 | (86.8) | 31 | (81.6) | 32 | (84.2) | 36 | (92.3) | 55 | (84.6) | 32 | (50.0) | 58 | (89.2) | 57 | (89.1) |
| pre- | rate: n (%) | | | | | | | | | | | | | | | | |

| | | | | | | | | | |
|---|---|---|---|---|---|---|---|---|---|
| vaccination | (95% CI) | (71.9; 95.6) | (65.7; 92.3) | (68.7; 94.0) | (79.1; 98.4) | (73.5; 92.4) | (37.2; 62.8) | (79.1; 95.6) | (78.8; 95.5) |
| (D 28 or | | | | | | | | | |
| D 56 §/D 0) | | | | | | | | | |

n: number of subjects experiencing the endpoint listed in the first two columns
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
* D 28 or D 56: at 28 days after the last vaccination.
GMTR (Geometric means of titer ratio): geometric mean of the individual titer ratios (post-vaccination/pre-vaccination).
Seroconversion: subjects with a pre-vaccination HAI titer <10 (1/dil) and a post-vaccination titer >=40 (1/dil) or a pre-vaccination titer >=10 (1/dil) and a >=4-fold increase in post-vaccination.

TABLE 34
Comparison of QIV-HD to QIV-SD using GMTs by HAI method at 28 days after
the last vaccination (US subjects 36 months-less than 5 years) - IAS

| | | US (36 months-<5 years) | | | | | | | |
|---|---|---|---|---|---|---|---|---|---|
| | | | | | | | | QIV-HD/QIV-SD | |
| Investigational | Antigen/ | QIV-HD | | | QIV-SD | | | GMT | |
| dosage | strain | M | GMT | (95% CI) | M | GMT | (95% CI) | ratio | (95% CI) |
| QIV-HD | A/H1N1 | 32 | 698 | (397; 1227) | 65 | 1301 | (1010; 1676) | 0.54 | (0.32; 0.91) |
| (30 μg) | A/H3N2 | 32 | 462 | (253; 845) | 65 | 297 | (203; 435) | 1.56 | (0.79; 3.07) |
| | B/Victoria | 32 | 334 | (206; 541) | 65 | 420 | (315; 559) | 0.80 | (0.47; 1.34) |
| | B/Yamagata | 32 | 613 | (392; 959) | 65 | 640 | (489; 838) | 0.96 | (0.59; 1.56) |
| QIV-HD | A/H1N1 | 28 | 742 | (455; 1212) | 65 | 1301 | (1010; 1676) | 0.57 | (0.35; 0.94) |
| (45 μg) | A/H3N2 | 28 | 883 | (515; 1515) | 65 | 297 | (203; 435) | 2.97 | (1.52; 5.82) |
| | B/Victoria | 28 | 353 | (211; 590) | 65 | 420 | (315; 559) | 0.84 | (0.49; 1.45) |
| | B/Yamagata | 27 | 585 | (338; 1013) | 65 | 640 | (489; 838) | 0.91 | (0.53; 1.56) |
| QIV-HD | A/H1N1 | 40 | 651 | (440; 964) | 65 | 1301 | (1010; 1676) | 0.50 | (0.32; 0.78) |
| (60 μg) | A/H3N2 | 40 | 704 | (418; 1185) | 65 | 297 | (203; 435) | 2.37 | (1.27; 4.44) |
| | B/Victoria | 40 | 441 | (318; 612) | 65 | 420 | (315; 559) | 1.05 | (0.67; 1.63) |
| | B/Yamagata | 40 | 816 | (577; 1154) | 65 | 640 | (489; 838) | 1.27 | (0.83; 1.96) |

M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.

TABLE 35
Comparison of QIV-HD to QIV-SD using seroconversion rate by HAI method at 28 days
after the last vaccination (US subjects 36 months-less than 5 years) - IAS

| | | US (36 months-less than 5 years) | | | | | | | |
|---|---|---|---|---|---|---|---|---|---|
| | | | | | | | | QIV-HD minus QIV-SD | |
| Investigational | Antigen/ | QIV-HD | | | QIV-SD | | | Difference | |
| dosage | strain | n/M | % | (95% CI) | n/M | % | (95% CI) | of % | (95% CI) |
| QIV-HD | A/H1N1 | 21/28 | 75.0 | (55.1; 89.3) | 55/65 | 84.6 | (73.5; 92.4) | −9.62 | (−29.19; 6.69) |
| (30 μg) | A/H3N2 | 20/28 | 71.4 | (51.3; 86.8) | 32/64 | 50.0 | (37.2; 62.8) | 21.43 | (−0.56; 39.29) |
| | B/Victoria | 24/28 | 85.7 | (67.3; 96.0) | 58/65 | 89.2 | (79.1; 95.6) | −3.52 | (−21.56; 9.54) |
| | B/Yamagata | 24/28 | 85.7 | (67.3; 96.0) | 57/64 | 89.1 | (78.8; 95.5) | −3.35 | (−21.42; 9.80) |
| QIV-HD | A/H1N1 | 21/27 | 77.8 | (57.7; 91.4) | 55/65 | 84.6 | (73.5; 92.4) | −6.84 | (−26.58; 8.93) |
| (45 μg) | A/H3N2 | 24/27 | 88.9 | (70.8; 97.6) | 32/64 | 50.0 | (37.2; 62.8) | 38.89 | (18.18; 52.83) |
| | B/Victoria | 25/27 | 92.6 | (75.7; 99.1) | 58/65 | 89.2 | (79.1; 95.6) | 3.36 | (−13.51; 14.56) |
| | B/Yamagata | 23/25 | 92.0 | (74.0; 99.0) | 57/64 | 89.1 | (78.8; 95.5) | 2.94 | (−14.91; 14.45) |
| QIV-HD | A/H1N1 | 33/38 | 86.8 | (71.9; 95.6) | 55/65 | 84.6 | (73.5; 92.4) | 2.23 | (−13.49; 15.21) |
| (60 μg) | A/H3N2 | 31/38 | 81.6 | (65.7; 92.3) | 32/64 | 50.0 | (37.2; 62.8) | 31.58 | (12.43; 46.62) |
| | B/Victoria | 32/38 | 84.2 | (68.7; 94.0) | 58/65 | 89.2 | (79.1; 95.6) | −5.02 | (−20.63; 7.88) |
| | B/Yamagata | 36/39 | 92.3 | (79.1; 98.4) | 57/64 | 89.1 | (78.8; 95.5) | 3.25 | (−10.54; 14.41) |

n: number of subjects experiencing seroconversion.
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
Seroconversion: subjects with a pre-vaccination HAI titer <10 (1/dil) and a post-vaccination titer >=40 (1/dil) or a pre-vaccination titer >=10 (1/dil) and a >=4-fold increase in post-vaccination.

TABLE 36
Summary of SN antibody response for each antigen (US subjects 36 months-less than 5 years) - IAS

| | | US (36 months-less than 5 years) | | | | | | | | | | | | | | | |
|---|---|---|---|---|---|---|---|---|---|---|---|---|---|---|---|---|---|
| | | QIV-HD (30 μg) | | | | | | | | QIV-HD (45 μg) | | | | | | | |
| | | (N = 32) | | | | | | | | (N = 28) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 27 | | 26 | | 26 | | 26 | | 25 | | 23 | | 25 | | 23 | |
| vaccination | GMT | 575 | | 119 | | 26.7 | | 85.3 | | 282 | | 152 | | 22.9 | | 84.7 | |
| (D 0) | (95% CI) | (236; 1400) | | (75.0; 188) | | (15.1; 47.1) | | (50.0; 146) | | (105; 757) | | (99.9; 231) | | (11.6; 45.1) | | (38.9; 185) | |
| | Subject with | 2 | (7.4) | 0 | | 6 | (23.1) | 0 | | 3 | (12.0) | 0 | | 9 | (36.0) | 2 | (8.7) |
| | titers <10 | | | | | | | | | | | | | | | | |
| | (1/dil): n (%) | | | | | | | | | | | | | | | | |
| | (95% CI) | (0.9; 24.3) | | (0; 13.2) | | (9.0; 43.6) | | (0; 13.2) | | (2.5; 31.2) | | (0; 14.8) | | (18.0; 57.5) | | (1.1; 28.0) | |
| | Subject with | 23 | (85.2) | 22 | (84.6) | 8 | (30.8) | 18 | (69.2) | 18 | (72.0) | 20 | (87.0) | 7 | (28.0) | 12 | (52.2) |
| | titers >=40 | | | | | | | | | | | | | | | | |
| | (1/dil): n (%) | | | | | | | | | | | | | | | | |
| | (95% CI) | (66.3; 95.8) | | (65.1; 95.6) | | (14.3; 51.8) | | (48.2; 85.7) | | (50.6; 87.9) | | (66.4; 97.2) | | (12.1; 49.4) | | (30.6; 73.2) | |
| Post- | M | 32 | | 32 | | 32 | | 32 | | 27 | | 27 | | 27 | | 27 | |
| vaccination | GMT | 6756 | | 589 | | 396 | | 1049 | | 6903 | | 1049 | | 438 | | 1019 | |
| (D 28 or | (95% CI) | (3499; 13047) | | (361; 960) | | (225; 695) | | (621; 1772) | | (4091; 11647) | | (635; 1732) | | (229; 838) | | (550; 1888) | |
| D 56 *) | Subject with | 0 | | 0 | | 0 | | 0 | | 0 | | 0 | | 0 | | 0 | |
| | titers <10 | | | | | | | | | | | | | | | | |
| | (1/dil): n (%) | | | | | | | | | | | | | | | | |
| | (95% CI) | (0; 10.9) | | (0; 10.9) | | (0; 10.9) | | (0; 10.9) | | (0; 12.8) | | (0; 12.8) | | (0; 12.8) | | (0; 12.8) | |
| | Subject with | 32 | (100) | 31 | (96.9) | 30 | (93.8) | 31 | (96.9) | 27 | (100) | 27 | (100) | 26 | (96.3) | 27 | (100) |
| | titers >=40 | | | | | | | | | | | | | | | | |
| | (1/dil): n (%) | | | | | | | | | | | | | | | | |
| | (95% CI) | (89.1; 100) | | (83.8; 99.9) | | (79.2; 99.2) | | (83.8; 99.9) | | (87.2; 100) | | (87.2; 100) | | (81.0; 99.9) | | (87.2; 100) | |
| Post- | M | 27 | | 26 | | 26 | | 26 | | 24 | | 22 | | 24 | | 22 | |
| vaccination | GMTR | 13.2 | | 4.41 | | 16.8 | | 15.1 | | 25.0 | | 5.48 | | 16.0 | | 10.7 | |
| response | (95% CI) | (6.29; 27.9) | | (3.09; 6.30) | | (9.74; 29.1) | | (9.20; 24.8) | | (11.5; 54.2) | | (3.54; 8.48) | | (10.5; 24.4) | | (6.60; 17.3) | |
| based on | Subjects with | 24 | (88.9) | 21 | (80.8) | 25 | (96.2) | 24 | (92.3) | 22 | (91.7) | 20 | (90.9) | 24 | (100) | 20 | (90.9) |
| pre- | 2-Fold | | | | | | | | | | | | | | | | |
| vaccination | rise^{†}: n (%) | | | | | | | | | | | | | | | | |
| (D 28 or | (95% CI) | (70.8; 97.6) | | (60.6; 93.4) | | (80.4; 99.9) | | (74.9; 99.1) | | (73.0; 99.0) | | (70.8; 98.9) | | (85.8; 100) | | (70.8; 98.9) | |
| D 56 */D 0) | Subjects with | 19 | (70.4) | 13 | (50.0) | 22 | (84.6) | 22 | (84.6) | 20 | (83.3) | 11 | (50.0) | 22 | (91.7) | 18 | (81.8) |
| | 4-Fold | | | | | | | | | | | | | | | | |
| | rise^{†}: n (%) | | | | | | | | | | | | | | | | |

| | | | | | | | | |
|---|---|---|---|---|---|---|---|---|
| (95% CI) | (49.8; 86.2) | (29.9; 70.1) | (65.1; 95.6) | (65.1; 95.6) | (62.6; 95.3) | (28.2; 71.8) | (73.0; 99.0) | (59.7; 94.8) |

| | | US (36 months-<5 years) | | | | | | | | | | | | | | | |
|---|---|---|---|---|---|---|---|---|---|---|---|---|---|---|---|---|---|
| | | QIV-HD (60 μg) | | | | | | | | QIV-SD | | | | | | | |
| | | (N = 42) | | | | | | | | (N = 66) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 38 | | 38 | | 37 | | 37 | | 64 | | 64 | | 64 | | 63 | |
| vaccination | GMT | 194 | | 126 | | 24.7 | | 86.8 | | 452 | | 141 | | 30.1 | | 91.6 | |
| (D 0) | (95% CI) | (87.5; 430) | | (87.4; 182) | | (15.0; 40.4) | | (55.5; 136) | | (274; 748) | | (110; 182) | | (21.2; 42.6) | | (64.1; 131) | |
| | Subject with | 6 | (15.8) | 0 | | 13 | (35.1) | 1 | (2.7) | 3 | (4.7) | 0 | | 12 | (18.8) | 3 | (4.8) |
| | titers <10 | | | | | | | | | | | | | | | | |
| | (1/dil): n (%) | | | | | | | | | | | | | | | | |
| | (95% CI) | (6.0; 31.3) | | (0; 9.3) | | (20.2; 52.5) | | (0.1; 14.2) | | (1.0; 13.1) | | (0; 5.6) | | (10.1; 30.5) | | (1.0; 13.3) | |
| | Subject with | 25 | (65.8) | 31 | (81.6) | 14 | (37.8) | 27 | (73.0) | 58 | (90.6) | 57 | (89.1) | 27 | (42.2) | 44 | (69.8) |
| | titers >=40 | | | | | | | | | | | | | | | | |
| | (1/dil): n (%) | | | | | | | | | | | | | | | | |
| | (95% CI) | (48.6; 80.4) | | (65.7; 92.3) | | (22.5; 55.2) | | (55.9; 86.2) | | (80.7; 96.5) | | (78.8; 95.5) | | (29.9; 55.2) | | (57.0; 80.8) | |
| Post- | M | 40 | | 39 | | 39 | | 39 | | 63 | | 64 | | 63 | | 63 | |
| vaccination | GMT | 5741 | | 830 | | 611 | | 1428 | | 12366 | | 388 | | 542 | | 896 | |
| (D 28 or | (95% CI) | (3631; 9079) | | (516; 1336) | | (397; 940) | | (954; 2138) | | (9085; 16833) | | (284; 531) | | (382; 768) | | (662; 1213) | |
| D 56 *) | Subject with | 0 | | 0 | | 0 | | 0 | | 0 | | 0 | | 1 | (1.6) | 0 | |
| | titers <10 | | | | | | | | | | | | | | | | |
| | (1/dil): n (%) | | | | | | | | | | | | | | | | |
| | (95% CI) | (0; 8.8) | | (0; 9.0) | | (0; 9.0) | | (0; 9.0) | | (0; 5.7) | | (0; 5.6) | | (0; 8.5) | | (0; 5.7) | |
| | Subject with | 40 | (100) | 38 | (97.4) | 37 | (94.9) | 39 | (100) | 63 | (100) | 62 | (96.9) | 60 | (95.2) | 62 | (98.4) |
| | titers >=40 | | | | | | | | | | | | | | | | |
| | (1/dil): n (%) | | | | | | | | | | | | | | | | |
| | (95% CI) | (91.2; 100) | | (86.5; 99.9) | | (82.7; 99.4) | | (91.0; 100) | | (94.3; 100) | | (89.2; 99.6) | | (86.7; 99.0) | | (91.5; 100) | |
| Post- | M | 37 | | 36 | | 36 | | 36 | | 61 | | 62 | | 61 | | 60 | |
| vaccination | GMTR | 33.0 | | 7.01 | | 28.0 | | 20.2 | | 27.7 | | 2.57 | | 18.7 | | 9.68 | |
| response | (95% CI) | (16.2; 67.5) | | (4.91; 10.0) | | (16.7; 47.1) | | (12.5; 32.6) | | (16.7; 45.7) | | (2.05; 3.21) | | (13.4; 25.9) | | (7.40; 12.7) | |
| based on | Subjects with | 32 | (86.5) | 33 | (91.7) | 36 | (100) | 34 | (94.4) | 54 | (88.5) | 34 | (54.8) | 59 | (96.7) | 57 | (95.0) |
| pre- | 2-Fold | | | | | | | | | | | | | | | | |
| vaccination | rise^{†}: n (%) | | | | | | | | | | | | | | | | |
| (D 28 or | (95% CI) | (71.2; 95.5) | | (77.5; 98.2) | | (90.3; 100) | | (81.3; 99.3) | | (77.8; 95.3) | | (41.7; 67.5) | | (88.7; 99.6) | | (86.1; 99.0) | |
| D 56 */D 0) | Subjects with | 30 | (81.1) | 23 | (63.9) | 29 | (80.6) | 30 | (83.3) | 48 | (78.7) | 16 | (25.8) | 55 | (90.2) | 50 | (83.3) |
| | 4-Fold | | | | | | | | | | | | | | | | |
| | rise^{†}: n (%) | | | | | | | | | | | | | | | | |

| | | | | | | | | |
|---|---|---|---|---|---|---|---|---|
| (95% CI) | (64.8; 92.0) | (46.2; 79.2) | (64.0; 91.8) | (67.2; 93.6) | (66.3; 88.1) | (15.5; 38.5) | (79.8; 96.3) | (71.5; 91.7) |

n: number of subjects experiencing the endpoint listed in the first two columns
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
* D 28 or D 56: at 28 days after the last vaccination.
^{†}X-Fold rise: the computed value (=post-vaccination computed value/pre-vaccination computed value) >=X.
GMTR (Geometric means of titer ratio): geometric mean of the individual titer ratios (post-vaccination/pre-vaccination).

2.4.4—Subjects 6 to Less than 36 Months of Age (Groups 3 [Stage 2], 5 [Stage 3], and 6 and 7 [Stage 4]—US)

HAI GMTs

At baseline (pre-vaccination), GMTs were similar between the QIV-HD (30 μg, 45 μg, 60 μg) and QIV-SD group for the A/H1N1, B/Victoria, and B/Yamagata strains. For the A/H3N2, GMTs were similar between the QIV-HD (45 μg and 60 μg) and QIV-SD group and tended to be lower for the QIV-HD (30 μg) group (Table 37).

At 28 days after last vaccination, all GMTs significantly increased compared to baseline. GMTs for the QIV-HD (30 μg, 45 μg, 60 μg) groups and QIV-SD group were 303, 248, 603, and 142, respectively, for the A/H1N1; 149, 239, 505, and 161, respectively, for the A/H3N2 strain; 167, 186, 276, and 135, respectively, for the B/Victoria strain; and 320, 343, 556, and 290, respectively, for the B/Yamagata strain (Table 37). GMTs for the QIV-HD (30 μg, 45 μg, 60 μg) groups were higher compared to QIV-SD group with QIV-HD (60 μg) having the highest GMTs for all influenza strains.

HAI GMTRs (Geometric Mean of Individual Titer Ratios of Post-Vaccination/Pre-Vaccination)

At 28 days after last vaccination, GMTRs for the QIV-HD (30 μg, 45 μg, 60 μg) groups and QIV-SD group were 30.6, 35.1, 44.2, and 9.41, respectively, for the A/H1N1; 20.2, 18.6, 22.8, and 9.72, respectively, for the A/H3N2 strain; 16.5, 18.6, 34.2, and 15.4, respectively, for the B/Victoria strain; and 23.0, 21.7, 50.3, and 21.1, respectively, for the B/Yamagata strain. GMTRs for all 4 influenza strains for the QIV-HD (60 μg) group were higher compared to QIV-HD (30 μg, 45 μg) groups and QIV-SD group. GMTRs for the QIV-HD (30 μg, 45 μg) groups were higher compared to QIV-SD group for the A/H1N1 and A/H3N2 strain and similar for the B/Victoria and B/Yamagata strains (Table 37).

HAI GMTs (Comparison of QIV-HD to QIV-SD)

The GMT ratio for QIV-HD (30 μg, 45 μg, 60 μg) and QIV-SD group was 2.13, 1.75, and 4.24, respectively, for the A/H1N1 strain; 0.93, 1.49, and 3.14, respectively, for the A/H3N2 strain; 1.23, 1.38, and 2.04, respectively, for the B/Victoria strain; and 1.10, 1.18, and 1.92, respectively, for the B/Yamagata strain (Table 38). The GMT ratio between QIV-HD (60 μg) group and QIV-SD group was higher compared to that of QIV-HD (30 μg and 45 μg) groups for all 4 influenza strains.

HAI GMTs in Previously Unvaccinated Subjects (Comparison of QIV-HD to QIV-SD)

The GMT ratio between QIV-HD (30 μg, 45 μg, 60 μg) groups post-dose 1 and QIV-SD post-dose 2 was much lower than those for QIV-HD (30 μg, 45 μg, 60 μg) post-dose 2 and QIV-SD post-dose 2, ranging from: 0.15 to 0.90 for the 3 QIV-HD groups post-dose 1 compared to QIV-SD post-dose 2; 0.93 to 2.42 for QIV-HD (30 μg) group post-dose 2 compared to QIV-SD post-dose 2; 1.81 to 3.29 for QIV-HD (45 μg) group post-dose 2 compared to QIV-SD post-dose 2; and 1.87 to 4.47 for QIV-HD (60 μg) group post-dose 2 compared to QIV-SD post-dose 2 (Table 39). Compared to QIV-HD (30 μg), QIV-HD (60 μg) group had higher GMT ratios and compared to QIV-HD (45 μg), QIV-HD (60 μg) had higher GMT ratios except for the B/Yamagata strain (Table 39).

Despite the low number of previously vaccinated subjects, a similar trend of higher GMT ratios for QIV-HD (60 μg) group is observed (Table 40).

HAI Seroconversion Rates

At 28 days after last vaccination, the seroconversion rates for QIV-HD (30 μg, 45 μg, 60 μg) groups and QIV-SD group were 91.7%, 84.6%, 84.8%, and 56.1%, respectively, for the A/H1N1 strain; 91.7%, 92.0%, 87.0%, and 71.2%, respectively, for the A/H3N2 strain; 79.2%, 84.0%, 91.3%, and 75.8%, respectively, for the B/Victoria strain; and 100.0%, 88.0%, 95.7%, and 92.4, respectively, for the B/Yamagata strain

The seroconversion rates for the QIV-HD (30 μg, 45 μg, 60 μg) were higher compared with QIV-SD against all 4 vaccine strains, except for B/Yamagata strain for the QIV-HD (45 μg). The differences in the seroconversion rates between QIV-HD (30 μg, 45 μg, 60 μg) and QIV-SD ranged from 28.55 to 35.61, for the A/H1N1 strain, 15.74 to 20.79 for the A/H3N2 strain, 3.41 to 15.55 for the B/Victoria strain, and −4.42 to 7.58 for the B/Yamagata strain (Table 41).

For the previously unvaccinated subjects, the seroconversion rates for QIV-HD (30 μg, 45 μg, 60 μg) groups post-dose 1 were much lower than those for QIV-SD post-dose 2 and QIV-HD (30 μg, 45 μg, 60 μg) post-dose 2, ranging from: 23.1% to 66.7% for the 3 QIV-HD groups post-dose 1; 47.8% to 93.5% for the QIV-SD group post-dose 2; and 76.9% to 100.0% for the 3 QIV-HD groups post-dose 2 (Table 42 and Table 43).

SN GMTs (Baseline and after Last Vaccination)

At baseline (pre-vaccination), GMTs were similar between the QIV-HD (30 μg, 45 μg, 60 μg) compared with QIV-SD against all 4 influenza strains (Table 44).

At 28 days after last vaccination, all GMTs significantly increased compared to baseline. GMTs for the QIV-HD (30 μg, 45 μg, 60 μg) groups and QIV-SD group were 2700, 2000, 4535, and 663, respectively, for the A/H1N1 strain; 237, 360, 623, and 271, respectively, for the A/H3N2 strain; 208, 237, 332, and 126, respectively, for the B/Victoria strain; and 354, 480, 660, and 340, respectively, for the B/Yamagata strain (Table 44). Compared to QIV-SD group, GMTs for QIV-HD (30 μg, 45 μg, 60 μg) groups were higher for all 4 influenza strains with QIV-HD (60 μg) inducing higher SN GMTs (Table 44).

SN GMTR (Geometric Means of Individual Titer Ratios of Post-Vaccination/Pre-Vaccination)

GMTRs for the QIV-HD (30 μg, 45 μg, 60 μg) groups and QIV-SD groups were 79.9, 165, 170, and 19.8, respectively, for the A/H1N1 strain; 5.56, 5.85, 7.13, and 4.04, respectively, for the A/H3N2 strain; 20.2, 22.4, 35.8, and 14.5, respectively, for the B/Victoria strain; and 14.6, 18.0, 22.7, and 11.6, respectively, for the B/Yamagata strain (Table 44). GMTRs for the QIV-HD (30 μg, 45 μg, 60 μg) groups tended to increase with the increase in dose and were higher compared to QIV-SD group (Table 44).

SN: Subjects with 4-Fold Rise

At 28 days after last vaccination, the number of subjects for the QIV-HD (30 μg, 45 μg, 60 μg) groups and QIV-SD group with a 4-fold rise in titer was 19/26 (95.0%), 23/27 (100%), 41/54 (95.3%), and 46/71 (76.7%), respectively, for the A/H1N1 strain; 13/26 (65.0%), 13/27 (59.1%), 27/54 (65.9%), and 30/71 (50.8%), respectively, for the A/H3N2 strain; 18/26 (90.0%), 19/27 (82.6%), 40/54 (93.0%), and 45/71 (76.3%), respectively, for the B/Victoria strain; and 17/26 (89.5%), 22/27 (95.7%), 39/54 (90.7%), and 44/71 (74.6%), respectively, for the B/Yamagata strain (Table 44). The number of subjects with a 4-fold rise was similar between the 3 QIV-HD (30 μg, 45 μg, 60 μg) groups and higher compared to QIV-SD group for all 4 influenza strains (Table 44).

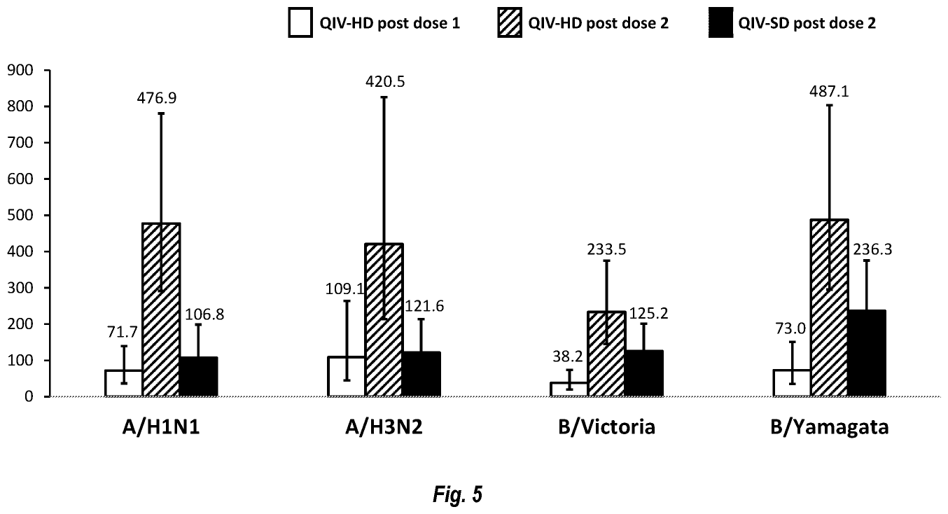

As shown in FIG. 5, two doses of QIV-HD (60 μg) administered one month apart generated higher GMTs than one dose of QIV-HD. Further, QIV-HD (60 μg) generated significantly higher GMTs after the second dose compared to QIV-SD post-second dose. This data suggests that 2 doses of QIV-HD (60 μg) may be particularly beneficial in previously unvaccinated subjects (e.g., subjects 6 to less than 36 months of age).

TABLE 37
Summary of HAI antibody response for each antigen (US subjects 6-less than 36 months) - IAS

| | | US (6-less than 36 months) | | | | | | | | | | | | | | | |
|---|---|---|---|---|---|---|---|---|---|---|---|---|---|---|---|---|---|
| | | QIV-HD (30 μg) | | | | | | | | QIV-HD (45 μg) | | | | | | | |
| | | (N = 26) | | | | | | | | (N = 27) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 24 | | 24 | | 24 | | 24 | | 27 | | 26 | | 26 | | 26 | |
| vaccination | GMT | 10.4 | | 7.38 | | 9.58 | | 13.9 | | 6.98 | | 12.4 | | 9.74 | | 15.3 | |
| (D 0) | (95% CI) | (5.89; 18.5) | | (5.17; 10.5) | | (6.50; 14.1) | | (7.90; 24.6) | | (4.82; 10.1) | | (6.92; 22.2) | | (6.20; 15.3) | | (8.47; 27.7) | |
| | Subject with | 18 | (75.0) | 18 | (75.0) | 13 | (54.2) | 12 | (50.0) | 22 | (81.5) | 17 | (65.4) | 17 | (65.4) | 14 | (53.8) |
| | titers <10 | | | | | | | | | | | | | | | | |
| | (1/dil): n (%) | | | | | | | | | | | | | | | | |
| | (95% CI) | (53.3; 90.2) | | (53.3; 90.2) | | (32.8; 74.4) | | (29.1; 70.9) | | (61.9; 93.7) | | (44.3; 82.8) | | (44.3; 82.8) | | (33.4; 73.4) | |
| | Subject with | 5 | (20.8) | 2 | (8.3) | 1 | (4.2) | 5 | (20.8) | 1 | (3.7) | 7 | (26.9) | 4 | (15.4) | 6 | (23.1) |
| | titers >=40 | | | | | | | | | | | | | | | | |
| | (1/dil): n (%) | | | | | | | | | | | | | | | | |
| | (95% CI) | (7.1; 42.2) | | (1.0; 27.0) | | (0.1; 21.1) | | (7.1; 42.2) | | (0.1; 19.0) | | (11.6; 47.8) | | (4.4; 34.9) | | (9.0; 43.6) | |
| Post- | M | 25 | | 25 | | 25 | | 25 | | 26 | | 25 | | 25 | | 25 | |
| vaccination | GMT | 303 | | 149 | | 167 | | 320 | | 248 | | 239 | | 186 | | 343 | |
| (D 28 or | (95% CI) | (157; 585) | | (88.0; 253) | | (92.6; 301) | | (207; 495) | | (135; 457) | | (117; 488) | | (96.2; 361) | | (171; 687) | |
| D 56 §) | Subject with | 0 | | 1 | (4.0) | 0 | | 0 | | 1 | (3.8) | 0 | | 2 | (8.0) | 0 | |
| | titers <10 | | | | | | | | | | | | | | | | |
| | (1/dil): n (%) | | | | | | | | | | | | | | | | |
| | (95% CI) | (0; 13.7) | | (0.1; 20.4) | | (0; 13.7) | | (0; 13.7) | | (0.1; 19.6) | | (0; 13.7) | | (1.0; 26.0) | | (0; 13.7) | |
| | Subject with | 23 | (92.0) | 23 | (92.0) | 22 | (88.0) | 25 | (100) | 22 | (84.6) | 23 | (92.0) | 21 | (84.0) | 22 | (88.0) |
| | titers >=40 | | | | | | | | | | | | | | | | |
| | (1/dil): n (%) | | | | | | | | | | | | | | | | |
| | (95% CI) | (74.0; 99.0) | | (74.0; 99.0) | | (68.8; 97.5) | | (86.3; 100) | | (65.1; 95.6) | | (74.0; 99.0) | | (63.9; 95.5) | | (68.8; 97.5) | |
| Post- | M | 24 | | 24 | | 24 | | 24 | | 26 | | 25 | | 25 | | 25 | |
| vaccination | GMTR | 30.6 | | 20.2 | | 16.5 | | 23.0 | | 35.1 | | 18.6 | | 18.6 | | 21.7 | |
| response | (95% CI) | (17.9; 52.6) | | (12.9; 31.6) | | (8.87; 30.6) | | (14.3; 36.8) | | (20.3; 60.8) | | (12.5; 27.8) | | (10.6; 32.9) | | (12.9; 36.4) | |
| based on | Seroconversion | 22 | (91.7) | 22 | (91.7) | 19 | (79.2) | 24 | (100) | 22 | (84.6) | 23 | (92.0) | 21 | (84.0) | 22 | (88.0) |
| pre- | rate: n (%) | | | | | | | | | | | | | | | | |
| vaccination | (95% CI) | (73.0; 99.0) | | (73.0; 99.0) | | (57.8; 92.9) | | (85.8; 100) | | (65.1; 95.6) | | (74.0; 99.0) | | (63.9; 95.5) | | (68.8; 97.5) | |
| (D 28 or | | | | | | | | | | | | | | | | | |
| D 56 §/D 0) | | | | | | | | | | | | | | | | | |
| | | US (6-<36 months) | | | | | | | | | | | | | | | |
| | | QIV-HD (60 μg) | | | | | | | | QIV-SD | | | | | | | |
| | | (N = 54) | | | | | | | | (N = 71) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 52 | | 52 | | 52 | | 52 | | 69 | | 69 | | 69 | | 69 | |
| vaccination | GMT | 12.3 | | 20.5 | | 8.24 | | 10.8 | | 14.6 | | 17.0 | | 9.00 | | 13.7 | |
| (D 0) | (95% CI) | (7.07; 21.4) | | (11.9; 35.6) | | (6.12; 11.1) | | (7.71; 15.0) | | (9.25; 23.0) | | (11.0; 26.5) | | (6.80; 11.9) | | (9.79; 19.2) | |
| | Subject with | 42 | (80.8) | 31 | (59.6) | 39 | (75.0) | 34 | (65.4) | 46 | (66.7) | 42 | (60.9) | 50 | (72.5) | 40 | (58.0) |
| | titers <10 | | | | | | | | | | | | | | | | |
| | (1/dil): n (%) | | | | | | | | | | | | | | | | |
| | (95% CI) | (67.5; 90.4) | | (45.1; 73.0) | | (61.1; 86.0) | | (50.9; 78.0) | | (54.3; 77.6) | | (48.4; 72.4) | | (60.4; 82.5) | | (45.5; 69.8) | |
| | Subject with | 10 | (19.2) | 17 | (32.7) | 5 | (9.6) | 9 | (17.3) | 13 | (18.8) | 20 | (29.0) | 10 | (14.5) | 14 | (20.3) |
| | titers >=40 | | | | | | | | | | | | | | | | |
| | (1/dil): n (%) | | | | | | | | | | | | | | | | |
| | (95% CI) | (9.6; 32.5) | | (20.3; 47.1) | | (3.2; 21.0) | | (8.2; 30.3) | | (10.4; 30.1) | | (18.7; 41.2) | | (7.2; 25.0) | | (11.6; 31.7) | |
| Post- | M | 47 | | 47 | | 47 | | 47 | | 68 | | 68 | | 68 | | 68 | |
| vaccination | GMT | 603 | | 505 | | 276 | | 556 | | 142 | | 161 | | 135 | | 290 | |
| (D 28 or | (95% CI) | (386; 943) | | (296; 864) | | (175; 437) | | (373; 831) | | (84.2; 241) | | (100; 259) | | (90.0; 203) | | (195; 432) | |
| D 56 §) | Subject with | 0 | | 1 | (2.1) | 0 | | 0 | | 7 | (10.3) | 3 | (4.4) | 3 | (4.4) | 2 | (2.9) |
| | titers <10 | | | | | | | | | | | | | | | | |
| | (1/dil): n (%) | | | | | | | | | | | | | | | | |
| | (95% CI) | (0; 7.5) | | (0.1; 11.3) | | (0; 7.5) | | (0; 7.5) | | (4.2; 20.1) | | (0.9; 12.4) | | (0.9; 12.4) | | (0.4; 10.2) | |
| | Subject with | 44 | (93.6) | 44 | (93.6) | 43 | (91.5) | 45 | (95.7) | 45 | (66.2) | 54 | (79.4) | 53 | (77.9) | 64 | (94.1) |
| | titers >=40 | | | | | | | | | | | | | | | | |
| | (1/dil): n (%) | | | | | | | | | | | | | | | | |
| | (95% CI) | (82.5; 98.7) | | (82.5; 98.7) | | (79.6; 97.6) | | (85.5; 99.5) | | (53.7; 77.2) | | (67.9; 88.3) | | (66.2; 87.1) | | (85.6; 98.4) | |
| Post- | M | 46 | | 46 | | 46 | | 46 | | 66 | | 66 | | 66 | | 66 | |
| vaccination | GMTR | 44.2 | | 22.8 | | 34.2 | | 50.3 | | 9.41 | | 9.72 | | 15.4 | | 21.1 | |
| response | (95% CI) | (26.8; 73.0) | | (15.4; 33.8) | | (23.5; 49.9) | | (34.1; 74.3) | | (5.94; 14.9) | | (7.04; 13.4) | | (10.8; 22.0) | | (15.0; 29.7) | |
| based on | Seroconversion | 39 | (84.8) | 40 | (87.0) | 42 | (91.3) | 44 | (95.7) | 37 | (56.1) | 47 | (71.2) | 50 | (75.8) | 61 | (92.4) |
| pre- | rate: n (%) | | | | | | | | | | | | | | | | |

| | | | | | | | | | |
|---|---|---|---|---|---|---|---|---|---|
| vaccination | (95% CI) | (71.1; 93.7) | (73.7; 95.1) | (79.2; 97.6) | (85.2; 99.5) | (43.3; 68.3) | (58.7; 81.7) | (63.6; 85.5) | (83.2; 97.5) |
| (D 28 or | | | | | | | | | |
| D 56 §/D 0) | | | | | | | | | |

n: number of subjects experiencing the endpoint listed in the first two columns
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
* D 28 or D 56: at 28 days after the last vaccination.
GMTR (Geometric means of titer ratio): geometric mean of the individual titer ratios (post-vaccination/pre-vaccination).
Seroconversion: subjects with a pre-vaccination HAI titer <10 (1/dil) and a post-vaccination titer >=40 (1/dil) or a pre-vaccination titer >=10 (1/dil) and a >=4-fold increase in post-vaccination.

TABLE 38
Comparison of QIV-HD to QIV-SD using GMTs by HAI method at 28 days after
the last vaccination (US subjects 6-less than 36 months) - IAS

| | | US (6-less than 36 months) | | | | | | | |
|---|---|---|---|---|---|---|---|---|---|
| | | | | | | | | QIV-HD/QIV-SD | |
| Investigational | Antigen/ | QIV-HD | | | QIV-SD | | | GMT | |
| dosage | strain | M | GMT | (95% CI) | M | GMT | (95% CI) | ratio | (95% CI) |
| QIV-HD | A/H1N1 | 25 | 303 | (157; 585) | 68 | 142 | (84.2; 241) | 2.13 | (0.83; 5.47) |
| (30 μg) | A/H3N2 | 25 | 149 | (88.0; 253) | 68 | 161 | (100; 259) | 0.93 | (0.40; 2.15) |
| | B/Victoria | 25 | 167 | (92.6; 301) | 68 | 135 | (90.0; 203) | 1.23 | (0.58; 2.62) |
| | B/Yamagata | 25 | 320 | (207; 495) | 68 | 290 | (195; 432) | 1.10 | (0.55; 2.22) |
| QIV-HD | A/H1N1 | 26 | 248 | (135; 457) | 68 | 142 | (84.2; 241) | 1.75 | (0.69; 4.38) |
| (45 μg) | A/H3N2 | 25 | 239 | (117; 488) | 68 | 161 | (100; 259) | 1.49 | (0.61; 3.60) |
| | B/Victoria | 25 | 186 | (96.2; 361) | 68 | 135 | (90.0; 203) | 1.38 | (0.64; 2.98) |
| | B/Yamagata | 25 | 343 | (171; 687) | 68 | 290 | (195; 432) | 1.18 | (0.55; 2.54) |
| QIV-HD | A/H1N1 | 47 | 603 | (386; 943) | 68 | 142 | (84.2; 241) | 4.24 | (2.05; 8.76) |
| (60 μg) | A/H3N2 | 47 | 505 | (296; 864) | 68 | 161 | (100; 259) | 3.14 | (1.53; 6.44) |
| | B/Victoria | 47 | 276 | (175; 437) | 68 | 135 | (90.0; 203) | 2.04 | (1.10; 3.77) |
| | B/Yamagata | 47 | 556 | (373; 831) | 68 | 290 | (195; 432) | 1.92 | (1.08; 3.41) |

M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.

TABLE 39
Comparison of QIV-HD at 28 days after each vaccination to QIV-SD at 28 days after the last vaccination
using GMTs by HAI method (US previously unvaccinated subjects 6-less than 36 months) - IAS

US (6-less than 36 months, previously unvaccinated)

QIV-HD†/QIV-SD‡

| Investigational | Antigen/ | QIV-HD† | | | QIV-SD‡ | | | GMT | |
|---|---|---|---|---|---|---|---|---|---|
| dosage | strain | M | GMT | (95% CI) | M | GMT | (95% CI) | ratio | (95% CI) |
| QIV-HD | A/H1N1 | 14 | 37.1 | (11.3; 122) | 48 | 107 | (57.4; 199) | 0.35 | (0.10; 1.26) |
| (30 μg) | A/H3N2 | 14 | 25.6 | (7.33; 89.6) | 48 | 122 | (69.2; 214) | 0.21 | (0.06; 0.71) |
| | B/Victoria | 14 | 20.5 | (5.49; 76.5) | 48 | 125 | (77.9; 201) | 0.16 | (0.06; 0.49) |
| | B/Yamagata | 14 | 35.3 | (11.2; 111) | 48 | 236 | (149; 376) | 0.15 | (0.05; 0.42) |
| QIV-HD | A/H1N1 | 12 | 53.4 | (16.3; 175) | 48 | 107 | (57.4; 199) | 0.50 | (0.13; 1.93) |
| (45 μg) | A/H3N2 | 12 | 101 | (20.3; 501) | 48 | 122 | (69.2; 214) | 0.83 | (0.22; 3.15) |
| | B/Victoria | 12 | 42.4 | (11.2; 161) | 48 | 125 | (77.9; 201) | 0.34 | (0.11; 1.04) |
| | B/Yamagata | 12 | 110 | (25.4; 476) | 48 | 236 | (149; 376) | 0.47 | (0.15; 1.44) |
| QIV-HD | A/H1N1 | 38 | 71.7 | (36.8; 140) | 48 | 107 | (57.4; 199) | 0.67 | (0.27; 1.66) |
| (60 μg) | A/H3N2 | 38 | 109 | (45.0; 264) | 48 | 122 | (69.2; 214) | 0.90 | (0.33; 2.42) |
| | B/Victoria | 38 | 38.2 | (19.8; 73.9) | 48 | 125 | (77.9; 201) | 0.31 | (0.14; 0.67) |
| | B/Yamagata | 38 | 73.0 | (35.3; 151) | 48 | 236 | (149; 376) | 0.31 | (0.14; 0.70) |
| | | US (6-less than 36 months, previously unvaccinated) | | | | | | | |
| | | | | | | | | QIY-HD‡/QIV-SD‡ | |
| Investigational | Antigen/ | QIV-HD‡ | | | QIV-SD‡ | | | GMT | |
| dosage | strain | M | GMT | (95% CI) | M | GMT | (95% CI) | ratio | (95% CI) |
| QIV-HD | A/H1N1 | 13 | 259 | (85.8; 779) | 48 | 107 | (57.4; 199) | 2.42 | (0.66; 8.89) |
| (30 μg) | A/H3N2 | 13 | 152 | (71.3; 323) | 48 | 122 | (69.2; 214) | 1.25 | (0.40; 3.90) |
| | B/Victoria | 13 | 116 | (40.4; 334) | 48 | 125 | (77.9; 201) | 0.93 | (0.33; 2.62) |
| | B/Yamagata | 13 | 280 | (133; 592) | 48 | 236 | (149; 376) | 1.19 | (0.46; 3.09) |
| QIV-HD | A/H1N1 | 15 | 351 | (173; 713) | 48 | 107 | (57.4; 199) | 3.29 | (1.02; 10.57) |
| (45 μg) | A/H3N2 | 14 | 244 | (90.0; 660) | 48 | 122 | (69.2; 214) | 2.00 | (0.63; 6.35) |
| | B/Victoria | 14 | 226 | (95.1; 538) | 48 | 125 | (77.9; 201) | 1.81 | (0.68; 4.80) |
| | B/Yamagata | 14 | 538 | (208; 1393) | 48 | 236 | (149; 376) | 2.28 | (0.86; 6.05) |
| QIV-HD | A/H1N1 | 33 | 477 | (291; 781) | 48 | 107 | (57.4; 199) | 4.47 | (1.92; 10.37) |
| (60 μg) | A/H3N2 | 33 | 420 | (214; 826) | 48 | 122 | (69.2; 214) | 3.46 | (1.45; 8.23) |
| | B/Victoria | 33 | 234 | (145; 375) | 48 | 125 | (77.9; 201) | 1.87 | (0.94; 3.70) |
| | B/Yamagata | 33 | 487 | (295; 804) | 48 | 236 | (149; 376) | 2.06 | (1.04; 4.09) |

M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
†28 days post-vaccination 1 (V03)
‡28 days post-vaccination 2 (V05)

TABLE 40
Comparison of QIV-HD to QIV-SD using GMTs by HAI method at 28 days after vaccination
(US previously vaccinated subjects 6-less than 36 months) - IAS

| | | US (6-less than 36 months, previously vaccinated) | | | | | | | |
|---|---|---|---|---|---|---|---|---|---|
| | | | | | | | | QIV-HD/QIV-SD | |
| Investigational | Antigen/ | QIV-HD | | | QIV-SD | | | GMT | |
| dosage | strain | M | GMT | (95% CI) | M | GMT | (95% CI) | ratio | (95% CI) |
| QIV-HD | A/H1N1 | 12 | 359 | (151; 855) | 20 | 283 | (104; 771) | 1.27 | (0.31; 5.19) |
| (30 μg) | A/H3N2 | 12 | 147 | (61.4; 351) | 20 | 315 | (130; 760) | 0.47 | (0.13; 1.68) |
| | B/Victoria | 12 | 247 | (140; 434) | 20 | 163 | (69.4; 382) | 1.52 | (0.48; 4.80) |
| | B/Yamagata | 12 | 370 | (215; 636) | 20 | 477 | (217; 1048) | 0.78 | (0.27; 2.26) |
| QIV-HD | A/H1N1 | 11 | 155 | (48.8; 493) | 20 | 283 | (104; 771) | 0.55 | (0.12; 2.55) |
| (45 μg) | A/H3N2 | 11 | 234 | (68.9; 792) | 20 | 315 | (130; 760) | 0.74 | (0.18; 3.10) |
| | B/Victoria | 11 | 146 | (44.6; 475) | 20 | 163 | (69.4; 382) | 0.89 | (0.22; 3.56) |
| | B/Yamagata | 11 | 193 | (65.1; 574) | 20 | 477 | (217; 1048) | 0.41 | (0.11; 1.45) |
| QIV-HD | A/H1N1 | 14 | 1050 | (389; 2831) | 20 | 283 | (104; 771) | 3.70 | (0.91; 15.08) |
| (60 μg) | A/H3N2 | 14 | 780 | (307; 1979) | 20 | 315 | (130; 760) | 2.48 | (0.70; 8.78) |
| | B/Victoria | 14 | 410 | (129; 1301) | 20 | 163 | (69.4; 382) | 2.52 | (0.66; 9.67) |
| | B/Yamagata | 14 | 761 | (373; 1554) | 20 | 477 | (217; 1048) | 1.60 | (0.54; 4.69) |

M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.

TABLE 41
Comparison of QIV-HD to QIV-SD using seroconversion rate by HAI method at 28
days after the last vaccination (US subjects 6-less than 36 months) - IAS

| | | US (6-less than 36 months) | | | | | | | |
|---|---|---|---|---|---|---|---|---|---|
| | | | | | | | | QIV-HD minus QIV-SD | |
| Investigational | Antigen/ | QIV-HD | | | QIV-SD | | | Difference | |
| dosage | strain | n/M | % | (95% CI) | n/M | % | (95% CI) | of % | (95% CI) |
| QIV-HD | A/H1N1 | 22/24 | 91.7 | (73.0; 99.0) | 37/66 | 56.1 | (43.3; 68.3) | 35.61 | (14.76; 49.01) |
| (30 μg) | A/H3N2 | 22/24 | 91.7 | (73.0; 99.0) | 47/66 | 71.2 | (58.7; 81.7) | 20.45 | (0.52; 33.74) |
| | B/Victoria | 19/24 | 79.2 | (57.8; 92.9) | 50/66 | 75.8 | (63.6; 85.5) | 3.41 | (−18.08; 19.78) |
| | B/Yamagata | 24/24 | 100.0 | (85.8; 100.0) | 61/66 | 92.4 | (83.2; 97.5) | 7.58 | (−6.88; 16.54) |
| QIV-HD | A/H1N1 | 22/26 | 84.6 | (65.1; 95.6) | 37/66 | 56.1 | (43.3; 68.3) | 28.55 | (7.17; 43.68) |
| (45 μg) | A/H3N2 | 23/25 | 92.0 | (74.0; 99.0) | 47/66 | 71.2 | (58.7; 81.7) | 20.79 | (1.34; 33.97) |
| | B/Victoria | 21/25 | 84.0 | (63.9; 95.5) | 50/66 | 75.8 | (63.6; 85.5) | 8.24 | (−12.35; 23.27) |
| | B/Yamagata | 22/25 | 88.0 | (68.8; 97.5) | 61/66 | 92.4 | (83.2; 97.5) | −4.42 | (−22.89; 7.48) |
| QIV-HD | A/H1N1 | 39/46 | 84.8 | (71.1; 93.7) | 37/66 | 56.1 | (43.3; 68.3) | 28.72 | (11.48; 42.93) |
| (60 μg) | A/H3N2 | 40/46 | 87.0 | (73.7; 95.1) | 47/66 | 71.2 | (58.7; 81.7) | 15.74 | (−0.06; 29.47) |
| | B/Victoria | 42/46 | 91.3 | (79.2; 97.6) | 50/66 | 75.8 | (63.6; 85.5) | 15.55 | (1.00; 28.25) |
| | B/Yamagata | 44/46 | 95.7 | (85.2; 99.5) | 61/66 | 92.4 | (83.2; 97.5) | 3.23 | (−7.83; 12.73) |

n: number of subjects experiencing seroconversion.
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
Seroconversion: subjects with a pre-vaccination HAI titer <10 (1/dil) and a post-vaccination titer >=40 (1/dil) or a pre-vaccination titer >=10 (1/dil) and a >=4-fold increase in post-vaccination.

TABLE 42
Comparison of QIV-HD to QIV-SD using seroconversion rate by HAI method at 28 days
after vaccination (US previously vaccinated subjects 6-less than 36 months) - IAS

| | | US (6-less than 36 months, previously vaccinated) | | | | | | | |
|---|---|---|---|---|---|---|---|---|---|
| | | | | | | | | QIV-HD minus QIV-SD | |
| Investigational | Antigen/ | QIV-HD | | | QIV-SD | | | Difference | |
| dosage | strain | n/M | % | (95% CI) | n/M | % | (95% CI) | of % | (95% CI) |
| QIV-HD | A/H1N1 | 11/11 | 100.0 | (71.5; 100.0) | 15/20 | 75.0 | (50.9; 91.3) | 25.00 | (−4.34; 46.87) |
| (30 μg) | A/H3N2 | 10/11 | 90.9 | (58.7; 99.8) | 14/20 | 70.0 | (45.7; 88.1) | 20.91 | (−11.64; 44.04) |
| | B/Victoria | 9/11 | 81.8 | (48.2; 97.7) | 15/20 | 75.0 | (50.9; 91.3) | 6.82 | (−25.77; 32.28) |
| | B/Yamagata | 11/11 | 100.0 | (71.5; 100.0) | 18/20 | 90.0 | (68.3; 98.8) | 10.00 | (−16.87; 30.10) |
| QIV-HD | A/H1N1 | 8/11 | 72.7 | (39.0; 94.0) | 15/20 | 75.0 | (50.9; 91.3) | −2.27 | (−34.66; 25.75) |
| (45 μg) | A/H3N2 | 10/11 | 90.9 | (58.7; 99.8) | 14/20 | 70.0 | (45.7; 88.1) | 20.91 | (−11.64; 44.04) |
| | B/Victoria | 9/11 | 81.8 | (48.2; 97.7) | 15/20 | 75.0 | (50.9; 91.3) | 6.82 | (−25.77; 32.28) |
| | B/Yamagata | 9/11 | 81.8 | (48.2; 97.7) | 18/20 | 90.0 | (68.3; 98.8) | −8.18 | (−38.57; 15.78) |
| QIV-HD | A/H1N1 | 10/13 | 76.9 | (46.2; 95.0) | 15/20 | 75.0 | (50.9; 91.3) | 1.92 | (−28.57; 28.38) |
| (60 μg) | A/H3N2 | 12/13 | 92.3 | (64.0; 99.8) | 14/20 | 70.0 | (45.7; 88.1) | 22.31 | (−7.61; 45.10) |
| | B/Victoria | 12/13 | 92.3 | (64.0; 99.8) | 15/20 | 75.0 | (50.9; 91.3) | 17.31 | (−11.80; 40.07) |
| | B/Yamagata | 13/13 | 100.0 | (75.3; 100.0) | 18/20 | 90.0 | (68.3; 98.8) | 10.00 | (−13.92; 30.10) |

n: number of subjects experiencing seroconversion.
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
Seroconversion: subjects with a pre-vaccination HAI titer <10 (1/dil) and a post-vaccination titer >=40 (1/dil) or a pre-vaccination titer >=10 (1/dil) and a >=4-fold increase in post-vaccination.)

TABLE 43
Comparison of QIV-HD at 28 days after each vaccination to QIV-SD at 28 days after the last vaccination using
seroconversion rate by HAI method (US previously unvaccinated subjects 6-less than 36 months) - IAS

| | | US (6-less than 36 months, previously unvaccinated) | | | | | | | |
|---|---|---|---|---|---|---|---|---|---|
| | | | | | | | | QIV-HD† minus QIV-SD‡ | |
| Investigational | Antigen/ | QIV-HD† | | | QIV-SD‡ | | | Difference | |
| dosage | strain | n/M | % | (95% CI) | n/M | % | (95% CI) | of % | (95% CI) |
| QIV-HD | A/H1N1 | 4/13 | 30.8 | (9.1; 61.4) | 22/46 | 47.8 | (32.9; 63.1) | −17.06 | (−39.95; 13.10) |
| (30 μg) | A/H3N2 | 3/13 | 23.1 | (5.0; 53.8) | 33/46 | 71.7 | (56.5; 84.0) | −48.66 | (−67.14; −17.96) |
| | B/Victoria | 4/13 | 30.8 | (9.1; 61.4) | 35/46 | 76.1 | (61.2; 87.4) | −45.32 | (−65.99; −15.02) |
| | B/Yamagata | 4/13 | 30.8 | (9.1; 61.4) | 43/46 | 93.5 | (82.1; 98.6) | −62.71 | (−81.30; −33.69) |
| QIV-HD | A/H1N1 | 8/12 | 66.7 | (34.9; 90.1) | 22/46 | 47.8 | (32.9; 63.1) | 18.84 | (−12.13; 42.69) |
| (45 μg) | A/H3N2 | 7/12 | 58.3 | (27.7; 84.8) | 33/46 | 71.7 | (56.5; 84.0) | −13.41 | (−41.97; 13.11) |
| | B/Victoria | 5/12 | 41.7 | (15.2; 72.3) | 35/46 | 76.1 | (61.2; 87.4) | −34.42 | (−58.90; −4.54) |
| | B/Yamagata | 8/12 | 66.7 | (34.9; 90.1) | 43/46 | 93.5 | (82.1; 98.6) | −26.81 | (−54.75; −4.41) |
| QIV-HD | A/H1N1 | 21/37 | 56.8 | (39.5; 72.9) | 22/46 | 47.8 | (32.9; 63.1) | 8.93 | (−12.24; 28.93) |
| (60 μg) | A/H3N2 | 20/37 | 54.1 | (36.9; 70.5) | 33/46 | 71.7 | (56.5; 84.0) | −17.69 | (−36.79; 2.96) |
| | B/Victoria | 17/37 | 45.9 | (29.5; 63.1) | 35/46 | 76.1 | (61.2; 87.4) | −30.14 | (−48.09; −9.11) |
| | B/Yamagata | 19/37 | 51.4 | (34.4; 68.1) | 43/46 | 93.5 | (82.1; 98.6) | −42.13 | (−58.16; −23.37) |

| US (6-less than 36 months, previously unvaccinated) | |
|---|---|
| | QIV-HD‡ minus QIV-SD‡ |

| Investigational | Antigen/ | QIV-HD‡ | | | QIV-SD‡ | | | Difference | |
|---|---|---|---|---|---|---|---|---|---|
| dosage | strain | n/M | % | (95% CI) | n/M | % | (95% CI) | of % | (95% CI) |
| QIV-HD | A/H1N1 | 11/13 | 84.6 | (54.6; 98.1) | 22/46 | 47.8 | (32.9; 63.1) | 36.79 | (6.49; 54.40) |
| (30 μg) | A/H3N2 | 12/13 | 92.3 | (64.0; 99.8) | 33/46 | 71.7 | (56.5; 84.0) | 20.57 | (−7.29; 36.19) |
| | B/Victoria | 10/13 | 76.9 | (46.2; 95.0) | 35/46 | 76.1 | (61.2; 87.4) | 0.84 | (−28.12; 21.29) |
| | B/Yamagata | 13/13 | 100.0 | (75.3; 100.0) | 43/46 | 93.5 | (82.1; 98.6) | 6.52 | (−16.69; 17.50) |

| US (6-less than 36 months, previously unvaccinated) | |
|---|---|
| | QIV-HD† minus QIV-SD‡ |

| Investigational | Antigen/ | QIV-HD† | | | QIV-SD‡ | | | Difference | |
|---|---|---|---|---|---|---|---|---|---|
| dosage | strain | n/M | % | (95% CI) | n/M | % | (95% CI) | of % | (95% CI) |
| QIV-HD | A/H1N1 | 14/15 | 93.3 | (68.1; 99.8) | 22/46 | 47.8 | (32.9; 63.1) | 45.51 | (18.43; 60.26) |
| (45 μg) | A/H3N2 | 13/14 | 92.9 | (66.1; 99.8) | 33/46 | 71.7 | (56.5; 84.0) | 21.12 | (−5.55; 36.56) |
| | B/Victoria | 12/14 | 85.7 | (57.2; 98.2) | 35/46 | 76.1 | (61.2; 87.4) | 9.63 | (−17.91; 27.01) |
| | B/Yamagata | 13/14 | 92.9 | (66.1; 99.8) | 43/46 | 93.5 | (82.1; 98.6) | −0.62 | (−25.32; 11.83) |
| QIV-HD | A/H1N1 | 29/33 | 87.9 | (71.8; 96.6) | 22/46 | 47.8 | (32.9; 63.1) | 40.05 | (19.36; 55.58) |
| (60 μg) | A/H3N2 | 28/33 | 84.8 | (68.1; 94.9) | 33/46 | 71.7 | (56.5; 84.0) | 13.11 | (−6.08; 29.73) |
| | B/Victoria | 30/33 | 90.9 | (75.7; 98.1) | 35/46 | 76.1 | (61.2; 87.4) | 14.82 | (−2.78; 30.05) |
| | B/Yamagata | 31/33 | 93.9 | (79.8; 99.3) | 43/46 | 93.5 | (82.1; 98.6) | 0.46 | (−13.74; 12.28) |

n: number of subjects experiencing seroconversion.
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
†28 days post-vaccination 1 (V03);
‡28 days post-vaccination 2 (V05)
Seroconversion: subjects with a pre-vaccination HAI titer <10 (1/dil) and a post-vaccination titer >=40 (1/dil) or a pre-vaccination titer >=10 (1/dil) and a >=4-fold increase in post-vaccination.

TABLE 44
Summary of SN antibody response for each antigen (US subjects 6-less than 36 months) - IAS

| | | US (6-less than 36 months) | | | | | | | | | | | | | | | |
|---|---|---|---|---|---|---|---|---|---|---|---|---|---|---|---|---|---|
| | | QIV-HD (30 μg) | | | | | | | | QIV-HD (45 μg) | | | | | | | |
| | | (N = 26) | | | | | | | | (N = 27) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 23 | | 22 | | 22 | | 22 | | 25 | | 25 | | 25 | | 25 | |
| vaccination | GMT | 34.5 | | 39.4 | | 8.36 | | 27.2 | | 14.8 | | 67.8 | | 10.9 | | 30.4 | |
| (D 0) | (95% CI) | (13.5; 87.8) | | (27.0; 57.5) | | (5.63; 12.4) | | (15.4; 47.9) | | (7.48; 29.4) | | (43.1; 107) | | (6.65; 18.0) | | (17.1; 54.2) | |
| | Subject with | 9 | (39.1) | 0 | | 15 | (68.2) | 4 | (18.2) | 15 | (60.0) | 0 | | 15 | (60.0) | 5 | (20.0) |
| | titers <10 | | | | | | | | | | | | | | | | |
| | (1/dil): n (%) | | | | | | | | | | | | | | | | |
| | (95% CI) | (19.7; 61.5) | | (0; 15.4) | | (45.1; 86.1) | | (5.2; 40.3) | | (38.7; 78.9) | | (0; 13.7) | | (38.7; 78.9) | | (6.8; 40.7) | |
| | Subject with | 8 | (34.8) | 9 | (40.9) | 1 | (4.5) | 8 | (36.4) | 6 | (24.0) | 16 | (64.0) | 4 | (16.0) | 9 | (36.0) |
| | titers >=40 | | | | | | | | | | | | | | | | |
| | (1/dil): n (%) | | | | | | | | | | | | | | | | |
| | (95% CI) | (16.4; 57.3) | | (20.7; 63.6) | | (0.1; 22.8) | | (17.2; 59.3) | | (9.4; 45.1) | | (42.5; 82.0) | | (4.5; 36.1) | | (18.0; 57.5) | |
| Post- | M | 22 | | 23 | | 23 | | 22 | | 25 | | 24 | | 25 | | 25 | |
| vaccination | GMT | 2700 | | 237 | | 208 | | 354 | | 2000 | | 360 | | 237 | | 480 | |
| (D 28 or | (95% CI) | (1262; 5775) | | (153; 365) | | (113; 381) | | (216; 580) | | (958; 4173) | | (171; 755) | | (111; 507) | | (231; 999) | |
| D 56 *) | Subject with | 0 | | 0 | | 0 | | 0 | | 0 | | 0 | | 2 | (8.0) | 0 | |
| | titers <10 | | | | | | | | | | | | | | | | |
| | (1/dil): n (%) | | | | | | | | | | | | | | | | |
| | (95% CI) | (0; 15.4) | | (0; 14.8) | | (0; 14.8) | | (0; 15.4) | | (0; 13.7) | | (0; 14.2) | | (1.0; 26.0) | | (0; 13.7) | |
| | Subject with | 22 | (100) | 22 | (95.7) | 20 | (87.0) | 22 | (100) | 25 | (100) | 24 | (100) | 20 | (80.0) | 22 | (88.0) |
| | titers >=40 | | | | | | | | | | | | | | | | |
| | (1/dil): n (%) | | | | | | | | | | | | | | | | |
| | (95% CI) | (84.6; 100) | | (78.1; 99.9) | | (66.4; 97.2) | | (84.6; 100) | | (86.3; 100) | | (85.8; 100) | | (59.3; 93.2) | | (68.8; 97.5) | |
| Post- | M | 20 | | 20 | | 20 | | 19 | | 23 | | 22 | | 23 | | 23 | |
| vaccination | GMTR | 79.9 | | 5.56 | | 20.2 | | 14.6 | | 165 | | 5.85 | | 22.4 | | 18.0 | |
| response | (95% CI) | (31.1; 205) | | (3.77; 8.21) | | (11.4; 35.8) | | (8.73; 24.4) | | (73.8; 368) | | (3.60; 9.51) | | (11.6; 43.4) | | (10.3; 31.7) | |
| based on | Subjects with | 19 | (95.0) | 18 | (90.0) | 20 | (100) | 19 | (100) | 23 | (100) | 19 | (86.4) | 21 | (91.3) | 22 | (95.7) |
| pre- | 2-Fold | | | | | | | | | | | | | | | | |
| vaccination | rise^{†}: n (%) | | | | | | | | | | | | | | | | |
| (D 28 or | (95% CI) | (75.1; 99.9) | | (68.3; 98.8) | | (83.2; 100) | | (82.4; 100) | | (85.2; 100) | | (65.1; 97.1) | | (72.0; 98.9) | | (78.1; 99.9) | |
| D 56 */D 0) | Subjects with | 19 | (95.0) | 13 | (65.0) | 18 | (90.0) | 17 | (89.5) | 23 | (100) | 13 | (59.1) | 19 | (82.6) | 22 | (95.7) |
| | 4-Fold | | | | | | | | | | | | | | | | |
| | rise^{†}: n (%) | | | | | | | | | | | | | | | | |
| | (95% CI) | (75.1; 99.9) | | (40.8; 84.6) | | (68.3; 98.8) | | (66.9; 98.7) | | (85.2; 100) | | (36.4; 79.3) | | (61.2; 95.0) | | (78.1; 99.9) | |
| | | US (6-less than 36 months) | | | | | | | | | | | | | | | |
| | | QIV-HD (60 μg) | | | | | | | | QIV-SD | | | | | | | |
| | | (N = 54) | | | | | | | | (N = 71) | | | | | | | |
| | | A/H1N1 | | A/H3N2 | | B/Victoria | | B/Yamagata | | A/H1N1 | | A/H3N2 | | B/Victoria | | B/Yamagata | |
| Pre- | M | 48 | | 48 | | 48 | | 48 | | 65 | | 64 | | 64 | | 64 | |
| vaccination | GMT | 23.8 | | 75.2 | | 9.61 | | 28.1 | | 29.8 | | 71.9 | | 10.7 | | 30.1 | |
| (D 0) | (95% CI) | (10.6; 53.5) | | (49.6; 114) | | (6.71; 13.8) | | (20.5; 38.7) | | (15.7; 56.5) | | (52.4; 98.7) | | (7.66; 14.9) | | (22.1; 41.1) | |
| | Subject with | 31 | (64.6) | 1 | (2.1) | 34 | (70.8) | 4 | (8.3) | 39 | (60.0) | 2 | (3.1) | 41 | (64.1) | 8 | (12.5) |
| | titers <10 | | | | | | | | | | | | | | | | |
| | (1/dil): n (%) | | | | | | | | | | | | | | | | |
| | (95% CI) | (49.5; 77.8) | | (0.1; 11.1) | | (55.9; 83.0) | | (2.3; 20.0) | | (47.1; 72.0) | | (0.4; 10.8) | | (51.1; 75.7) | | (5.6; 23.2) | |
| | Subject with | 11 | (22.9) | 29 | (60.4) | 6 | (12.5) | 12 | (25.0) | 22 | (33.8) | 41 | (64.1) | 8 | (12.5) | 20 | (31.3) |
| | titers >=40 | | | | | | | | | | | | | | | | |
| | (1/dil): n (%) | | | | | | | | | | | | | | | | |
| | (95% CI) | (12.0; 37.3) | | (45.3; 74.2) | | (4.7; 25.2) | | (13.6; 39.6) | | (22.6; 46.6) | | (51.1; 75.7) | | (5.6; 23.2) | | (20.2; 44.1) | |
| Post- | M | 47 | | 44 | | 47 | | 47 | | 65 | | 64 | | 65 | | 64 | |
| vaccination | GMT | 4535 | | 623 | | 332 | | 660 | | 663 | | 271 | | 126 | | 340 | |
| (D 28 or | (95% CI) | (2665; 7716) | | (396; 981) | | (197; 560) | | (426; 1022) | | (336; 1308) | | (182; 401) | | (80.9; 195) | | (223; 516) | |
| D 56 *) | Subject with | 0 | | 0 | | 0 | | 0 | | 4 | (6.2) | 0 | | 7 | (10.8) | 1 | (1.6) |
| | titers <10 | | | | | | | | | | | | | | | | |
| | (1/dil): n (%) | | | | | | | | | | | | | | | | |
| | (95% CI) | (0; 7.5) | | (0; 8.0) | | (0; 7.5) | | (0; 7.5) | | (1.7; 15.0) | | (0; 5.6) | | (4.4; 20.9) | | (0; 8.4) | |
| | Subject with | 47 | (100) | 44 | (100) | 41 | (87.2) | 46 | (97.9) | 51 | (78.5) | 59 | (92.2) | 50 | (76.9) | 59 | (92.2) |
| | titers >=40 | | | | | | | | | | | | | | | | |
| | (1/dil): n (%) | | | | | | | | | | | | | | | | |
| | (95% CI) | (92.5; 100) | | (92.0; 100) | | (74.3; 95.2) | | (88.7; 99.9) | | (66.5; 87.7) | | (82.7; 97.4) | | (64.8; 86.5) | | (82.7; 97.4) | |
| Post- | M | 43 | | 41 | | 43 | | 43 | | 60 | | 59 | | 59 | | 59 | |
| vaccination | GMTR | 170 | | 7.13 | | 35.8 | | 22.7 | | 19.8 | | 4.04 | | 14.5 | | 11.6 | |
| response | (95% CI) | (84.6; 340) | | (4.90; 10.4) | | (22.1; 58.1) | | (14.7; 35.0) | | (11.2; 35.1) | | (3.03; 5.39) | | (9.94; 21.2) | | (8.16; 16.4) | |
| based on | Subjects with | 41 | (95.3) | 36 | (87.8) | 42 | (97.7) | 42 | (97.7) | 53 | (88.3) | 42 | (71.2) | 53 | (89.8) | 55 | (93.2) |
| pre- | 2-Fold | | | | | | | | | | | | | | | | |
| vaccination | rise^{†}: n (%) | | | | | | | | | | | | | | | | |
| (D 28 or | (95% CI) | (84.2; 99.4) | | (73.8; 95.9) | | (87.7; 99.9) | | (87.7; 99.9) | | (77.4; 95.2) | | (57.9; 82.2) | | (79.2; 96.2) | | (83.5; 98.1) | |
| D 56 */D 0) | Subjects with | 41 | (95.3) | 27 | (65.9) | 40 | (93.0) | 39 | (90.7) | 46 | (76.7) | 30 | (50.8) | 45 | (76.3) | 44 | (74.6) |
| | 4-Fold | | | | | | | | | | | | | | | | |
| | rise^{†}: n (%) | | | | | | | | | | | | | | | | |

| | | | | | | | | |
|---|---|---|---|---|---|---|---|---|
| (95% CI) | (84.2; 99.4) | (49.4; 79.9) | (80.9; 98.5) | (77.9; 97.4) | (64.0; 86.6) | (37.5; 64.1) | (63.4; 86.4) | (61.6; 85.0) |

n: number of subjects experiencing the endpoint listed in the first two columns
M: number of subjects with available data for the considered endpoint
Strains contained in QIV-HD are tested. A/H1N1 = A/Michigan/45/2015 X-275; A/H3N2 = A/Singapore/INFIMH-16-0019/2016 IVR-186; B/Victoria = B/Maryland/15/2016 BX-69A; B/Yamagata = B/Phuket/3073/2013.
* D 28 or D 56: at 28 days after the last vaccination.
^{†}X-Fold rise: the computed value (=post-vaccination computed value/pre-vaccination computed value) >=X.
GMTR (Geometric means of titer ratio): geometric mean of the individual titer ratios (post-vaccination/pre-vaccination).

2.4.5—Overall Immunogenicity Conclusions

Executive Summary:

This study showed that QIV-HD containing 60 μg HA/strain provides improved immunogenicity without negatively impacting the benefit-risk balance of this vaccine in children and adolescents. As expected, the overall reactogenicity was greater for QIV-HD than for QIV-SD. The largest effects in terms of immunogenicity occurred in the youngest children (6 months through 3 years of age). Immunogenicity was also higher after the second vaccination with QIV-HD confirming that two doses improve the immune response in previously unvaccinated children.

This study indicated that an antigen dose of 60 μg, especially in the youngest children, offers improved immunogenicity against all 4 influenza strains. Despite a total antigen of 240 μg, safety and reactogenicity appear similar to QIV-SD, even in the youngest children.

Details:

In US subjects 6 months to less than 18 years of age who received QIV-HD 30 μg or QIV-HD 45 μg compared to subjects who received QIV-SD, the GMT ratios were slightly higher for A/H3N2. On the other hand, the QIV-HD 60 μg group trended towards higher GMT ratios for all strains. This trend was most pronounced in subjects 6 to less than 36 months of age (Table 38) who received QIV-HD 60 μg, where the GMT ratios ranged from 1.92 to 4.24 for each strain, when compared to QIV-SD. The GMTRs (geometric mean of individual titer ratios of post-vaccination/pre-vaccination) for both the HAI and the SN assays also showed a similar trend with the 6 to less than 36 months old subjects receiving the QIV-HD 60 μg dose formulation generating the highest immune response. Seroconversion rates for the QIV-HD 30 μg, 45 μg, and 60 μg groups were also higher than the seroconversion rates for the QIV-SD group, but there was not a clear trend towards increasing seroconversion rates with increasing QIV-HD dose formulations. However, seroconversion rates are more variable and considered as less informative than GMTs in terms of making a dose selection decision. Lastly, hemagglutination inhibition (HAI) antibody titers may be utilized as a correlate of protection for inactivated influenza vaccines in children. In particular, Black et al. describes a 1:629 cutoff in HAI titers as prediction for 90% protection rates (Black et al. (2011) Pediatr Infect Dis J 30(12):1081-5). As shown in Table 45, the QIV-HD 60 μg group trended towards higher number of subjects with HAI titers of ≥1:629.

TABLE 45

| Strain | Vaccine Group | All | 9-17 yr | 5-8 yr | 3-<5 yr | 6 m-<3 yr | 6 m-<2 yr |
|---|---|---|---|---|---|---|---|
| H1N1 | QIV-HD 30 μg | 59.48% | 89.66% | 50.00% | 56.25% | 40.00% | 42.86% |
| | QIV-HD 45 μg | 62.28% | 79.31% | 64.52% | 64.29% | 38.46% | 11.11% |
| | QIV-HD 60 μg | 71.03% | 96.67% | 71.43% | 67.50% | 57.45% | 62.50% |
| | QIV-SD | 66.97% | 85.71% | 82.61% | 80.00% | 32.35% | 19.35% |
| H3N2 | QIV-HD 30 μg | 57.76% | 68.97% | 86.67% | 50.00% | 20.00% | 14.29% |
| | QIV-HD 45 μg | 69.03% | 89.66% | 77.42% | 71.43% | 32.00% | 0.00% |
| | QIV-HD 60 μg | 67.36% | 83.33% | 92.59% | 62.50% | 46.81% | 45.83% |
| | QIV-SD | 45.45% | 57.14% | 48.89% | 47.69% | 33.82% | 16.13% |
| B/Victoria | QIV-HD 30 μg | 40.52% | 55.17% | 40.00% | 37.50% | 28.00% | 21.43% |
| | QIV-HD 45 μg | 46.90% | 65.52% | 51.61% | 39.29% | 28.00% | 22.22% |
| | QIV-HD 60 μg | 53.79% | 76.67% | 60.71% | 47.50% | 40.43% | 37.50% |
| | QIV-SD | 39.37% | 57.14% | 43.48% | 38.46% | 26.47% | 19.35% |
| B/Yamagata | QIV-HD 30 μg | 66.38% | 96.55% | 73.33% | 56.25% | 36.00% | 35.71% |
| | QIV-HD 45 μg | 63.39% | 82.76% | 74.19% | 55.56% | 36.00% | 33.33% |
| | QIV-HD 60 μg | 72.92% | 93.10% | 75.00% | 77.50% | 55.32% | 54.17% |
| | QIV-SD | 57.73% | 88.10% | 71.11% | 52.31% | 35.29% | 25.81% |

### Example 3—Safety of QIV-HD in Children 6 Months Through 35 Months of Age

### 3.1. General Study Design

A sentinel safety cohort of 100 US subjects were enrolled in an uncontrolled, open-label design without a comparator vaccine to evaluate the safety of QIV-HD.

Vaccination

For the Sentinel Safety Cohort, eligible subjects received QIV-HD as follows:
Subjects who were previously vaccinated against influenza received 1 dose of the QIV-HD on Day (D) 0, with no comparator vaccine.
Subjects who were not previously vaccinated against influenza received 2 doses of the QIV-HD with no comparator vaccine. Each dose was administered 28 days apart (at D0 and D28).

Note: Previously unvaccinated subjects are defined as subjects who have not received at least 2 doses of seasonal influenza vaccine in a prior influenza season. These subjects received 2 doses of study vaccine at least 28 days apart after enrolling in the study. Subjects who have received only one dose of any influenza vaccine in the past or subjects whose vaccination history is unknown also are considered as previously unvaccinated when enrolling and receive 2 doses of study vaccine at least 28 days apart. Previously vaccinated subjects are defined as subjects who have received at least 2 doses of seasonal influenza vaccine in prior influenza seasons. These subjects received only 1 dose of study vaccine after enrolling in the study.

Collection of Safety Data

All subjects were observed for 30 minutes after vaccination, and any unsolicited systemic adverse events (AEs) occurring during that time were recorded as immediate unsolicited systemic AEs.

Solicited reactions were collected through day 7 after each vaccination, and unsolicited AEs were collected through day 28 after each vaccination in all subjects.

### 3.2. Results

Tables 46 and 47 provide disposition and a demographic summary of sentinel subjects.

TABLE 46
Disposition of sentinel subjects- Enrolled Subjects

| | | QIV-HD | |
|---|---|---|---|
| | | (N = 100) | |
| Timepoint | | n (%) | |
| V01 (D 0) | Planned | 100 | |
| | Enrolled | 100 | (100) |
| | Randomized | 0 | |
| | Vaccinated | 100 | (100) |
| V02 (D 28) | Present at visit | 100 | (100) |
| | Vaccinated | 41 | (41.0) |
| V03 (D 56) | Present at visit | 41 | (41.0) |

| | | |
|---|---|---|
| Early termination | Discontinued | 0 |
| | Adverse event | 0 |
| | Lost to follow-up | 0 |
| | Protocol deviation | 0 |
| | Withdrawal by subject | 0 |

n: number of subjects fulfilling the items listed in the first two columns

TABLE 47
Summary of sentinel subject demographics

| | QIV-HD | | QIV-HD | | | |
|---|---|---|---|---|---|---|
| | Previously | | Previously | | QIV-HD | |
| | Unvaccinated | | vaccinated | | Overall | |
| Characteristic | (N = 41) | | (N = 59) | | (N = 100) | |
| Sex, n (%) | | | | | | |
| Male | 17 | (41.5) | 29 | (49.2) | 46 | (46.0) |
| Female | 24 | (58.5) | 30 | (50.8) | 54 | (54.0) |
| Sex ratio: | | | | | | |
| Male/Female | 0.71 | | 0.97 | | 0.85 | |
| Age (years) | | | | | | |
| Mean (SD) | 1.5 | (0.52) | 1.9 | (0.23) | 1.7 | (0.43) |
| Min; Max | 0.6; 2.0 | | 1.1; 2.0 | | 0.6; 2.0 | |
| Median | 1.7 | | 2.0 | | 1.9 | |
| Q1; Q3 | 0.9; 2.0 | | 1.8; 2.0 | | 1.5; 2.0 | |
| Age group: n (%) | | | | | | |
| <24 months | 30 | (73.2) | 21 | (35.6) | 51 | (51.0) |
| 6-11 months | 12 | (29.3) | 0 | | 12 | (12.0) |
| 12-23 months | 18 | (43.9) | 21 | (35.6) | 39 | (39.0) |
| >=24 months | 11 | (26.8) | 38 | (64.4) | 49 | (49.0) |
| Racial origin: n (%) | | | | | | |
| American Indian or | 1 | (2.4) | 0 | | 1 | (1.0) |
| Alaskan Native | | | | | | |
| Asian | 0 | | 0 | | 0 | |
| Black or African | 8 | (19.5) | 12 | (20.3) | 20 | (20.0) |
| American | | | | | | |
| Native Hawaiian or | 0 | | 0 | | 0 | |
| Other Pacific | | | | | | |
| Islander | | | | | | |
| White | 27 | (65.9) | 44 | (74.6) | 71 | (71.0) |
| Mixed Origin | 3 | (7.3) | 3 | (5.1) | 6 | (6.0) |
| Not Reported | 2 | (4.9) | 0 | | 2 | (2.0) |
| Other | 0 | | 0 | | 0 | |
| Ethnicity, n (%) | | | | | | |
| Hispanic or Latino | 12 | (29.3) | 10 | (16.9) | 22 | (22.0) |
| Not Hispanic or | 29 | (70.7) | 49 | (83.1) | 78 | (78.0) |
| Latino | | | | | | |

| | | | |
|---|---|---|---|
| Not reported | 0 | 0 | 0 |
| Unknown | 0 | 0 | 0 |

n: number of subjects fulfilling the items listed in the first two columns
Q1; Q3: first quartile; third quartile

Tables 48A, 48B, and 48C provide safety overview after any vaccination, vaccination 1, and vaccination 2, respectively: immediate AE information (within 30 minutes after vaccination), solicited injection site and systemic reactions (up to 7 days after vaccination), unsolicited AEs/ARs and AE leading to study discontinuation (up to 28 days after vaccination), and SAEs including AESIs (throughout the study). n is the number of subjects experiencing the endpoint listed in the first column. M is the number of subjects with available data for the relevant endpoint. Percentages are based on M.

TABLE 48A
Safety overview after any vaccinations in sentinel subjects

QIV-HD
(N = 100)

| Subjects experiencing at least one: | n/M | % |
|---|---|---|
| Within 28 days after any vaccination | | |
| Immediate unsolicited AE | 0/100 | 0 |
| Immediate unsolicited AR | 0/100 | 0 |
| Solicited reaction | 65/100 | 65.0 |
| Solicited injection site reaction | 51/100 | 51.0 |
| Solicited systemic reaction | 46/100 | 46.0 |
| Unsolicited AE | 25/100 | 25.0 |
| Unsolicited AR | 2/100 | 2.0 |
| AE leading to study discontinuation | 0/100 | 0 |
| SAE | 1/100 | 1.0 |
| Death | 0/100 | 0 |
| AESI | 0/100 | 0 |
| During the reported period | | |
| SAE | 1/100 | 1.0 |
| Death | 0/100 | 0 |
| AESI | 0/100 | 0 |

TABLE 48B
Safety overview after vaccination 1 in sentinel subjects

QIV-HD
(N = 100)

| Subjects experiencing at least one: | n/M | % |
|---|---|---|
| Within 28 days after vaccination 1 | | |
| Immediate unsolicited AE | 0/100 | 0 |
| Immediate unsolicited AR | 0/100 | 0 |
| Solicited reaction | 61/100 | 61.0 |
| Solicited injection site reaction | 46/100 | 46.0 |
| Solicited systemic reaction | 45/100 | 45.0 |
| Unsolicited AE | 20/100 | 20.0 |
| Unsolicited AR | 1/100 | 1.0 |
| AE leading to study discontinuation | 0/100 | 0 |
| SAE | 1/100 | 1.0 |
| Death | 0/100 | 0 |
| AESI | 0/100 | 0 |
| During the reported period | | |
| SAE | 1/100 | 1.0 |
| Death | 0/100 | 0 |
| AESI | 0/100 | 0 |

TABLE 48C
Safety overview after vaccination 2 in sentinel subjects

QIV-HD
(N = 41)

| Subjects experiencing at least one: | n/M | % |
|---|---|---|
| Within 28 days after vaccination 2 | | |
| Immediate unsolicited AE | 0/41 | 0 |
| Immediate unsolicited AR | 0/41 | 0 |
| Solicited reaction | 24/41 | 58.5 |
| Solicited injection site reaction | 17/41 | 41.5 |
| Solicited systemic reaction | 17/41 | 41.5 |
| Unsolicited AE | 7/41 | 17.1 |
| Unsolicited AR | 1/41 | 2.4 |
| AE leading to study discontinuation | 0/41 | 0 |
| SAE | 0/41 | 0 |
| Death | 0/41 | 0 |
| AESI | 0/41 | 0 |
| During the reported period | | |
| SAE | 0/41 | 0 |
| Death | 0/41 | 0 |
| AESI | 0/41 | 0 |

Table 49 provides a summary of vaccination-related SAE and grade 3 fever after vaccination.

TABLE 49
Summary of vaccination-related SAE and grade 3
fever after vaccination 1 in sentinel subjects

QIV-HD
(N = 100)

| Subjects experiencing: | n/M | % |
|---|---|---|
| Vaccination-related SAE (including AESIs) | 0/100 | 0 |
| Grade 3 fever | 0/96 | 0 |

SAE: serious adverse event;
AESI: adverse events of special interest
n: number of subjects fulfilling the item listed in the first column
M: For SAE M is the number of subjects in the expanded safety analysis set and for fever M is the number of vaccinated subjects with at least one available record for fever
Percentages are based on M

Table 50A, 50B, and 50C provide a summary of solicited reactions within 7 days after any vaccination, vaccination 1, and vaccination 2, respectively. n is the number of subjects experiencing the endpoint listed in the first column. M is the number of subjects with available data for the relevant endpoint. Percentages are based on M.

TABLE 50A
Summary of solicited reactions within 7 days
after any vaccinations in sentinel subjects

QIV-HD
(N = 100)

| Subjects experiencing at least one: | n/M | % |
|---|---|---|
| Solicited reaction | 65/100 | 65.0 |
| Grade 3 solicited reaction | 10/100 | 10.0 |
| Solicited injection site reaction | 51/100 | 51.0 |
| Grade 3 solicited injection site reaction | 5/100 | 5.0 |
| Solicited systemic reaction | 46/100 | 46.0 |
| Grade 3 solicited systemic reaction | 6/100 | 6.0 |

TABLE 50B
Summary of solicited reactions within 7 days
after vaccination 1 in sentinel subjects

QIV-HD
(N = 100)

| Subjects experiencing at least one: | n/M | % |
|---|---|---|
| Solicited reaction | 65/100 | 65.0 |
| Grade 3 solicited reaction | 5/100 | 5.0 |
| Solicited injection site reaction | 46/100 | 46.0 |
| Grade 3 solicited injection site reaction | 3/100 | 3.0 |
| Solicited systemic reaction | 45/100 | 45.0 |
| Grade 3 solicited systemic reaction | 2/100 | 2.0 |

TABLE 50C
Summary of solicited reactions within 7 days
after vaccination 2 in sentinel subjects

QIV-HD
(N = 41)

| Subjects experiencing at least one: | n/M | % |
|---|---|---|
| Solicited reaction | 24/41 | 58.5 |
| Grade 3 solicited reaction | 5/41 | 12.2 |
| Solicited injection site reaction | 17/41 | 41.5 |
| Grade 3 solicited injection site reaction | 2/41 | 4.9 |
| Solicited systemic reaction | 17/41 | 41.5 |
| Grade 3 solicited systemic reaction | 4/41 | 9.8 |

Table 51A, 51B, and 51C summarizes unsolicited AEs and ARs within 28 days after any vaccination, vaccination 1, and vaccination 2, respectively. n is the number of subjects experiencing the endpoint listed in the first column. n AEs are the number of AEs.

TABLE 51A
Summary of unsolicited AEs and ARs within 28 days
after any vaccinations in sentinel subjects

| | QIV-HD | | |
|---|---|---|---|
| | (N = 100) | | |
| Subjects experiencing at least one: | n | % | n AEs |
| Immediate unsolicited AE | 0 | 0 | 0 |
| Grade 3 immediate unsolicited AE | 0 | 0 | 0 |
| Immediate unsolicited AR | 0 | 0 | 0 |
| Grade 3 immediate unsolicited AR | 0 | 0 | 0 |
| Unsolicited AE | 25 | 25.0 | 44 |
| Grade 3 unsolicited AE | 1 | 1.0 | 1 |
| Unsolicited AR | 2 | 2.0 | 2 |
| Grade 3 unsolicited AR | 0 | 0 | 0 |
| Unsolicited injection site AR | 0 | 0 | 0 |
| Grade 3 unsolicited injection site AR | 0 | 0 | 0 |
| SAE | 1 | 1.0 | 1 |
| Grade 3 SAE | 0 | 0 | 0 |

TABLE 51B
Summary of unsolicited AEs and ARs within 28
days after vaccination 1 in sentinel subjects

| | QIV-HD | | |
|---|---|---|---|
| | (N = 100) | | |
| Subjects experiencing at least one: | n | % | n AEs |
| Immediate unsolicited AE | 0 | 0 | 0 |
| Grade 3 immediate unsolicited AE | 0 | 0 | 0 |
| Immediate unsolicited AR | 0 | 0 | 0 |
| Grade 3 immediate unsolicited AR | 0 | 0 | 0 |
| Unsolicited AE | 20 | 20.0 | 28 |
| Grade 3 unsolicited AE | 0 | 0 | 0 |
| Unsolicited AR | 1 | 1.0 | 1 |
| Grade 3 unsolicited AR | 0 | 0 | 0 |
| Unsolicited injection site AR | 0 | 0 | 0 |
| Grade 3 unsolicited injection site AR | 0 | 0 | 0 |
| SAE | 1 | 1.0 | 1 |
| Grade 3 SAE | 0 | 0 | 0 |

TABLE 51C
Summary of unsolicited AEs and ARs within 28
days after vaccination 2 in sentinel subjects

| | QIV-HD | | |
|---|---|---|---|
| | (N = 100) | | |
| Subjects experiencing at least one: | n | % | n AEs |
| Immediate unsolicited AE | 0 | 0 | 0 |
| Grade 3 immediate unsolicited AE | 0 | 0 | 0 |
| Immediate unsolicited AR | 0 | 0 | 0 |
| Grade 3 immediate unsolicited AR | 0 | 0 | 0 |
| Unsolicited AE | 7 | 17.1 | 16 |
| Grade 3 unsolicited AE | 1 | 2.4 | 1 |
| Unsolicited AR | 1 | 2.4 | 1 |
| Grade 3 unsolicited AR | 0 | 0 | 0 |
| Unsolicited injection site AR | 0 | 0 | 0 |
| Grade 3 unsolicited injection site AR | 0 | 0 | 0 |
| SAE | 0 | 0 | 0 |
| Grade 3 SAE | 0 | 0 | 0 |

Table 52 summarizes unsolicited AEs and ARs within 28 days after any vaccinations in sentinel subjects by age group. n is the number of subjects experiencing the endpoint listed in the first column. M is the number of subjects with available data for the relevant endpoint. Percentages are based on M. n AEs are the number of AEs.

TABLE 52
Summary of unsolicited AEs and Ars within 28 days after
any vaccinations in sentinel subjects by age group

| | | QIV-HD | | |
|---|---|---|---|---|
| Age | | (N = 100) | | |
| group | Subjects experiencing at least one: | n/M | % | n AEs |
| 6-11 | Immediate unsolicited AE | 0/12 | 0 | 0 |
| months | Grade 3 immediate unsolicited AE | 0/12 | 0 | 0 |
| | Immediate unsolicited AR | 0/12 | 0 | 0 |
| | Grade 3 immediate unsolicited AR | 0/12 | 0 | 0 |
| | Unsolicited AE | 6/12 | 50.0 | 11 |
| | Grade 3 unsolicited AE | 0/12 | 0 | 0 |
| | Unsolicited AR | 1/12 | 8.3 | 1 |
| | Grade 3 unsolicited AR | 0/12 | 0 | 0 |
| | Unsolicited injection site AR | 0/12 | 0 | 0 |
| | Grade 3 unsolicited injection site AR | 0/12 | 0 | 0 |
| | SAE | 0/12 | 0 | 0 |
| | Grade 3 SAE | 0/12 | 0 | 0 |
| 12-24 | Immediate unsolicited AE | 0/39 | 0 | 0 |
| months | Grade 3 immediate unsolicited AE | 0/39 | 0 | 0 |
| | Immediate unsolicited AR | 0/39 | 0 | 0 |
| | Grade 3 immediate unsolicited AR | 0/39 | 0 | 0 |
| | Unsolicited AE | 10/39 | 25.6 | 22 |
| | Grade 3 unsolicited AE | 1/39 | 2.6 | 1 |
| | Unsolicited AR | 0/39 | 0 | 0 |
| | Grade 3 unsolicited AR | 0/39 | 0 | 0 |
| | Unsolicited injection site AR | 0/39 | 0 | 0 |
| | Grade 3 unsolicited injection site | 0/39 | 0 | 0 |
| | AR | | | |
| | SAE | 1/39 | 2.6 | 1 |
| | Grade 3 SAE | 0/39 | 0 | 0 |
| >=24 | Immediate unsolicited AE | 0/49 | 0 | 0 |
| months | Grade 3 immediate unsolicited AE | 0/49 | 0 | 0 |
| | Immediate unsolicited AR | 0/49 | 0 | 0 |
| | Grade 3 immediate unsolicited AR | 0/49 | 0 | 0 |
| | Unsolicited AE | 9/49 | 18.4 | 11 |
| | Grade 3 unsolicited AE | 0/49 | 0 | 0 |
| | Unsolicited AR | 1/49 | 2.0 | 1 |
| | Grade 3 unsolicited AR | 0/49 | 0 | 0 |
| | Unsolicited injection site AR | 0/49 | 0 | 0 |
| | Grade 3 unsolicited injection site | 0/49 | 0 | 0 |
| | AR | | | |
| | SAE | 0/49 | 0 | 0 |
| | Grade 3 SAE | 0/49 | 0 | 0 |

Table 53 summarizes unsolicited AEs and ARs within 28 days after any vaccinations in sentinel subjects by previous influenza vaccination. n is the number of subjects experiencing the endpoint listed in the first column. M is the number of subjects with available data for the relevant endpoint. Percentages are based on M. n AEs are the number of AEs.

TABLE 53
Summary of unsolicited AEs and Ars within
28 days after any vaccinations in sentinel
subjects by previous influenza vaccination

| Previous | | QIV-HD | | |
|---|---|---|---|---|
| vacci- | | (N = 100) | | |
| nation | Subjects experiencing at least one: | n/M | % | n AEs |
| Previously | Immediate unsolicited AE | 0/41 | 0 | 0 |
| un- | Grade 3 immediate unsolicited AE | 0/41 | 0 | 0 |
| vaccinated | Immediate unsolicited AR | 0/41 | 0 | 0 |
| | Grade 3 immediate unsolicited AR | 0/41 | 0 | 0 |
| | Unsolicited AE | 14/41 | 34.1 | 26 |
| | Grade 3 unsolicited AE | 1/41 | 2.4 | 1 |
| | Unsolicited AR | 2/41 | 4.9 | 2 |
| | Grade 3 unsolicited AR | 0/41 | 0 | 0 |
| | Unsolicited injection site AR | 0/41 | 0 | 0 |
| | Grade 3 unsolicited injection site | 0/41 | 0 | 0 |
| | AR | | | |
| | SAE | 1/41 | 2.4 | 1 |
| | Grade 3 SAE | 0/41 | 0 | 0 |
| Previously | Immediate unsolicited AE | 0/59 | 0 | 0 |
| vaccinated | Grade 3 immediate unsolicited AE | 0/59 | 0 | 0 |
| | Immediate unsolicited AR | 0/59 | 0 | 0 |
| | Grade 3 immediate unsolicited AR | 0/59 | 0 | 0 |
| | Unsolicited AE | 11/59 | 18.6 | 18 |
| | Grade 3 unsolicited AE | 0/59 | 0 | 0 |
| | Unsolicited AR | 0/59 | 0 | 0 |
| | Grade 3 unsolicited AR | 0/59 | 0 | 0 |
| | Unsolicited injection site AR | 0/59 | 0 | 0 |
| | Grade 3 unsolicited injection site | 0/59 | 0 | 0 |
| | AR | | | |
| | SAE | 0/59 | 0 | 0 |
| | Grade 3 SAE | 0/59 | 0 | 0 |

We claim:

1. A method for immunizing a pediatric subject against influenza virus comprising administering to the pediatric subject a QIV-HD vaccine, wherein the pediatric subject is aged 6 months to less than 18 years.

2. A method for immunizing a pediatric subject against influenza virus comprising administering to the pediatric subject a QIV-HD vaccine comprising:
a. about 30 μg, about 45 μg, or about 60 μg of hemagglutinin from an H1N1 influenza A virus strain per dose;
b. about 30 μg, about 45 μg, or about 60 μg of hemagglutinin from an H3N2 influenza A virus strain per dose;
c. about 30 μg, about 45 μg, or about 60 μg of hemagglutinin from a Yamagata lineage of influenza B virus strain per dose; and
d. about 30 μg, about 45 μg, or about 60 μg of hemagglutinin from a Victoria lineage of influenza B virus strain per dose; and
wherein the pediatric subject is aged 6 months to less than 18 years.

3. The method of claim 1 or 2, wherein the method prevents influenza virus infection in the pediatric subject.

4. The method of claim 1 or 2, wherein the method raises a protective immune response in the pediatric subject.

5. The method of claim 4, wherein the immune response is an antibody response.

6. The method of claim 1 or 2, wherein the vaccine comprises about 30 μg of hemagglutinin from an H1N1 influenza A virus strain per dose, about 30 μg of hemagglutinin from an H3N2 influenza A virus strain per dose; about 30 μg of hemagglutinin from a Yamagata lineage of influenza B virus strain per dose; and about 30 μg of hemagglutinin from a Victoria lineage of influenza B virus strain per dose.

7. The method of claim 1 or 2, wherein the vaccine comprises about 45 μg of hemagglutinin from an H1N1 influenza A virus strain per dose, about 45 μg of hemagglutinin from an H3N2 influenza A virus strain per dose; about 45 μg of hemagglutinin from a Yamagata lineage of influenza B virus strain per dose; and about 45 μg of hemagglutinin from a Victoria lineage of influenza B virus strain per dose.

8. The method of claim 1 or 2, wherein the vaccine comprises about 60 μg of hemagglutinin from an H1N1 influenza A virus strain per dose, about 60 μg of hemagglutinin from an H3N2 influenza A virus strain per dose; about 60 μg of hemagglutinin from a Yamagata lineage influenza B virus strain per dose; and about 60 μg of hemagglutinin from a Victoria lineage of influenza B virus strain per dose.

9. A method for immunizing a pediatric subject against influenza virus comprising administering to the pediatric subject aged 6 months to less than 18 years a QIV-HD vaccine comprising:
a. about 60 μg of hemagglutinin from an H1N1 influenza A virus strain per dose;
b. about 60 μg of hemagglutinin from an H3N2 influenza B virus strain per dose;
c. about 60 μg of hemagglutinin from a Yamagata lineage of influenza B virus strain per dose; and
d. about 60 μg of hemagglutinin from a Victoria lineage of influenza B virus strain per dose.

10. The method of any one of claims 1-9, wherein the vaccine is administered intramuscularly.

11. The method of any one of claims 1-10, wherein the pediatric subject is
a. 6 months to less than 36 months of age;
b. 3 years to less than 5 years of age;
c. 5 years to less than 9 years of age; and/or
d. 9 years to less than 18 years of age.

12. The method of any one of claims 1-11, wherein the pediatric subject is 6 months to less than 24 months of age.

13. The method of any one of claims 1-12, wherein the vaccine is administered once or twice to the same subject.

14. The method of claim 13, wherein the dose is administered in a volume of 0.70 mL.

15. The method of any one of claims 1-14, wherein the vaccine is administered in a prefilled syringe.

16. The method of any one of claims 1-15, wherein the pediatric subject has not been previously vaccinated against influenza.

17. The method of claim 16, wherein the previously unvaccinated subject is 6 months to less than 9 years of age and is provided two doses of vaccine.

18. The method of claim 17, wherein the two doses of vaccine are provided about 28 days apart.

19. The method of any one of claims 1-15, wherein the subject has been previously vaccinated against influenza.

20. The method of claim 19, wherein the subject is administered a single dose of vaccine.

21. The method of any one of claims 1-20, wherein administration reduces the incidence of laboratory confirmed influenza-like illness as compared to vaccination of a similar-aged subject with QIV-SD, wherein confirmed influenza-like illness is the occurrence of fever greater than or equal to 38° C. for at least 24 hours and at least one of cough, sputum production, wheezing, difficulty breathing, nasal congestion, rhinorrhea, pharyngitis, otitis, vomiting, diarrhea, sore throat, chills (shivering), tiredness (fatigue), headache, and myalgia (muscle aches).

22. The method of any one of claims 1-21, wherein administration reduces the occurrence of laboratory-confirmed influenza-like illness caused by viral types/subtypes antigenically similar to those contained in the vaccine composition.

23. The method of any one of claims 1-22, wherein administration reduces the occurrence of acute otitis media (AOM), acute lower respiratory tract infection (ALRI, e.g., pneumonia), hospitalization, and/or medication use.

24. The method of any one of claims 1-23, wherein two doses of the vaccine are administered to subjects who are unvaccinated for influenza, and wherein administration of the two doses of the vaccine results in higher geometric mean titers (GMTs) against each of the strains used to vaccinate as compared to vaccination with QIV-SD.

25. The method of any one of claims 1-24, wherein administration of the vaccine results in higher seroneutralization geometric mean titers (GMTs) against each of the strains used to vaccinate as compared to vaccination with QIV-SD.

26. The method of any one of claims 1-25, wherein the administration results in a higher geometric mean HI antibody titer (GMT) ratio (QIV-HD/QIV-SD) than the GMT ratios of TIV-HD/TIV-SD in adults aged 65 or older.

27. The method of any one of claims 24-26, wherein the subject is aged 6 months to less than 3 years.

28. The method of any one of claims 1-27, wherein the vaccine is produced in avian eggs.

29. The method of any one of claims 1-27, wherein the vaccine is not produced in avian eggs.

30. The method of any one of claims 1-27, wherein the vaccine is made by recombinant DNA techniques.

31. The method of any one of claims 1-27, wherein the vaccine is inactivated or live attenuated.

32. The method of claim 31, wherein the vaccine is inactivated.

33. The method of claim 31, wherein the vaccine is live attenuated.

34. The method of any one of claims 1-33, wherein the vaccine is a split virus vaccine.

35. The method of any one of claims 1-34, wherein the vaccine contains adjuvant.

36. The method of any one of claims 1-34, wherein the vaccine does not contain adjuvant.

37. The method of any one of claims 1-36, wherein the pediatric subject is immune compromised.

38. The method of any one of claims 1-37, wherein the pediatric subject is high risk.

39. The method of any one of claims 1-38, wherein the pediatric subject has or had asthma, diabetes, heart disease, HIV, AIDS, or cancer.

40. The method of any one of claims 1-39, wherein the vaccine is safe and well tolerated in the pediatric subject.
